(19) United States
(12) Patent Application Publication
(10) Pub. No.: US 2023/0126271 A1
(43) Pub. Date: Apr. 27, 2023

(54) DEGRADER-ANTIBODY CONJUGATES AND METHODS OF USING SAME

(71) Applicant: Anglex, Inc., Concord, MA (US)

(72) Inventors: Paul A. Jaminet, Sudbury, MA (US); Shou-Ching S. Jaminet, Sudbury, MA (US); Edward Ha, New York, NY (US); Manish S. Hudlikar, Brookline, MA (US); Gabriel Gonzalez, Needham, MA (US)

(21) Appl. No.: 17/935,690

(22) Filed: Sep. 27, 2022

Related U.S. Application Data
(63) Continuation of application No. PCT/US2021/024535, filed on Mar. 26, 2021.
(60) Provisional application No. 63/000,991, filed on Mar. 27, 2020.
(60) Provisional application No. 63/044,699, filed on Jun. 26, 2020.

(51) Int. Cl. A61K 47/68; C07D 471/16; C07D 495/16

(52) U.S. Cl. CPC A61K 47/6849; A61K 47/6891; C07D 471/16; C07D 495/16

(57) ABSTRACT

Degrader-antibody conjugates (DACs) are described, comprising anti-TM4SF1 antibodies, and antigen-binding fragments thereof. Degrader molecules as described comprise a ubiquitin E3 ligase binding group (E3LB) and a protein binding group (PB). Linkers may be utilized between the antibodies and the degrader molecules (L1) and between the ubiquitin E3 ligase binding group (E3LB) and the protein binding group (PB) of the degrader molecule (L2). Methods of use of said DACs are also described.

### CROSS-REFERENCE

This application is a continuation of International Application Serial No. PCT/US2021/024535, filed Mar. 26, 2021, which claims the benefit of U.S. Provisional Application No. 63/000,991, filed Mar. 27, 2020, and U.S. Provisional Application No. 63/044,699, filed Jun. 26, 2020, all of which are incorporated by reference herein in their entireties.

### SEQUENCE LISTING

The instant application contains a Sequence Listing which has been submitted electronically in XML format and is hereby incorporated by reference in its entirety. Said XML copy, created on Sep. 26, 2022, is named 52628-709.301_SL.xml and is 243,075 bytes in size.

### BACKGROUND

There remains a need in the art for cancer therapeutics, and in particular therapeutics with improved therapeutic margins that can regress primary tumors as well as invasive tumor cells and metastases.

Cancer therapies designed to destroy tumor blood vessels have in the past failed in clinical trials due to toxicity. Examples include the vascular disrupting agents such as Combretastatin (CA4P). See, e.g., Grisham et al. Clinical trial experience with CA4P anticancer therapy: focus on efficacy, cardiovascular adverse events, and hypertension management. Gynecol Oncol Res Pract. 2018; 5:1. CA4P reduced overall survival from 16.2 to 13.6 months in the Phase II FALCON study, and seven patients have experienced heart attacks while being treated with CA4P. Id. As coronary heart disease and stroke are leading causes of death, any vascular targeted toxic therapy may lead to a risk of lethal toxicity.

TM4SF1 is an endothelial marker with a functional role in angiogenesis. See. e.g., Shih et al. The L6 protein TM4SF1 is critical for endothelial cell function and tumor angiogenesis. Cancer Res. 2009; 69(8):3272-7.

### SUMMARY OF THE INVENTION

One embodiment provides a heterobifunctional compound that comprises:
(a) an anti-TM4SF1 antibody; and
(b) a degrader molecule.
   In some embodiments, the degrader molecule comprises a single-ligand molecule that directly interacts with a target protein to induce degradation of the target protein. In some embodiments, the single-ligand molecule is an SERD, an SARD, an IAPP antagonist, a Boc3Arg-linked ligand, or any combinations thereof. In some embodiments, the degrader molecule comprises a single-ligand molecule that interacts with an E3 ubiquitin ligases to modulate substrate selectivity of the E3 ubiquitin ligase. In some embodiments, the degrader molecule comprises a chimeric degrader molecule. In some embodiments, the chimeric degrader molecule comprises a specific and nongenetic inhibitor of apoptosis protein (IAP)-dependent protein eraser (SNIPER). In some embodiments, the degrader molecule comprises a ubiquitin E3 ligase binding group (E3LB) and a target protein binding group (PB). In some embodiments, the E3LB comprises a protein identified in any one of Tables 1-15.

In some embodiments, the PB comprises a peptide or a small molecule that binds to a protein selected from the group consisting of an intracellular protein, an extracellular protein, a cell surface protein, a disease-causing or a disease-related protein, a TNF-receptor-associated death-domain protein (TRADD), receptor interacting protein (RIP), TNF-receptor-associated factor 2 (TRAF2), IK-alpha, IK-beta, IK-epsilon, PLCγ, IQGAP1, Rac1, MEK1/2, ERK1/2, PI4K230, Akt1/2/3, Hsp90, GSK-3β, an HDAC protein, FoxO1, HDAC6, DP-1, E2F, ABL, AMPK, BRK, BRSK I, BRSK2, BTK, CAMKK1, CAMKK alpha, CAMKK beta, Rb, Suv39HI, SCF, p191NK4D, GSK-3, pi 8 INK4, myc, cyclin E, CDK2, CDK9, CDG4/6, Cycline D, p16 INK4A, cdc25A, BMI1, SCF, Akt, CHK1/2, C 1 delta, CK1 gamma, C 2, CLK2, CSK, DDR2, DYRK1A/2/3, EF2K, EPH-A2/A4/B/B2/B3/B4, EIF2A 3, Smad2, Smad3, Smad4, Smad7, p53, p21 Cip1, PAX, Fyn, CAS, C3G, SOS, Tal, Raptor, RACK-1, CRK, Rap1, Rac, KRas, NRas, HRas, GRB2, FAK, PI3K, spred, Spry, mTOR, MPK, LKB1, PAK 1/2/4/5/6, PDGFRA, PYK2, Src, SRPK1, PLC, PKC, PKA, PKB alpha/beta, PKC alpha/gamma/zeta, PKD, PLK1, PRAK, PRK2, WAVE-2, TSC2, DAPK1, BAD, IMP, C-TAK1, TAK1, TAO1, TBK1, TESK1, TGFBR1, TIE2, TLK1, TrkA, TSSK1, TTBK1/2, TTK, Tpl2/cot1, MEK1, MEK2, PLDL Erk1, Erk2, Erk5, Erk8, p90RSK, PEA-15, SRF, p27 KIP1, TIF 1a, HMGN1, ER81, MKP-3, c-Fos, FGF-R1, GCK, GSK3 beta, HER4, HIPK1/2/3/, IGF-1R, cdc25, UBF, LAMTOR2, Stat1, StaO, CREB, JAK, Src, PTEN, NF-kappaB, HECTH9, Bax, HSP70, HSP90, Apaf-1, Cyto c, BCL-2, Bcl-xL, Smac, XIAP, Caspase-9, Caspase-3, Caspase-6, Caspase-7, CDC37, TAB, IKK, TRADD, TRAF2, R1P1, FLIP, TAK1, JNK1/2/3, Lck, A-Raf, B-Raf, C-Raf, MOS, MLK1/3, MN 1/2, MSK1, MST2/3/4, MPSK1, MEKK1, ME K4, MEL, ASK1, MINK1, MKK 1/2/3/4/617, NE 2a/6/7, NUAK1, OSR1, SAP, STK33, Syk, Lyn, PDK1, PHK, PIM 1/2/3, Ataxin-1, mTORC1, MDM2, p21 Waf1, Cyclin D1, Lamln A, Tpl2, Myc, catenin, Wnt, IKK-beta, IKK-gamma, IKK-alpha, IKK-epsilon, ELK, p65RelA, IRAKI, IRA 2, IRAK4, IRR, FADD, TRAF6, TRAF3, MKK3, MKK6, ROCK2, RSK1/2, SGK 1, SmMLCK, SIK2/3, ULK1/2, VEGFR1, WNK 1, YES1, ZAP70, MAP4K3, MAP4K5, MAPK1b, MAPKAP-K2 K3, p38 alpha/beta/delta/gamma MAPK, Aurora A, Aurora B, Aurora C, MCAK, Clip, MAPKAPK, FAK, MARK 1/2/3/4, Muc1, SHC, CXCR4, Gap-1, Myc, beta-catenin/TCF, Cbl, BRM, Mcl-1, BRD2, BRD3, BRD4, BRDt, BRD7, BRD9, AR, RAS, ErbB3, EGFR, IRE1, HPK1, RIPK2, PDE4, ERRα, FKBP12, brd9, c-Met, Sirt1, Sirt2, Sirt3, Sirt4, Sirt5, Sirt6, Sirt7, flt3, BTK. ALK, TRIM24, GSPT1, IKZF1 (Ikaros), IKZF3 (Aiolos), CK1-alpha, TACC3, p85, MetAP-2, DHFR, BET, CRABP-I/II, HIF1-alpha, PCAF, GCN5L2 (GCN5), SMARCA2, SMARCA4, PBRM1, HER2, Akt, Hsp90, HDAC15, HDAC14, HDAC3, HDAC8, HDAC4, HDAC5, HDAC6, HDAC7, HDAC9, HDAC150, HDAC151, DNMT1, DNMT3a, DNMT3b, MeCP2, MBD1, MBD2, MBD4, KAISO (ZBTB33), ZBTB4, ZBTB38, UHRF1, UHRF2, TET1, TET2, TET3, HATI, HTATIP (TIP60), MYST1 (MOF), MYST2 (HBO1), MYST3 (MOZ), MYST4 (MORF), P300 (EP300, KAT3B), CBP (CREBBP, KAT3A), NCOA1 (SRC1), NCOA2 (TIF2), NCOA3 (AIB1, ACTR), ATF-2 (CREB2, CREBP1), TFIIIC, TAF1 (TAFII250), CLOCK (KIAA0334), CIITA (MHC2TA), MGEA5 (NCOAT), CDY, KMT1A, KMT1B, KMT1C, KMT1E, KMT2A, KMT2B, KMT2C, KMT2D, KMT2E, KMT2F, EZH1, EZH2, KMT3A, WHSC1, WHSC1L1, PRDM1, PRDM2, PRDM3, PRDM4, PRDM5, PRDM9, PRDM14, PRDM16, KMT3C, KMT3E, SMYD4, DOT1L, SET8, SUV4-20H2, SetD6, SET7/9, PRMT1, PRMT2, PRMT4, PRMT5, PRMT6, PRMT7, PRMT8, PRMT9, HP1, Chd1, WDR5, BPTF, L3MBTL1, ING2, BHC80, JMJD2A, KDM1A, KDM1B, KDM2A, KDM2B, KDM3A, KDM3C, KDM4A, KDM4B, KDM4C, KDM4D, KDM5A, KDM5B, KDM5C, KDM5D, JARID2, KDM6A, KDM6B, KDM6C, KDM7A, KDM7C, KDM7B, JMJD5, RSBN1, JMJD6, PADI4, K-Ras, PI3K, BTK, B-Raf, ERK, MEK, P65 (RELA), p50 (NFKB1) of NFkB, Ras, Raf, eNOS, a Smad family protein, Smad2/3/4, and ERalpha, variants thereof, mutants thereof, splice variants thereof, indels thereof, and fusions thereof.

In some embodiments, the PB comprises a PLCγ inhibitor; an IQGAP1 inhibitor; a Rac1 inhibitor; an MEK1/2 inhibitor; an ERK1/2 inhibitor; a PI4K230 inhibitor; an Akt1 inhibitor; an Akt2 inhibitor; an Akt3 inhibitor; a GSK-3β inhibitor; an HDAC6a inhibitor; a Heat Shock Protein 90 (HSP90) inhibitor; a kinase inhibitor; a Phosphatase inhibitor; an MDM2 inhibitor; a compound targeting Human Bromodomain and Extra Terminal Motif Domain family proteins; an HDAC inhibitor; a human lysine methyltransferase inhibitor; an angiogenesis inhibitor; an immunosuppressive compound; a compound targeting the aryl hydrocarbon receptor (AHR), a REF receptor kinase, a FKBP, an Androgen Receptor (AR), an Estrogen receptor (ER), a Thyroid Hormone Receptor, a HIV Protease, a HIV Integrase, a HCV Protease, an Acyl-protein Thioesterase-1 (APT), an Acyl-protein Thioesterase-2 (APT2), a pharmaceutically acceptable salt of any thereof, an enantiomer of any thereof, a solvate of any thereof, or a polymorph of any thereof.

In some embodiments, the degrader molecule further comprises a linker (L2) between the E3LB and the PB. In some embodiments, the linker L2 links the E3LB and the PB via a covalent bond. In some embodiments, the linker L2 comprises an alkyl linker or a PEG linker. In some embodiments, the linker L2 comprises the alkyl linker, wherein the alkyl linker comprises the formula (alkyl)ₙ, wherein n is the number of alkyl carbon, and wherein=1-12. In some embodiments, the linker L2 comprises the PEG linker, wherein the PEG linker comprises the formula (PEG)ₙ, wherein n is the number of PEG repeating unit, and wherein n=1-4. In some embodiments, the linker L2 comprises one or more covalently connected structural units of A (e.g., -A₁ . . . A_{q}-), wherein A₁ is a group coupled to at least one of a E3LB, a PB, or a combination thereof. In some embodiments, A₁ links a E3LB, a PB, or a combination thereof directly to another E3LB, PB, or combination thereof. In some embodiments, A₁ links a EL3B, a PB, or a combination thereof indirectly to another E3LB, PB, or combination thereof through A_{q}. In some embodiments, the q is an integer greater than or equal to 0. In some embodiments, q is an integer greater than or equal to 1. In some embodiments, q is greater than or equal to 2, A_{q} is a group which is connected to an E3LB moiety, and A₁ and A_{q} are connected via structural units of A (number of such structural units of A: q-2). In some embodiments, q is 2, A_{q} is a group which is connected to A₁ and to an E3LB moiety. In some embodiments, q is 1, the structure of the linker group L2 is -A₁-, and A₁ is a group which is connected to an E3LB moiety and a PB moiety. In some embodiments, q is an integer from 1 to 100, 1 to 90, 1 to 80, 1 to 70, 1 to 60, 1 to 50, 1 to 40, 1 to 30, 1 to 20, or 1 to 10. In some embodiments, the heterobifunctional compound further comprises a linker (L1) between the degrader molecule and the anti-TM4SF1 antibody. In some embodiments, the linker L1 comprises a cleavable linker or a non-cleavable linker.

In some embodiments, the linker L1 comprises the cleavable linker and wherein the cleavable linker comprises a disulfide linker, a glutathione cleavable linker, or a combination thereof. In some embodiments, the linker L1 comprises a MC (6-maleimidocaproyl), a MCC (a maleimidomethyl cyclohexane-1-carboxylate), a MP (maleimidopropanoyl), a val-cit (valine-citrulline), a val-ala (valine-alanine), an ala-phe (alanine-phenylalanine), a PAB (p-aminobenzyloxycarbonyl), a SPP (N-Succinimidyl 4-(2-pyridylthio) pentanoate), 2,5-dioxopyrrolidin-1-yl 4-(pyridin-2-ylthio)hexanoate, 2,5-dioxopyrrolidin-1-yl 5-methyl-4-(pyridin-2-ylthio)hexanoate, 2,5-dioxopyrrolidin-1-yl 5-methyl-4-(pyridin-2-ylthio)heptanoate, 2,5-dioxopyrrolidin-1-yl 5-ethyl-4-(pyridin-2-ylthio)heptanoate, 2,5-dioxopyrrolidin-1-yl 4-cyclopropyl-4-(pyridin-2-ylthio)butanoate, 2,5-dioxopyrrolidin-1-yl 4-cyclobutyl-4-(pyridin-2-ylthio)butanoate, 2,5-dioxopyrrolidin-1-yl 4-cyclopentyl-4-(pyridin-2-ylthio)butanoate, 2,5-dioxopyrrolidin-1-yl 4-cyclohexyl-4-(pyridin-2-ylthio)butanoate, a SMCC (N-Succinimidyl 4-(N-maleimidomethyl)cyclohexane-1 carboxylate), or a SIAB (N-Succinimidyl (4-iodo-acetyl)aminobenzoate).

In some embodiments, the linker L1 is derived from a cross-linking reagent, wherein the cross-linking reagent comprises N-succinimidyl-3-(2-pyridyldithio)propionate (SPDP), 2,5-dioxopyrrolidin-1-yl 3-cyclopropyl-3-(pyridin-2-yldisulfaneyl)propanoate, 2,5-dioxopyrrolidin-1-yl 3-cyclobutyl-3-(pyridin-2-yldisulfaneyl)propanoate, N-succinimidyl 4-(2-pyridyldithio)pentanoate (SPP), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl 4-(2-pyridyldithio)butanoate (SPDB), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC), or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In some embodiments, the linker L1 comprises a peptidomimetic linker. In some embodiments, the peptidomimetic linker comprises the formula -Str-(PM)-Sp, wherein Str is a stretcher unit covalently attached to Ab; Sp is a bond or spacer unit covalently attached to a degrader moiety; and PM is a non-peptide chemical moiety selected from the group consisting of:

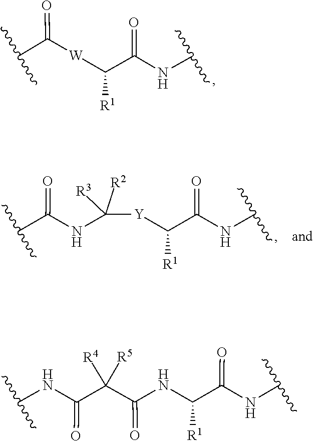

W is —NH-heterocycloalkyl- or -heterocycloalkyl-; Y is heteroarylene, arylene, —C(═O)C₁-C₆ alkylene, C₁-C₆ alkylene-NH—, C₁-C₆ alkylene-NH—CH₂—, C₁-C₆ alkylene-N(CH₃)—CH₂—, C₁-C₆ alkenylene or C₁-C₆ alkylenylene; each R¹ is independently C₁-C₁₀ alkyl, C₁-C₁₀ alkenyl, (C₁-C₁₀ alkyl)NHC(═NH)NH₂ or (C₁-C₁₀ alkyl)NHC(═O)NH₂; R² and R³ are each independently H, C₁-C₁₀ alkyl, C₁-C₁₀ alkenyl, arylalkyl or heteroarylalkyl, or R³ and R² together with atoms attached thereto form a C₃-C₇ cycloalkyl; and R⁴ and R⁵ are each independently C₁-C₁₀ alkyl, C₁-C₁₀ alkenyl, arylalkyl, heteroarylalkyl, (C₁-C₁₀ alkyl)OCH₂—, or R⁴ and R₅ together with atoms attached thereto form a C₃-C₇ cycloalkyl ring. In some embodiments, the linker L1 comprises a non-peptidomimetic linker. In some embodiments, the non-peptidomimetic linker comprises has the structure:

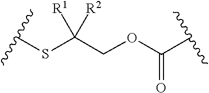

wherein, R¹ and R² are independently selected from H and C₁-C₆ alkyl, or R¹ and R² form a 3, 4, 5, or 6-membered cycloalkyl or heterocyclyl group.

In some embodiments, the anti-TM4SF1 antibody or an antigen binding fragment thereof comprising a modified IgG Fc region, wherein the modified IgG Fc region comprises one or more substitutions relative to a wild-type IgG Fc region. In some embodiments, the wild-type IgG Fc region is a wild-type IgG1, IgG2, IgG3, or IgG4 Fc region. In some embodiments, the wild-type Fc region is the IgG1 Fc region, and wherein the modified IgG Fc region comprises an IgG1 Fc region comprising mutation at one or more positions selected from the group consisting of E233, L234, L235, G237, M252, S254, 1250, T256, D265, N297, K322, P331, M428, and N434 of the wild-type IgG1 Fc region; as numbered by the EU index as set forth in Kabat. In some embodiments, the IgG1 Fc region comprises the mutation at position N297. In some embodiments, the mutation at position N297 comprises N297C. In some embodiments, the IgG1 Fc region comprises the mutation at position E233. In some embodiments, the mutation at position E233 comprises E233P. In some embodiments, the IgG1 Fc region comprises the mutation at position L234. In some embodiments, the mutation at position L234 comprises L234A. In some embodiments, the IgG1 Fc region comprises the mutation at position L235. In some embodiments, the mutation at position L235 comprises L235A. In some embodiments, the IgG1 Fc region comprises the mutation at position G237. In some embodiments, the mutation at position G237 comprises G237A. In some embodiments, the IgG1 Fc region comprises the mutation at position M252. In some embodiments, the mutation at position M252 comprises M252Y. In some embodiments, the IgG1 Fc region comprises the mutation at position S254. In some embodiments, the mutation at position S254 comprises S254T. In some embodiments, the IgG1 Fc region comprises the mutation at position 1256. In some embodiments, the mutation at position T256 comprises T256E. In some embodiments, the IgG1 Fc region comprises the mutation at position M428. In some embodiments, the mutation at position M428 comprises M428L.

In some embodiments, the IgG1 Fc region comprises the mutation at position N434. In some embodiments, the mutation at position N434 comprises N434S or N434A. In some embodiments, the IgG1 Fc region comprises the mutation at position 1250. In some embodiments, the mutation at position 1250 comprises T250Q. In some embodiments, the IgG1 Fc region comprises the mutation at position D265. In some embodiments, the mutation at position D265 comprises D265A. In some embodiments, the IgG1 Fc region comprises the mutation at position K322. In some embodiments, the mutation at position K322 comprises K322A. In some embodiments, the IgG1 Fc region comprises the mutation at position P331. In some embodiments, the mutation at position P331 comprises P331G. In some embodiments, the IgG1 Fc region comprises T250Q and M428L. In some embodiments, the IgG1 Fc region comprises M428L and N434S. In some embodiments, the IgG1 Fc region comprises L234A, L235A, and G237A. In some embodiments, the IgG1 Fc region comprises L234A, L235A, G237A, and P331G. In some embodiments, the IgG1 Fc region comprises L234A, L235A, G237A, N297C, and P331G. In some embodiments, the IgG1 Fc region comprises E233P, L234A, L235A, G237A, and P331G. In some embodiments, the IgG1 Fc region comprises E233P, L234A, L235A, G237A, and N297C. In some embodiments, the IgG1 Fc region comprises L234A, L235A, G237A, N297C, K322A, and P331G. In some embodiments, the IgG1 Fc region comprises E233P, L234A, L235A, G237A, D265A, N297C, K322A, and P331G. In some embodiments, the IgG1 Fc region comprises E233P and D265A. In some embodiments, the IgG1 Fc region comprises M252Y, S254T, and T256E. In some embodiments, the IgG1 Fc region comprises M252Y, S254T, T256E, and N297C. In some embodiments, the IgG1 Fc region comprises an amino acid sequence selected from the group consisting of SEQ ID Nos. 87-88, 135-145, and 151-153. In some embodiments, the IgG1 Fc region exhibits one or more of the following properties: (i) reduced or ablated binding with C1q, (ii) reduced or ablated binding to an Fc receptor, and (ii) reduced or ablated ADCC or CDC effector function. In some embodiments, the wild-type Fc region is the IgG4 Fc region, and wherein the modified IgG Fc region comprises an IgG4 Fc region comprising mutation at one or more positions selected from the group consisting of S228, F234, L235, G237, P238, F243, 1250, M252, S254, T256, E258, D259, V264, D265, K288, 1299, T307, V308, Q311, K322, L328, P329, A330, P331, T356, K370, A378, R409, V427, M428, H433, N434, H435, and N297, of the wild-type IgG4 Fc region, as numbered by the EU index as set forth in Kabat. In some embodiments, the IgG4 Fc region comprises the mutation at position S228. In some embodiments, the mutation at position S228 is S228P. In some embodiments, the IgG4 Fc region comprising the mutation at position F234. In some embodiments, the mutation at position F234 is F234A. In some embodiments, the IgG4 Fc region comprises the mutation at position L235.

In some embodiments, the mutation at position L235 is L235E. In some embodiments, the IgG4 Fc region comprises mutations S228P and L235E. In some embodiments, the IgG4 Fc region comprises mutations S228P, L235E, and N297C. In some embodiments, the IgG4 Fc region comprises mutations S228P, F234A, L235E, and N297C. In some embodiments, the IgG4 Fc region comprises mutations M428L and N434S. In some embodiments, the IgG4 Fc region comprises mutations L235E and F234A. In some embodiments, the IgG4 Fc region comprises mutations S228P, L235E, and N297C. In some embodiments, the IgG4 Fc region comprises mutations S228P, F234A, L235A, G237A, and P238S. In some embodiments, the IgG4 Fc region comprises mutations F243A and V264A. In some embodiments, the IgG4 Fc region comprises mutations S228P and L235A. In some embodiments, the IgG4 Fc region comprises mutations M252Y and M428L; D259I and V308F; or N434S. In some embodiments, the IgG4 Fc region comprises mutations T307Q and N434S; M428L and V308F; Q311V and N434S; H433K and N434F; E258F and V427T; or T256D, Q311V, and A378V. In some embodiments, the IgG4 Fc region comprises one or more of the following properties: (i) reduced or ablated binding with C1q; (ii) reduced or ablated binding to an Fc receptor; and (iii) reduced or ablated ADCC or CDC effector function. In some embodiments, the anti-TM4SF1 antibody comprising the IgG4 Fc region comprises an amino acid sequence selected from the group consisting of SEQ ID Nos. 146-150, and 154-155. In some embodiments, the anti-TM4SF1 antibody or an antigen-binding fragment thereof comprises: a) a heavy chain comprising a CDR3 domain comprising an amino acid sequence that has at least 75% identity to a sequence selected from the group consisting of SEQ ID NO: 8, 20, 32, 44, 56, 68, 80, 96, 118, 119, 120, or 121; a CDR2 domain comprising an amino acid sequence that has at least 75% identity to a sequence selected from the group consisting of SEQ ID NO: 7, 19, 31, 43, 55, 67, 79, 95, 116, or 117; and a CDR1 domain comprising an amino acid sequence that has at least 75% identity to a sequence selected from the group consisting of SEQ ID NO: 6, 18, 30, 42, 54, 66, 78, 94, or 115; and b) a light chain comprising a CDR3 domain comprising an amino acid sequence that has at least 75% identity to a sequence selected from the group consisting of SEQ ID NO: 14, 26, 38, 50, 62, 74, 86, 110, or 129; a CDR2 domain comprising an amino acid sequence that has at least 75% identity to a sequence selected from the group consisting of SEQ ID NO: 13, 25, 37, 49, 61, 73, 85, 109, or 128; and a CDR1 domain comprising an amino acid sequence that has at least 75% identity to a sequence selected from the group consisting of SEQ ID NO: 12, 24, 36, 48, 60, 72, or 84, 107, 108, 124, 125, 126, or 127.

In some embodiments, the heavy chain comprises an amino acid sequence that has at least 75% identity to SEQ ID NO: 3, 15, 27, 39, 51, 63, 75, 90, 92, 112, 114, 130, or 132, and a light chain comprises an amino acid sequence that has at least 75% identity to SEQ ID NO: 9, 21, 33, 45, 57, 69, 81, 97, 99, 101, 122, 131, or 133. In some embodiments, the heavy chain comprises an amino acid sequence as set forth in any one of: SEQ ID NO: 3, 15, 27, 39, 51, 63, 75, 90, 92, 112, 114, 130, or 132, and a light chain comprises an amino acid sequence as set forth in any one of: SEQ ID NO: 9, 21, 33, 45, 57, 69, 81, 97, 99, 101, 122, 131, or 133.

In some embodiments, the degrader molecule comprises a compound having a structure selected from the group consisting of:

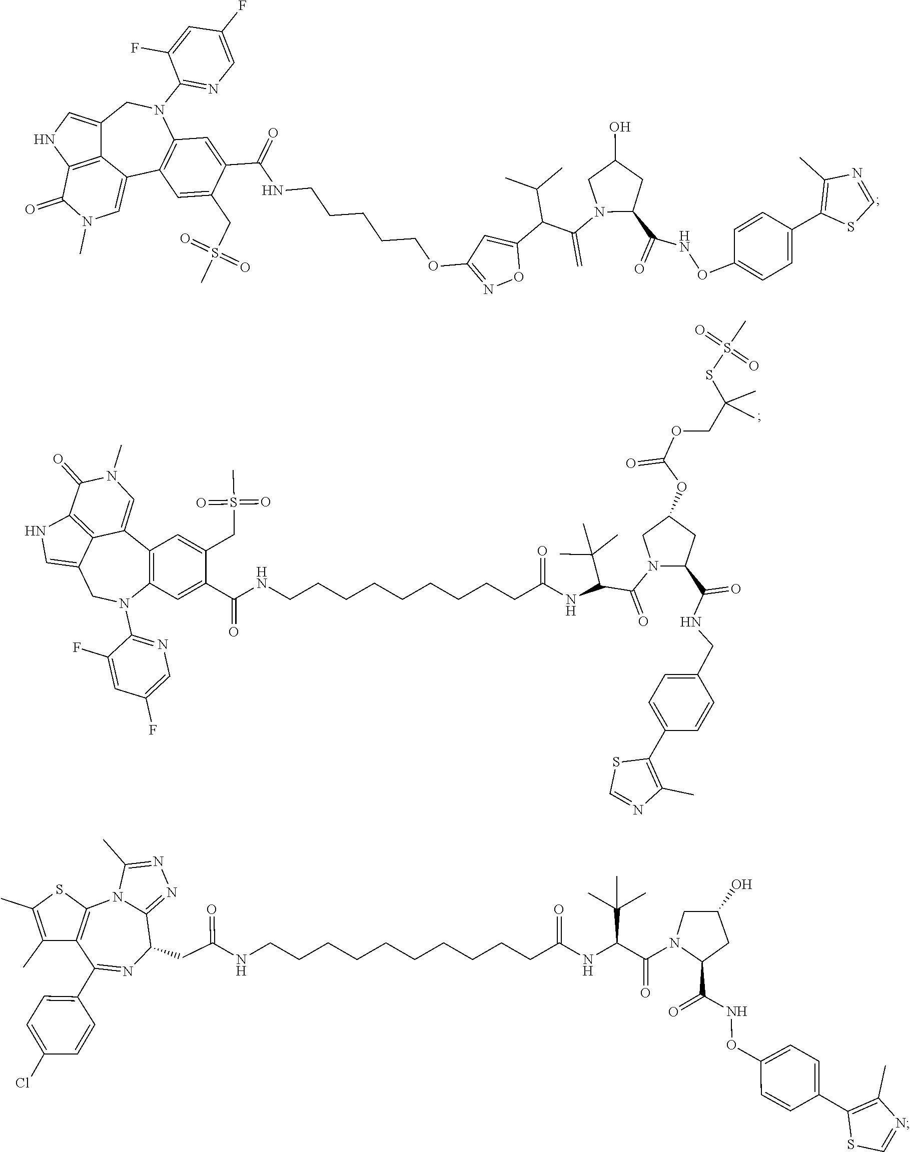

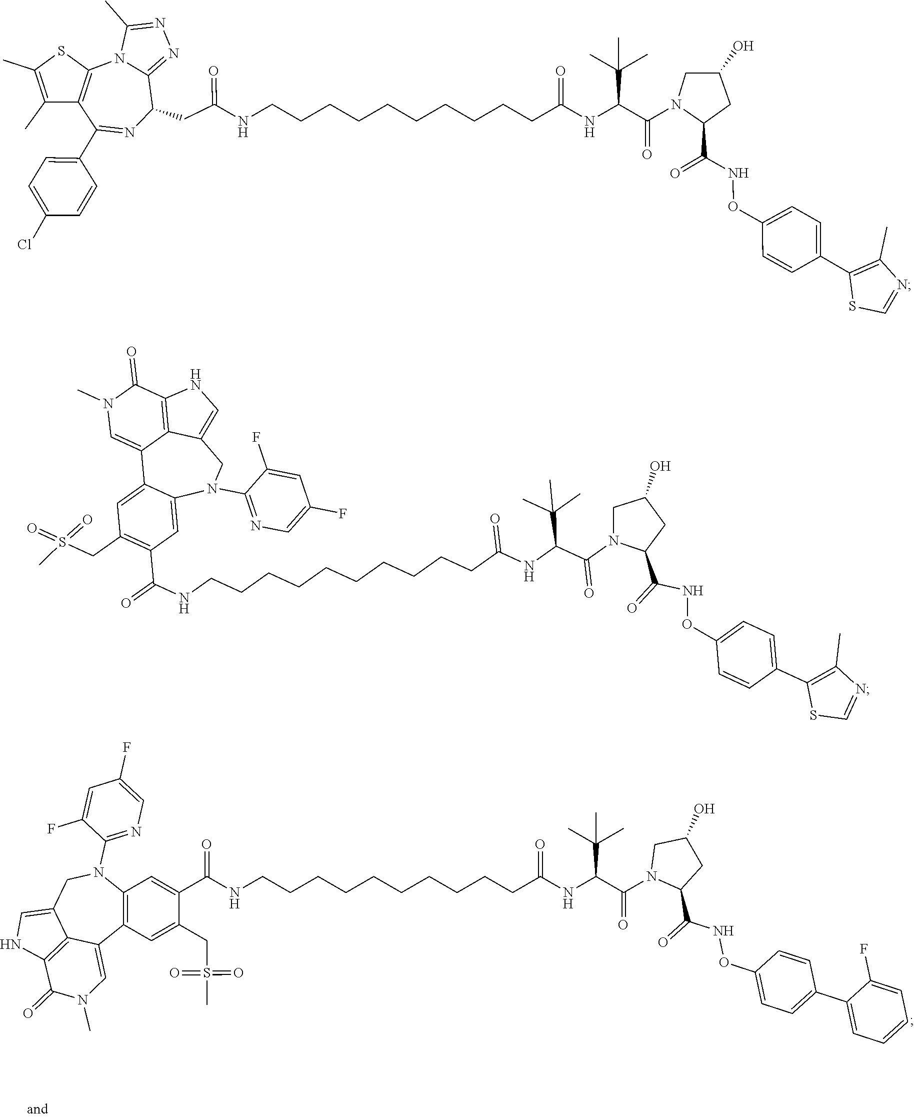 [SMILES: Cc1ncsc1-c1ccc(ONC(=O)[C@@H]2C[C@@H](O)CN2C(=O)[C@@H](NC(=O)CCCCCCCCCCNC(=O)C[C@@H]2N=C(c3ccc(Cl)cc3)c3c(sc(C)c3C)-n3c(C)nnc32)C(C)(C)C)cc1 ; Cc1ncsc1-c1ccc(ONC(=O)[C@@H]2C[C@@H](O)CN2C(=O)[C@@H](NC(=O)CCCCCCCCCCNC(=O)c2cc3c(cc2CS(C)(=O)=O)-c2cn(C)c(=O)c4[nH]cc(c24)CN3c2ncc(F)cc2F)C(C)(C)C)cc1 ; Cn1cc2c3c(c[nH]c3c1=O)CN(c1ncc(F)cc1F)c1cc(C(=O)NCCCCCCCCCCC(=O)N[C@H](C(=O)N3C[C@H](O)C[C@H]3C(=O)NOc3ccc(-c4ccccc4F)cc3)C(C)(C)C)c(CS(C)(=O)=O)cc1-2]

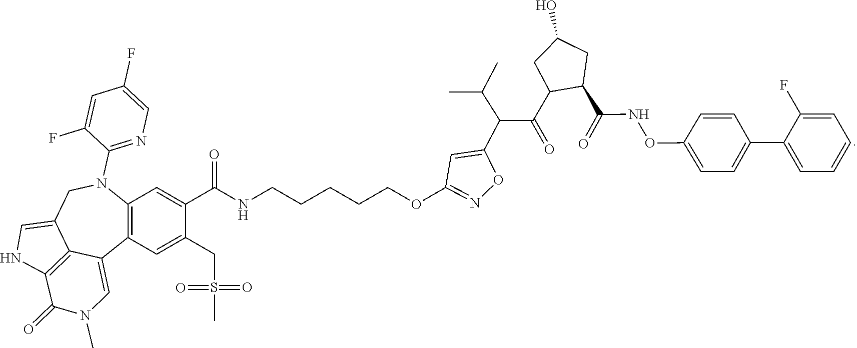 [SMILES: CC(C)C(C(=O)C1C[C@H](O)C[C@H]1C(=O)NOc1ccc(-c2ccccc2F)cc1)c1cc(OCCCCCNC(=O)c2cc3c(cc2CS(C)(=O)=O)-c2cn(C)c(=O)c4[nH]cc(c24)CN3c2ncc(F)cc2F)no1]

One embodiment provides a method of treating or preventing a disease or disorder in a subject, wherein said disease or disorder is characterized by an endothelial cell (EC)-cell interaction, said method comprising administering to said heterobifunctional compound according to any of the above embodiments. In some embodiments, the EC-cell interaction comprises one or more of EC-mesenchymal stem cell, EC-fibroblast, EC-smooth muscle cell, EC-tumor cell, EC-leukocyte, EC-adipose cell, EC-platelet (thrombocyte), EC-erythrocyte, EC-pericyte, and EC-neuronal cell interactions. In some embodiments, the disease or disorder is at least one of: (i) a disease characterized by pathological angiogenesis; (ii) a disease of impaired wound healing; (iii) a cardiovascular disease, (iv) an infection, and (v) a cancer. In some embodiments, the disease or disorder is the disease characterized by pathological angiogenesis, and wherein the disease characterized by pathological angiogenesis is age-related macular degeneration. In some embodiments, the disease or disorder is the disease characterized by impaired wound healing, and wherein the disease characterized by impaired wound healing is a diabetic ulcer. In some embodiments, the disease or disorder is the cardiovascular disease, and wherein the cardiovascular disease is atherosclerosis. In some embodiments, the disease or disorder is the infection, and wherein the infection is caused by a virus. In some embodiments, the virus is a coronavirus. In some embodiments, the disease or disorder is the cancer, and wherein the cancer is selected from the group consisting of: breast cancer, lung cancer, colon cancer, prostate cancer, pancreatic cancer, liver cancer, gastric cancer, renal cancer, bladder cancer, uterine cancer, cervical cancer, ovarian cancer, glioblastoma, angiosarcoma, osteosarcoma, soft tissue sarcoma.

One embodiment provides a method of treating or preventing inflammation in a subject, said method comprising administering to said subject a heterobifunctional compound according to any of the above embodiments. One embodiment provides a method of treating or preventing inflammation in a subject, said method comprising inhibiting interactions between endothelial cells and immune cells or inhibiting interactions between endothelial cell and platelets. One embodiment provides a method of treating or preventing inflammation in a subject, said method comprising inhibiting chemokine secretion by endothelial cells or inhibiting the endothelial response to cytokines and other molecules, such as TGF-beta. One embodiment provides a method of treating cardiovascular disease in a subject, said method involving administering to said subject a compound capable of degrading Brd4. One embodiment provides a method of treating a lymphatic or a hematogenous metastasis in a subject comprising administering to the subject a heterobifunctional compound according to any of the above embodiments. One embodiment provides a method of treating inflammatory disease or disorder in a subject, the method comprising administering a heterobifunctional compound comprising a degrader molecule and an anti-TM4SF1 antibody or an antigen binding fragment thereof. wherein the degrader molecule targets one or more proteins for degradation, wherein the one or more protein for degradation is selected from the group consisting of: Akt, Hsp90, HDAC6, K-Ras, PI3K, BTK, B-Raf, ERK, MEK, P65 (RELA), p50 (NFKB1) of NFkB, Ras, Raf, eNOS, a Smad family protein, Smad2/3/4, and combinations thereof. In some embodiments, the inflammatory disease or disorder is a pathological angiogenesis. In some embodiments, subject is a human.

One embodiment provides a method of treating cancer in a subject, the method comprising administering a heterobifunctional compound according to any of the above embodiments, in combination with an immunomodulatory agent. In some embodiments, the immunomodulatory agent comprises an agent that binds to a protein selected from the group consisting of: A2AR, B7-H3, B7-H4, BTLA, CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, and VISTA.

One embodiment provides a heterobifunctional compound that comprises:
(a) an anti-TM4SF1 antibody; and
(b) a degrader molecule, wherein the degrader molecule comprises the following structure:

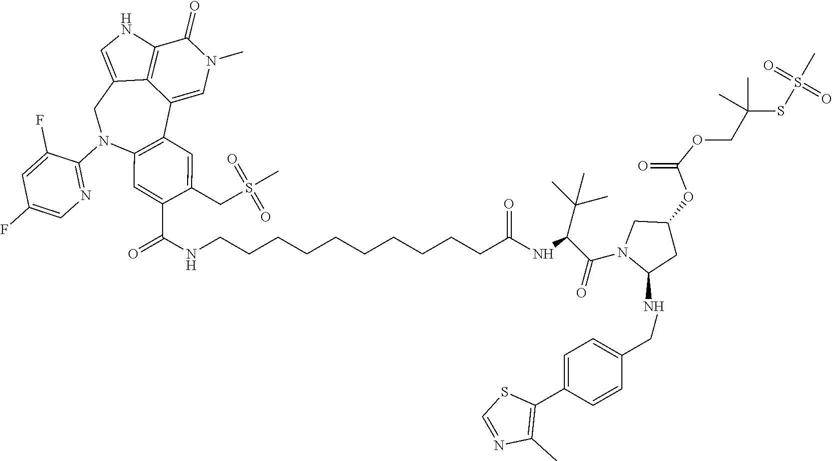 [SMILES: Cc1ncsc1-c1ccc(CN[C@@H]2C[C@@H](OC(=O)OCC(C)(C)SS(C)(=O)=O)CN2C(=O)[C@@H](NC(=O)CCCCCCCCCCNC(=O)c2cc3c(cc2CS(C)(=O)=O)-c2cn(C)c(=O)c4[nH]cc(c24)CN3c2ncc(F)cc2F)C(C)(C)C)cc1]

In some embodiments, the heterobifunctional compound of comprises a degrader to antibody ration (DAR) or about 2.0. In some embodiments, the anti-TM4SF1 antibody comprises an IgG1 Fc region comprising the following mutations: M252Y, S254T, T256E, and N297C, as numbered by the EU index as set forth in Kabat. In some embodiments, the anti-TM4SF1 antibody comprises: a heavy chain comprising a CDR3 domain comprising an amino acid sequence that has at least 75% identity to a sequence selected from the group consisting of SEQ ID NO: 8, 20, 32, 44, 56, 68, 80, 96, 118, 119, 120, or 121; a CDR2 domain comprising an amino acid sequence that has at least 75% identity to a sequence selected from the group consisting of SEQ ID NO: 7, 19, 31, 43, 55, 67, 79, 95, 116, or 117; and a CDR1 domain comprising an amino acid sequence that has at least 75% identity to a sequence selected from the group consisting of SEQ ID NO: 6, 18, 30, 42, 54, 66, 78, 94, or 115; and a light chain comprising a CDR3 domain comprising an amino acid sequence that has at least 75% identity to a sequence selected from the group consisting of SEQ ID NO: 14, 26, 38, 50, 62, 74, 86, 110, or 129; a CDR2 domain comprising an amino acid sequence that has at least 75% identity to a sequence selected from the group consisting of SEQ ID NO: 13, 25, 37, 49, 61, 73, 85, 109, or 128; and a CDR1 domain comprising an amino acid sequence that has at least 75% identity to a sequence selected from the group consisting of SEQ ID NO: 12, 24, 36, 48, 60, 72, or 84, 107, 108, 124, 125, 126, or 127.

One embodiment provides a method of treating cancer in a subject, the method comprising administering a heterobifunctional compound according to any one of the above embodiments. In some embodiments, the method comprises administering the heterobifunctional compound in combination with an immunomodulatory agent. In some embodiments, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A, FIG. 1B, and FIG. 1C provide structures of exemplary Brd4 degrader compounds for conjugation to anti-TM4SF1 antibodies or antigen binding fragments thereof.

FIG. 2 provides various structures of exemplary Brd4 degrader compounds for conjugation to anti-TM4SF1 antibodies or antigen binding fragments thereof.

FIG. 3 provides a synthesis scheme for conjugation of a Brd4 degrader to anti-TM4SF1 antibodies or antigen binding fragments thereof.

FIG. 4 provides the structure of an exemplary degrader antibody conjugate, comprising a Brd4 degrader and an anti-TM4SF1 antibody.

FIG. 5 provides a synthesis scheme for conjugation of a Brd4 degrader to anti-TM4SF1 antibodies or antigen binding fragments thereof.

FIG. 6 provides the structure of an exemplary degrader antibody conjugate, comprising a Brd4 degrader and an anti-TM4SF1 antibody.

FIG. 7 provides a synthesis scheme for conjugation of a Brd4 degrader to anti-TM4SF1 antibodies or antigen binding fragments thereof.

FIG. 8 provides the structure of an exemplary degrader antibody conjugate, comprising a Brd4 degrader and an anti-TM4SF1 antibody.

FIG. 9 provides a synthesis scheme for conjugation of a Brd4 degrader to anti-TM4SF1 antibodies or antigen binding fragments thereof.

FIG. 10 provides the structure of an exemplary degrader antibody conjugate, comprising a Brd4 degrader and an anti-TM4SF1 antibody.

FIG. 11 provides a synthesis scheme for conjugation of a Brd4 degrader to anti-TM4SF1 antibodies or antigen binding fragments thereof.

FIG. 12 provides the structure of an exemplary degrader antibody conjugate, comprising a Brd4 degrader and an anti-TM4SF1 antibody.

FIG. 13 provides a synthesis scheme for conjugation of a BCL-XL degrader to anti-TM4SF1 antibodies or antigen binding fragments thereof.

FIG. 14 provides a synthesis scheme for conjugation of a BCL-XL degrader to anti-TM4SF1 antibodies or antigen binding fragments thereof.

FIG. 15 provides a synthesis scheme for conjugation of a BCL-XL degrader to anti-TM4SF1 antibodies or antigen binding fragments thereof.

FIG. 16 provides structures of exemplary AKT degraders for conjugation to anti-TM4SF1 antibodies or antigen binding fragments thereof.

FIG. 17 provides the structures of an exemplary Brd4 degrader compound for conjugation to an anti-TM4SF1 antibody or an antigen binding fragment thereof using a dimethylmethanesulfonate linker, to form a degrader antibody conjugate (DAC).

FIG. 18 shows the results of the nuclear Brd4 ratio normalized to the control, as evaluated for concentrations of an exemplary DAC (A07-YTEC-Brd4 degrader compound 1) at 1.33 pM, 13.33 pM, 133.33 pM, 1.33 nM, and 13.33 nM after four-hour incubation in endothelial cells.

FIG. 19 shows an image of the Brd4 degradation by an exemplary anti-TM4SF1 degrader antibody conjugate (A07-YTEC-Brd4 degrader compound 1 at 133.33 pM (0.13333 nM); lower portion of the figure), compared to a control (upper portion of the figure).

FIG. 20 shows the results of a day-5 viability study for an in vitro assay conducted using endothelial cells and one of the following exemplary DACs: A07-YTEC-S-SO2Me-Alkyl-Brd4 degrader compound 1 at DAR of 5.5 (DAC15), A07-YTEC-S-SO2Me-Alkyl-Brd4 degrader compound 1 at DAR of 4.5 (DAC14) and A07-YTEC-PEG4Ahx-DM1.

FIG. 21 shows the results of a day-5 viability study for an in vitro assay conducted using pancreatic carcinoma cells and one of the following exemplary DACs: A07-YTEC-S-SO2Me-Alkyl-Brd4 degrader compound 1 at DAR of 5.5 (DAC15) and A07-YTEC-S-SO2Me-Alkyl-Brd 4 degrader compound 1 at DAR of 4.5 (DAC14), and A07-YTEC-PEG4Ahx-DM1.

FIG. 22 shows the results of a day-5 viability study for an in vitro assay conducted using adenocarcinomic human alveolar basal epithelial cells and one of the following exemplary DACs: A07-YTEC-S-SO2Me-Alkyl-Brd 4 degrader compound 1 at DAR of 5.5 (DAC15) and A07-YTEC-S-SO2Me-Alkyl-Brd 4 degrader compound 1 at DAR of 4.5 (DAC14) and A07-YTEC-PEG4Ahx-DM1.

FIG. 23 provides a spectrum showing the drug to antibody (DAR) ratio of an exemplary anti-TM4SF1 antibody degrader conjugate (DAC15), having a DAR of about 5.5.

FIG. 24 provides a spectrum showing the drug to antibody (DAR) ratio of an exemplary anti-TM4SF1 antibody degrader conjugate (DAC14), having a DAR of about 4.5.

FIG. 25 provides a chromatogram generated using a size exclusion column and an exemplary anti-TM4SF1 antibody conjugated to a degrader (DAC15), with a DAR of about 5.5.

FIG. 26 provides a chromatogram generated using a size exclusion column and an exemplary anti-TM4SF1 antibody conjugated to a degrader (DAC14), with a DAR of 4.5.

FIG. 27 shows the structure of an exemplary Brd4 degrader, used in degrader antibody conjugates tested in the cell killing and in vivo tumor regression studies provided herein.

FIG. 28 show A07-YTEC-S-SO2Me-Alkyl-Brd4 degrader conjugate at DAR of 5.5 (DAC15) or 4.5 (DAC14). BRD4 levels were quantified through Western Blot signal intensity at either 4 hours or 24 hours post treatment of the DAC.

FIG. 29 shows provides a spectrum showing the drug to antibody (DAR) ratio of an exemplary anti-TM4SF1 antibody degrader conjugate (DAC13).

FIG. 30A-30B provides a spectrum showing the drug to antibody (DAR) ratio (FIG. 30A) and an SEC spectrum (FIG. 30B) of an exemplary anti-TM4SF1 antibody degrader conjugate at DAR of 5.5 (DAC15).

FIG. 31A-31B provides a spectrum showing the drug to antibody (DAR) ratio (FIG. 31A) and an SEC spectrum (FIG. 31B) of an exemplary anti-TM4SF1 antibody degrader conjugate at DAR of 4.5 (DAC14).

FIG. 32A-32B provides a spectrum showing the drug to antibody (DAR) ratio (FIG. 32A) and an SEC spectrum (FIG. 32B) of an exemplary anti-TM4SF1 antibody degrader conjugate at DAR of 1.0 (DAC12).

FIG. 33A-33B provides a spectrum showing the drug to antibody (DAR) ratio (FIG. 33A) and an SEC spectrum (FIG. 33B) of an exemplary anti-TM4SF1 antibody degrader conjugate at DAR of 1.6 (DAC11).

FIG. 34A-34B provides a spectrum showing the drug to antibody (DAR) ratio (FIG. 34A) and an SEC spectrum (FIG. 34B) of an exemplary anti-TM4SF1 antibody degrader conjugate at DAR of 1.9 (DAC9).

FIG. 35A-35B provides a spectrum showing the drug to antibody (DAR) ratio (FIG. 35A) and an SEC spectrum (FIG. 35B) of an exemplary anti-TM4SF1 antibody degrader conjugate at DAR of 1.8 (DAC8).

FIG. 36 provides a schematic of an exemplary anti-TM4SF1 antibody degrader conjugate at a DAR of 2.0 through site specific conjugation.

FIG. 37A-37B provides a spectrum showing the drug to antibody (DAR) ratio (FIG. 37A) and an SEC spectrum (FIG. 37B) of an exemplary anti-TM4SF1 antibody degrader conjugate at DAR of 2.0 through site-specific conjugation.

FIG. 38 shows BRD4 protein degradation in HUVEC and A549 was quantified 24 hours treatment with the exemplary anti-TM4SF1 degrader conjugate at DAR 1.9.

FIG. 39 shows HUVEC cells treated with the exemplary anti-TM4SF1 DACs of DAR at 1.9 or DAR 5.0, and the free BRD4 degrader compound 1.

FIG. 40 shows the effect of tumor volume after treatment with exemplary anti-TM4SF1 degrader conjugates.

### DETAILED DESCRIPTION OF THE INVENTION

The disclosure, in several embodiments, provides degrader-antibody conjugates (DAC) comprising a degrader molecule and an anti-TM4SF1 antibody or an antigen binding fragment thereof. Degraders are chimeric molecules capable of triggering the degradation of an unwanted protein through intracellular proteolysis. The degraders, in some instances, contain two moieties, one that targets the unwanted protein and another that engages an E3 ubiquitin ligase. Degraders facilitate the ubiquitination of the unwanted protein by the E3 ubiquitin ligase, which leads to the subsequent degradation of the unwanted protein by the proteasome.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are used in the art. Standard techniques are used for chemical syntheses, chemical analyses, pharmaceutical preparation, formulation, and delivery, and treatment of patients.

That the present disclosure may be more readily understood, select terms are defined below. The terms “transmembrane-4 L six family member-1” or “TM4SF1”, as used herein refer to a polypeptide of the transmembrane 4 superfamily/tetraspanin family, which is highly expressed on tumor vasculature endothelial cells (ECs), tumor cells (TCs), ECs of developing retinal vasculature, and angiogenic blood vessels. TM4SF1 has two extracellular loops (ECL1 and ECL2) that are separated by four transmembrane domains (M1, M2, M3, and M4), the N- and C-termini, and the intracellular loop (ICL). ECL2 contains two N-glycosylation sites. The amino acid sequence of human TM4SF1 (hTM4SF1) is described in SEQ ID NO: 90 (see also NCBI Ref Seq No. NP_055035.1).

The term “antibody”, as used herein, means any antigen-binding molecule comprising at least one complementarity determining region (CDR) that specifically binds to or interacts with a particular antigen (e.g., TM4SF1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-TMS4F1 antibody (or antigen-binding portion thereof) may be identical to the human germline sequences, or may be naturally or artificially modified. An amino acid consensus sequence may be defined based on a side-by-side analysis of two or more CDRs.

The term “intact antibody” refers to an antibody comprising four polypeptide chains, two heavy (H) chains and two light (L) chains inter-connected by disulfide bonds. In one embodiment, the anti-TM4SF1 antibody is an intact antibody. In one embodiment, the intact antibody is an intact human IgG1, IgG2 or IgG4 isotype. In certain embodiments, the anti-TM4SF1 antibody, or antigen-binding fragment thereof, is a human IgG1, IgG2, or IgG4 isotype.

The terms “antigen-binding portion” of an antibody, “antigen-binding fragment,” or “antibody-fragment,” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from intact antibody molecules using any suitable standard techniques such as proteolytic digestion or recombinant genetic engineering techniques involving the manipulation and expression of DNA encoding antibody variable and optionally constant domains. Such DNA is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide.

The term “variable region” or “variable domain” of an antibody, or fragment thereof, as used herein refers to the portions of the light and heavy chains of antibody molecules that include amino acid sequences of complementarity determining regions (CDRs; i.e., CDR-1, CDR-2, and CDR-3), and framework regions (FRs). VH refers to the variable domain of the heavy chain. VL refers to the variable domain of the light chain. According to the methods used in this disclosure, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The term “complementarity determining regions” or “CDRs” as used herein refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but may nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “framework regions” (hereinafter FR) as used herein refers to those variable domain residues other than the CDR residues. Each variable domain typically has four FRs identified as FR1, FR2, FR3 and FR4. Common structural features among the variable regions of antibodies, or functional fragments thereof, are disclose herein and in literature. The DNA sequence encoding a particular antibody can generally be found following methods such as those described in Kabat, et al. 1987 Sequence of Proteins of Immunological Interest, U.S. Department of Health and Human Services, Bethesda Md., which is incorporated herein as a reference. In addition, a general method for cloning functional variable regions from antibodies can be found in Chaudhary, V. K., et al., 1990 Proc. Natl. Acad. Sci. USA 87:1066, which is incorporated herein as a reference.

The term “Fc region” herein is used to define a C-terminal region of an antibody heavy chain, including, for example, native sequence Fc regions, recombinant Fc regions, and variant Fc regions. Although the boundaries of the Fc region of an antibody heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system as in Kabat et al.) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Further, a composition of intact antibodies in this disclosure may comprise antibody populations with extension of residues after the C-terminal lysine, K447.

The term “humanized antibody” as used herein refers to an antibody or a variant, derivative, analog or fragment thereof, which immunospecifically binds to an antigen of interest (e.g., human TM4SF1), and which comprises a framework (FR) region having substantially the amino acid sequence of a human antibody and a complementary determining region (CDR) having substantially the amino acid sequence of a non-human antibody. Humanized forms of non-human (e.g., murine) antibodies are chimeric immunoglobulins that contain minimal sequences derived from non-human immunoglobulin. In general, a humanized antibody may comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are described in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

The term “monoclonal antibody” as used herein refers to an antibody obtained from a population of substantially homogeneous antibodies, i.e., the individual antibodies comprising the population are identical except for possible mutations, e.g., naturally occurring mutations that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal-antibody preparation is directed against a single epitope on an antigen.

The term “chimeric antibody” as used herein refers to antibodies (immunoglobulins) that have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “epitope” as used herein refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “binding affinity” generally refers to the strength of the sum total of noncovalent interactions between a single binding site of a molecule (e.g., a binding protein such as an antibody) and its binding partner (e.g., an antigen). The affinity of a binding molecule X (e.g., anti-TM4SF1 antibody) for its binding partner Y (e.g., human TM4SF1) can generally be represented by the dissociation constant (K_{D}). Affinity can be measured by common methods in the art, including those described herein. Low-affinity antibodies generally bind antigen slowly and tend to dissociate readily, whereas high-affinity antibodies generally bind antigen faster and tend to remain bound longer. A variety of methods of measuring binding affinity can be used for purposes of the present disclosure. Specific illustrative embodiments include the following. In one embodiment, the “K_{D}” or “K_{D} value” may be measured by assays such as, for example, a binding assay. The K_{D} may be measured in a RIA, for example, performed with the Fab version of an antibody of interest and its antigen (Chen et al., 1999, J. Mol Biol 293:865-81). The K_{D} may also be measured by using FACS or surface plasmon resonance assays by BIACORE, using, for example, a BIACORE 2000 or a BIACORE 3000, or by biolayer interferometry using, for example, the OCTET QK384 system. In certain embodiments, the K_{D} of an anti-TM4SF1 antibody is determined using a standard flow cytometry assay with HUVEC cells. An “on-rate” or “rate of association” or “association rate” or “kₒₙ” and an “off-rate” or “rate of dissociation” or “dissociation rate” or “k_{off}” may also be determined with the same surface plasmon resonance or biolayer interferometry techniques described above using, for example, a BIACORE 2000 or a BIACORE 3000, or the OCTET QK384 system.

The term “kₒₙ”, as used herein, is intended to refer to the on rate constant for association of an antibody to the antigen to form the antibody/antigen complex, as is known in the art.

The term “k_{off}”, as used herein, is intended to refer to the off rate constant for dissociation of an antibody from the antibody/antigen complex, as is known in the art.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “cancer” as used herein, refers to or describes the physiological condition in mammals that is typically characterized by unregulated cell growth.

The term “cancer which is associated with a high risk of metastasis”, as used herein, refers to a cancer that is associated with at least one factor known to increase the risk that a subject having the cancer may develop metastatic cancer. Examples of factors associated with increased risk for metastasis include, but are not limited to, the number of cancerous lymph nodes a subject has at the initial diagnosis of cancer, the size of the tumor, histological grading, and the stage of the cancer at initial diagnosis.

The term “hematogenous metastasis” as used herein refers to the ability of cancer cells to penetrate the walls of blood vessels, after which they are able to circulate through the bloodstream (circulating tumor cells) to other sites and tissues in the body.

The term “lymphatic metastasis” as used herein refers to the ability of cancer cells to penetrate lymph vessels and drain into blood vessels.

In the context of the disclosure, the term “treating” or “treatment”, as used herein, means reversing, alleviating, inhibiting the progress of, or preventing the disorder or condition to which such term applies, or one or more symptoms of such disorder or condition. By the term “treating cancer” as used herein is meant the inhibition of the growth and/or proliferation of cancer cells. In one embodiment, the compositions and methods described herein are used to treat metastasis in a subject having metastatic cancer.

The term “preventing cancer” or “prevention of cancer” refers to delaying, inhibiting, or preventing the onset of a cancer in a mammal in which the onset of oncogenesis or tumorigenesis is not evidenced but a predisposition for cancer is identified whether determined by genetic screening, for example, or otherwise. The term also encompasses treating a mammal having premalignant conditions top the progression of, or cause regression of, the premalignant conditions towards malignancy. Examples of premalignant conditions include hyperplasia, dysplasia, and metaplasia. In some embodiments, preventing cancer is used in reference to a subject who is in remission from cancer.

A variety of cancers, including malignant or benign and/or primary or secondary, may be treated or prevented with a method according to the disclosure. Examples of such cancers are known to those skilled in the art and listed in standard textbooks such as the Merck Manual of Diagnosis and Therapy (published by Merck).

The term “subject” as used herein, refers to a mammal (e.g., a human).

The term “administering” as used herein refers to a method of giving a dosage of an antibody or fragment thereof, or a composition (e.g., a pharmaceutical composition) to a subject. The method of administration can vary depending on various factors (e.g., the binding protein or the pharmaceutical composition being administered and the severity of the condition, disease, or disorder being treated).

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “manufacturability,” as used herein, refers to the stability of a particular protein during recombinant expression and purification of that protein. Manufacturability is believed to be due to the intrinsic properties of the molecule under conditions of expression and purification. Examples of improved manufacturability characteristics include uniform glycosylation of a protein, increased cell titer, growth and protein expression during recombinant production of the protein, improved purification properties, less propensity of aggregation or non-aggregation, and improved stability, including, but not limited to, thermal stability and stability at low pH. In some embodiments are provided TM4SF1 binding proteins that demonstrate the manufacturability, along with retention of in vitro and in vivo activity, compared with other TM4SF1 antibodies. In some embodiments, humanization of a parent TM4SF1 binding protein, by making amino acid substitutions in the CDR or framework regions, can confer additional manufacturability benefits.

In some embodiments are provided TM4SF1 binding proteins that demonstrate improved developability characteristics, including, but not limited to improved purification yield, for example, after protein A purification or size exclusion chromatography, improved homogeneity after purification, improved thermal stability. In some cases, the improvement is with respect to an anti-TM4SF1 antibody produced by a hybridoma mouse cell line 8G4-5-13-13F (PTA-120523), as determined by HLA molecule binding.

In some examples, binding affinity is determined by Scatchard analysis, which comprises generating a Scatchard plot, which is a plot of the ratio of concentrations of bound ligand to unbound ligand versus the bound ligand concentration.

The term “vascular toxicity” refers to any effect of an anti-TM4SF1 antibody or antigen binding thereof or a heterobifunctional compound comprising the same which leads to vascular injury either directly due to the antibody or the degrader compound effects on antigen-bearing cells or indirectly through activation of the immune system and resulting inflammation. Such vascular injury may include, but is not limited to, damage or inflammation affecting vascular endothelial cells or underlying smooth muscle cells or pericytes or the basement membrane of any blood vessel, including the endocardium (lining of the heart). Such vascular injury may affect arteries, including major arteries such as the aorta, elastic arteries (such as the aorta), muscuar arteries of varying sizes, such as coronary artery, pulmonary artery, carotid artery, arterioles, capillaries, arteries of the brain or retina; venues, veins; or it may affect angiogenic vessels including vessels serving hair follicles, the digestive tract, and bone marrow. Such vascular injury may include microvascular dysfunction or damage in the heart, lung, kidney, retina, brain, skin, liver, digestive tract, bone marrow, endocrine glands, testes or ovaries, endometrium, and other target organs and may include renal, retinal or cerebrovascular circulation dysfunction.

The term “antibody-dependent cell-mediated cytotoxicity (ADCC)” as used herein refers to the killing of an antibody-coated target cell by a cytotoxic effector cell through a nonphagocytic process, characterized by the release of the content of cytotoxic granules or by the expression of cell death-inducing molecules. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). PBMC-based ADCC assays and natural kill cell-based ADCC assays can be used to detect ADCC. The readout in these assays is endpoint-driven (target cell lysis).

The term “complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (See. e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164: 4178-84). Antibodies (or fragments) with little or no CDC activity may be selected for use.

The term “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Examples of antibody effector functions include: C1q binding and complement dependent cytotoxicity (CDC); Fc receptor binding; antibody-dependent cell-mediated cytotoxicity (ADCC); phagocytosis (ADCP); down regulation of cell surface receptors (e.g. B cell receptor); and B cell activation.

The terms “reduce” or “ablate” as used herein refers to the ability to cause an overall decrease preferably of 20% or greater, more preferably of 50% or greater, and most preferably of 75%, 85%, 90%, 95%, or greater. Reduce or ablate can refer to binding affinity of two molecules, for example the binding of immunoglobulins to C1q or to Fc receptors; or can refer to the symptoms of the disorder (e.g., cancer) being treated, such as the presence or size of metastases or the size of the primary tumor.

The term “reduced ADCC/CDC function,” as used herein refers to a reduction of a specific effector function, e.g. ADCC and/or CDC, in comparison to a control (for example an antibody with a Fc region not including the mutation(s)), by at least about 5%, at least about 10%, at least about 15%, at least about 20%, at least about 30%, at least about 40%, at least about 50%, at least about 60%, at least about 70%, at least about 80% at least, at least about 90% or more.

For all amino acid positions discussed in the present disclosure, in the context of antibodies or antigen binding fragments thereof, numbering is according to the EU index. The “EU index” or “EU index as in Kabat et al.” or “EU numbering scheme” refers to the numbering of the EU antibody (See Edelman et al., 1969; Kabat et al., 1991).

### Heterobifunctional Compounds

Provided herein are heterobifunctional degrader-antibody conjugate (DAC) compositions that result in the ubiquitination of a target protein and subsequent degradation of the protein. The heterobifunctional compositions comprise an antibody and a degrader. The degrader comprises an E3 ubiquitin ligase binding (E3LB) moiety (where the E3LB moiety recognizes a E3 ubiquitin ligase protein) and a protein binding moiety (PB) that recognizes a target protein.

The terms “residue,” “moiety” or “group” refers to a component that is covalently bound or linked to another component. For example, a “residue of a degrader” refers to a degrader that is covalently linked to one or more groups such as a Linker (L2), which itself can be optionally further linked to an antibody via linker (L1).

In one aspect provided herein, a Degrader-Antibody Conjugate (DAC) described herein comprises an anti-TM4SF antibody or an antigen-binding fragments thereof conjugated via a linker (L1) to a degrader; wherein the degrader comprises a ubiquitin E3 ligase binding group (“E3LB”), a linker (“L2”) and a protein binding group (“PB”).

An exemplary general formula of a DAC is Ab-(L1-D)p, where D is degrader having the structure E3LB-L2-PB; wherein, E3LB is an E3 ligase binding group covalently bound to L2; L2 is a linker covalently bound to E3LB and PB; PB is a protein binding group covalently bound to L2; Ab is an antibody covalently bound to L1; L1 is a linker, covalently bound to Ab and to D; and p has a value from about 1 to about 50. The variable p reflects that an antibody can be connected to one or more L1-D groups. In one embodiment, p is from about 1 to 8. In one instance, p is about 1, about 2, about 3, about 4, about 5, about 6, about 7, or about 8.

Anti-TM4SF1 Antibodies (Abs)

TM4SF1 is a small plasma membrane glycoprotein (NCBI Ref Seq No. N P_055035.1) with tetraspanin topology but not homology (Wright et al. Protein Sci. 9: 1594-1600, 2000). It forms TM4SF1-enriched domains (TMED) on plasma membranes, where, like genuine tetraspanins, it serves as a molecular facilitator that recruits functionally related membrane and cytosolic molecules (Shih et al. Cancer Res. 69: 3272-3277, 2009; Zukauskas et al., Angiogenesis. 14: 345-354, 2011), and plays important roles in cancer cell growth (Hellstrom et al. Cancer Res. 46: 391 7-3923, 1986), motility (Chang et al. Int J Cancer. 1 16: 243-252, 2005), and metastasis (Richman et al. Cancer Res. 5916s-5920s, 1995). The amino acid sequence of human TM4SF1 protein (NCBI RefSeq No. NP_055035.1) is shown below as SEQ ID NO: 134.

(SEQ ID NO: 134)
MCYGKCARCI GHSLVGLALL CIAANILLYF PNGETKYASE
NHLSRFVWFF SGIVGGGLLM LLPAFVFIGL EQDDCCGCCG
HENCGKRCAM LSSVLAALIG IAGSGYCVIV
AALGLAEGPLCLDSLGQWNYTFASTEGQYLLDTSTWSECTEPK
HIVEWNVSLFSILLALGGIEFILCLIQVINGVLGGIC
GFCCSHQQQY DC

One embodiment of the disclosure provides heterobifunctional compounds comprising an anti-TM4SF1 antibody or an antigen binding fragment thereof, wherein the anti-TM4SF1 antibody or antigen binding fragment thereof comprises a modified Fc region, such as a modified IgG region (e.g., IgG1, IgG2, IgG3, IgG4) comprising one or more mutations. In some cases, said one or more mutations in the Fc region leads to improvements in a heterobifunctional compound comprising such a modified Fc region, in areas of improvement such as: 1) reduction of effector functions, 2) half-life modulation, 3) stability, and 4) downstream processes. In some cases, the modified Fc region can comprise one or more mutations that may reduce or ablate interactions between the antibodies and the immune system. Key interactions may include interactions of the antibody Fc with Fcγ receptors on white blood cells and platelets, and with C1q of the complement system leading to complement dependent cytotoxicity.

The present disclosure provides, in some cases, a heterobifunctional compound comprising an anti-TM4SF1 antibody or an antigen binding fragment thereof that includes immune ablating mutations, for example, in the Fc region which in such cases is a modified Fc region, for example, a modified IgG Fc region. In some embodiments, the modified Fc region comprises a modification at position N297. In some embodiments, the modified Fc region comprises a modified IgG Fc region (e.g., a modified IgG1, IgG2, IgG3, or IgG4 Fc region) comprising one or more mutations at positions E233, L234 or F234, L235, G237, P238, F243, T250, M252, S254, T256, E258, D259, V264, D265, K288, N297, T299, T307, V308, Q311, K322, L328, P329, A330, P331, T356, K370, A378, R409, V427, M428, H433, N434, and H435, or any combinations thereof. In some embodiments, the Fc region comprises an extension of residues at its C-terminus, such that positive charge is maintained at the C-terminus (e.g., in some cases, if the anti-TM4SF1 antibody or antigen binding fragment comprises two heavy chains then at least one heavy chain comprises an extension of residues at the C-terminus). Such extension of residues can comprises addition of one or more amino acids, such as, arginine, lysine, proline, or any combinations thereof. In some examples, the extended C-terminus of the Fc regions leads to reduced CDC function of the anti-TM4SF1 antibody or antigen binding fragment thereof, and that of a heterobifunctional compound comprising the anti-TM4SF1 antibody or antigen binding fragment thereof. Such an effect is seen, in some cases, by addition of KP residues after K447 of Fc in IgG1 or IgG4, alone or in combination with other mutations (e.g., K322A, P331G-IgG1).

In some embodiments, an anti-TM4SF1 antibody or an antigen binding fragment thereof can comprise an antibody with reduced effector function, including substitution of one or more of Fc region residues 238, 265, 269, 270, 297, 327 and 329 (See, e.g., U.S. Pat. No. 6,737,056). In some cases, such mutations in the Fc region may comprise substitutions at two or more of amino acid positions 265, 269, 270, 297 and 327, for example, substitution of residues 265 and 297 to alanine (DANA mutations, i.e., D265A and N297A) (See, e.g., U.S. Pat. No. 7,332,581). In some cases, mutations in the Fc region may comprises substitutions at one or more amino acid positions E233, L234, L235, G237, D265, N297, K322, and P331. In some cases, mutations in the Fc region may comprises at least one of E233P, L234A, L235A, G237A, D265A, N297A, K322A, and P331G, or any combinations thereof. For instance, the mutations in the Fc region can comprise L234A/L235A/G237A (IgG1), or F234A/L235E (IgG4), and an anti-TM4SF1 antibody or antigen binding fragment comprising such mutations may exhibit altered FcgRI interactions.

In some embodiments, an anti-TM4SF1 antibody or antigen binding fragment thereof may include an Fc variant comprising the following mutations: an amino acid substitution at position M428 and N434 (M428L, N434S) (See. e.g., U.S. Pat. No. 9,803,023). In some embodiments, an anti-TM4SF1 antibody or antigen binding fragment thereof may include an Fc variant comprising the following mutations: an amino acid substitution at position 1250 and M428 (T250Q, M428L) (See, e.g., U.S. Pat. No. 9,803,023).

In some embodiments, the TM4SF1 antibody or antigen binding fragment thereof may comprise mutations D265A and N297A. In some cases, the proline at position 329 (P329) of a wild-type human Fc region may be substituted with glycine or arginine or an amino acid residue large enough to destroy the proline sandwich within the Fc/Fcy receptor interface, that is formed between the P329 of the Fc and tryptophan residues W87 and WHO of FcgRIII (See. e.g., Sondermann et al., Nature 406, 267-273 (20 Jul. 2000)). In a further embodiment, the mutations in the Fc region may comprise one or more amino acid substitutions such as S228P (IgG4), E233P, L234A, L235A, L235E, N297A, N297D, or P331S and in still in other embodiments: L234A and L235A of the human IgG1 Fc region or S228P and F234A, L235A, or L235E of the human IgG4 Fc region.

In some embodiments, an anti-TM4SF1 antibody or antigen binding fragment thereof may include a modified Fc region which is an Fc variant of a wild-type human IgG Fc region wherein P329 of the human IgG Fc region substituted with glycine and wherein the Fc variant comprises at least two further amino acid substitutions at L234A and L235A of the human IgG1 Fc region or S228P and L235E of the human IgG4 Fc region, and wherein the residues are numbered according to the EU numbering (See. e.g., U.S. Pat. No. 8,969,526). The polypeptide comprising the P329G, L234A and L235A substitutions may exhibit a reduced affinity to the human FcyRIIIA and FcyRIIA, for down-modulation of ADCC to at least 20% of the ADCC induced by the polypeptide comprising the wildtype human IgG Fc region, and/or for down-modulation of ADCP (See, e.g., U.S. Pat. No. 8,969,526).

In some embodiments, an anti-TM4SF1 antibody or antigen binding fragment thereof may include an Fc variant comprising triple mutations: an amino acid substitution at position P329, a L234A and a L235A mutation (P329/LALA) (See, e.g., U.S. Pat. No. 8,969,526).

Certain anti-TM4SF1 antibodies or antigen binding fragments of this disclosure, in some embodiments, can comprise mutations that exhibit improved or diminished binding to FcRs. (See, e.g., U.S. Pat. No. 6,737,056; WO 2004/056312, and Shields et al., J. Biol. Chem. 9(2): 6591-6604 (2001).)

In some instances, an anti-TM4SF1 antibody or antigen binding fragment may include an Fc region with one or more amino acid substitutions which improve ADCC, e.g., substitutions at positions 298, 333, and/or 334 of the Fc region. Alterations may be made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. (2000) J. Immunol. 164: 4178-4184.

Antibodies with increased half-lives and improved binding to the neonatal Fc receptor (FcRn). FcRn, named after its function for the transfer of maternal IgGs to the fetus, also serves to prevent antibodies from being degraded in lysosomes, by capturing them in endosomes and returning them to circulation. (See, e.g., Guyer et al., J. Immunol. 117:587 (1976) and Kim et al., J. Immunol. 24:249 (1994)), are described in US2005/0014934. Without being bound by any particular theory, it is contemplated that antibodies with improved binding to FcRn detach from TM4SF1 and bind to FcRn, which then recycles the antibody back to circulation, thus reducing vascular toxicity. In some embodiments herein are provided anti-TM4SF1 antibodies or antigen binding fragments that comprise an Fc region with one or more substitutions that enhance FcRn recycling. In some embodiments herein are provided anti-TM4SF1 antibodies or antigen binding fragments thereof that comprise an Fc region with one or more substitutions therein which improve binding of the Fc region to FcRn, such as, substitutions at one or more of positions: 238, 250, 252, 254, 256, 265, 272, 286, 303, 305, 307, 311, 312, 317, 340, 356, 360, 362, 376, 378, 380, 382, 413, 428, 424, 434, and 435, e.g., substitution of Fc region residue 434 (U.S. Pat. No. 7,371,826) according to EU numbering. See also Duncan &amp; Winter, Nature 322:738-40 (1988); U.S. Pat. Nos. 5,648,260; 5,624,821; US2005/0014934 and WO 94/29351 concerning other examples of Fc region variants, the entirety of which are incorporated herein by reference.

In some embodiments, provided herein are anti-TM4SF1 antibodies or antigen binding fragments thereof that have pH dependent FcRn binding affinities. Without being bound by any particular theory, it is contemplated that anti-TM4SF1 antibodies or antigen binding fragments thereof with pH dependent FcRn binding affinity detach from FcRn at pH >7, and bind to FcRn at pH 6. Accordingly, FcRn in acidic pH subcellular organelles, e.g. endosomes, binds such antibodies and carries the antibodies back to the cell membrane, and release the antibodies into plasma at pH >7, recycling the antibody and avoiding lysosomal release of payloads conjugated to the antibody.

In certain embodiments, herein are provided anti-TM4SF1 antibodies or antigen binding fragments thereof that comprise an Fc region with one or more substitutions therein which modulate FcRn recycling. In some embodiments herein are provided anti-TM4SF1 antibodies or antigen binding fragments thereof that comprise one or more substitutions that enhance FcRn binding at acidic pH, e.g., pH 6, and does not affect FcRn binding at neutral or basic pH, e.g. pH 7. In some embodiments, an anti-TM4SF1 antibody or antigen binding fragment thereof may comprise substitutions at one or more of positions 250, 252, 254, 256, 428, and 434 according to EU numbering. In some embodiments, an anti-TM4SF1 antibody or antigen binding fragment thereof may include an Fc variant comprising one or more of substitutions T250Q, M252Y, S254T, T256E, M428L, and N434S. In some embodiments, an anti-TM4SF1 antibody or antigen binding fragment thereof may include an IgG1 Fc variant comprising substitutions T250Q and M428L (the “QL mutant”). In some embodiments, an anti-TM4SF1 antibody or antigen binding fragment thereof may include an IgG4 Fc variant comprising substitutions T250Q and M428L (the “QL mutant”). In some embodiments, an anti-TM4SF1 antibody or antigen binding fragment thereof may include an IgG1 Fc variant comprising substitutions M252Y, S254T, and T256E (the “YTE mutant”). In some embodiments, an anti-TM4SF1 antibody or antigen binding fragment thereof may include an IgG1 Fc variant comprising substitutions M428L and N434S (the “LS mutant”). In some embodiments, an anti-TM4SF1 antibody or antigen binding fragment thereof may include an IgG4 Fc variant comprising substitutions M428L and N434S (the “LS mutant”). Effects of amino acid substitutions in the Fc region that modulate FcRn recycling are described in, e.g. Hamblett et al., Mol. Pharm. 13(7): 2387-96 (2016); Dall'Acqua et al., J. Biol. Chem. 281(33): 23514-24 (2006), Hinton et al., J. Biol. Chem. 279(8): 6213-6 (2003), Hinton et al., J. Immunol., 176(1): 346-56 (2006), US20080181887, U.S. Pat. No. 7,361,740, and EP2235059, the entirety of which are incorporated herein by reference.

In certain embodiments, an anti-TM4SF1 antibody, or antigen-binding fragment thereof, is an IgG1 isotype and comprises an Fc region comprising one or more substitutions selected from the group consisting of T250Q, M252Y, S254T, T256E, M428L, and N434S. In some embodiments, an anti-TM4SF1 antibody, or antigen binding fragment thereof, is an IgG4 isotype and comprises an Fc region comprising one or more substitutions selected from the group consisting of T250Q, M252Y, S254T, T256E, M428L, and N434S. In some embodiments, an anti-TM4SF1 antibody or antigen binding fragment thereof is an IgG1 isotype and comprises an Fc region comprising substitutions T250Q and M428L. In some embodiments, an anti-TM4SF1 antibody or antigen binding fragment thereof is an IgG1 isotype and comprises an Fc variant comprising substitutions M252Y, S254T, and T256E. In some embodiments, an anti-TM4SF1 antibody or antigen binding fragment thereof is an IgG4 isotype and comprises an Fc variant comprising substitutions M252Y, S254T, and T256E. In some embodiments, an anti-TM4SF1 antibody or antigen binding fragment thereof is an IgG1 isotype and comprises an Fc variant comprising substitutions M428L and N434S. In some embodiments, an anti-TM4SF1 antibody or antigen binding fragment thereof is an IgG4 isotype and comprises an Fc variant comprising substitutions M428L and N434S.

In certain embodiments, the heterobifunctional compounds disclosed herein exhibit reduced vascular toxicity, reduced lysosomal toxicity, improved efficacy, and/or improved therapeutic margin. In some embodiments, the heterobifunctional compounds disclosed herein comprise anti-TM4SF1 antibodies or antigen binding fragments thereof comprising mutated Fc regions that have increased FcRn binding affinity and increased serum half life. In certain embodiments, an anti-TM4SF1 antibody or antigen binding fragment thereof comprising mutated Fc regions have serum half life of at least 10 days, at least 15 days, at least 20 days, at least 25 days, at least 30 days, at least 35 days, at least 40 days, at least 50 days, at least 60 days, at least 70 days, at least 80 days, at least 90 days, at least 100 days or more. In some embodiments,

In certain embodiments, the heterobifunctional compounds of this disclosure exhibit reduced vascular toxicity, improved therapeutic margin, or both. In certain embodiments the heterobifunctional compounds of this disclosure comprise anti-TM4SF1 antibodies or antigen binding fragments thereof comprising mutated Fc regions that have reduced or ablated affinity for an Fc ligand responsible for facilitating effector function compared to an antibody having the same amino acid sequence as the antibody of the disclosure but not comprising the addition, substitution, or deletion of at least one amino acid residue to the Fc region (also referred to herein as an “unmodified antibody”).

In one embodiment, an anti-TM4SF1 antibody, or antigen-binding fragment thereof comprises an Fc region comprising at least two mutations that reduce or ablate ADCC and/or CDC effector function of the antibody, or antigen-binding fragment thereof. In further embodiments, the anti-TM4SF1 antibody, or antigen-binding fragment thereof, comprises an Fc region comprising at least three, at least four, at least five, at least six, at least seven, at least eight, at least nine, at least ten or more mutations that reduce or ablate ADCC and/or CDC effector function of the antibody, or antigen-binding fragment thereof.

In certain embodiments, an anti-TM4SF1 antibody, or antigen-binding fragment thereof, is an IgG1 isotype and comprises an Fc region comprising one or more mutations selected from the group consisting of E233P, L234V, L234A, L235A, G236Delta (deletion), G237A, V263L, N297A, N297D, N297G, N297Q, K322A, A327G, P329A, P329G, P329R, A330S, P331A, P331G, and P331S.

In one embodiment, an anti-TM4SF1 antibody, or antigen-binding fragment thereof, is an IgG1 isotype and comprises an Fc region comprising an L234A/L235A mutation, with or without a G237A mutation. In one embodiment, the anti-TM4SF1 antibody, or antigen-binding fragment thereof, is an IgG1 isotype and comprises an Fc region comprising L234A, L235A, and G237A mutations.

In one embodiment, an anti-TM4SF1 antibody, or antigen-binding fragment thereof, is an IgG1 isotype and comprises an Fc region comprising an A327G/A330S/P33IS mutation.

In one embodiment, an anti-TM4SF1 antibody, or antigen-binding fragment thereof, is an IgG1 isotype and comprises an Fc region comprising an E233P/L234V/L235A/delta G236 (deletion) mutation, which provides reduced binding to FcγRI (also referred to herein as FcgRI), FcγRIIA (also referred to herein as FcgRIIA), FcγRIIIA (also referred to herein as FcgRIIIAI) and reduced ADCC and CDC effector function, as described, for example, in An Z et al. Mabs 2009 November-Ec; 1(6):572-9, incorporated by reference in its entirety herein.

In one embodiment, an anti-TM4SF1 antibody, or antigen-binding fragment thereof, is an IgG1 isotype and comprises an Fc region comprising an N297× mutation, where x=A, D, G, Q.

In one embodiment, an anti-TM4SF1 antibody, or antigen-binding fragment thereof, is an IgG1 isotype and comprises an Fc region comprising an A327G/A330S/P33IS mutation.

In one embodiment, an anti-TM4SF1 antibody, or antigen-binding fragment thereof, is an IgG1 isotype and comprises an Fc region comprising a mutation in one or more of K322A, P329A, and P331A, which provides reduced binding to C1q, as described, for example, in Canfield & Morrison. J Exp Med (1991) 173(6):1483-91.10.1084, incorporated by reference in its entirety herein.

In one embodiment, an anti-TM4SF1 antibody, or antigen-binding fragment thereof, is an IgG1 isotype and comprises an Fc region comprising a V263L mutation, which provides enhanced binding to FcγRIIB (also referred to herein as FcgRIIB) and enhanced ADCC, as described in, for example, Hezareh et al. J Virol. 2001 December; 75(24):12161-8, incorporated by reference in its entirety herein.

In other embodiments, an anti-TM4SF1 antibody or antigen-binding fragment thereof is an IgG1 isotype and comprises an Fc region comprising a L234A/L235A, G237A or L235E mutation.

In other embodiments, an anti-TM4SF1 antibody, or antigen-binding fragment thereof, is an IgG1 isotype and comprises an Fc region comprising a L234F, L235E or P331S mutation.

In certain embodiments, an anti-TM4SF1 antibody, or antigen-binding fragment thereof, is an IgG2 isotype and comprises an Fc region comprising a one or more mutations selected from the group consisting of V234A, G237A, P238S, H268A or H268Q, V309L, A330S and P331S.

In one embodiment, an anti-TM4SF1 antibody, or antigen-binding fragment thereof, is an IgG2 isotype and comprises an Fc region comprising an A330S/P33IS mutation.

In one embodiment, an anti-TM4SF1 antibody, or antigen-binding fragment thereof, is an IgG2 isotype and comprises an Fc region comprising an A330S/P33IS, V234A/G237A/P238S/H268A/V309L/A330S/P33IS or H268Q/V309L/A330S/P33IS mutation.

In other embodiments, an anti-TM4SF1 antibody, or antigen-binding fragment thereof, is an IgG4 isotype and comprises an Fc region comprising a one or more mutations selected from the group consisting of S228P, E233P, F234A, F234V, L235E, L235A, G236Delta (deletion), N297A, N297D, N297G, N297Q, P329G, P329R.

In certain embodiments, an anti-TM4SF1 antibody, or antigen-binding fragment thereof, is an IgG4 isotype and comprises an Fc region comprising an S228P mutation, which provides reduced Fab-arm exchange and reduced aggregation, as described for example in Chappel et al. Proc Natl Acad Sci USA (1991) 88(20):9036-40, incorporated by reference in its entirety herein.

In one embodiment, an anti-TM4SF1 antibody, or antigen-binding fragment thereof, is an IgG4 isotype and comprises an Fc region comprising an S228P/L235E mutation.

In one embodiment, an anti-TM4SF1 antibody, or antigen-binding fragment thereof, is an IgG4 isotype and comprises an Fc region comprising an S228P/E233P/F234V/L235A/delta G236 (deletion) mutation.

In one embodiment, an anti-TM4SF1 antibody, or antigen-binding fragment thereof, is an IgG4 isotype and comprises an Fc region comprising an N297× mutation, where x=A, D, G, Q.

In one embodiment, an anti-TM4SF1 antibody, or antigen-binding fragment thereof, is an IgG4 isotype and comprises an Fc region comprising an S228P/F234A/L235A mutation.

In one embodiment, an anti-TM4SF1 antibody, or antigen-binding fragment thereof, is an IgG4 isotype and comprises an Fc region comprising a L235E mutation, which provides reduced binding to FcγRI, FcγRIIA, FcγRIIIA and reduced ADCC and CDC effector activity, as described in, for example, Saxena et al. Front Immunol. 2016 Dec. 12; 7:580.

In other embodiments, an anti-TM4SF1 antibody, or antigen-binding fragment thereof, is an IgG4 isotype and comprises an Fc region comprising a S228P/F234A/L235A or E233P/L235A/G236Delta mutation.

In one embodiment, an anti-TM4SF1 antibody, or antigen-binding fragment thereof, is an IgG4 isotype and comprises an Fc region comprising at least a S228P mutation. See. e.g., Angal et al. (Mol Immunol. 1993 January; 30(1):105-8) describe an analysis of the hinge sequences of human IgG4 heavy chains to determine that the presence of serine at residue 241 (according to EU numbering system, and now corresponding to residue 228 in Kabat numbering) as the cause of heterogeneity of the inter-heavy chain disulphide bridges in the hinge region in a proportion of secreted human IgG4. Silva et al. (J Biol Chem. 2015 Feb. 27; 290(9):5462-9) describe the S228P mutation in human IgG4 that prevents in vivo and in vitro IgG4 Fab-arm exchange.

In other embodiments, an anti-TM4SF1 antibody, or antigen-binding fragment thereof, is an IgG4 isotype and comprises an Fc region comprising a L235E or S228P mutation.

In other embodiments, the anti-TM4SF1 antibody, or antigen-binding fragment thereof, is an IgG4 or IgG1 isotype and comprises an Fc region comprising a N297A, N297D or N297G mutation.

In other embodiments, an anti-TM4SF1 antibody, or antigen-binding fragment thereof, is an IgG4 or IgG1 isotype and comprises an Fc region comprising a P329G, P329R mutation.

In one exemplary embodiment, the mutated Fc region of any IgG isotype comprises one or more mutations at positions 234, 235, 236, 237, 297, 318, 320, 322 (as described in WO1988007089, incorporated by reference in its entirety herein). Other possible mutations in the Fc region, including substitutions, deletions and additions are also described in, for example, US20140170140, WO2009100309, US20090136494 and U.S. Pat. No. 8,969,526, incorporated by reference in their entireties herein.

In vitro and/or in vivo cytotoxicity assays can be conducted to confirm the reduction or ablation of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, RII and RIII. Non-limiting examples of in vitro assays to assess ADCC activity of a molecule of interest is described in U.S. Pat. No. 5,500,362 (see, e.g. Hellstrom, I., et al., Proc. Nat'l Acad. Sci. USA 83 (1986) 7059-7063) and Hellstrom, I., et al., Proc. Nat'l Acad. Sci. USA 82 (1985) 1499-1502; U.S. Pat. No. 5,821,337 (see Bruggemann, M., et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, et al., Proc. Nat'l Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743). FcRn binding and in vivo clearance/half-life determinations can also be performed using methods in the art (see, e.g., Petkova, S. B., et al., Int'l. Immunol. 18(12) (2006) 1759-1769).

In some embodiments, the mutated Fc region of any IgG isotype comprises a mutation at position L328, such as L328M, L328D, L328E, L328N, L328Q, L328F, L328I, L328V, L328T, L328H, L328A (see e.g., US20050054832)

In one embodiment, antibodies, or antigen-binding fragments thereof, of the disclosure exhibit reduced or ablated ADCC effector function as compared to unmodified antibodies. In another embodiment, antibodies, or antigen-binding fragments thereof, of the disclosure exhibit reduced ADCC effector function that is at least 2 fold, or at least 3 fold, or at least 5 fold or at least 10 fold or at least 50 fold or at least 100 fold less than that of an unmodified antibody. In still another embodiment, antibodies of the disclosure exhibit ADCC effector function that is reduced by at least 10%, or at least 20%, or by at least 30%, or by at least 40%, or by at least 50%, or by at least 60%, or by at least 70%, or by at least 80%, or by at least 90%, or by at least 100%, relative to an unmodified antibody. In a further aspect of the disclosure the reduction or down-modulation of ADCC effector function induced by the antibodies, or antigen-binding fragments thereof, of the present disclosure, is a reduction to 0, 2.5, 5, 10, 20, 50 or 75% of the value observed for induction of ADCC by unmodified antibodies. In certain embodiments, the reduction and/or ablation of ADCC activity may be attributed to the reduced affinity of the antibodies, or antigen-binding fragments thereof, of the disclosure for Fc ligands and/or receptors.

CDR Substitutions that Modulate pH-Dependent TM-4SF1 Binding of an Anti-TM4SF1 Antibody or Antigen Binding Fragment Thereof

One embodiment of the disclosure provides a heterobifunctional compound comprising an anti-TM4SF1 antibody or an antigen binding fragment thereof, wherein the anti-TM4SF1 antibody or antigen binding fragment thereof exhibit pH dependent binding affinity to TM4SF1. In some instances, an anti-TM4SF1 antibody or antigen binding fragment thereof binds to TM4SF1 with higher affinity at certain pH range as compared to other pH ranges. For example, an anti-TM4SF1 antibody or antigen binding fragment thereof may bind to TM4SF1 with different affinity at an acidic pH than at a neutral pH or a basic pH. In some embodiments, an anti-TM4SF1 antibody or antigen binding fragment thereof binds to TM4SF1 with higher affinity at an acidic pH than at a neutral or basic pH. In some embodiments, an anti-TM4SF1 antibody or antigen binding fragment thereof binds to TM4SF1 with lower affinity at an acidic pH than at a neutral or basic pH. In some embodiments, an anti-TM4SF1 antibody or antigen binding fragment thereof binds to TM4SF1 at acidic pH and dissociates from TM4SF1 at neutral or basic pH. In some embodiments, an anti-TM4SF1 antibody or antigen binding fragment thereof binds to TM4SF1 at pH7 or higher and detaches from TM4SF1 at pH6 or lower. In subcellular compartments such as plasma, cytosol, and nucleus, the pH is neutral or basic. In lysosomes or endosomes, the pH is acidic. Without being bound by any theory, an anti-TM4SF1 antibody or antigen binding fragment thereof in some instances binds to the antigen and subsequently internalized in the membrane of an endosome. A pH-dependent anti-TM4SF1 antibody or antigen binding fragment thereof can detach from TM4SF1 in an endosome and bind to FcRn receptors within the endosome, and can be recycled by the FcRn receptor back into circulation rather than degraded in a lysosome that the endosome progresses to. Accordingly, a pH dependent anti-TM4SF1 antibody or antigen binding fragment thereof can bind to TM4SF1 antigen multiple times. Accordingly, a pH dependent anti-TM4SF1 antibody and a compound comprising the same (along with a payload, such as a degrader compound as described herein) can be recycled by FcRn receptors, without releasing a payload in the lysosome.

Target-mediated drug disposition, or TMDD, occurs when an antigen carries a bound antibody and/or any associated payload (such as a degrader compound, as described herein) to the lysosome, wherein the payload is released. Lysosome toxicity related to TMDD as described in Grimm et al., J. Pharmacokinet. Pharmacodyn. 36(5): 407-20 (2009) is incorporated herein by reference in its entirety. In some embodiments, provided herein are heterobifunctional compounds comprising an anti-TM4SF1 antibody or antigen binding fragment thereof that exhibit reduced vascular toxicity, increased serum half-life, and/or improved therapeutic margin. In some embodiments, an anti-TM4SF1 antibody or antigen binding fragment thereof comprises one or more histidine amino acid residue substitutions in CDR residues. Not intended to be bound by any particular theory, the introduction of a histidine residue at a suitable position of an anti-TM4SF1 antibody may allow pH-regulatable binding affinity to TM4SF1. For example, a pH-dependent anti-TM4SF1 antibody may dissociate from TM4SF1 in acidic lysosome or endosome environment, and subsequently be recycled into circulation via FcRn binding. As compared to an otherwise comparable wild type anti-TM4SF1 antibody or antigen binding fragment thereof, a pH-dependent ant-TM4SF1 antibody may exhibit increased serum half-life and reduced degradation rate or payload release rate in lysosomes. In some cases, a pH-dependent anti-TM4SF1 antibody or antigen binding fragment thereof may demonstrate increased half-life, reduced vascular toxicity, improved therapeutic window, and/or improved or at least about equivalent in vivo potency.

Disclosed herein are methods of making a heterobifunctional compound comprising an anti-TM4SF1 antibody or antigen binding fragment thereof that has increased half-life and/or pharmacodynamic effect by regulating antibody-TM4SF1 binding affinity in a pH dependent manner, comprising selecting for antibody CDR histidine residues or other residues that optimize the microenvironment affecting pKa of the antibody, such that the anti-TM4SF1 antibody or antigen binding fragment thereof has a Kd ratio and/or Koff ratio at pH6.0/pH7.4 that is at least 2, 3, 4, 8, 10, 16, or more, or ranges between 2, 3, 4, 8, 10, 16, or more. In some embodiments, the method comprises introducing amino acid substitutions into an anti-TM4SF1 antibody or antigen binding fragment thereof to achieve TM4SF1 affinity with a KD at pH 7.4 of at least about 100 nM as measured at 25° C. In certain embodiments, said method comprises generating an antibody library enriched for histidines in CDR residues or other residues that optimize the microenvironment affecting pKa. In some embodiments, the antibody library comprises anti-TM4SF1 antibodies or antigen binding fragments thereof with histidine residues introduced into a CDR position. In some embodiments, the antibody library comprises a series of anti-TM4SF1 antibodies or antigen binding fragments thereof, wherein each anti-TM4SF1 antibody in the antibody library comprises a single histidine substitution at a different CDR position. In some embodiments, the antibody library comprises a series of anti-TM4SF1 antibodies or antigen binding fragments thereof, each comprising 1, 2, 3, 4, 5, 6, 7, 8, 9, 10, 11, 12, 13, 14, 15, 16, or 17 mutations to histidine residues. In some embodiments, every CDR position is mutated to histidine in at least one of the TM4SF1 antibodies or antigen fragments of the antibody library.

In some embodiments, an anti-TM4SF1 antibody or antigen binding fragment thereof comprises 1, 2, 3, 4, 5, or more histidine substitutions in a CDR region. A histidine residue can be engineered into different positions of an anti-TM4SF1 antibody light chain (LC) or heavy chain (HC) for pH dependent binding affinity. Accordingly, in some embodiments, provided herein are heterobifunctional compounds with histidine engineered anti-TM4SF1 antibody or antigen binding fragment thereof. In some embodiments, an anti-TM4SF1 antibody or antigen binding fragment thereof comprises one or more histidine residues in CDR1, CDR2, and/or CDR3 of the light chain variable region (VL). In some embodiments, an anti-TM4SF1 antibody or antigen binding fragment thereof comprises one or more histidine residues in CDR1 of the light chain variable region (VL). In some embodiments, an anti-TM4SF1 antibody or antigen binding fragment thereof comprises one or more histidine residues in CDR2 of the light chain variable region (VL). In some embodiments, an anti-TM4SF1 antibody or antigen binding fragment thereof comprises one or more histidine residues in CDR3 of the light chain variable region (VL). In some embodiments, an anti-TM4SF1 antibody or antigen binding fragment thereof comprises one or more histidine residues in CDR1, CDR2, and/or CDR3 of the heavy chain variable region (VH). In some embodiments, an anti-TM4SF1 antibody or antigen binding fragment thereof comprises one or more histidine residues in CDR1 of the heavy chain variable region (VH). In some embodiments, an anti-TM4SF1 antibody or antigen binding fragment thereof comprises one or more histidine residues in CDR2 of the heavy chain variable region (VH). In some embodiments, an anti-TM4SF1 antibody or antigen binding fragment thereof comprises one or more histidine residues in CDR3 of the heavy chain variable region (VH). Accordingly, in some embodiments, the heterobifunctional compounds of the present disclosure comprise a histidine engineered anti-TM4SF1 antibody or antigen binding fragment thereof.

In some embodiments, an anti-TM4SF1 antibody or antigen binding fragment thereof comprises one or more histidine residues in CDR1, CDR2, and/or CDR3 of the light chain, for instance, in one or more of positions 30 (S30H), 92 (S92H), and 93 (N93H) of SEQ ID NO: 101 or SEQ ID NO: 131. In some embodiments, an anti-TM4SF1 antibody or antigen binding fragment thereof comprises one or more histidine residues in CDR1, CDR2, and/or CDR3 of the heavy chain, for instance in one or more of positions 28 (T28H), 31 (N31H), 32 (Y32H), 52 (N52H), 54 (Y54H), 57 (N57H), 100 (Q100H), and 101 (Y101H), of SEQ ID NO: 92 or SEQ ID NO: 130.

### Substitution at Position N297(Asn 297) and Conjugation of One or More Degraders to an Anti-TM4SF1 Antibody or Antigen Binding Fragment Thereof

Human IgG molecules have a conserved glycosylation site at each N297 residue in the CH2 domain, making these pendant N-glycans a convenient target for site-specific conjugation. This glycosylation site is sufficiently far from the variable region that conjugation of drug moieties to attached glycans should not impact antigen binding. In some embodiments of this disclosure, degrader compounds are linked to the glycans, using exemplary methods that include oxidative cleavage of the vicinal diol moieties contained in these glycans with periodate to generate aldehydes that can be reductively aminated and conjugated to hydrazide and aminooxy compounds. (See. e.g., O' Shannessy, et al. (1984) Immunol. Lett. 8:273-77).

Another method may include increasing the fucosylation of the N-acetylglucosamine residues in these glycans. Oxidation of these fucose residues can produce carboxylic acid and aldehyde moieties that can be used to link drugs and fluorophores to these specific sites on the antibody (See, e.g., Zuberbuhler, et al. (2012) Chem. Commun. 48:7100-02). Another method may include modifying sialic acid in these glycans (as well as increasing the sialic acid content in these glycans) followed by oxidation of the sialic acid and conjugation with aminooxy-drugs to form oxime-linked conjugates (See, e.g., Zhou, et al. (2014) Bioconjugate Chem. 25:510-20).

Alternatively, a sialyltransferase may be used to incorporate a modified sialic acid residue containing a bioorthogonal functional group into these glycans. The bioorthogonal functional group may then be modified to attach degrader compounds to the site of the glycan (See. e.g. Li, et al. (2014) Angew. Chem. Int. 53:7179-82). Another approach to modifying these glycan sites is the use of glycosyltransferases to link galactose, or galactose analogues containing ketones or azides, to the N-acetylglucosamine in these glycans, and linking drugs or radionucleotides to the galactose molecules (See, e.g. Khidekel, et al., (2003) J. Am. Chem. Soc. 125: 16162-63; Clark, et al., (2008) J. Am. Chem. Soc. 130: 11576-77; Boeggeman, et al. (2007) Bioconjugate Chem. 18:806-14). Another approach relies on the introduction of modified sugars into these glycans at the time of expression of the antibody by metabolic oligosaccharide engineering (See, e.g. Campbell, et al. (2007) Mol. BioSyst. 3: 187-94; Agard, et al., (2009) Acc. Chem. Res. 42:788-97).

In some embodiments, the anti-TM4SF1 antibody or antigen binding fragment thereof is conjugated to a degrader compound, by site-specific conjugation. Several native or engineered amino acids, including cysteines and glutamines, can be selected as the sites for conjugation.

In some instances, a cysteine residue can be engineered into different positions of antibody heavy chain (HC) or light chain (LC) for coupling, such as at position N297, i.e., N297C. Thus, in some embodiments, the DACs of the present disclosure comprise a cysteine engineered anti-TM4SF1 antibody or an antigen binding fragment thereof.

The introduction of a cysteine residue at a suitable position of the anti-TM4SF1 antibody may allow control of the site of conjugation and the obtained site-specific conjugates may be more homogeneous than the conjugates obtained via wild-type conjugation, i.e. conjugation via reduced interchain cysteines. In some cases, the DACs comprising at least one conjugation via cysteine may demonstrate at least equivalent in vivo potency, improved pharmacokinetics (PK), and an expanded therapeutic window compared to wild-type conjugates. The DAC, in some embodiments, comprises a cleavable dipeptide linker (i.e., valine-alanine) and degrader compound, which is linked to a cysteine at heavy chain position N297C in the Fc part of the anti-TM4SF1 antibody or antigen binding fragment thereof. In some cases, the DACs have an average degrader-to-antibody ratio (DAR) of greater than or equal to 1, such as a DAR of about 2, 6, 10 etc.

Without being bound by any particular theory, it is contemplated that site-specific conjugation through unpaired cysteine can be relatively simple and scalable. For instance, the degrader compounds coupling can be done without the need of special reagents. In some cases, DACs prepared through site-specific cysteines can show stronger in vivo antitumor activities and could be better tolerated than the conventional conjugates. In some embodiments, position N297 of the anti-TM4SF1 antibody or an antigen binding fragment thereof can be mutated to cysteine, i.e., N297C, and the cysteine residue can be conjugated to a degrader compound. In some instances, the N297C mutation is combined with additional mutations in nearby residues, to add stabilizing residues (e.g., arginine, lysine) and/or remove glutamic acid. In some cases, one or more positions from residue 292-303 are modified, in addition to N297C. The sequence for positions 292-303 can be REEQYCSTYRVV (SEQ ID NO: 163) (in IgG1), and REEQFCSTYRVV (SEQ ID NO: 164) (in IgG4).

In some embodiments, the anti-TM4SF1 antibody or antigen binding fragment thereof is conjugated to a degrader compounds, by site-specific conjugation through a glutamine residue. In some cases, microbial transglutaminase (mTG) can be used to transfer an amine containing drug-linker or a reactive spacer into Q295 residue in the heavy chain of an anti-TM4SF1 antibody or an antigen binding fragment thereof, for example, a deglycosylated anti-TM4SF1 antibody or an antigen binding fragment thereof. The conjugation can be optimized using a two-step chemoenzymatic approach whereby a reactive spacer containing a bioorthogonal azido or thiol functional linker is attached to the antibody by mTG and subsequently reacted with either dibenzocyclooctynes (DBCO) or maleimide containing MMAE. By using strain-promoted azide-alkyne cycloaddition (SPAAC) or thiol-maleimide chemistry, DACs can be generated with DAR, for example, at about 2.

In some instances, the anti-TM4SF1 antibody or antigen binding fragment thereof is conjugated to a degrader compound, by site-specific conjugations through a glutamine residue (e.g., Q295) as well as cysteine at position 297, N297C. This combination of mutations can open up two conjugation handles in the anti-TM4SF1 antibody or an antigen binding fragment thereof, and DACs of highers DAR can be obtained. The cysteine conjugation can be, for example, to maleimide, haloacetamide, or another partner. Bioconjugation modality and method may be optimized for improved DAC stability and efficacy. In some embodiments, one or more degrader compounds are conjugated to anti-TM4SF1 antibodies or antigen binding fragments via maleimide, e.g., cysteine-maleimide conjugation. Other functional groups besides maleimide, which in some instances are reactive with an anti-TM4SF1 antibody, such as a thiol group of a cysteine engineered anti-TM4SF1 antibody, include iodoacetamide, bromoacetamide, vinyl pyridine, disulfide, pyridyl disulfide, isocyanate, and isothiocyanate. In some embodiments, the degrader compounds are conjugated to anti-TM4SF1 antibodies or antigen binding fragments thereof via acetamide. For example, a degrader may be conjugated to an anti-TM4SF1 antibody or antigen binding fragment thereof via bromoacetamide conjugation.

In some embodiments, the anti-TM4SF1 antibodies and antigen binding fragments thereof, of the disclosure are specific to the ECL2 domain of TM4SF1. The amino acid sequence of human TM4SF1 ECL2 domain is EGPLCLDSLGQWNYTFASTEGQYLLDTSTWSECTEPKHIVEWNVSLFS (SEQ ID NO: 162).

As described in Table 16 below, included in the disclosure are novel antibodies that are specific to TM4SF1. The antibodies described in Table 16 are monoclonal murine antibodies AGX-A03, AGX-A04, AGX-A05, AGX-A07, AGX-A08, AGX-A09, and AGX-A 11, each of which were identified in the screen described in the Examples and bind the ECL2 region of TM4SF1. Further provided in Table 16 below are humanized antibodies h AGX-A07 and h AGX-A01.

In some embodiments, the anti-TM4SF1 antibodies or antigen-binding fragments thereof, comprise an IgG heavy chain constant region comprising an amino acid sequence set forth in SEQ ID NO: 87 or 88, or a sequence that is at least about 60%, at least about 65%, at least about 70%, at least about 75%, at least about, at least about 80%, at least about 85%, at least about 90%, at least about 95%, at least about 96%, at least about 97%, at least about 98%, at least about 99%, or 100% identical to SEQ ID NO: 73 or 74.

In another embodiment, the anti-TM4SF1 antibody or antigen-binding fragment thereof, comprises a light chain constant region comprising the amino acid sequence set forth in SEQ ID NO: 89, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 89.

In another embodiment, the anti-TM4SF1 antibody or antigen-binding fragment thereof, comprises a heavy chain variable domain comprising the amino acid sequence set forth in SEQ ID NO: 3, 15, 27, 39, 51, 63, or 75, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 3, 15, 27, 39, 51, 63, or 75.

In another embodiment, the anti-TM4SF1 antibody or antigen-binding fragment thereof is humanized and, comprises a heavy chain comprising the amino acid sequence set forth in SEQ ID NO: 90 or 92 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 90 or 92.

In another embodiment, the anti-TM4SF1 antibody or antigen-binding fragment thereof is humanized and, comprises a heavy chain comprising the amino acid sequence set forth in SEQ ID NO: 112 or 114, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 112 or 114.

In another embodiment, the anti-TM4SF1 antibody or antigen-binding fragment thereof, comprises a light chain variable domain comprising the amino acid sequence set forth in SEQ ID NO: 9, 21, 33, 45, 57, 69, or 81, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 9, 21, 33, 45, 57, 69, or 81.

In another embodiment, the anti-TM4SF1 antibody or antigen-binding fragment thereof is humanized and, comprises a light chain variable domain comprising the amino acid sequence set forth in SEQ ID NO: 97, 99, 101, 103, or 105 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, 101, 103 or 105. In another embodiment, the antibody or antigen-binding fragment thereof is humanized and, comprises a light chain variable domain comprising the amino acid sequence set forth in SEQ ID NO: 97, 99, or 101 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, or 101.

In another embodiment, the anti-TM4SF1 antibody or antigen-binding fragment thereof is humanized and, comprises a light chain variable domain comprising the amino acid sequence set forth in SEQ ID NO: 122, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 122.

In some embodiments, the anti-TM4SF1 antibody or antigen binding fragment thereof comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 6, 18, 30, 42, 54, 66, or 78. In some embodiments, the anti-TM4SF1 antibody or antigen binding fragment thereof comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 7, 19, 31, 43, 55, 67, or 79. In some embodiments, the anti-TM4SF1 antibody or antigen binding fragment thereof comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 8, 20, 32, 44, 56, 68, or 80.

In some embodiments, the anti-TM4SF1 antibody or antigen binding fragment thereof comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 12, 24, 36, 48, 60, 72, or 84. In some embodiments, the anti-TM4SF1 antibody or antigen binding fragment thereof comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 13, 25, 37, 49, 61, 73, or 85. In some embodiments, the anti-TM4SF1 antibody or antigen binding fragment thereof comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 14, 26, 38, 50, 62, 74, or 86.

In some embodiments, the anti-TM4SF1 antibody or antigen binding fragment thereof is humanized and comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 94 or SEQ ID NO: 115. In some embodiments, the anti-TM4SF1 antibody or antigen binding fragment thereof is humanized and comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 95, SEQ ID NO: 116, or SEQ ID NO: 117. In some embodiments, the anti-TM4SF1 antibody or antigen binding fragment thereof is humanized and comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 96, SEQ ID NO: 118, SEQ ID NO: 119, SEQ ID NO: 120, or SEQ ID NO: 121.

In some embodiments, the anti-TM4SF1 antibody or antigen binding fragment thereof is humanized and comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 107, SEQ ID NO: 108, SEQ ID NO: 124, SEQ ID NO: 125, SEQ ID NO: 126, or SEQ ID NO: 127. In some embodiments, the anti-TM4SF1 antibody or antigen binding fragment thereof is humanized comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 109 or SEQ ID NO: 128. In some embodiments, the anti-TM4SF1 antibody or antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, SEQ ID NO: 111, or SEQ ID NO: 129. In some embodiments, the anti-TM4SF1 antibody or antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, or SEQ ID NO: 129.

The amino acid sequences of murine monoclonal antibody AGX-A03 are described in Table 16. Specifically, the heavy chain CDR sequences are set forth in SEQ ID Nos: 6, 7, and 8 (CDR1, CDR2, and CDR3), and the light chain CDR amino acid sequences are set forth in SEQ ID Nos: 12, 13, and 14 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 6, 7, and 8 and/or a light chain variable region comprising CDRs as set forth in the amino acid sequences of SEQ ID Nos: 12, 13, and 14. Included in the disclosure are humanized antibodies or antigen binding fragments comprising the CDRs of AGX-A03. Further, the heavy chain variable amino acid sequences and the light chain variable amino acid sequences of AGX-A03 are described in SEQ ID NOS: 3 and 9, respectively.

The amino acid sequences of murine monoclonal antibody AGX-A04 are described in Table 16. Specifically, the heavy chain CDR sequences are set forth in SEQ ID Nos: 18, 19, and 20 (CDR1, CDR2, and CDR3), and the light chain CDR amino acid sequences are set forth in SEQ ID Nos: 24, 25, and 26 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 18, 19, and 20 and/or a light chain variable region comprising CDRs as set forth in the amino acid sequences of SEQ ID Nos: 24, 25, and 26. Included in the disclosure are humanized antibodies or antigen binding fragments comprising the CDRs of AGX-A04. Further, the heavy chain variable amino acid sequences and the light chain variable amino acid sequences of AGX-A04 are described in SEQ ID NOS: 15 and 21, respectively.

The amino acid sequences of murine monoclonal antibody AGX-A05 are described in Table 16. Specifically, the heavy chain CDR sequences are set forth in SEQ ID Nos: 30, 31, and 32 (CDR1, CDR2, and CDR3), and the light chain CDR amino acid sequences are set forth in SEQ ID Nos: 36, 37, and 38 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 30, 31, and 32 and/or a light chain variable region comprising CDRs as set forth in the amino acid sequences of SEQ ID Nos: 36, 37, and 38. Included in the disclosure are humanized antibodies or antigen binding fragments comprising the CDRs of AGX-A05. Further, the heavy chain variable amino acid sequences and the light chain variable amino acid sequences of AGX-A05 are described in SEQ ID NOS: 27 and 33, respectively. The amino acid sequences of murine monoclonal antibody AGX-A07 are described in Table 16. Specifically, the heavy chain CDR sequences are set forth in SEQ ID Nos: 42, 43, and 44 (CDR1, CDR2, and CDR3), and the light chain CDR amino acid sequences are set forth in SEQ ID Nos: 48, 49, and 50 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 42, 43, and 44 and/or a light chain variable region comprising CDRs as set forth in the amino acid sequences of SEQ ID Nos: 48, 49, and 50. Included in the disclosure are humanized antibodies or antigen binding fragments comprising the CDRs of AGX-A07. Further, the heavy chain variable amino acid sequences and the light chain variable amino acid sequences of AGX-A07 are described in SEQ ID NOs: 39 and 45, respectively.

In one embodiment, a humanized AGX-A07 (h AGX-A07) antibody or antigen binding fragments thereof is provided, comprising a heavy chain sequence as forth in the amino acid sequence of SEQ ID NO: 90. In some embodiments, the humanized AGX-A07 antibody or antigen binding fragments thereof is a humanized mutated AGX-A07 (hm AGX-A07) antibody or antigen binding fragments thereof, comprising a heavy chain sequence comprising one or more substitutions in the sequence as set forth in the amino acid sequence of SEQ ID NO: 90. As shown in Table 16, the heavy chain sequence set forth in SEQ ID NO: 90 is also referred to herein as AGX-A07 H2. In some embodiments, the humanized AGX-A07 antibody or antigen binding fragments thereof is a humanized mutated AGX-A07 antibody or antigen binding fragments thereof, comprising a heavy chain sequence comprising one or more substitutions in the sequence as set forth in the amino acid sequence of SEQ ID NO: 90, wherein the one or more substitutions are in amino acid positions 1, 44, and 80 of SEQ ID NO: 90. In some cases, the humanized mutated AGX-A07 antibody or antigen binding fragments thereof comprises an E 1Q (glutamic acid to glutamine substitution at position 1 of the heavy chain, SEQ ID NO: 90). In some cases, the humanized mutated AGX-A07 antibody or antigen binding fragments thereof comprises a D44G (aspartate to glycine substitution at position 44 of the heavy chain, SEQ ID NO: 90). In some cases, the humanized mutated AGX-A07 antibody or antigen binding fragments thereof comprises a F80Y (phenyl alanine to tyrosine substitution at position 80 of the heavy chain, SEQ ID NO: 90). In some embodiments, a humanized mutated AGX-A07 antibody or antigen binding fragments is provided, comprising a heavy chain sequence as forth in the amino acid sequence of SEQ ID NO: 92. As shown in Table 16, the heavy chain sequence set forth in SEQ ID NO: 92 is also referred to herein as AGX-A07 H2v1. In some embodiments, humanized AGX-A07 antibodies or antigen binding fragments are provided, comprising a light chain sequence as forth in the amino acid sequence of SEQ ID NO: 97. As shown in Table 16, the light chain sequence set forth in SEQ ID NO: 97 is also referred to herein as AGX-A07 L5. In some embodiments, the humanized AGX-A07 antibody or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97. In some embodiments, the humanized AGX-A07 antibodies or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97, wherein the one or more substitutions are in amino acid positions 3, 26, 62, and 90 of SEQ ID NO: 97. In some cases, the humanized mutated AGX-A07 antibody or antigen binding fragments thereof comprises an I3V (isoluecine to valine substitution at position 3 of the light chain, SEQ ID NO: 97). In some cases, the humanized mutated AGX-A07 antibody or antigen binding fragments thereof comprises a N26Q (asparagine to glutamine substitution at position 26 of the light chain, SEQ ID NO: 97). In some cases, the humanized mutated AGX-A07 antibody or antigen binding fragments thereof comprises a N26S (asparagine to serine substitution at position 26 of the light chain, SEQ ID NO: 97). In some cases, the humanized mutated AGX-A07 antibody or antigen binding fragments thereof comprises a G62S (glycine to serine substitution at position 62 of the light chain, SEQ ID NO: 97). In some cases, the humanized mutated AGX-A07 antibody or antigen binding fragments thereof comprises a W90Y (tryptophan to tyrosine substitution at position 90 of the light chain, SEQ ID NO: 97). In some embodiments, humanized mutated AGX-A07 antibodies or antigen binding fragments are provided, comprising a light chain sequence as forth in an amino acid sequence selected from the group consisting of SEQ ID NO: 99, SEQ ID NO: 101, SEQ ID NO: 103, and SEQ ID NO: 105.

As shown in Table 16, the light chain sequence set forth in SEQ ID NO: 99 is also referred to herein as AGX-A07 L5v1, the light chain sequence set forth in SEQ ID NO: 101 is also referred to herein as AGX-A07 L5v2, the light chain sequence set forth in SEQ ID NO: 103 is also referred to herein as AGX-A07 L5v3, and the light chain sequence set forth in SEQ ID NO: 105 is also referred to herein as AGX-A07 L5v4. Exemplary coding sequence for the heavy chain of a humanized AGX-A07 antibody or antigen binding fragment thereof is provided in SEQ ID NO: 91. Exemplary coding sequence for the heavy chain of a humanized mutated AGX-A07 antibody or antigen binding fragment thereof is provided in SEQ ID NO: 93. Exemplary coding sequence for the light chain of a humanized AGX-A07 antibody or antigen binding fragment thereof is provided in SEQ ID NO: 98 (AGX-A07 L5). Exemplary coding sequences for the light chain of a humanized mutated AGX-A07 antibody or antigen binding fragment thereof are provided in SEQ ID NO: 100 (AGX-A07 L5v1), SEQ ID NO: 102 (AGX-A07 L5v2), SEQ ID NO: 104 (AGX-A07 L5v3), and SEQ ID NO: 106 (AGX-A07 L5v4).

In one embodiment, a humanized AGX-A07 antibody or antigen binding fragments thereof is provided, comprising a heavy chain variable domain sequence as forth in the amino acid sequence of SEQ ID NO: 130 or SEQ ID NO: 132. In some embodiments, the humanized AGX-A07 antibody or antigen binding fragments thereof is a humanized mutated AGX-A07 antibody or antigen binding fragments thereof, comprising a heavy chain variable domain sequence comprising one or more substitutions in the sequence as set forth in the amino acid sequence of SEQ ID NO: 130 or SEQ ID NO: 132. In one embodiment, a humanized AGX-A07 antibody or antigen binding fragments thereof is provided, comprising a light chain variable domain sequence as forth in the amino acid sequence of SEQ ID NO: 131 or SEQ ID NO: 133. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 131 or SEQ ID NO: 133.

In some embodiments, the humanized AGX-A07 antibody or antigen binding fragment thereof is a humanized mutated AGX-A07 antibody or antigen binding fragment thereof comprising a light chain variable domain sequence comprising the sequence as set forth in the amino acid sequence of SEQ ID NO: 131 and a heavy chain variable domain sequence comprising the sequence as set forth in the amino acid sequence of SEQ ID NO: 130. In some embodiments, the humanized AGX-A07 antibody or antigen binding fragment thereof is a humanized mutated AGX-A07 antibody or antigen binding fragments thereof, comprising a light chain variable domain sequence comprising one or more substitutions in the sequence as set forth in the amino acid sequence of SEQ ID NO: 131 and a heavy chain variable domain sequence comprises one or more substitutions in the sequence as set forth in the amino acid sequence of SEQ ID NO: 130. In some embodiments, the humanized AGX-A07 antibody or antigen binding fragments thereof is a humanized mutated AGX-A07 antibody or antigen binding fragments thereof comprising a light chain variable domain sequence comprising the sequence as set forth in the amino acid sequence of SEQ ID NO: 133 and a heavy chain variable domain sequence comprising the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 133 and a heavy chain variable domain sequence comprises one or more substitutions in the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof comprising a heavy chain sequence comprising the sequence as set forth in the amino acid sequence of SEQ ID NO: 156, or a sequence comprising one of more substitutions in the amino acid sequence of SEQ ID NO: 156.

In some cases, the humanized AGX-A07 antibodies or antigen binding fragments thereof comprise heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3). In some cases, the humanized mutated AGX-A07 antibodies or antigen binding fragments thereof comprises heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3).

In some cases, the humanized mutated AGX-A07 antibodies or antigen binding fragments thereof comprise heavy chain CDR1 sequence as set forth in SEQ ID NO: 94, or a heavy chain CDR1 sequence comprising one or more substitutions in the sequences as set forth in SEQ ID NO: 94. In some cases, the humanized mutated AGX-A07 antibodies or antigen binding fragments thereof comprise a heavy chain CDR2 sequence as set forth in SEQ ID NO: 95, or a heavy chain CDR2 sequence comprising one or more substitutions in the sequences as set forth in SEQ ID NO: 95. In some cases, the humanized mutated AGX-A07 antibodies or antigen binding fragments thereof comprise a heavy chain CDR3 sequence as set forth in SEQ ID NO: 96, or a heavy chain CDR3 sequence comprising one or more substitutions in the sequences as set forth in SEQ ID NO: 96.

In some cases, the humanized AGX-A07 antibodies or antigen binding fragments thereof comprise light chain CDR sequences as set forth in SEQ ID Nos: 107, 109, and 110 (CDR1, CDR2, and CDR3), or CDR sequences comprising one or more substitutions in the sequences as set forth in SEQ ID Nos: 107, 109, and 110 (CDR1, CDR2, and CDR3). In some cases, the humanized AGX-A07 antibodies or antigen binding fragments thereof comprise light chain CDR sequences as set forth in SEQ ID Nos: 107, 109, and 111 (CDR1, CDR2, and CDR3), or CDR sequences comprising one or more substitutions in the sequences as set forth in SEQ ID Nos: 107, 109, and 111 (CDR1, CDR2, and CDR3). In some cases, the humanized AGX-A07 antibodies or antigen binding fragments thereof comprise light chain CDR sequences as set forth in SEQ ID Nos: 108, 109, and 110 (CDR1, CDR2, and CDR3), or CDR sequences comprising one or more substitutions in the sequences as set forth in SEQ ID Nos: 108, 109, and 110 (CDR1, CDR2, and CDR3). In some cases, the humanized AGX-A07 antibodies or antigen binding fragments thereof comprise light chain CDR sequences as set forth in SEQ ID Nos: 108, 109, and 111 (CDR1, CDR2, and CDR3), or CDR sequences comprising one or more substitutions in the sequences as set forth in SEQ ID Nos: 108, 109, and 111 (CDR1, CDR2, and CDR3).

In some cases, the humanized mutated AGX-A07 antibodies or antigen binding fragments thereof comprise light chain CDR1 sequence as set forth in SEQ ID Nos: 107 or 108, or light chain CDR1 sequence comprising one or more substitutions in the sequences as set forth in SEQ ID Nos: 107 or 108. In some cases, the humanized mutated AGX-A07 antibodies or antigen binding fragments thereof comprise light chain CDR2 sequence as set forth in SEQ ID NO: 109, or light chain CDR2 sequence comprising one or more substitutions in the sequences as set forth in SEQ ID NO: 109. In some cases, the humanized mutated AGX-A07 antibodies or antigen binding fragments thereof comprise light chain CDR3 sequence as set forth in SEQ ID Nos: 110 or 111, or light chain CDR1 sequence comprising one or more substitutions in the sequences as set forth in SEQ ID Nos: 110 or 111. In some cases, the humanized mutated AGX-A07 antibodies or antigen binding fragments thereof comprise light chain CDR3 sequence as set forth in SEQ ID NO: 110, or light chain CDR1 sequence comprising one or more substitutions in the sequences as set forth in SEQ ID Nos: 110.

In some embodiments, the humanized mutated AGX-A07 comprises a heavy chain variable region comprising the following amino acid substitutions: Q1E, D44G, F80Y in SEQ ID NO: 132 (also referred to herein as AGX-A07 H2), and a light chain variable region comprising the following amino acid substitutions: I3V, N26Q, G62S in SEQ ID NO: 133 (also referred to herein as AGX-A07 L5). In some embodiments, the humanized mutated AGX-A07 comprises a heavy chain variable region comprising the following amino acid substitutions: Q1E, D44G, F80Y in SEQ ID NO: 132, and a light chain variable region comprising the following amino acid substitutions: I3V, N26Q, G62S in SEQ ID NO: 133, wherein the heavy chain comprises CDR1 (SEQ ID NO: 94), CDR2 (SEQ ID NO: 95), and CDR3 (SEQ ID NO: 96), and the light chain comprises CDR1 (SEQ ID NO: 108), CDR2 (SEQ ID NO: 109), and CDR3 (SEQ ID NO: 110). In some embodiments, the humanized mutated AGX-A07 is AGX-A07 H2v1L5v2 and comprises a heavy chain comprising the amino acid sequence as set forth in SEQ ID NO: 130 (also referred to herein as AGX-A07 H2v1), and a light chain comprising the amino acid sequence as set forth in SEQ ID NO: 131 (also referred to herein as AGX-A07 L5v2). In some embodiments, the humanized mutated AGX-A07 comprises a heavy chain comprising the amino acid sequence as set forth in SEQ ID NO: 92, and a light chain comprising the amino acid sequence as set forth in SEQ ID NO: 101.

The amino acid sequences of murine monoclonal antibody AGX-A08 are described in Table 16. Specifically, the heavy chain CDR sequences are set forth in SEQ ID Nos: 54, 55, and 56 (CDR1, CDR2, and CDR3), and the light chain CDR amino acid sequences are set forth in SEQ ID Nos: 60, 61, and 62 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 54, 55, and 56 and/or a light chain variable region comprising CDRs as set forth in the amino acid sequences of SEQ ID Nos: 60, 61, and 62. Included in the disclosure are humanized antibodies or antigen binding fragments comprising the CDRs of AGX-A08. Further, the heavy chain variable amino acid sequences and the light chain variable amino acid sequences of AGX-A08 are described in SEQ ID NOs: 51 and 57, respectively.

The amino acid sequences of murine monoclonal antibody AGX-A09 are described in Table 16. Specifically, the heavy chain CDR sequences are set forth in SEQ ID Nos: 66, 67, and 68 (CDR1, CDR2, and CDR3), and the light chain CDR amino acid sequences are set forth in SEQ ID Nos: 72, 73, and 74 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 66, 67, and 68 and/or a light chain variable region comprising CDRs as set forth in the amino acid sequences of SEQ ID Nos: 72, 73, and 74. Included in the disclosure are humanized antibodies or antigen binding fragments comprising the CDRs of AGX-A09. Further, the heavy chain variable amino acid sequences and the light chain variable amino acid sequences of AGX-A09 are described in SEQ ID NOs: 63 and 69, respectively.

The amino acid sequences of murine monoclonal antibody AGX-A11 are described in Table 16. Specifically, the heavy chain CDR sequences are set forth in SEQ ID Nos: 78, 79, and 80 (CDR1, CDR2, and CDR3), and the light chain CDR amino acid sequences are set forth in SEQ ID Nos: 84, 85, and 86 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 78, 79, and 80 and/or a light chain variable region comprising CDRs as set forth in the amino acid sequences of SEQ ID Nos: 84, 85, and 862. Included in the disclosure are humanized antibodies or antigen binding fragments comprising the CDRs of AGX-A11. Further, the heavy chain variable amino acid sequences and the light chain variable amino acid sequences of AGX-A11 are described in SEQ ID NOS: 75 and 81, respectively.

The amino acid sequences of a humanized antibody AGX-A01 (h AGX-A01) are described in Table 16. As shown in Table 16, the heavy chain sequence set forth is SEQ ID NO: 112 is also referred to herein as AGX-A01 H1. Specifically, the heavy chain CDR sequences are set forth in SEQ ID Nos: 115, 116, and 118 (CDR1, CDR2, and CDR3) and the light chain CDR amino acid sequences are set forth in SEQ ID Nos: 124, 128, and 129 (CDR1, CDR2, and CDR3). Further, exemplary heavy chain amino acid sequence and the light chain amino acid sequence of the humanized AGX-A01 are described in SEQ ID Nos: 112 and 122, respectively. Exemplary coding sequences for the heavy chain and the light chain of the humanized AGX-A01 are described in SEQ ID Nos: 113 and 123, respectively

In some embodiments, the humanized AGX-A01 antibody or antigen binding fragments thereof is a humanized mutated AGX-A01 (hm AGX-A01) antibody or antigen binding fragments thereof, comprising a heavy chain sequence comprising one or more substitutions in the sequence as set forth in the amino acid sequence of SEQ ID NO: 112. In some embodiments, the humanized AGX-A01 antibody or antigen binding fragments thereof is a humanized mutated AGX-A01 antibody or antigen binding fragments thereof, comprising a heavy chain sequence comprising one or more substitutions in the sequence as set forth in the amino acid sequence of SEQ ID NO: 112, wherein the one or more substitutions are in amino acid positions 63 and 106 of SEQ ID NO: 112. In some cases, the humanized mutated AGX-A01 antibody or antigen binding fragments thereof comprises a G63S (glycine to serine substitution at position 63 of the heavy chain, SEQ ID NO: 112). In some cases, the humanized mutated AGX-A01 antibody or antigen binding fragments thereof comprises a D106E (aspartate to glutamic acid substitution at position 106 of the heavy chain, SEQ ID NO: 112). In some cases, the humanized mutated AGX-A01 antibody or antigen binding fragments thereof comprises a D106S (aspartate to serine substitution at position 106 of the heavy chain, SEQ ID NO: 112). In some embodiments, a humanized mutated AGX-A01 antibody or antigen binding fragments is provided, comprising a heavy chain sequence as forth in the amino acid sequence of SEQ ID NO: 114. As shown in Table 16, the heavy chain sequence set forth is SEQ ID NO: 114 is also referred to herein as AGX-A01 H1v1.

In some embodiments, humanized AGX-A01 antibodies or antigen binding fragments are provided, comprising a light chain sequence as forth in the amino acid sequence of SEQ ID NO: 122. As shown in Table 16, the light chain sequence set forth is SEQ ID NO: 122 is also referred to herein as AGX-A01 L10. In some embodiments, the humanized AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, 86, and 90 of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, and 86 of SEQ ID NO: 122. In some cases, the humanized mutated AGX-A01 antibody or antigen binding fragments thereof comprises an A1E (alanine to glutamic acid substitution at position 1 of the light chain, SEQ ID NO: 122). In some cases, the humanized mutated AGX-A01 antibody or antigen binding fragments thereof comprises a N33S (asparagine to serine substitution at position 33 of the light chain, SEQ ID NO: 122). In some cases, the humanized mutated AGX-A01 antibody or antigen binding fragments thereof comprises a M42Q (methionine to glutamine substitution at position 42 of the light chain, SEQ ID NO: 122). In some cases, the humanized mutated AGX-A01 antibody or antigen binding fragments thereof comprises a V51L (valine to leucine substitution at position 51 of the light chain, SEQ ID NO: 122). In some cases, the humanized mutated AGX-A01 antibody or antigen binding fragments thereof comprises a D86E (aspartate to glutamic acid substitution at position 86 of the light chain, SEQ ID NO: 122). In some cases, the humanized mutated AGX-A01 antibody or antigen binding fragments thereof comprises an I90V (isoleucine to valine substitution at position 90 of the light chain, SEQ ID NO: 122).

In some cases, the humanized AGX-A01 antibodies or antigen binding fragments thereof comprise heavy chain CDR sequences as set forth in SEQ ID Nos: 115 (CDR1); 116 (CDR2); and 118 (CDR3), or CDR sequences comprising one or more substitutions in the sequences as set forth in SEQ ID Nos: 115 (CDR1); 116 (CDR2); and 118 (CDR3). In some cases, the humanized mutated AGX-A01 antibodies or antigen binding fragments thereof comprise heavy chain CDR sequences as set forth in SEQ ID Nos: 115 (CDR1); 116 or 117 (CDR2); and 118, 119, 120, or 121 (CDR3), or CDR sequences comprising one or more substitutions in the sequences as set forth in SEQ ID Nos: 115 (CDR1); 116 or 117 (CDR2); and 118, 119, 120, or 121 (CDR3).

In some cases, the humanized mutated AGX-A01 antibodies or antigen binding fragments thereof comprise heavy chain CDR1 sequence as set forth in SEQ ID NO: 115, or a heavy chain CDR1 sequence comprising one or more substitutions in the sequences as set forth in SEQ ID NO: 115. In some cases, the humanized mutated AGX-A01 antibodies or antigen binding fragments thereof comprise a heavy chain CDR2 sequence as set forth in SEQ ID NO: 116, or a heavy chain CDR2 sequence comprising one or more substitutions in the sequences as set forth in SEQ ID NO: 116. In some cases, the humanized mutated AGX-A01 antibodies or antigen binding fragments thereof comprise a heavy chain CDR2 sequence as set forth in SEQ ID NO: 117, or a heavy chain CDR2 sequence comprising one or more substitutions in the sequences as set forth in SEQ ID NO: 117. In some cases, the humanized mutated AGX-A01 antibodies or antigen binding fragments thereof comprise a heavy chain CDR3 sequence as set forth in a sequence selected from SEQ ID Nos: 118, 119, 120 and 121, or a heavy chain CDR3 sequence comprising one or more substitutions in a sequence selected from SEQ ID Nos: 118, 119, 120, and 121.

In some cases, the humanized AGX-A01 antibodies or antigen binding fragments thereof comprise light chain CDR sequences as set forth in SEQ ID Nos: 124 (CDR1); 128 (CDR2); and 129 (CDR3), or CDR sequences comprising one or more substitutions in the sequences as set forth in SEQ ID Nos: 124 (CDR1); 128 (CDR2); and 129 (CDR3). In some cases, the humanized mutated AGX-A01 antibodies or antigen binding fragments thereof comprise light chain CDR sequences as set forth in SEQ ID Nos: 124, 125, 126, or 127 (CDR1); 128 (CDR2); and 129 (CDR3), or CDR sequences comprising one or more substitutions in the sequences as set forth in SEQ ID Nos: 124, 125, 126, or 127 (CDR1); 128 (CDR2); and 129 (CDR3).

In some cases, the humanized mutated AGX-A01 antibodies or antigen binding fragments thereof comprise light chain CDR1 sequence as set forth in SEQ ID Nos: 125, 126, 127, or 128, or light chain CDR1 sequence comprising one or more substitutions in the sequences as set forth in SEQ ID Nos: 125, 126, 127, or 128. In some cases, the humanized mutated AGX-A01 antibodies or antigen binding fragments thereof comprise light chain CDR2 sequence as set forth in SEQ ID NO: 129, or light chain CDR2 sequence comprising one or more substitutions in the sequences as set forth in SEQ ID NO: 129. In some cases, the humanized mutated AGX-A01 antibodies or antigen binding fragments thereof comprise light chain CDR3 sequence as set forth in SEQ ID Nos: 130, or light chain CDR1 sequence comprising one or more substitutions in the sequences as set forth in SEQ ID Nos: 130.

In one embodiment, the disclosure provides an anti-TM4SF1 antibody, or antigen-binding fragment thereof, that comprises a heavy chain variable domain encoded by a nucleic acid sequence as set forth in SEQ ID NO: 3, and a light chain variable domain encoded by a nucleic acid sequence as set forth in SEQ ID NO: 9. In one embodiment, the disclosure provides an anti-TM4SF1 antibody, or antigen-binding fragment thereof, that comprises a heavy chain variable domain encoded by a nucleic acid sequence as set forth in SEQ ID NO: 15, and a light chain variable domain encoded by a nucleic acid sequence as set forth in SEQ ID NO: 21 In one embodiment, the disclosure provides an anti-TM4SF1 antibody, or antigen-binding fragment thereof, that comprises a heavy chain variable domain encoded by a nucleic acid sequence as set forth in SEQ ID NO: 27, and a light chain variable domain encoded by a nucleic acid sequence as set forth in SEQ ID NO: 33. In one embodiment, the disclosure provides an anti-TM4SF1 antibody, or antigen-binding fragment thereof, that comprises a heavy chain variable domain encoded by a nucleic acid sequence as set forth in SEQ ID NO: 39, and a light chain variable domain encoded by a nucleic acid sequence as set forth in SEQ ID NO: 45. In one embodiment, the disclosure provides an anti-TM4SF1 antibody, or antigen-binding fragment thereof, that comprises a heavy chain variable domain encoded by a nucleic acid sequence as set forth in SEQ ID NO: 51, and a light chain variable domain encoded by a nucleic acid sequence as set forth in SEQ ID NO: 57. In one embodiment, the disclosure provides an anti-TM4SF1 antibody, or antigen-binding fragment thereof, that comprises a heavy chain variable domain encoded by a nucleic acid sequence as set forth in SEQ ID NO: 63, and a light chain variable domain encoded by a nucleic acid sequence as set forth in SEQ ID NO: 69. In one embodiment, the disclosure provides an anti-TM4SF1 antibody, or antigen-binding fragment thereof, that comprises a heavy chain variable domain encoded by a nucleic acid sequence as set forth in SEQ ID NO: 75, and a light chain variable domain encoded by a nucleic acid sequence as set forth in SEQ ID NO: 81. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 105. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 105.

In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, SEQ ID NO: 103, SEQ ID NO: 105, or SEQ ID NO: 122. In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, or SEQ ID NO: 122.

In one embodiment, the disclosure includes an anti-TM4SF1 antibody which is an IgG and comprises four polypeptide chains including two heavy chains each comprising a heavy chain variable domain and heavy chain constant regions CH1, CH2 and CH3, and two light chains each comprising a light chain variable domain and a light chain constant region (CL). In certain embodiments, the antibody is a human IgG1, IgG2, or an IgG4. In certain embodiments, the antibody is a human IgG1. In other embodiments, the antibody is an IgG2. The heavy and light chain variable domain sequences may contain CDRs as set forth in Table 16.

Complementarity determining regions (CDRs) are known as hypervariable regions both in the light chain and the heavy chain variable domains. The more highly conserved portions of variable domains are called the framework (FR). CDRs and framework regions (FR) of a given antibody may be identified using the system described by Kabat et al. supra; Lefranc et al., supra and/or Honegger and Pluckthun, supra. Also familiar to those in the art is the numbering system described in Kabat et al. (1991, NIH Publication 91-3242, National Technical Information Service, Springfield, Va.). In this regard Kabat et al. defined a numbering system for variable domain sequences, including the identification of CDRs, that is applicable to any antibody.

One or more CDRs may be incorporated into a molecule either covalently or noncovalently to make it an antigen binding protein.

An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest. The CDR3, in particular, is known to play an important role in antigen binding of an antibody or antibody fragment.

In one embodiment, the disclosure provides an anti-TM4SF1 antibody, or an antigen-binding fragment thereof, comprising a heavy chain comprising a CDR3 domain as set forth in any one of SEQ ID NO: 8, SEQ ID NO: 20, SEQ ID NO: 32, SEQ ID NO: 44, SEQ ID NO: 56, SEQ ID NO: 68, or SEQ ID NO: 80 and comprising a variable domain comprising an amino acid sequence that has at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR3 domain as set forth in any one of SEQ ID NO: 14, SEQ ID NO: 26, SEQ ID NO: 38, SEQ ID NO: 50, SEQ ID NO: 62, SEQ ID NO: 74, or SEQ ID NO: 86, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR3 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR2 domain as set forth in any one of SEQ ID NO: 7, SEQ ID NO: 19, SEQ ID NO: 31, SEQ ID NO: 43, SEQ ID NO: 55, SEQ ID NO: 67, or SEQ ID NO: 79 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR2 domain as set forth in any one of SEQ ID NO: 13, SEQ ID NO: 25, SEQ ID NO: 37, SEQ ID NO: 49, SEQ ID NO: 61, SEQ ID NO: 73, or SEQ ID NO: 85, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR2 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR1 domain as set forth in any one of SEQ ID NO: 6, SEQ ID NO: 18, SEQ ID NO: 30, SEQ ID NO: 42, SEQ ID NO: 54, SEQ ID NO: 66, or SEQ ID NO: 78 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 45, SEQ ID NO: 69, or SEQ ID NO: 81. In one embodiment, the disclosure provides an anti-TM4SF1 antibody, or an antigen-binding fragment thereof, comprising a light chain comprising a CDR1 domain as set forth in any one of SEQ ID NO: 12, SEQ ID NO: 24, SEQ ID NO: 36, SEQ ID NO: 48, SEQ ID NO: 60, SEQ ID NO: 72, or SEQ ID NO: 84, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence a set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR1 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent.

In some embodiments, an anti-TM4SF1 antibody of this disclosure comprises a heavy chain comprising an Fc region, wherein said Fc region comprises a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153; or wherein said Fc region comprises a sequence comprising one or more substitutions in a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153. For instance, in some embodiments, an anti-TM4SF1 antibody of this disclosure comprises an Fc region, wherein said Fc region comprises a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153.

In some embodiments, an anti-TM4SF1 antibody of this disclosure comprises a heavy chain comprising a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156; or wherein said heavy chain comprises a sequence comprising one or more substitutions in a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156. For instance, in some embodiments, an anti-TM4SF1 antibody of this disclosure comprises a heavy chain comprising a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156.

The anti-TM4SF1 antibodies and fragments described in Table 16 may also be humanized. Various methods for humanizing non-human antibodies are disclosed in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (See. e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6 I) and VH subgroup III (VHIII). In another method, human germline genes are used as the source of the framework regions.

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human framework regions that may be used for humanization include but are not limited to: framework regions selected using the “best-fit” method (see, e.g., Sims, et al., J. Immunol. 151 (1993) 2296); framework regions derived from the consensus sequence of human antibodies of a particular subgroup of light or heavy chain variable regions (see, e.g., Carter, et al., Proc. Natl. Acad. Sci. USA, 89 (1992) 4285; and Presta, et al., J. Immunol., 151 (1993) 2623); human mature (somatically mutated) framework regions or human germline framework regions (see, e.g., Almagro, and Fransson, Front. Biosci. 13 (2008) 1619-1633); and framework regions derived from screening FR libraries (see, e.g., Baca, et al., J. Biol. Chem. 272 (1997) 10678-10684 and Rosok, et al., J. Biol. Chem. 271 (1996) 22611-22618).

Humanized antibodies and methods of making them are reviewed, e.g., in Almagro, and Fransson, Front. Biosci. 13 (2008) 1619-1633, and are further described, e.g., in Riechmann, et al., Nature 332 (1988) 323-329; Queen, et al., Proc. Nat'l Acad. Sci. USA 86 (1989) 10029-10033; U.S. Pat. Nos. 5,821,337, 7,527,791, 6,982,321, and 7,087,409; Kashmiri, et al., Methods 36 (2005) 25-34 (describing SDR (a-CDR) grafting); Padlan, Mol. Immunol. 28 (1991) 489-498 (describing “resurfacing”); Dall'Acqua, et al., Methods 36 (2005) 43-60 (describing “FR shuffling”); and Osboum, et al., Methods 36 (2005) 61-68 and Klimka, et al., Br. J. Cancer, 83 (2000) 252-260 (describing the “guided selection” approach to FR shuffling).

In one embodiment, an anti-TM4SF1 antibody, or antigen-binding fragment thereof, of the disclosure binds to cynomolgus TM4SF1 with a K_{D} about 1×10⁻⁶ M or less.

An anti-TM4SF1 antibody, or antigen-binding fragment thereof, of the disclosure, in certain embodiments, binds to an epitope on the ECL2 loop of human TM4SF1 with a K_{D} about 5×10⁻⁸ M or less as determined in a standard flow cytometry assay using HUVEC cells.

An anti-TM4SF1 antibody, or antigen-binding fragment thereof, of the disclosure, in certain embodiments, binds to human TM4SF1 with a K_{D} of about 1×10⁻⁸ M or less in a standard flow cytometry assay using HUVEC cells.

An anti-TM4SF1 antibody, or antigen-binding fragment thereof, of the disclosure, in certain embodiments, binds to human TM4SF1 with a K_{D} of about 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰ M to about 2×10⁻¹¹ M, about 2×10⁻¹¹ M to about 2×10⁻¹² M, about 3×10⁻¹³ M to about 3×10⁻³ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰ M to about 3×10⁻¹¹ M, about 3×10⁻¹¹ M to about 3×10⁻¹² M, about 4×10⁻¹³ M to about 4×10⁻⁴ M, about 4×10⁻⁴ M to about 4×10⁻⁵ M, about 4×10⁻⁵ M to about 4×10⁻⁶ M, about 4×10⁻⁶ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹¹ M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰ M to about 5×10⁻¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the K_{D} is determined in a standard flow cytometry assay using HUVEC cells.

An anti-TM4SF1 antibody, or antigen-binding fragment thereof, of the disclosure, in certain embodiments, binds to human TM4SF1 with a K_{D} of about 5×10⁻¹⁰ M or less in a standard flow cytometry assay using HUVEC cells.

An anti-TM4SF1 antibody, or antigen-binding fragment thereof, of the disclosure, in certain embodiments, binds to cynomolgus TM4SF1 with a K_{D} about 1×10⁻⁶ M or less in a standard flow cytometry assay using HEK293 overexpressing cells. In one embodiment, the HEK293 cells are transfected to express cynomolgus TM4SF1. In a further embodiment, HEK293 cells express cynomolgus TM4SF1 at about 600 mRNA copies per 10⁶ copies 18S rRNA.

Methods of determining the K_{D} of an antibody or antibody fragment are known in the art. For example, surface plasmon resonance may be used to determine the K_{D} of the antibody to the antigen (e.g., using a BIACORE 2000 or a BIACORE 3000 (BIAcore, Inc., Piscataway, N.J.) at 25° C. with immobilized antigen or Fc receptor CM5 chips at about 10 response units (RU)). In certain embodiments FACS or flow cytometry is used to determine the K_{D}, whereby cells, such as HEK293 cells or HUVEC cells, that express TM4SF1 are used to bind the antibody or fragment and measure the K_{D} according to standard methods. Affinity determination of antibodies using flow cytometry is described, for example, in Geuijen et al (2005) J Immunol Methods. 302(1-2):68-77. In certain embodiments, FACS is used to determine affinity of antibodies.

In one embodiment, the disclosure features an anti-TM4SF1 antibody or antigen binding fragment thereof, having CDR amino acid sequences described herein with conservative amino acid substitutions, such that the anti-TM4SF1 antibody or antigen binding fragment thereof comprises an amino acid sequence of a CDR that is at least 95% identical (or at least 96% identical, or at least 97% identical, or at least 98% identical, or at least 99% identical) to a CDR amino acid sequence set forth in Table 16. A “conservative amino acid substitution” is one in which an amino acid residue is substituted by another amino acid residue having a side chain (R group) with similar chemical properties (e.g., charge or hydrophobicity). In general, a conservative amino acid substitution may not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are disclosed herein or in literature. See. e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The disclosure further features in one aspect an anti-TM4SF1 antibody, or antigen-binding fragment thereof, that binds to an epitope on the ECL2 loop of human TM4SF1 with a K_{D} of about 5×10⁻⁸ M or less as determined in a standard flow cytometry assay using HUVEC cells, wherein the anti-TM4SF1 antibody, or antigen-binding fragment thereof, comprises a light chain variable region comprising a human IgG framework region and comprises a heavy chain variable region comprising a human IgG framework region. In one embodiment, the anti-TM4SF1 antibody, or antigen-binding fragment thereof, is humanized. In one embodiment, the anti-TM4SF1 antibody, or antigen-binding fragment thereof, cross reacts with cynomolgus TM4SF1.

In another aspect of the disclosure, the anti-TM4SF1 antibody, or antigen-binding fragment thereof, is a humanized anti-TM4SF1 antibody, or antigen-binding fragment thereof, that binds to an epitope on the ECL2 loop of human TM4SF1 with a K_{D} about 5×10⁻⁸ M or less as determined in a standard flow cytometry assay using HUVEC cells. In one embodiment, the anti-TM4SF1 antibody, or antigen-binding fragment thereof, binds to cynomolgus TM4SF1 with a K_{D} about 1×10⁻⁶ M or less in a standard flow cytometry assay using HEK293 overexpressing cells. In one embodiment, the anti-TM4SF1 antibody, or antigen-binding fragment thereof, binds to human TM4SF1 with a K_{D} of about 1×10⁻⁸ M or less in a standard flow cytometry assay using HUVEC cells. In one embodiment, the anti-TM4SF1 antibody, or antigen-binding fragment thereof, binds to human TM4SF1 with a K_{D} of 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰ M to about 2×10⁻¹¹ M, about 2×10⁻¹¹ M to about 2×10⁻¹² M, about 3×10⁻³ M to about 3×10⁻⁴ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰M to about 3×10⁻¹¹ M, about 3×10⁻¹¹ M to about 3×10⁻¹² M, about 4×10⁻³ M to about 4×10⁻⁴ M, about 4×10⁻⁴ M to about 4×10⁻⁵ M, about 4×10⁻⁶ M to about 4×10⁻⁶ M, about 4×10⁻⁷ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹² M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰ M to about 5×10⁻¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the K_{D} is determined in a standard flow cytometry assay using HUVEC cells. In one embodiment, the anti-TM4SF1 antibody, or antigen-binding fragment thereof, binds to human TM4SF1 with a K_{D} of about 5×10⁻¹⁰ M or less in a standard flow cytometry assay using TM4SF1 expressing HUVEC cells.

In one embodiment, binding of an anti-TM4SF1 antibody, or antigen binding fragment, of the disclosure to human TM4SF1 is not dependent on glycosylation of the ECL2 loop of human TM4SF1, i.e., binding of the antibody is independent of glycosylation of TM4SF1 within the ECL2 loop (SEQ ID NO: 77).

The anti-TM4SF1 antibodies, or antigen-binding fragments thereof, of the disclosure may be any of any isotype (for example, but not limited to IgG, IgM, and IgE). In certain embodiments, antibodies, or antigen-binding fragments thereof, of the disclosure are IgG isotypes. In a specific embodiment, antibodies, or antigen-binding fragments thereof, of the disclosure are of the IgG1, IgG2 or IgG4 isotype. In certain embodiments, the anti-TM4SF1 antibody, or antigen-binding fragment thereof, are human IgG1, human IgG2, or human IgG4 isotype.

IgG2 is naturally the lowest in ADCC and/or CDC activity (An et al., MAbs. 2009 November-December; 1(6): 572-579). Accordingly, in certain embodiments it IgG2 is advantageously used. However, IgG2 has two extra cysteines (leading to 4 inter-hinge disulfide bonds) which make it prone to aggregation via formation of inter-antibody disulfide bonds. In a related embodiment, mutations to the IgG2 cysteines are made to decrease aggregation.

The present disclosure provides antibody fragments that bind to TM4SF1. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments may be possible. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

In certain embodiments, the antigen binding fragment is selected from the group consisting of a Fab, a Fab′, a F(ab′)2, an Fv, and an scFv.

Anti-TM4SF1 antibodies (and fragments) that, for example, have a high affinity for human TM4SF1, can be identified using screening techniques. For example, monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized using, for example, the ECL2 loop of human TM4SF1 or cells expressing TM4SF1 (whereby the ECL2 loop is expressed on the cell surface), to elicit lymphocytes that produce or are capable of producing antibodies that may specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically may include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, Va.), and those derived from MOPC-21 and MPC-11 mouse tumors available from the Salk Institute Cell Distribution Center (San Diego, Calif.). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, TM4SF1 (e.g., a soluble form of the ECL2 loop or cells expressing said loop) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-TM4SF1 antibodies can be obtained by designing a suitable antigen screening procedure to select for the phage clone of interest followed by construction of a full length anti-TM4SF1 antibody clone using VH and/or VL sequences (e.g., the Fv sequences), or various CDR sequences from VH and VL sequences, from the phage clone of interest and suitable constant region (e.g., Fc) sequences described in Kabat et al., supra.

Screening of anti-TM4SF1 antibodies can be performed using binding assays known in the art and described herein for determining whether the antibody has a therapeutic affinity for the ECL2 loop of TM4SF1. The ability of the antibody to inhibit or decrease metastatic cell activity can be measured using standard assays in the art, as well as those described herein. Preclinical assays require use of an animal model of metastasis, commonly of one of three types: (i) injection of metastatic mouse tumor cells such as B16F10 melanoma TCs into mice, commonly via tail vein injection to generate lung metastases, via portal vein or intrasplenic injection to generate liver metastases, or via left ventricular cardiac injection to generate bone and other metastases; (ii) orthotopic transplantation of metastatic tumor cells or intact tumor fragments into mice, which methods often require later surgical resection of the primary tumor to prevent morbidity associated with primary tumor growth; and (iii) genetically engineered mouse models of spontaneous metastasis, of which the most common is the MMTV-Pyt (mouse mammary tumor virus-polyomavirus middle T Antigen) mouse mammary carcinoma model which provides a highly realistic mouse model of human cancer metastasis; greater than 85% of hemizygous MMTV-PyMT females spontaneously develop palpable mammary tumors which metastasize to the lung at age to 8-16 weeks. Quantifying the metastatic burden in the lung, either by live animal imaging or direct counting of metastatic nodules in the lungs of sacrificed animals, as a function of the degree of TM4SF1 immunoblockade and achieving a therapeutic level, e.g., at least a 50% reduction in lung metastasis, would be indicative, for example, of a therapeutic antibody that could be used in the methods of the disclosure. Further, cross-species reactivity assays are known in the art. Examples of assays that can be used are described, for example, in Khanna and Hunter (Carcinogenesis. 2005 March; 26(3):513-23) and Saxena and Christofori (Mol Oncol. 2013 April; 7(2):283-96), incorporated by reference in their entireties herein.

In some embodiments, an anti-TM4SF1 antibody or an antigen binding fragment thereof is cysteine engineered for conjugation by reduction and reoxidation. Cysteine engineered antibodies, in some embodiments, are made reactive for conjugation with linker-degrader intermediates described herein, by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, Mass.) followed by re-formation of the inter-chain disulfide bonds (re-oxidation) with a mild oxidant such as dehydroascorbic acid.

In some instances, the cysteine engineered anti-TM4SF1 antibodies are reduced, for example, with about a 50 fold excess of DTT overnight in 50 mM Tris, pH 8.0 with 2 mM EDTA at room temperature, which removes Cys and glutathione adducts as well as reduces interchain disulfide bonds in the antibody. Removal of the adducts is in some instances monitored by reverse-phase LCMS using a PLRP-S column. The reduced cysteine engineered antibody can then be diluted and acidified by addition to at least about four volumes of 10 mM sodium succinate, pH 5 buffer. Alternatively, the antibody is diluted and acidified by adding to at least four volumes of 10 mM succinate, pH 5 and titration with 10% acetic acid until pH is approximately five. The pH-lowered and diluted cysteine engineered antibody is subsequently loaded onto a HiTrap S cation exchange column, washed with several column volumes of 10 mM sodium acetate, pH 5 and eluted with 50 mM Tris, pH 8.0, 150 mM sodium chloride. Disulfide bonds are reestablished between cysteine residues present in the parent Mab by carrying out reoxidation. The eluted reduced cysteine engineered antibody described above is treated with 15× dehydroascorbic acid (DHAA) for about 3 hours or, alternatively, with 200 nM to 2 mM aqueous copper sulfate (CuSO₄) at room temperature overnight. Other oxidants, i.e. oxidizing agents, and oxidizing conditions, which are known in the art may be used. Ambient air oxidation may also be effective. This mild, partial reoxidation step forms intrachain disulfides efficiently with high fidelity. Reoxidation can bemonitored by reverse-phase LCMS using a PLRP-S column. The reoxidized cysteine engineered antibody can be diluted with succinate buffer as described above to reach pH of approximately 5 and purification on an S column may be carried out as described above with the exception that elution was performed with a gradient of 10 mM succinate, pH 5, 300 mM sodium chloride (buffer B) in 10 mM succinate, pH 5 (buffer A). To the eluted antibody, EDTA is added to a final concentration of 2 mM and concentrated, if necessary, to reach a final concentration of more than 5 mg/mL. The resulting cysteine engineered antibody, ready for conjugation, can be stored at −20° C. or −80° C. in aliquots. Liquid chromatography/Mass Spectrometric Analysis was performed on a 6200 series TOF or QTOF Agilent LC/MS. Samples are, in some instances, chromatographed on a PRLP-S®, 1000 A, microbore column (50 mm×2.1 mm, Polymer Laboratories, Shropshire, UK) heated to 80° C. A linear gradient from 30-40% B (solvent A: 0.05% TFA in water, solvent B: 0.04% TFA in acetonitrile) was used and the eluent was directly ionized using the electrospray source. Data were collected and deconvoluted by the MassHunter software (Agilent). Prior to LC/MS analysis, antibodies or conjugates (50 micrograms) were treated with PNGase F (2 units/ml; PROzyme, San Leandro, Calif.) for 2 hours at 37° C. to remove N-linked carbohydrates.

Alternatively, antibodies or conjugates are partially digested with LysC (0.25 pg per 50 pg (microgram) antibody or conjugate) for 15 minutes at 37° C. to give a Fab and Fc fragment for analysis by LCMS. Peaks in the deconvoluted LCMS spectra are assigned and quantitated. Degrader-to-antibody ratios (DAR) are calculated by calculating the ratio of intensities of the peak or peaks corresponding to Degrader-conjugated antibody relative to all peaks observed.

Degrader Compounds

Degraders are heterobifunctional small molecules that can bind to both a target protein and a ubiquitin ligase, resulting in ubiquitination and degradation of the target. A degrader reagent comprises a ligand for the target protein (a protein binding (PB) domain) and a ligand for an E3 ligase recognition domain (E3LB). Once the degrader has induced a sufficient degree of ubiquitination of the target, it is then recognized and degraded by a proteasome. In some instances, the protein binding domain is connected to the E3LB by a linker. Degraders can induce rapid and sustained degradation, induce a robust inhibition of downstream signals, and display enhanced target selectivity. Degraders permit the selective intracellular removal of undesirable proteins. Moreover, a single degrader molecule can engage in multiple rounds of binding to target protein molecules, thereby allowing degraders to function as catalysts for the selective destruction of proteins.

A degrader as provided herein has a structure E3LB-L2-PB; where E3LB is an E3 ligase binding group, L2 is a linker, and PG is a protein binding group. In some instances, the E3LB is covalently bound to L2. In some instances, L2 is covalently bound to the protein binding group (PB).

A degrader antibody conjugate (DAC) can comprise a single antibody where the single antibody can have more than one degrader, each degrader covalently linked to the antibody through a linker L1. The “Degrader loading” is the average number of degrader moieties per antibody. Degrader loading may range from 1 to 8 degrader (D) per antibody (Ab). Tat is, in the PAC formula, Ab-(L1-D)ₚ, p has a value from about 1 to about 50, from about 1 to about 8, from about 1 to about 5, from about 1 to about 4, or from about 1 to about 3. Each degrader covalently linked to the antibody through linker L1 can be the same or different degrader and can have a linker of the same type or different type as any other L1 covalently linked to the antibody. In one embodiment, Ab is a cysteine engineered antibody and p is about 2.

For some DACs, p may be limited by the number of attachment sites on the antibody. For example, an antibody may have only one or several cysteine thiol groups, or may have only one or several sufficiently reactive thiol groups through which a linker may be attached. Another reactive site on an Ab to connect L1-Ds are the amine functional group of lysine residues. Values of p include values from about 1 to about 50, from about 1 to about 8, from about 1 to about 5, from about 1 about 4, from about 1 to about 3, and where p is equal to 2. In some embodiments, the subject matter described herein is directed to any the DACs, wherein p is about 1, 2, 3, 4, 5, 6, 7, or 8.

Generally, fewer than the theoretical maximum of degrader moieties is conjugated to an antibody during a conjugation reaction. An antibody may contain, for example, many lysine residues that do not react with the linker L-Degrader group (L1-D) or linker reagent. Only the most reactive lysine groups may react with an amine-reactive linker reagent. Also, only the most reactive cysteine thiol groups may react with a thiol-reactive linker reagent or linker L1-Degrader group. Generally, antibodies do not contain many, if any, free and reactive cysteine thiol groups which may be linked to a Degrader moiety. Most cysteine thiol residues in the antibodies of the compounds exist as disulfide bridges and must be reduced with a reducing agent such as dithiothreitol (DTT) or TCEP, under partial or total reducing conditions. However, the Degrader loading (Degrader/antibody ratio, “PAR”) of a PAR may be controlled in several different manners, including: (i) limiting the molar excess of linker L-Degrader group or linker reagent relative to antibody, (ii) limiting the conjugation reaction time or temperature, and (iii) partial or limiting reductive conditions for cysteine thiol modification.

Degraders used in the DAC, can include but are not limited to those disclosed in the PROTAC-DB (See Gaoqi Weng, et. al. PROTAC-DB: an online database of PROTACs. Nucleic Acids Research, 2020; accessed Mar. 26, 2021).

E3 Ligase Recognition Domain (E3LB)

E3 ubiquitin ligases confer substrate specificity for ubiquitination. There are known ligands which bind to these ligases. As described herein, an E3 ubiquitin ligase binding group is a peptide or small molecule that can bind an E3 ubiquitin ligase.

Representative examples of E3 ubiquitin ligases include, but are not limited to, von Hippel-Lindau (VHL); cereblon, XIAP, E3A; MDM2; Anaphase-promoting complex (APC); UBR5 (EDD1); SOCS/BC-box/eloBC/CUL5/RING; LNXp80; CBX4; CBLL1; HACE1; HECTD1; HECTD2; HECTD3; HECW1; HECW2; HERC1; HERC2; HERC3; HERC4; HUWE1; ITCH; NEDD4; NEDD4L; PPIL2; PRPF19; PIAS1; PIAS2; PIAS3; PIAS4; RANBP2; RNF4; RBX1; SMURF1; SMURF2; STUB1; TOPOR5; TRIP12; UBE3A; UBE3B; UBE3C; UBE4A; UBE4B; UBOX5; UBR5; WWP1; WWP2; Parkin; A20/TNFAIP3; AMFR/gp78; ARA54; beta-TrCP1/BTRC; BRCA1; CBL; CHIP/STUB1; E6; E6AP/UBE3A; F-box protein 15/FBXO15; FBXW7/Cdc4; GRAII/RNF128; HOIP/RNF31; cIAP-1/HIAP-2; cIAP-2/HIAP-1; cIAP (pan); ITCH/AIP4; KAP1; MARCH8; Mind Bomb 1/MIB1; Mind Bomb 2/MIB2; MuRF1/TRIM63; NDFIP1; NEDD4; NleL; Parkin; RNF2; RNF4; RNF8; RNF168; RNF43; SART1; Skp2; SMURF2; TRAF-1; TRAF-2; TRAF-3; TRAF-4; TRAF-5; TRAF-6; TRIM5; TRIM21; TRIM32; UBR5; and ZNRF3.

Tables 1-15 List Exemplary E3 Ligases that May be Utilized in the Degrader Molecules Described Herein.

TABLE 1
E3 Ligases, HECT type

| Name | UniProt | Genbank | LLRefseq | Unigene | Domains |
|---|---|---|---|---|---|
| EDD/HYD | O95071 | AF006010 | 51366NM_015902 | Hs.492445 | HECT; UBA; ZFUBR1; pab |
| FL721156 | Q5T447 | AK096462 | 79654NM_024602 | Hs.525084 | DOC1; HECT |
| HACE1/ | Q5VU99 | BC034982 | 57531NM_020771 | Hs.434340 | Ank; HECT |
| KIAA1320 | | | | | |
| HECTD1 | Q9ULT8 | BC011658 | 25831NM_015382 | Hs.210850 | HECT; MIBHERC2; |
| HECTD2 | Q5U5R9 | BC040187 | 143279NM_182765; | HS.66378 | HECT |
| | | | NM_173497 | | |
| HECW1/ | Q9HCC7 | AB048365 | 23072NM_015052 | Hs.164453 | C2; HECT; WW |
| NEDL1 | | | | | |
| HECW2/ | Q9P2P5 | AB037722 | 57520NM_020760 | Hs.314436 | C2; HECT; WW |
| KIAA1301 | | | | | |
| HERC1/P532 | Q15751 | U50078 | 8925NM_003922 | Hs.210385 | HECT; SPRY; WD; RCC |
| HERC2 | 095714 | AF071172 | 8924NM_004667 | Hs.434890 | DOC1; HECT; HERC2; |
| | | | | | MIBHERC2; UBA; ZZ; RCC |
| HERC3 | Q15034 | D25215 | 8916NM_014606 | Hs.35804 | HECT; RCC |
| HERC4 | Q5VXS9 | BC039600 | 26091NM_015601 | Hs.51891 | HECT; RCC |
| HERCS/ | Q9UII4 | AB027289 | 51191NM_016323 | Hs.26663 | HECT; RCC |
| CEBP1 | | | | | |
| HERC6 | Q8IVU3 | BC042047 | 55008NM_017912 | Hs.529317 | HECT; RCC |
| ITCH | Q96702 | AB056663 | 83737NM_031483 | Hs.472509 | C2; HECT; WW |
| KIAA0317 | 015033 | AB002315 | 9870NM_014821 | Hs.497417 | HECT |
| KIAA0614/ | Q9Y4D8 | AB014514 | noneXM 497354 | Hs.7314 | HECT; SPRY |
| FL730092 | | | | | |
| KIAA1333/ | Q9NXCO | AK000340 | 55632NM_017769 | Hs.509008 | HECT; RF |
| FL720333 | | | | | |
| NEDD4 | P46934 | D42055 | 4734NM_198400 | Hs.1565 | C2; HECT; WW |
| | | | NM_006154; | | |
| NEDD4L | Q7Z5F1 | AY112985 | 23327NM_015277 | Hs.185677 | C2; HECT; WW |
| SMURF1 | Q9HCE7 | AF199364 | 57154NM_020429; | Hs.189329 | C2; HECT; WW |
| | | | NM_181349 | | |
| SMURF2 | Q9HAU4 | AF310676 | 64750NM_022739 | Hs.515011 | C2; HECT; WW |
| TRIP12 | Q14669 | D28476 | 9320NM_004238 | Hs.368985 | HEAT/ARM; HECT; WWE |
| UBE3A/ | Q05086 | X98031 | 7337NM_000462; | Hs.22543 | HECT |
| E6AP | | | NM_130838; | | |
| | | | NM_130839 | | |
| UBE3B | Q9BXZ4 | AF251046 | 89910NM_130466; | Hs.374067 | HECT; IQ |
| | | | NM_183415; | | |
| | | | NM_183414 | | |
| UBE3C/ | Q15386 | D13635 | 9690NM_014671 | Hs.118351 | HECT; IQ; IRF3CT |
| KIAA0010 | | | | | |
| UREB1/ | Q7Z6Z7 | CR456813 | noneNM_031407 | Hs.136905 | HECT; UBA; UIM; WWE |
| LASU1 | | | | | |
| WWP1 | Q9HOMO | AY043361 | 11059NM_007013 | Hs.533440 | C2; HECT; WW |
| WWP2 | 000308 | BC064531 | 11060NM_007014; | Hs.408458 | HECT; WW |
| | | | NM_199423; | | |
| | | | NM_199424 | | |

TABLE 2
E3 Ligases, RING type

| Name | UniProt | Genbank | LL | Refseq | Unigene | Domains |
|---|---|---|---|---|---|---|
| AMFR | Q9UKV5 | AF124145 | 267 | NM_138958 | Hs.295137 | RF; CUE1; DER3 |
| | | | | NM_001144 | | |
| ANACPC11 | Q9NYG5 | AF247565 | 51529 | NM_001002249; | Hs.534456 | RF |
| | | | | NM_016746; | | |
| | | | | NM_016746 | | |
| | | | | NM_001002248; | | |
| | | | | NM_0001002244 | | |
| | | | | NM_001002247; | | |
| | | | | NM_0001002245 | | |
| BARD1 | Q99728 | U76638 | 580 | NM_000465 | Hs.54089 | RF; Ank; BRCT |
| BFAR/BAR | Q9NZS9 | BC003054 | 51283 | NM_016561 | Hs.435556 | RF; SAM |
| BIRC2/cIAP1 | Q13490 | BC016174 | 329 | NM_001166 | Hs.503704 | RF; BIR; CARD |
| BIRC3/cIAP2 | Q13489 | U37546 | 330 | NM_001165; | Hs.127799 | RF; BIR; CARD |
| | | | | NM_182962 | | |
| BIRC4/XIAP | P98170 | U45880 | 331 | NM_001167 | Hs.356076 | RF; BIR |
| BIRC7/Livin | Q96CA5 | BC014475 | 79444 | NM_139317 | Hs.256126 | RF; BIR |
| BIRC8/ILP2 | Q96P09 | AF164682 | 112401 | NM_033341 | Hs.348263 | RF; BIR |
| BRAP | Q7Z569 | AF035620 | 8315 | NM_006768 | Hs.530940 | RF; ZFu |
| BRCA1 | P38398 | U14680 | 672 | NM_007294; | Hs.194143 | RF; BRCT |
| | | | | NM_007304; | | |
| | | | | NM_007299; | | |
| | | | | NM_007300; | | |
| | | | | NM_007302; | | |
| | | | | NM_007306; | | |
| | | | | NM_007296; | | |
| | | | | NM_007301; | | |
| | | | | NM_007305; | | |
| | | | | NM_007297; | | |
| | | | | NM_007303; | | |
| | | | | NM_007295 | | |
| | | | | NM_007298 | | |
| C13orf7 | Q5W0B1 | BC028586 | 79596 | NM_024546 | Hs.93956 | RF |
| C16orf28/FLJ12623 | Q9H9P5 | AK022685 | 65259 | NM_023076 | Hs.161279 | RF |
| C17orf29 | Q63HN8 | BX647946 | 57674 | NM_020914 | Hs.195642 | RF |
| C18orf23/FLJ45559 | Q6ZSG1 | AK127467 | 147341 | NM_152470 | Hs.501114 | RF |
| CBL | P22681 | X57110 | 867 | NM_005188 | Hs.504096 | RF; UBA |
| CBLB | Q13191 | U26710 | 868 | NM_170662 | Hs.430589 | RF; UBA |
| CBLC | Q9ULV8 | AF117646 | 23624 | NM_012116 | Hs.466907 | RF |
| CBLL1 | Q8TAJ4 | BCO27460 | 79872 | NM_024814 | Hs.432792 | RF |
| CGRRF1 | Q99675 | U66469 | 10668 | NM_006568 | Hs.59106 | RF |
| CHFR | Q96EP1 | AF170724 | 55743 | NM_018223 | Hs.507336 | RF; FHA |
| CNOT4 | Q95628 | U71267 | 4850 | NM_013316; | Hs.490224 | RF; RBD |
| | | | | NM_001008225 | | |
| DKFZp547C195 | Q6P2E0 | BC064581 | 257160 | NM_207343 | Hs.380110 | RF |
| DKFZp761H1710 | Q9H0X6 | AL136540 | 83459 | NM_031297 | Hs.512767 | RF |
| DTX1 | Q86Y01 | BC048216 | 1840 | NM_004416 | Hs.372152 | RF; WWE |
| DTX2 | Q86UW9 | AK023924 | 113878 | NM_020892 | Hs.187058 | RF; WWE |
| DTX3 | Q8N919 | AK092085 | 196403 | NM_178502 | Hs.32374 | RF |
| DTX3L/BBAP | Q8TDB6 | BC060509 | 151636 | NM_138287 | Hs.518201 | RF |
| DTX4/KIAA0937 | Q9Y2E6 | AB023154 | none | XM_166213 | Hs.523696 | RF; WWE |
| DZIP3 | Q86Y13 | AB014575 | 9666 | NM_014648 | Hs.409210 | RF |
| FLJ10520 | Q5XKR3 | BC002574 | none | None | Hs.77510 | RF |
| FLJ12270/KIAA1923 | Q96PW5 | AB067510 | 79726 | NM_030581 | Hs.280951 | RF; GIUEY; WD |
| FLJ12875 | Q969V5 | BC014010 | 79594 | NM_024544 | Hs.10101 | RF |
| FLJ16581 | Q6ZWI9 | AK122906 | none | XM_498131 | Hs.448264 | RF; SPRY |
| FLJ20225 | Q9NXI6 | AK000232 | 54546 | NM_019062 | Hs.124835 | RF |
| FLJ20315/URCC | Q65ZA4 | AB081837 | 54894 | NM_017763 | Hs.500398 | RF |
| FLJ23749 | Q8TEA0 | AK074329 | 91694 | NM_152271 | Hs.180178 | RF |
| FLJ31951 | Q8IVP7 | BC042684 | 153830 | NM_144726 | Hs.349306 | RF; DER3 |
| FLJ35757 | Q8NA82 | AK093076 | 162333 | NM_152598 | Hs.446268 | RF |
| FLJ36180 | Q8N9V2 | AK093499 | 339976 | NM_178556 | Hs.348618 | RF; BBOX; SPRY |
| FLJ38628 | Q96GF1 | BC009504 | 91445 | NM_152267 | Hs.517553 | RF |
| FLJ45273 | Q6ZSR4 | AK127206 | 164832 | NM_198461 | Hs.30646 | RF |
| FLJ46380 | Q6ZRF8 | AK128246 | 388591 | NM_207396 | Hs.512336 | RF |
| HOZFP | Q86VG1 | BC051193 | 152518 | NM_152995 | Hs.351839 | RF |
| KIAA0804 | O94896 | AB018347 | 23355 | NM_015303; | Hs.269263 | RF |
| | | | | NM_001009921 | | |
| KIAA1333/FLJ20333 | Q9NXC0 | AK000340 | 55632 | NM_017769 | Hs.509008 | RF; HECT |
| KIAA1404 | Q9P2E3 | AK002139 | 57169 | NM_021035 | Hs.371794 | RF; SEN1 |
| KIAA1542 | Q9P1Y6 | AB040975 | 57661 | NM_020901 | Hs.325838 | RF |
| KIAA1972 | Q96DX4 | BC013173 | 89970 | NM_133368 | Hs.460885 | RF; SPRY |
| KIAA1991 | Q8NCN4 | AB082522 | none | XM_495886 | Hs.369437 | RF |
| LNX | Q8TBB1 | BC022983 | 84708 | NM_032622 | Hs.407755 | RF; PDZ |
| LNX2 | Q8N448 | BC036755 | 222484 | NM_153371 | Hs.132359 | RF; PDZ |
| LOC149603 | Q6PJR0 | BC012758 | none | XM_047499 | Hs.356377 | RF |
| LOC285498 | Q8IY99 | BC036250 | 285498 | NM_194439 | Hs.248290 | RF |
| LOC493829 | Q8N4X6 | BC033211 | 493829 | NM_001008274 | Hs.535455 | RF; BBOX |
| LOC51136/FLJ25783 | Q8N7D0 | AK098649 | 51136 | NM_016125 | Hs.531701 | RF |
| LOC51255 | Q9P0P0 | AF151072 | 51255 | NM_016494 | Hs.11156 | RF |
| LRSAM1/TAL | Q6UWE0 | AY358830 | 90678 | NM_138361; | Hs.495188 | RF; LRR; SAM |
| | | | | NM_001005373; | | |
| | | | | NM_001005374 | | |
| M96/MTF2 | Q9Y483 | AJ010014 | 22823 | NM_007358 | Hs.31016 | RF |
| MAP3K1 | Q13233 | AF042838 | none | XM_042066 | Hs.508461 | RF; kinase |
| MARCH1 | Q8TCQ1 | AL713759 | 55016 | NM_017923 | Hs.136900 | RF |
| MARCH2/MARCH-II | Q9P0N8 | AF151074 | 51257 | NM_016496; | Hs.445113 | RF |
| | | | | NM_001005416; | | |
| | | | | NM_001005415 | | |
| MARCH3/MARCH-III | Q86UD3 | BC047569 | 115123 | NM_178450 | Hs.132441 | RF |
| MARCH5/RNF153 | Q9NX47 | AK000452 | 54708 | NM_017824 | Hs.549165 | RF |
| MARCH6/KIAA0597 | 060337 | AB011169 | 10299 | NM_005885 | Hs.432862 | RF |
| MARCH7/AXOT | Q9H992 | BC065014 | 64844 | NM_022826 | Hs.529272 | RF |
| MARCH8/MIR | Q8TC72 | BC025394 | 220972 | NM_001002265; | Hs.499489 | RF |
| | | | | NM_145021; | | |
| | | | | NM_001002266 | | |
| MARCH9/MARCH-IX | Q86VN5 | BC050397 | 92979 | NM_138396 | Hs.65377 | RF; RGG |
| | | | | NM_006878; | | |
| MDM2 | Q00987 | M92424 | 4193 | NM_006881; | Hs.369849 | RF; MBL; NZF; ZFrn |
| | | | | NM_006880; | | |
| | | | | NM_002392; | | |
| | | | | NM_006882; | | |
| | | | | NM_006879 | | |
| MDM4 | O15151 | AF007111 | 4194 | NM_002393 | Hs.497492 | RF; MBL; NZF; ZFrn |
| MGC4734 | Q96D59 | BC013036 | 138065 | NM_145051 | Hs.211374 | RF |
| MGRN1 | Q86W76 | BC050389 | 23295 | NM_015246 | Hs.526494 | RF |
| MIB1/MIB | Q86YT6 | AY149908 | 57534 | NM_020774 | Hs.140903 | RF; Ank; MIBOREP; |
| | | | | ZZ | | MIBHERC2; |
| MID1 | O15344 | Y13667 | 4281 | NM_000381; | Hs.27695 | RF; POSTRFBBOX; |
| | | | | NM_033290; | | SPRY |
| | | | | NM_033291 | | |
| MID2 | Q9UJV3 | Y18880 | 11043 | NM_052817; | Hs.12256 | RF; POSTRFBBOX; |
| | | | | NM_012216 | | SPRY |
| MKRN1 | Q9UHC7 | BC025955 | 23608 | NM_013446 | Hs.490347 | RF; ZF_MAKORIN |
| MKRN2 | Q9H000 | BC015715 | 23609 | NM_014160 | Hs.279474 | RF; ZF_MAKORIN |
| MKRN3 | Q13064 | U19107 | 7681 | NM_005664 | Hs.72964 | RF; ZF_MAKORIN |
| MNAB/MASNAB | Q9HBD2 | AF255303 | 54542 | NM_018835 | Hs.533499 | RF ZF_CCCH |
| MNAT1 | P51948 | X87843 | 4331 | NM_002431 | Hs.509523 | RF |
| YCBP2 | Q6PIB6 | BC037971 | 23077 | NM_015057 | Hs.151411 | RF |
| MYLIP | Q8WY64 | AF006003 | 29116 | NM_013262 | Hs.484738 | RF; BAND_41 |
| NEURL | O76050 | U87864 | 9148 | NM_004210 | Hs.549085 | RF; NEURALIZED |
| NFX1 | Q12986 | U15306 | 4799 | NM_002504; | Hs.413074 | RF; DNABIND_JAG |
| | | | | NM_147133; | | |
| | | | | NM_147134 | | |
| NHLRC1/Malin | Q6VVB1 | BK001510 | 378884 | NM_198586 | Hs.348351 | RF |
| NSMCE1/NSE1 | Q8WV22 | BC018938 | 197370 | NM_145080 | Hs.284295 | RF |
| PCGF1/NSPC1 | Q9BSM1 | BC004952 | 84759 | NM_032673 | Hs.316750 | RF |
| PCGF2/RNF110 | P35227 | D13969 | 7703 | NM_007144 | Hs.371617 | RF |
| PCGF4/BMI1 | P35226 | L13689 | 648 | NM_005180 | Hs.380403 | RF |
| PCGFS | Q86SE9 | BC051845 | 84333 | NM_032373 | Hs.500512 | RF |
| PCGF6/hMBLR | Q9BYE7 | AB047006 | 84108 | NM_032154 | Hs.335808 | RF |
| PDZRN3/KIAA1095 | Q9UPQ7 | AB029018 | 23024 | NM_015009 | Hs.434900 | RF; PDZ; ZFt |
| PEX10 | O60683 | AF060502 | 5192 | NM_153818; | Hs.546273 | RF |
| | | | | NM_002617 | | |
| PEX12 | O00623 | U91521 | 5193 | NM_000286 | Hs.270532 | RF |
| PHF7 | Q9NSX7 | BC022002 | 51533 | NM_173341; | Hs.372719 | RF |
| PJA1 | Q8NG27 | AF262024 | 6421 | NM_022368; | Hs.522679 | RF |
| | | | | NM_145119 | | |
| PJA2 | Q8N1G5 | BC030826 | 9867 | NM_014819 | Hs.483036 | RF |
| PML | P29590 | AF230401 | 5371 | NM_033246; | Hs.526464 | RF; BBOX |
| | | | | NM_033239; | | |
| | | | | NM_033240; | | |
| | | | | NM_033242; | | |
| | | | | NM_033247; | | |
| | | | | NM_033238; | | |
| | | | | NM_033244; | | |
| | | | | NM_002675; | | |
| | | | | NM_033250; | | |
| | | | | NM_033249; | | |
| | | | | NM_033245 | | |
| PXMP3 | P28328 | M86852 | 5828 | NM_000318 | Hs.437966 | RF |
| RAD18 | Q9NS91 | AB035274 | 56852 | NM_020165 | Hs.375684 | RF; SAF; ZF_RAD18 |
| RAG1 | P15918 | M29474 | 5896 | NM_000448 | Hs.73958 | RF |
| RAPSN | Q13702 | Z33905 | 5913 | NM_005055; | Hs.81218 | RF; TPR |
| | | | | NM_032645 | | |
| RBBP6 | Q7Z6E9 | AB112074 | 5930 | NM_032626; | Hs.188553 | RF ZFc |
| | | | | NM_018703; | | |
| | | | | NM_006910 | | |
| RBX1 | P62877 | AF142059 | 9978 | NM_014248 | Hs.474949 | RF |
| RCHY1 | Q96PM5 | AF247041 | 25898 | NM_015436; | Hs.48297 | RF; ZF_CHYCT; |
| | | | | NM_001009922; | | ZF_HOT13 |
| | | | | NM_001008925 | | |
| RFFL | Q8TBY7 | BCO28424 | 117584 | NM_057178 | Hs.13680 | RF; FYVE |
| RFP | P14373 | J03407 | 5987 | NM_006510; | Hs.440382 | RF; BBOX; SPRY |
| | | | | NM_030950 | | |
| RFP2 | O60858 | AJ224819 | 10206 | NM_213590; | Hs.436922 | RF; BBOX |
| | | | | NM_052811; | | |
| | | | | NM_001007278; | | |
| | | | | NM_005798 | | |
| RFPL1 | O75677 | AJ00228 | 5988 | NM_021026 | Hs.167750 | RF; SPRY |
| RFPL2 | O75678 | BC051910 | 10739 | NM_006605 | Hs.157427 | RF; SPRY |
| RFPL3 | O75679 | AJ010232 | 10738 | NM_006604 | Hs.167751 | RF; SPRY |
| RFWD2/COP1 | Q8NHY2 | AF508940 | 64326; | NM_001001740 | Hs.523744 | RF; WD |
| | | | | NM_022457 | | |
| RING1 | Q06587 | Z14000 | 6015 | NM_002931 | Hs.202430 | RF |
| RKHD1 | Q86XN8 | AB107353 | 399664 | NM_203304 | Hs.436495 | RF; KH |
| RKHD2 | Q5U5Q3 | BC041122 | 51320 | NM_016626 | Hs.465144 | RF |
| RKHD3/KIAA2009 | Q8IVG2 | AB095929 | 84206 | NM_032246 | Hs.104744 | RF; KH |
| RNF10/RIE2 | Q9ULW4 | AB027196 | 9921 | NM_014868 | Hs.442798 | RF |
| RNF103 | 000237 | D76444 | 7844 | NM_005667 | Hs.469199 | RF |
| RNF11 | Q9Y3C5 | AB024703 | 26994 | NM_014372 | Hs.309641 | RF |
| RNF111 | Q6P9A4 | BC060862 | 54778 | NM_017610 | Hs.404423 | RF |
| RNF12 | Q9NVW2 | AJ271670 | 51132 | NM_016120; | Hs.122121 | RF |
| | | | | NM_183353 | | |
| RNF121 | Q96DB4 | BC009672 | 55298 | NM_194453; | Hs.368554 | RF |
| | | | | NM_194452; | | |
| | | | | NM_018320 | | |
| RNF122/FLJ12526 | Q9H9V4 | AK022588 | 79845 | NM_024787 | Hs.151237 | RF |
| RNF123/KPC1 | Q5XPI4 | AY744152 | 63891 | NM_022064 | Hs.517970 | RF; SPRY |
| RNF125 | Q96EQ8 | BC012021 | 54941 | NM_017831 | Hs.272800 | RF; ZF_ZNF313 |
| RNF126 | Q9BV68 | BC001442 | 55658 | NM_017876; | Hs.69554 | RF; ZF_CIP8 |
| | | | | NM_194460 | | |
| RNF127/FLJ34458 | Q8NB00 | AK091777 | 79836 | NM_024778 | Hs.144266 | RF; TPR |
| RNF128/GRAIL | Q96RF3 | AF394689 | 79589 | NM_194463; | Hs.496542 | RF; PA |
| | | | | NM_024539 | | |
| RNF13 | O43567 | AF037204 | 11342 | NM_183381; | Hs.12333 | RF; PA |
| | | | | NM_183384; | | |
| | | | | NM_007282; | | |
| | | | | NM_183383; | | |
| | | | | NM_183382 | | |
| RNF130 | Q86XS8 | AY083998 | 55819 | NM_018434 | Hs.484363 | RF; PA |
| RNF133 | Q8WVZ7 | BCO22038 | 168433 | NM_139175 | Hs.549267 | RF; PA |
| RNF135/MGC13061 | Q8IUD6 | AY598332 | 84282 | NM_032322; | Hs.29874 | RF; SPRY |
| | | | | NM_197939 | | |
| RNF138 | Q8WVD3 | BC018107 | 51444 | NM_198128; | Hs.302408 | RF; ZF_ZNF313 |
| | | | | NM_016271 | | |
| RNF139/TRC8 | O75485 | AF064801 | 11236 | NM_007218 | Hs.492751 | RF; DER3 |
| RNF141 | Q8WVD5 | BC018104 | 50862 | NM_016422 | Hs.44685 | RF |
| RNF146/Dactylidin | Q9NTX7 | AK027558 | 81847 | NM_030963 | Hs.267120 | RF; WWE |
| RNF148 | Q8N308 | BCO29264 | 378925 | NM_198085 | Hs.529656 | RF; PA |
| RNF149 | Q8NC42 | AK074985 | 284996 | NM_173647 | Hs.171802 | RF; PA |
| RNF150/KIAA1214 | Q9ULK6 | AB033040 | 57484 | NM_020724 | Hs.480825 | RF |
| RNF151 | Q8NHS5 | BC029501 | none | XM_370927 | Hs.99354 | RF; ZFt |
| RNF152 | Q8N8N0 | AK096495 | 220441 | NM_173557 | Hs.465316 | RF |
| RNF157/KIAA1917 | Q96PX1 | AB067504 | 114804 | NM_052916 | Hs.269891 | RF |
| RNF166 | Q96A37 | BC017226 | 115992 | NM_178841 | Hs.513804 | RF; ZF_ZNF313 |
| RNF167 | Q9H6Y7 | AK025329 | 26001 | NM_015528 | Hs.7158 | RF; PA |
| RNF168/FLJ35794 | Q8IYW5 | BC033791 | 165918 | NM_152617 | Hs.518396 | RF |
| RNF170 | Q86YC0 | BC044566 | 81790 | NM_030954 | Hs.491626 | RF |
| RNF175/LOC285533 | Q8N4F7 | BC034385 | 285533 | NM_173662 | Hs.388364 | RF |
| RNF180 | Q86T96 | AL832580 | 285671 | NM_178532 | Hs.98890 | RF |
| RNF182/MGC33993 | Q8N6D2 | BC030666 | 221687 | NM_152737 | Hs.111164 | RF |
| RNF2/DING | Q99496 | Y10571 | 6045 | NM_007212 | Hs.124186 | RF |
| RNF20 | Q5VTR2 | BC063115 | 56254 | NM_019592 | Hs.168095 | RF |
| RNF24 | Q9Y225 | AL096778 | 11237 | NM_007219 | Hs.114180 | RF |
| RNF25 | Q96BH1 | BC015612 | 64320 | NM_022453 | Hs.471403 | RF; GIUEV |
| RNF26 | Q9BY78 | AB055622 | 79102 | NM_032015 | Hs.524084 | RF |
| RNF32 | Q6FIB3 | CR533513 | 140545 | NM_030936 | Hs.490715 | RF; IQ |
| RNF34 | Q969K3 | AF306709 | 80196 | NM_194271; | Hs.292804 | RF; FYVE |
| | | | | NM_025126 | | |
| RNF36 | Q86WT6 | BC047945 | 140691 | NM_080745; | Hs.169810 | RF; POSTBBOX; |
| | | | | NM_182985 | | SPRY |
| RNF38 | Q9H0F5 | AF394047 | 152006 | NM_194330; | Hs.333503 | RF |
| | | | | NM_022781; | | |
| | | | | NM_194328; | | |
| | | | | NM_194331; | | |
| | | | | NM_94329; | | |
| | | | | NM_194332 | | |
| RNF39/HCGV | Q96QB5 | AF238315 | 80352 | NM_25236; | Hs.121178 | RF; SPRY |
| | | | | NM_70769; | | |
| | | | | NM_170770 | | |
| RNF3A | O15262 | AJ001019 | 10336 | NM_006315 | Hs.144309 | RF |
| RNF4 | P78317 | AB000468 | 6047 | NM_002938 | Hs.66394 | RF |
| RNF40/KIAA0661 | O75150 | AB014561 | 9810 | NM_194352; | Hs.65238 | RF |
| | | | | NM_014771 | | |
| RNF41 | O75598 | AF077599 | 10193 | NM_194358; | Hs.524502 | RF |
| | | | | NM_94359; | | |
| | | | | NM_005785 | | |
| RNF44 | Q7LOR7 | BC039833 | 22838 | NM_014901 | Hs.434888 | RF |
| RNFS/HsRmal | Q99942 | AB056869 | 6048 | NM_006913 | Hs.534342 | RF |
| RNF6 | Q9Y252 | AJ010347 | 6049 | NM_83044; | Hs.136885 | RF |
| | | | | NM_05977; | | |
| | | | | NM_83043; | | |
| | | | | NM_183045 | | |
| RNF7/ROC2 | Q9UBF6 | AF164679 | 9616 | NM_183237; | Hs.134623 | RF |
| | | | | NM_83063; | | |
| | | | | NM_014245 | | |
| RNF8 | 076064 | AB012770 | 9025 | NM_003958; | Hs.485278 | RF; FHA |
| | | | | NM_183078 | | |
| RP11-307C12.10 | Q5T197 | AK057347 | 149095 | NM_152494 | Hs.351431 | RF |
| RP4-678E16.1 | Q5VTB9 | BC034221 | 55182 | NM_018150 | Hs.456557 | RF |
| RP5-1198E17.5 | Q5TC82 | AB095945 | none | XM_086409 | Hs.495097 | RF; ZF_CCCH |
| SH3MD2 | Q7Z6J0 | BC053671 | 57630 | NM_020870 | Hs.301804 | RF; SH3 |
| SH3RF2/FLJ23654 | Q8TEC5 | AK074234 | 153769 | NM_152550 | Hs.443728 | RF; SH3 |
| SIAH1 | Q8IUQ4 | U63295 | 6477 | NM_001006610; | Hs.295923 | RF |
| | | | | NM_003031; | | |
| | | | | NM_001006611 | | |
| SIAH2 | O43255 | Y15268 | 6478 | NM_005067 | Hs.477959 | RF |
| SMARCA3/HIP116 | Q14527 | L34673 | 6596 | NM_139048; | Hs.3068 | RF |
| | | | | NM_003071 | | |
| SYVN1/HRD1 | Q8N6E8 | BC030530 | 84447 | NM_172230; | Hs.321535 | RF; DER3 |
| | | | | NM_032431 | | |
| TOPORS | Q9UNR9 | AF098300 | 10210 | NM_005802 | Hs.535961 | RF; ICP0 |
| TRAF2 | Q12933 | BC032410 | 7186 | NM_021138 | Hs.522506 | RF; ZFt; TRAF |
| TRAF3 | Q13114 | U21092 | 7187 | NM_145725; | Hs.510528 | RF; ZFt; TRAF |
| | | | | NM_003300; | | |
| | | | | NM_145726 | | |
| TRAF4 | Q9BUZ4 | BC001769 | 9618 | NM_04295; | Hs.8375 | RF; ZFt; TRAF |
| | | | | NM_145751 | | |
| TRAFS | O00463 | AB000509 | 7188 | NM_145759; | Hs.523930 | RF; ZFt; TRAF |
| | | | | NM_004619 | | |
| TRAF6 | Q9Y4K3 | U78798 | 7189 | NM_145803; | Hs.444172 | RF; ZFt; TRAF |
| | | | | NM_004620 | | |
| TRAF7 | Q6Q0C0 | AY569455 | 84231 | NM_032271; | Hs.334479 | RF; ZFt; WD |
| | | | | NM_206835 | | |
| TRIM10 | Q9UDY6 | AF220122 | 10107; | NM_052828; | Hs.274295 | RF; BBOX; |
| | | | | NM_006778 | | POSTBBOX; SPRY |
| TRIM11 | Q96F44 | AF327056 | 81559 | NM_145214 | Hs.13543 | RF; BBOX; |
| | | | | | | POSTBBOX; SPRY |
| TRIM15 | Q9C019 | AF220132 | 89870 | NM_033229; | Hs.309602 | RF; BBOX; |
| | | | | NM_052812 | | POSTBBOX; SPRY |
| TRIM17 | Q9Y577 | AF156271 | 51127 | NM_016102 | Hs.121748 | RF; BBOX; |
| | | | | | | POSTBBOX; SPRY |
| TRIM2 | Q9C040 | AF220018 | 23321 | NM_015271 | Hs.435711 | RF; BBOX; |
| | | | | | | POSTBBOX |
| TRIM21 | P19474 | M34551 | 6737 | NM_003141 | Hs.532357 | RF; BBOX; |
| | | | | | | POSTBBOX; SPRY |
| TRIM22 | Q8IYM9 | BC035582 | 10346 | NM_006074 | Hs.501778 | RF; BBOX; |
| | | | | | | POSTBBOX; SPRY |
| TRIM23 | P36406 | L04510 | 373 | NM_033227; | Hs.792 | RF; BBOX; GTPase |
| | | | | NM_001656; | | |
| | | | | NM_033228 | | |
| TRIM25 | Q14258 | D21205 | 7706 | NM_005082 | Hs.534366 | RF; SPRY |
| TRIM26 | Q12899 | U09825 | 7726 | NM_003449 | Hs.485041 | RF; BBOX; |
| | | | | | | POSTBBOX; SPRY |
| TRIMS | O75382 | AF045239 | 10612 | NM_006458; | Hs.159408 | RF; BBOX; |
| | | | | NM_033278 | | POSTBBOX |
| TRIM31 | Q9BZY9 | AF230386 | 11074 | NM_007028; | Hs.493275 | RF; BBOX; |
| | | | | NM_052816 | | POSTBBOX |
| TRIM32 | Q13049 | AL133284 | 22954 | NM_012210 | Hs.209217 | RF; BBOX |
| TRIM34 | Q9BYJ4 | AB039902 | 53840 | NM_001003827; | Hs.125300 | RF; BBOX; |
| | | | | NM_021616; | | POSTBBOX; SPRY |
| | | | | NM_130390; | | |
| | | | | NM_130389 | | |
| TRIM35 | Q9UPQ4 | AF492463 | 23087 | NM_015066; | Hs.104223 | RF; BBOX; SPRY |
| | | | | NM_171982 | | |
| TRIM36 | Q9NQ86 | AJ272269 | 55521 | NM_018700 | Hs.519514 | RF; BBOX; |
| | | | | | | POSTBBOX; SPRY |
| TRIM37 | O94972 | AB020705 | 4591 | NM_001005207 | Hs.412767 | RF; BBOX; TRAF |
| | | | | NM_015294; | | |
| TRIM38 | O00635 | U90547 | 10475 | NM_006355 | Hs.202510 | RF; BBOX; |
| | | | | | | POSTBBOX; SPRY |
| TRIM39/RNF23 | Q9HCM9 | AB046381 | 56658 | NM_172016; | Hs.413493 | RF; BBOX; |
| | | | | NM_021253 | | POSTBBOX; SPRY |
| TRIM4 | Q9C037 | AF220023 | 89122 | NM_057096; | Hs.50749 | RF; BBOX; |
| | | | | NM_057095; | | POSTBBOX; SPRY |
| | | | | NM_022820; | | |
| | | | | NM_033091; | | |
| | | | | NM_033017 | | |
| TRIM40/RNF35 | Q6P9F5 | BC060785 | 135644 | NM_138700 | Hs.509439 | RF; BBOX |
| TRIM41 | Q8WV44 | AB100366 | 90933 | NM_033549; | Hs.441488 | RF; BBOX; |
| | | | | NM_201627 | | POSTBBOX; SPRY |
| TRIM42 | Q8IWZ5 | AF521868 | 287015 | NM_152616 | Hs.343487 | RF; BBOX; |
| | | | | | | POSTBBOX |
| TRIM43 | Q96BQ3 | BC015353 | 129868 | NM_138800 | Hs.232026 | RF; BBOX; SPRY |
| TRIM45 | Q9H8W5 | AY669488 | 80263 | NM_025188 | Hs.301526 | RF; BBOX; |
| | | | | | | POSTBBOX |
| TRIM46 | Q7Z4K8 | AY251386 | 80128 | NM_025058 | Hs.287735 | RF; BBOX; |
| | | | | | | POSTBBOX; SPRY |
| TRIM47 | Q96LD4 | AY026763 | 91107 | NM_033452 | Hs.293660 | RF; BBOX; SPRY |
| TRIM48 | Q8IWZ4 | AF521869 | 79097 | NM_024114 | Hs.195715 | RF; BBOX; SPRY |
| TRIM49/RNF18 | Q9NS80 | AB037682 | 57093 | NM_020358 | Hs.534218 | RF; BBOX; SPRY |
| TRIM5 | Q9C035 | AF220025 | 85363 | NM_033034; | Hs.370515 | RF; BBOX; |
| | | | | NM_033093; | | POSTBBOX; SPRY |
| | | | | NM_033092 | | |
| TRIM50A | Q86XT4 | AY081948 | 135892 | NM_178125 | Hs.404810 | RF; BBOX; SPRY |
| TRIMSOB | Q86UV7 | AF498998 | none | XM_353628 | Hs.511015 | RF; BBOX |
| TRIM50C | Q86UV6 | AF498999 | 378108 | NM_198853 | Hs.534009 | RF; BBOX; |
| | | | | | | ZF_RAD18 |
| TRIM52 | Q96A61 | AK054802 | 84851 | NM_032765 | Hs.458412 | RF; BBOX |
| TRIM54/RNF30 | Q9BYV2 | AJ291714 | 57159 | NM_187841; | Hs.516036 | RF; BBOX; |
| | | | | NM_032546 | | POSTBBOX |
| TRIM55/RNF29 | Q9BYV6 | BC007750 | 84675 | NM_184087; | Hs.85524 | RF; BBOX; |
| | | | | NM_184085; | | POSTBBOX |
| | | | | NM_184086; | | |
| | | | | NM_033058 | | |
| TRIM56 | Q9BRZ2 | BC005847 | 81844 | NM_030961 | Hs.521092 | RF; BBOX; |
| | | | | | | POSTBBOX |
| TRIM58/BIA2 | Q8NG06 | AK096188 | 25893 | NM_015431 | Hs.323858 | RF; POSTBBOX; |
| | | | | | | SPRY |
| TRIM59/TSBF1 | Q8IWR1 | AY159379 | 286827 | NM_173084 | Hs.212957 | RF; BBOX |
| TRIM6 | Q9C030 | AF220030 | 117854 | NM_058166; | Hs.350518 | RF; BBOX; |
| | | | | NM_001003818 | | POSTBBOX; SPRY |
| TRIM60/FLJ35882 | Q8NA35 | AK093201 | 166655 | NM_152620 | Hs.368004 | RF; BBOX; |
| | | | | | | POSTBBOX; SPRY |
| TRIM61 | Q5EBN2 | BC089393 | 391712 | NM_001012414 | Hs.529351 | RF; BBOX |
| TRIM62 | Q9BVG3 | BC001222 | 55223 | NM_018207 | Hs.404997 | RF; BBOX; |
| | | | | | | POSTBBOX; SPRY |
| TRIM63/RNF28 | Q969Q1 | AF353673 | 84676 | NM_032588 | Hs.279709 | RF; BBOX; |
| | | | | | | POSTBBOX |
| TRIM65 | Q6PJ69 | BCO21259 | 201292 | NM_173547 | Hs.189823 | RF; BBOX; SPRY |
| TRIM67/TNL | Q7Z4K7 | AY253917 | 440730 | NM_001004342 | Hs.131295 | RF; BBOX; |
| | | | | | | POSTBBOX; SPRY |
| TRIM68 | Q6AZZ1 | BC075058 | 55128 | NM_018073 | Hs.523438 | RF; BBOX; SPRY |
| TRIM7 | Q9CO29 | AF396651 | 81786 | NM_203295; | Hs.487412 | RF; BBOX; |
| | | | | NM_203297; | | POSTBBOX; SPRY |
| | | | | NM_203294; | | |
| | | | | NM_203293; | | |
| | | | | NM_033342; | | |
| TRIM8 | Q9BZR9 | AF220034 | 81603 | NM_203296 | Hs.336810 | RF; POSTRF; BBOX |
| | | | | NM_030912 | | |
| TRIM9 | Q9CO26 | AF220037 | 114088 | NM_052978; | Hs.368928 | RF; BBOX; |
| | | | | NM_015163 | | POSTBBOX; SPRY |
| TRIP/TRAIP | Q9BWF2 | BC000310 | 10293 | NM_005879 | Hs.517972 | RF |
| TTC3 | P53804 | D83077 | 7267 | NM_001001894; | Hs.368214 | RF; TPR |
| | | | | NM_003316 | | |
| UBOXS/RNF37 | O94941 | AB020667 | 22888 | NM_199415; | Hs.129448 | RF |
| | | | | NM_014948 | | |
| UBR1 | Q8IWV7 | AY061886 | 197131 | NM_174916 | Hs.145209 | RF; CLPS; ZF UBR1 |
| UBR2/UBR1L2 | Q8IWV8 | AY061884 | 23304 | NM_015255 | Hs.529925 | RF; CLPS; ZF UBR1 |
| UHRF1/FLJ21925 | Q9H6S6 | AK025578 | 29128 | NM_013282 | Hs.108106 | RF |
| UHRF2 | Q659C8 | AL137728 | 115426 | NM_152896; | Hs.493401 | RF |
| | | | | NM_152306 | | |
| VPS11 | Q9H270 | AF308800 | 55823 | NM_021729 | Hs.234282 | RF; Clath |
| VPS18 | Q9P253 | AF308802 | 57617 | NM_020857 | Hs.23876 | RF; Clath |
| VPS41 | P49754 | U87309 | 27072 | NM_014396 | Hs.148721 | RF; Clath |
| | | | | NM_080631; | | |
| ZFPL1 | O95159 | AF030291 | 7542 | NM_006782 | Hs.155165 | RF |
| ZNF179 | Q9ULX5 | AB026054 | 7732 | NM_007148 | Hs.189482 | RF; GTPase |
| ZNF183 | O15541 | X98253 | 7737 | NM_006978 | Hs.458365 | RF ZF_CCCH |
| ZNF183L1 | Q8IZP6 | BC017585 | 140432 | NM_178861 | Hs.296045 | RF ZF_CCCH |
| ZNF294 | O94822 | AB018257 | 26046 | NM_015565 | Hs.288773 | RF; GIUEV |
| ZNF313 | Q9Y508 | AF265215 | 55905 | NM_018683 | Hs.144949 | RF; ZF ZNF313 |
| ZNF364 | Q9Y4L5 | AF419857 | 27246 | NM_014455 | Hs.523550 | RF; ZF CIP8 |
| ZNF598 | Q86UK7 | BC050477 | 90850 | NM_178167 | Hs.343828 | RF |
| ZNF645 | Q6DJY9 | BC074910 | 158506 | NM_152577 | Hs.132485 | RF; BBOX |
| ZNF650/UBR1L1 | Q6ZT12 | AK126998 | 130507 | NM_172070 | Hs.379548 | RF; UBR1CT |
| ZNRF1 | Q8ND25 | AL834440 | 84937 | NM_032268 | Hs.427284 | RF; ZF_RAD18 |
| ZNRF2 | Q8NHG8 | AF527533 | 223082 | NM_147128 | Hs.487869 | RF; ZF_RAD18 |
| ZNRF3/KIAA1133 | Q9ULT6 | AB051436 | none | XM 290972 | Hs.134473 | RF |
| ZNRF4/LOC148066 | Q8WWF5 | BC017592 | 148066 | NM_181710 | Hs.126496 | RF; PA |
| ZSWIM2 | Q8NEG5 | BC031094 | 151112 | NM_182521 | Hs.375054 | RF; ZZ |
| ZZANK1/Skeletrophin | Q8NI59 | AB074480 | 142678 | NM_080875 | Hs.135805 | RF; Ank; MIBOREP; |
| | | | | | | MIBHERC2; ZZ |
| ENSP00000280266 | ENSP00000280266 | ENST00000280266 | none | none | none | RF; BBOX; SPRY |
| ENSP00000344026 | ENSP00000344026 | ENST00000344287 | none | XM 292796 | Hs.451647 | RF; SPRY |
| ENSP00000348371 | ENSP00000348371 | ENST00000356071 | none | XM 373101 | Hs.356440 | RF; NEURALIZED |
| GENSCAN00000024511 | GENSCAN00000024511H | GENSCAN00000024511 | none | XM 497353 | Hs.131991 | RF; SPRY |
| DKFZp434E1818 | ENSP00000343122 | AL133632 | none | XM 372169 | Hs.512564 | RF |
| MKRN4 | Q13434 | U41315 | | | | RF; ZF MAKORIN |

TABLE 3
E3 Ligases, PARKIN-Finger (PF) type

| Name | UniProt | Genbank | LL | Refseq | Unigene | Domains |
|---|---|---|---|---|---|---|
| ANKIBI | Q9P2G1 | AB037807 | none | XM_377955 | Hs.83293 | PF1; PF2; PF3; Ank; |
| | | | | | | ARICT; |
| | | | | | | ARINT; UIM |
| ARIH1/UBCH7BP | Q9Y4X5 | BC051877 | 25820 | NM_005744 | Hs.268787 | PF1; PF2; PF3; |
| | | | | | | ARICT; ARINT |
| ARIH2/TRIAD1 | 095376 | AF099149 | 10425 | NM_006321 | Hs.241558 | PF1; PF2; PF3; |
| | | | | | | ARICT; ARINT |
| IBRDC1 | Q8TC41 | BC026087 | 154214 | NM_152553 | Hs.368639 | PF1; PF2; PF3 |
| IBRDC2 | Q7Z419 | AB076367 | 255488 | NM_182757 | Hs. 148741 | PF1; PF2; PF3 |
| IBRDC3 | Q6ZMZ0 | AK131439 | 127544 | NM_153341 | Hs.546478 | PF1; PF2; PF3 |
| PARC | Q8IWT3 | AJ318215 | 23113 | NM_015089 | Hs.485434 | PF1; PF2; PF3; |
| | | | | | | ARICT; ARINT; CULLIN; |
| | | | | | | DOC1; |
| | | | | | | HERC2 |
| PARK2 | O60260 | AB009973 | 5071 | NM_013987; | Hs.132954 | PF1; PF2; PF3; Ubiq |
| | | | | NM_004562; | | |
| | | | | NM_013988 | | |
| RNF14/ARA54 | Q9UBS8 | AB022663 | 9604 | NM_183401; | Hs.508993 | PF1; PF2; PF3; GIUEV |
| | | | | NM_183398; | | |
| | | | | NM_183399; | | |
| | | | | NM_183400; | | |
| | | | | NM_004290 | | |
| RNF144 | P50876 | 1379983 | 9781 | NM_014746 | Hs.22146 | PF1; PF2; PF3 |
| RNF19 | Q9NV58 | AB029316 | 25897 | NM_183419; | Hs.292882 | PF1; PF2; PF3 |
| | | | | NM_015435 | | |
| RNF31 | Q96EP0 | BC012077 | 55072 | NM_017999 | Hs.375217 | PF1; PF2; PF3; PUB; UBA; |
| | | | | | | ZFm; NZF |
| UBCE7IP1/TRIAD3 | Q9NWF9 | AF513717 | 54476 | NM_207116; | Hs.487458 | PF1; PF2; PF3 |
| | | | | NM_207111; | | |
| | | | | NM_019011 | | |
| UBCE7IP3/C200RF18 | Q9BYM8 | BC015219 | 10616 | NM_031229; | Hs.247280 | PF1; PF2; PF3; Ubiq; NZF |
| | | | | NM_006462; | | |
| | | | | NM_031227; | | |
| | | | | NM_031228 | | |
| GENSCAN00000039330H | GENSCAN00000039330H | | | | | PF1; PF2; PF3; |

TABLE 4
E3 Ligases, RING-variants type

| Name | UniProt | Type | Genbank | LL | Refseq | Unigene | Domains |
|---|---|---|---|---|---|---|---|
| C20orf43 | Q9BY42 | RINGvar | AF161518 | 51507 | NM_016407 | Hs.517134 | RFvar |
| FLJ13910 | Q9H871 | RINGvar | AK023972 | 64795 | NM_022780 | Hs.75277 | CTLH; RFvar; LISH |
| FLJ22318 | Q96G75 | RINGvar | AL713670 | 64777 | NM_022762 | Hs.519804 | CTLH; RFvar |
| MAEA | Q9BQ11 | RINGvar | BC006470 | 10296 | NM_005882 | Hs.139896 | CTLH; RFvar; LISH |
| NOSIP | Q96FD2 | RINGvar | BC011249 | 51070 | NM_015953 | Hs.7236 | RFvar |
| PPIL2 | Q13356 | RINGvar | U37219 | 23759 | NM_148175; | Hs.438587 | PPI2; RFvar |
| | | | | | NM_014337; | | |
| | | | | | NM_148176 | | |
| WDR59 | Q96PW5 | RINGvar | AB067510 | 79726 | NM_030581 | Hs.280951 | RFvar, WD, uev |
| FLJ20323 | Q7L551 | RINGvar | BC005883 | 54468 | NM_019005 | Hs.520215 | RFvar |
| JFP7 | Q96S15 | RINGvar | AL136863 | 84219 | NM_032259 | Hs.459632 | RFvar, WD |
| GTF2H2 | Q13888 | RINGvar | AF078847 | 2966 | NM_001515 | Hs.191356 | RFvar, vWFA |

TABLE 5
E3 Ligases, U-box type

| Name | UniProt | Genbank | LL | Refseq | Unigene | Domains |
|---|---|---|---|---|---|---|
| CHIP/STUB1 | Q9UNE7 | AF129085 | 10273 | NM_005861 | Hs.533771 | Ubox; TPR |
| PRP19/SNEV | Q9UMS4 | AJ131186 | 27339 | NM_014502 | Hs.502705 | Ubox; WD |
| UBE4B/UFD2 | O95155 | AF043117 | 10277 | NM_ 006048 | Hs.386404 | Ubox; |
| WDSAM1 | Q8N6N8 | BCO29520 | 151525 | NM_ 152528 | Hs.20848 | Ubox; SAM; WD |
| GENSCAN00000045262H | GENSCAN00000045262H | | | | | Ubox |
| (Ubox pseudogene) | | | | | | |

TABLE 6
E3 Ligases, A20-finger type

| Name | UniProt | Genbank | LL | Refseq | Unigene | Domains |
|---|---|---|---|---|---|---|
| C15orf16/Cezanne2 | Q8TE49 | AJ430383 | 161725 | NM_130901 | Hs.355236 | A20; OTU; UBAlike |
| RABGEF1/RABEX5 | Q9UJ41 | BC015330 | 27342 | NM_014504 | Hs.530053 | A20; VPS9 RIN |
| TEX27 | Q9H8U3 | AK023284 | 60685 | NM_021943 | Hs.36959 | A20; ZF UF |
| TNFAIP3/A20 | P21580 | M59465 | 7128 | NM_006290 | Hs.211600 | A20; OTU |
| ZA20D1/Cezannel | Q6GQQ9 | AJ293573 | 56957 | NM_020205 | Hs.98322 | A20; OTU; UBAlike |
| ZA20D2 | O76080 | AF062346 | 7763 | NM_006007 | Hs.406096 | A20; ZF UF |
| ZA20D3/AWP1 | Q9GZY3 | AF261138 | 54469 | NM_019006 | Hs.306329 | A20; ZF UF |

TABLE 7
E3 Ligases, PIAS-finger type

| Name | UniProt | Type | Genbank | LL | Refseq | Unigene | Domains |
|---|---|---|---|---|---|---|---|
| FLJ32440 | Q96MF7 | PIAS | AK057002 | 286053 | NM_173685 | Hs.388297 | PIAS |
| PIAS1 | 075925 | PIAS | AF167160 | 8554 | NM_016166 | Hs.162458 | PIAS; SAF |
| PIAS2/PIASx | 075928 | PIAS | AF077954 | 9063 | NM_004671 | Hs.514846 | PIAS; SAF |
| | | | | | NM_173206 | | |
| PIAS3 | Q9Y6X2 | PIAS | BC001154 | 10401 | NM_006099 | Hs.435761 | PIAS; SAF |
| PIAS4/PIASy | Q8N2W9 | PIAS | BCO29874 | 51588 | NM_015897 | Hs.105779 | PIAS; SAF |
| RAI17 | Q9ULJ6 | PIAS | AY235683 | 57178 | NM_020338 | Hs.193118 | PIAS |
| ZIMP7/ | Q8NF64 | PIAS | AK090415 | 83637 | NM_174929 | Hs.77978 | PIAS |
| DKFZp76112123 | | | | | NM_031449 | | |

| | | |
|---|---|---|
| FLJ13517 | Q9H8K2 | RNF138 |

TABLE 8
E3 Ligases, PHD-finger type

| Name | UniProt | Type | Genbank | LL | Refseq | Unigene | Domains |
|---|---|---|---|---|---|---|---|
| AIRE | O43918 | PHD | Z97990 | 326 | NM_000658; | Hs.129829 | PHD; SAND; SPC100 |
| | | | | | NM_000383; | | |
| | | | | | NM_000659 | | |

TABLE 9
E3 Ligases, Skp1-like type

| Name | UniProt | Type | Genbank | LL | Refseq | Unigene | Domains |
|---|---|---|---|---|---|---|---|
| SKP1A | P63208 | Skp1like | U33760 | 6500 | NM_170679; | Hs.171626 | Skp1 |
| | | | | | NM_006930 | | |
| TCEB1/Elongin C | Q15369 | Skp1like | L34587 | 6921 | NM_005648 | Hs.546305 | Skp1 |
| | P78561 | Skp1like | L49176 | | | | Skp1 |
| | | pseudogene | | | | | |
| | P78389 | Skpllike | L49173 | | | | Skp1 |
| | | pseudogene | | | | | |
| RP1-254P11.1-001 | Q9H575 | Skp1like | AL136318 | | | | Skp1 |
| Fos393471 | | pseudogene | | | | | |
| | O75863 | Skp1like | | | | | Skp1 |
| | | pseudogene | | | | | |

TABLE 10
E3 Ligases, Cullin type

| Name | UniProt | Type | Genbank | LL | Refseq | Unigene | Domains |
|---|---|---|---|---|---|---|---|
| ANAPC2 | Q9UJX6 | Cullin | BC032503 | 29882 | NM_013366 | Hs.533262 | CULLIN |
| CUL1 | Q13616 | Cullin | AF062536 | 8454 | NM_003592 | Hs.146806 | CULLIN |
| CUL2 | Q13617 | Cullin | AF126404 | 8453 | NM_003591 | Hs.82919 | CULLIN |
| CUL3 | Q13618 | Cullin | AF064087 | 8452 | NM_003590 | Hs.372286 | CULLIN |
| CUL4A | Q13619 | Cullin | AF077188 | 8451 | NM_003589; | Hs.339735 | CULLIN |
| | | | | | NM_001008895 | | |
| CUL4B | Q13620 | Cullin | AY365125 | 8450 | NM_003588 | Hs.102914 | CULLIN |
| CULS | Q93034 | Cullin | AF327710 | 8065 | NM_003478 | Hs.440320 | CULLIN |
| CULT | Q14999 | Cullin | D38548 | 9820 | NM_014780 | Hs.520136 | CULLIN; DOC1; HERC2 |
| PARC | Q8IWT3 | Cullin | A7318215 | 23113 | NM_015089 | Hs.485434 | ARICT; ARINT; CULLIN |
| | | | | | | | DOC1; HERC2; PF1; PF2; |
| | | | | | | | PF3 |

TABLE 11
E3 Ligases, F-box type

| Name | UniProt | Type | Genbank | LL | Refseq | Unigene | Domains |
|---|---|---|---|---|---|---|---|
| FBXL1/SKP2 | Q13309 | Fbox | AB050979 | 6502 | NM_032637; | Hs.23348 | FBOX; LRR |
| | | | | | NM_005983 | | |
| FBXL10 | Q8NHM5 | Fbox | AJ459424 | 84678 | NM_001005366 | Hs.524800 | FBOX; LRR; PHD; |
| | | | | | | | JMJC; |
| | | | | | | | ZF_DNAMET |
| FBXL11 | Q9Y2K7 | Fbox | AB023221 | 22992 | NM_012308 | Hs.124147 | FBOX; LRR; PHD; |
| | | | | | | | JMJC; |
| | | | | | | | ZF_DNAMET |
| FBXL12 | Q9NXK8 | Fbox | AK000195 | 54850 | NM_017703 | Hs.12439 | FBOX; LRR |
| FBXL13 | Q8NEE6 | Fbox | AK097537 | 222235 | NM_145032 | Hs.434284 | FBOX; LRR |
| FBXL14 | Q8N1E6 | Fbox | BC028132 | 144699 | NM_152441 | Hs.367956 | FBOX; LRR |
| FBXL15/FBXO37 | Q9H469 | Fbox | CR592302 | none | XM370575 | Hs.380081 | FBOX; LRR |
| FBXL16 | Q8N461 | Fbox weak | BC036680 | 146330 | NM_153350 | Hs.513244 | FBOX; LRR; |
| FBXL17/FBXO13 | Q9UF56 | Fbox | AK126722 | 64839 | NM_022824 | Hs.112143 | FBOX; LRR |
| FBXL18 | Q96ME1 | Fbox | AK057042 | 80028 | NM_024963 | Hs.487447 | FBOX; LRR |
| FBXL19 | Q6PCT2 | Fbox | AK098777 | 54620 | NM_019085 | Hs.152149 | FBOX; LRR; PHD; |
| | | | | | | | ZF DNAMET |
| FBXL2 | Q9UKC9 | Fbox | AF176518 | 25827 | NM_012157 | Hs.475872 | FBOX; LRR |
| FBXL20 | Q96IG2 | Fbox | BC007557 | 84961 | NM_032875 | Hs.462946 | FBOX; LRR |
| FBXL21 | Q9UKT6 | Fbox | AF129533 | 26223 | NM_012159 | Hs.167877 | FBOX; LRR |
| FBXL22 | Q6P050 | Fbox | BC065833 | 283807 | NM_203373 | Hs.549302 | FBOX; LRR |
| FBXL3 | Q9UKT7 | Fbox | AF129532 | 26224 | NM_012158 | Hs.508284 | FBOX; LRR |
| FBXL4 | Q9UKA2 | Fbox | AF174590 | 26235 | NM_012160 | Hs.536850 | FBOX; LRR |
| FBXLS | Q9UKA1 | Fbox | AF176700 | 26234 | NM_012161; | Hs.479208 | FBOX; LRR |
| | | | | | NM_033535 | | |
| FBXL6 | Q8N531 | Fbox | #NAME? | 26233 | NM_012162; | Hs.12271 | FBOX; LRR |
| | | | | | NM_024555 | | |
| FBXL7 | Q9UJT9 | Fbox | AF199356 | 23194 | NM_012304 | Hs.433057 | FBOX; LRR |
| FBXL8 | Q96CDO | Fbox | AK002140 | 55336 | NM_018378 | Hs.75486 | FBOX; LRR |
| FBXL9/LRRC29 | Q8WV35 | Fbox | BC018785 | 26231 | NM_012163; | Hs.461000; | FBOX; LRR |
| | | | | | NM_001004055 | | |
| FBXL1/SKP2 | Q13309 | Fbox | AB050979 | 6502 | NM_032637; | Hs.23348 | FBOX; LRR |
| | | | | | NM_005983 | | |
| FBXL10 | Q8NHM5 | Fbox | AJ459424 | 84678 | NM_0010053 66 | Hs.524800 | FBOX; LRR; PHD; |
| | | | | | | | JMJC; ZF_DNAMET |
| FBXL11 | Q9Y2K7 | Fbox | AB023221 | 22992 | NM_012308 | Hs.124147 | FBOX; LRR; PHD; |
| | | | | | | | JMJC; ZF_DNAMET |
| FBXL12 | Q9NXK8 | Fbox | AK000195 | 54850 | NM_017703 | Hs.12439 | FBOX; LRR |
| FBXL13 | Q8NEE6 | Fbox | AK097537 | 222235 | NM_145032 | Hs.434284 | FBOX; LRR |
| FBXL14 | Q8N1E6 | Fbox | BCO28132 | 144699 | NM_152441 | Hs.367956 | FBOX; LRR |
| FBXL15/FBXO37 | Q9H469 | Fbox | CR592302 | none | XM370575 | Hs.380081 | FBOX; LRR |
| FBXL16 | Q8N461 | Fbox weak | BC036680 | 146330 | NM_153350 | Hs.513244 | FBOX; LRR; |
| FBXL17/FBXO13 | Q9UF56 | Fbox | AK126722 | 64839 | NM_022824 | Hs.112143 | FBOX; LRR |
| FBXL18 | Q96ME1 | Fbox | AK057042 | 80028 | NM_024963 | Hs.487447 | FBOX; LRR |
| FBXL19 | Q6PCT2 | Fbox | AK098777 | 54620 | NM_019085 | Hs.152149 | FBOX; LRR; PHD; |
| | | | | | | | ZF_DNAMET |
| FBXL2 | Q9UKC9 | Fbox | AF176518 | 25827 | NM_012157 | Hs.475872 | FBOX; LRR |
| FBXL20 | Q96IG2 | Fbox | BC007557 | 84961 | NM_032875 | Hs.462946 | FBOX; LRR |
| FBXL21 | Q9UKT6 | Fbox | AF129533 | 26223 | NM_012159 | Hs.167877 | FBOX; LRR |
| FBXL22 | Q6P050 | Fbox | BC065833 | 283807 | NM_203373 | Hs.549302 | FBOX; LRR |
| FBXL3 | Q9UKT7 | Fbox | AF129532 | 26224 | NM_012158 | Hs.508284 | FBOX; LRR |
| FBXL4 | Q9UKA2 | Fbox | AF174590 | 26235 | NM_012160 | Hs.536850 | FBOX; LRR |
| FBXLS | Q9UKA1 | Fbox | AFI76700 | 26234 | NM_012161; | Hs.479208 | FBOX; LRR |
| | | | | | NM_033535 | | |
| FBXL6 | Q8N531 | Fbox | #NAME? | 26233 | NM_012162; | Hs.12271 | FBOX; LRR |
| | | | | | NM_024555 | | |
| FBXL7 | Q9UJT9 | Fbox | AF199356 | 23194 | NM_012304 | Hs.433057 | FBOX; LRR |
| FBXL8 | Q96CDO | Fbox | AK002140 | 55336 | NM_018378 | Hs.75486 | FBOX; LRR |
| FBXL9/LRRC29 | Q8WV35 | Fbox | BC018785 | 26231 | NM_012163; | Hs.461000 | FBOX; LRR |
| | | | | | NM_001004055 | | |
| FBX01/CCNF | P41002 | Fbox | BC012349 | 899 | NM_001761 | Hs.1973 | FBOX; CYCLIN; |
| | | | | | | | SEL1 |
| FBXOlO | Q9UK96 | Fbox | AFI76705 | none | XM_291314 | none | FBOX |
| FBXO11 | Q86XK2 | Fbox | BC012728 | 80204 | NM_012167; | Hs.549201 | FBOX; ZF_UBRI |
| | | | | | NM_025133 | | |
| | | | | | NM_018693 | | |
| FBXO15 | Q8NCQ5 | Fbox | BC029579 | 201456 | NM_152676 | Hs.465411 | FBOX |
| FBX016 | Q8IX29 | Fbox | AF453435 | 157574 | NM_172366 | Hs.532253 | FBOX |
| FBXO17/FBXO26 | Q96EF6 | Fbox | AF386743 | 115290 | NM_148169; | Hs.531770 | FBOX |
| | | | | | NM_024907 | | |
| FBXO18 | Q8NFZ0 | Fbox | AF380349 | 84893 | NM_178150; | Hs.498543 | FBOX |
| | | | | | NM_032807 | | |
| FBXO2 | Q9UK22 | Fbox | BC025233 | 26232 | NM_012168 | Hs.132753 | FBOX |
| FBXO21 | O94952 | Fbox | AF174601 | 23014 | NM_015002; | Hs.159699 | FBOX |
| | | | | | NM_033624 | | |
| FBXO22 | Q8NEZ5 | Fbox | AY005144 | 26263 | NM_147188; | Hs.458959 | FBOX |
| | | | | | NM_012170 | | |
| FBXO24 | 075426 | Fbox | AL136811 | 26261 | NM_033506; | Hs.283764 | FBOX; RCC |
| | | | | | NM_012172 | | |
| FBXO25 | Q8TCJO | Fbox | CR596773 | 26260 | NM_183420; | Hs.438454 | FBOX |
| | | | | | NM_183421; | | |
| | | | | | NM_012173 | | |
| FBXO27 | Q8NI29 | Fbox | BC014527 | 126433 | NM_178820 | Hs.187461 | FBOX; |
| FBXO28 | Q9NVF7 | Fbox | AK001628 | 23219 | NM_015176 | Hs.64691 | FBOX; |
| FBXO3 | Q9UK99 | Fbox | AK001943 | 26273 | NM_033406; | Hs.406787 | FBOX; |
| | | | | | NM_012175 | | |
| FBXO30 | Q8TB52 | Fbox | BC024326 | 84085 | NM_032145 | | Hs .421095 |
| FBXO31/FBXO14 | Q5XUX0 | Fbox | AY736035 | 79791 | NM_024735 | | Hs .549198 |
| FBXO32 | Q969P5 | Fbox | AY059629 | 114907 | NM_148177; | Hs.403933 | FBOX |
| FBXO33 | Q7Z6M2 | Fbox | BC053537 | 254170 | NM_203301 | Hs.324342 | FBOX |
| FBXO34 | Q9NWN3 | Fbox | BX248268 | 55030 | NM_017943 | Hs.525348 | FBOX |
| FBXO36 | Q8NEA4 | Fbox | BC033935 | 130888 | NM_174899 | Hs.140666 | FBOX |
| FBXO38 | Q6PIJ6 | Fbox | BC005849 | 81545 | NM_205836; | Hs.483772 | FBOX |
| | | | | | NM_030793 | | |
| FBXO39 | Q8N4B4 | Fbox | BC034782 | 162517 | NM_153230 | Hs.368364 | FBOX |
| FBXO4 | Q9UKT5 | Fbox | BC048098 | 26272 | NM_012176; | Hs.165575 | FBOX |
| | | | | | NM_033484 | | |
| FBXO40 | Q9UH90 | Fbox | AF204674 | 51725 | NM_016298 | Hs.272564 | FBOX; ZFt |
| FBXO41 | Q8TF61 | Fbox | AB075820 | none | XM_377742 | Hs.23158 | FBOX |
| FBXO42 | Q6P3S6 | Fbox | BC063864 | none | XM_048774 | Hs.522384 | FBOX; KELCH |
| FBXO43 | ENSP00000322 | Fbox | BCO28709 | none | XM_209918 | Hs.339577 | FBOX |
| | 600 | | | | | | |
| FBXO44 | Q9H4M3 | Fbox | AK055344 | 93611 | NM_183413; | Hs.519716 | FBOX |
| | | | | | NM_183412; | | |
| | | | | | NM_033182 | | |
| FBXO45 | ENSP00000310 | Fbox | AK025697 | none | XM_117294 | Hs.518526 | FBOX; SPRY |
| | 332 | | | | | | |
| FBXO46 | Q6PJ61 | Fbox | BCO21978 | none | XM_371179 | Hs.128702 | FBOX |
| FBXO5 | Q9UKT4 | Fbox | AF129535 | 26271 | NM_012177 | Hs.520506 | FBOX |
| FBXO6 | Q9NRD1 | Fbox | AF233223 | 26270 | NM_018438 | Hs.464419 | FBOX |
| FBXO7 | Q9Y3I1 | Fbox | AF233225 | 25793 | NM_012179 | Hs.5912 | FBOX;Ubiq |
| FBXO8 | Q9NRD0 | Fbox | AF233224 | 26269 | NM_012180 | Hs.76917 | FBOX; Sec7 |
| FBXO9 | Q9UK97 | Fbox | AF176704 | 26268 | NM_033481; | Hs.216653 | FBOX |
| | | | | | NM_033480; | | |
| | | | | | NM_012347 | | |
| FBXW1/BTRC | Q9Y297 | Fbox | AF101784 | 8945 | NM_033637; | Hs.500812 | FBOX; WD |
| | | | | | NM_003939 | | |
| FBXW10 | Q5XX13 | Fbox | AY729024 | 10517 | NM_031456 | Hs.310275 | FBOX; WD |
| FBXW11 | Q9UKB1 | Fbox | AFI76022 | 23291 | NM_033645; | Hs.484138 | FBOX; WD |
| | | | | | NM_033644; | | |
| | | | | | NM_012300 | | |
| FBXW12/FBX035 | Q6X9E4 | Fbox | AY247969 | 285231 | NM_207102 | Hs.288793 | FBOX |
| FBXW2 | Q9UKT8 | Fbox | BC018738 | 26190 | NM_012164 | Hs.494985 | FBOX; WD |
| FBXW3 | Q9UKB7 | Fbox | AF174606 | none | none | none | FBOX; WD |
| FBXW4/SHFM3 | P57775 | Fbox | AF281859 | 6468 | NM_022039 | Hs.500822 | FBOX; WD |
| FBXWS | Q969U6 | Fbox | BC014297 | 54461 | NM_018998; | Hs.522507 | FBOX; WD |
| | | | | | NM_178226; | | |
| | | | | | NM_178225 | | |
| FBXW7/FBXW6 | Q969H0 | Fbox | AF411971 | 55294; | NM_033632 | Hs.519029 | FBOX; WD |
| | | | | | NM_018315 | | |
| FBXW8/FBX029 | Q8N3Y1 | Fbox | BC037296 | 26259 | NM_153348; | Hs.435466 | FBOX; WD |
| | | | | | NM_018315 | | |
| FBXW9 | Q5XUX1 | Fbox | AY736034 | 84261 | NM_032301 | Hs.515154 | FBOX; WD |
| FBXW15 | Q8BI39 | Fbox | AK087669 | | | | FBOX |
| FBXW16 | Q8BIU6 | Fbox | AK085629 | | | | FBOX |
| FBXW17 | Q8CFE8 | Fbox | BC040428 | | | | FBOX |
| FBXW19 | Q8C2W8 | Fbox | AK087808 | | | | FBOX |
| FBXO12/FBXW14 | Q8C2Y5 | Fbox | AK087709 | | | | FBOX |

TABLE 12
E3 Ligases, SOCS-box type

| Name | UniProt | Genbank | LL | Refseq | Unigene | Domains |
|---|---|---|---|---|---|---|
| TULP4 | Q9NRJ4 | AF219946 | 56995 | NM_020245; | Hs.486993 | SOCS; TUBBY; WD |
| | | | | NM_001007466 | | |
| WSB1 | Q9Y6I7 | AF072880 | 26118 | NM_015626; | Hs.446017 | SOCS; WD |
| | | | | NM_134264; | | |
| | | | | NM_134265 | | |
| WSB2 | Q9NYS7 | AF229181 | 55884 | NM_018639 | Hs.506985 | SOCS; WD |
| ASB1 | Q9Y576 | AF156777 | 51665 | NM_016114 | Hs.516788 | SOCS; ANK |
| ASB2 | Q96Q27 | AB056723 | 51676 | NM_016150 | Hs.510327 | SOCS; ANK |
| ASB3 | Q9Y575 | AF156778 | 51130 | NM_145863; | Hs.40763 | SOCS; ANK |
| | | | | NM_016115 | | |
| ASB4 | Q9Y574 | AF156779 | 51666 | NM_016116; | Hs.127735 | SOCS; ANK |
| | | | | NM_145872 | | |
| ASBS | Q8WWX0 | AY057053 | 140458 | NM_080874 | Hs.352364 | SOCS; ANK |
| ASB6 | Q9NWX5 | AK000555 | 140459 | NM_177999; | Hs.125037 | SOCS; ANK |
| | | | | NM_017873 | | |
| ASB7 | Q9H672 | AF451994 | 140460 | NM_198243; | Hs.31845 | SOCS; ANK |
| | | | | NM_024708 | | |
| ASB8 | Q9H765 | AK024908 | 140461 | NM_024095 | Hs.432699 | SOCS; ANK |
| ASB9 | Q96DX5 | BC013172 | 140462 | NM_024087 | Hs.19404 | SOCS; ANK |
| ASB10 | Q8WXI3 | AF417920 | 136371 | NM_080871 | Hs.304273 | SOCS; ANK |
| ASB11 | Q8WXH4 | AF425642 | 140456 | NM_080873 | Hs.352183 | SOCS; ANK |
| ASB12 | Q8WXK4 | AF403030 | 142689 | NM_130388 | Hs.56281 | SOCS; ANK |
| ASB13 | Q8WXK3 | CR457302 | 79754 | NM_024701 | Hs.445899 | SOCS; ANK |
| ASB14 | Q8WXK2 | AF403032 | 142686 | NM_130387 | Hs.435978 | SOCS; ANK |
| ASB15 | Q8WXKI | AK125360 | 142685 | NM_080928 | Hs.97709 | SOCS; ANK |
| ASB16 | Q96NS5 | AK054727 | 92591 | NM_080863 | Hs.534517 | SOCS; ANK |
| ASB17 | Q8WXJ9 | AK098606 | 127247 | NM_080868 | Hs.125423 | SOCS; ANK |
| RAB40A | Q8WXH6 | AF422143 | 142684 | NM_080879 | Hs.549244 | SOCS; GTPase |
| RAB40B | Q12829 | U05227 | 10966 | NM_006822 | Hs.484068 | SOCS; GTPase |
| RAB40C | Q96S21 | BC028696 | 57799 | NM_021168 | Hs.459630 | SOCS; GTPase |
| SOCS1 | O15524 | AB005043 | 8651 | NM_003745 | Hs.50640 | SOCS; SH2 |
| SOCS2 | O14508 | AB004903 | 8835 | NM_003877 | Hs.485572 | SOCS; SH2 |
| SOCS3 | O14543 | AB006967 | 9021 | NM_003955 | Hs.527973 | SOCS; SH2 |
| SOCS4 | Q8WXH5 | AF424815 | 122809 | NM_199421; | Hs.532610 | SOCS; SH2 |
| | | | | NM_080867 | | |
| SOCS5 | O75159 | AF073958 | 9655 | NM_014011; | Hs.468426 | SOCS; SH2 |
| | | | | NM_144949 | | |
| SOCS6 | O14544 | AB006968 | 9306 | NM_004232 | Hs.44439 | SOCS; SH2 |
| SOCS7 | O14512 | AB005216 | 30837 | NM_014598 | Hs.514132 | SOCS; SH2 |
| CISH | Q9NSE2 | D83532 | 1154 | NM_013324; | Hs.8257 | SOCS; SH2 |
| | | | | NM_145071 | | |
| SSBI | Q96BD6 | BC015711 | 80176 | NM_025106 | Hs.8261 | SOCS; SPRY |
| SSB3 | Q96IE6 | BC007588 | 90864 | NM_080861 | Hs.7247 | SOCS; SPRY |
| SSB4 | Q96A44 | AK056367 | 92369 | NM_080862 | Hs.477752 | SOCS; SPRY |
| GRCC9/SSB2 | Q99619 | AF403027 | 84727 | NM_032641 | Hs.479856 | SOCS; SPRY |
| LOC196394 | Q8IY45 | BC037897 | 196394 | NM_207337 | Hs.131393 | SOCS; LRR |
| TCEB3 | Q14241 | L47345 | 6924 | NM_003198 | Hs.549069 | SOCS; TFIIS |
| TCEB3B | Q8IYF1 | BC036022 | 51224 | NM_016427 | Hs.375035 | SOCS; TFIIS |
| TCEB3C | Q8NG57 | AB076840 | 162699 | NM_145653 | Hs.515381 | SOCS; TFIIS |
| NEURL2 | Q9BRO9 | AK054821 | 140825 | NM_080749 | Hs.517094 | SOCS; Neuralized |
| VHL | P40337 | L15409 | 7428 | NM_000551; | Hs.421597 | SOCS; VHL HYPRO |
| | | | | NM_198156 | | |

TABLE 13
E3 Ligases, BTB type

| Name | UniProt | Genbank | LL | Refseq | Unigene | Domains |
|---|---|---|---|---|---|---|
| ABTBI | Q969K4 | AB053325 | 80325 | NM_172028; | Hs.107812 | ANK; BTB |
| | | | | NM_172027 | | |
| | | | | NM_032548 | | |
| ABTB2 | Q8N961 | AK095632 | 25841 | NM_145804 | Hs.23361 | ANK; BTB |
| ANKFYl | Q9P2R3 | AK025483 | 51479 | NM_016376; | Hs.513875 | ANK; FYVE; |
| | | | | NM_020740 | | BTB |
| APM-1 | O73453 | Y14591 | none | XM_113971 | Hs.515388 | BTB; ZFb |
| BACHI | O14867 | AB002803 | 571 | NM_001186; | Hs.154276 | bZIP; BTB |
| | | | | NM_206866 | | |
| BACH2 | Q9BYV9 | AF357835 | 60468 | NM_021813 | Hs.269764 | bZIP; BTB |
| BCL6/ZBTB27 | P41182 | Z21943 | 604 | NM_001706; | Hs.478588 | BTB; ZFb |
| | | | | NM_138931 | | |
| BCL6B/ZBTB28 | Q8N143 | AB076580 | 255877 | NM_181844 | Hs.22575 | BTB; ZFb |
| BKLHD5/KIAA1900 | Q96NJ5 | AK055292 | 114792 | NM_052904 | Hs.45056 | BTB; KELCH |
| BTBD1 | Q9H005 | AL136853 | 53339 | NM_025238 | Hs.459149 | BTB |
| BTBD11/FLJ42845 | Q6ZV99 | AK124835 | 121551 | NM_152322 | Hs.271272 | ANK; BTB |
| BTBD12 | Q8IY92 | BC036335 | none | none | Hs.513297 | BTB |
| BTBD14A | Q96BF6 | BC015649 | 138151 | NM_144653 | Hs.112895 | BTB |
| BTBD14B | Q96RE7 | AF395817 | 112939 | NM_052876 | Hs.531614 | BTB |
| BTBD2 | Q9BX70 | AF355797 | 55643 | NM_017797 | Hs.465543 | BTB |
| BTBD3 | Q9Y2F9 | AB023169 | 22903 | NM_014962; | Hs.244590 | BTB |
| | | | | NM_181443 | | |
| BTBD4 | Q86UZ6 | AK131482 | 140685 | NM_025224 | Hs.551578 | BTB; ZFb |
| BTBD5 | Q9NXS3 | BC012473 | 54813 | NM_017658 | Hs.174682 | BTB; KELCH |
| BTBD6 | Q96KE9 | AF353674 | 90135 | NM_033271 | Hs.7367 | BTB |
| BTBD7/KIAA1525 | Q9P203 | AB040958 | 55727 | NM_018167; | Hs.525549 | BTB |
| | | | | NM_001002860 | | |
| BTBD8 | Q5XKL5 | BC013922 | 284697 | NM_183242 | Hs.383108 | BTB |
| BTBD9 | Q96Q07 | AB067467 | 114781 | NM_152733 | Hs.116233 | BTB |
| C10orf87 | Q96LNO | AK058088 | 118663 | NM_144587 | Hs.422466 | BTB |
| Cl6orf44 | Q8N4N3 | BC033821 | 79786 | NM_024731 | Hs.222731 | BTB; KELCH |
| CCIN | Q13939 | AF333334 | 881 | NM_005893 | Hs.115460 | BTB; KELCH |
| CHC1L | O95199 | AF060219 | 1102 | NM_001268 | Hs.25447 | BTB; RCC |
| DRE1 | Q6TFL4 | AY422472 | 54809 | NM_017644 | Hs.407709 | BTB; KELCH |
| ENC1 | O14682 | AF059611 | 8507 | NM_003633 | Hs.1104925 | BTB; KELCH |
| ENC2/DKFZp434K111 | Q9H0H3 | AL136796 | 64410 | NM_022480 | Hs.498371 | BTB; KELCH |
| FLJ11078 | Q8TAP0 | BC026319 | 55295 | NM_018316 | Hs.250632 | BTB; KELCH |
| FLJ34960 | Q8N239 | AK092279 | 257240 | NM_153270 | Hs.448572 | BTB; KELCH |
| FLJ35036 | Q8NAP3 | AK092355 | none | XM_172341 | Hs.518301 | ZFb |
| FLJ43374 | Q6ZUS1 | AK125364 | 377007 | NM_198582 | Hs.1199821 | BTB |
| FRBZ1 | Q8IZ99 | AY163816 | 360023 | NM_194314 | Hs.529439 | BTB; ZFb |
| GAN/KLHL16 | Q9H2C0 | AF291673 | 8139 | NM_022041 | Hs.112569 | BTB; KELCH |
| GENSCAN00000050 | GENSCAN00000 | GENSCAN000 | none | XM_371078 | Hs.211870 | BTB |
| 86H | 050486H | 00050486 | | | | |
| GMCLI/GCL | Q96IK5 | BC007420 | 64395 | NM_178439 | Hs.293971 | BTB |
| GMCL1L | Q8NEA9 | BC033886 | 64396 | NM_022471 | Hs.484313 | BTB |
| HIC1/ZBTB29 | Q14526 | BC030208 | 3090 | NM_006497 | Hs.72956 | BTB; ZFb |
| HIC2/ZBTB30 | Q96JB3 | CR456377 | 23119 | NM_015094 | Hs.517434 | BTB; ZFb |
| HKR3 | P10074 | BC013573 | 3104 | NM_005341 | Hs.502330 | BTB; ZFb |
| HSPC063 | Q8NCP5 | BC030580 | 29068 | NM_014155 | Hs.178499 | BTB; ZFb |
| IBTK | Q9P2D0 | AB037838 | 25998 | NM_015525 | Hs.306425 | ANK; BTB; RCC |
| IPP | Q9Y573 | AF156857 | 3652 | NM_005897 | Hs.157180 | BTB; KELCH |
| IVNS1ABP | Q9Y6Y0 | AB020657 | 10625 | NM_016389; | Hs.497183 | BTB; KELCH |
| | | | | NM_006469 | | |
| KBTBD10 | 060662 | AF333387 | 10324 | NM_006063 | Hs.50550 | BTB; KELCH |
| KBTBD2 | Q8IY47 | BC032367 | 25948 | NM_015483 | Hs.372541 | BTB; KELCH |
| KBTBD3 | Q8NAB2 | BX640672 | 143879 | NM_198439; | Hs.101949 | BTB; KELCH |
| | | | | NM_152433 | | |
| KBTBD4 | Q9NVX7 | AK001749 | 55709 | NM_018095 | Hs.440695 | BTB,KELCH |
| | | | | NM_016506 | | |
| KBTBD5 | Q86S11 | AY177390 | 131377 | NM_152393 | Hs.350288 | BTB; KELCH |
| KBTBD6 | Q86V97 | BC000560 | 89890 | NM_152903 | Hs.534040 | BTB; KELCH |
| KBTBD7 | Q8WVZ9 | BC022033 | 84078 | NM_032138 | Hs.63841 | BTB; KELCH |
| KBTBD9 | Q96CT2 | BC013982 | none | XM_496546 | Hs.130593 | BTB; KELCH |
| KEAP1/KLHL19 | Q14145 | D50922 | 9817 | NM_203500; | Hs.465870 | BTB; KELCH |
| | | | | NM_012289 | | |
| KELCHL | Q96B68 | BC015923 | 84861 | NM_032775 | Hs.517419 | BTB; KELCH |
| K1AA0352 | O15060 | AB002350 | 9880 | NM_014830 | Hs.131212 | BTB; ZFb |
| KIAA0478 | Q9NUA8 | AK091019 | 9923 | NM_014870 | Hs.528723 | BTB; ZFb |
| KIAA0711 | O94819 | AB018254 | 9920 | NM_014867 | Hs.5333 | BTB; KELCH |
| KIAA1340 | Q9P2K6 | AK095405 | 57542 | NM_020782 | Hs.505104 | BTB; KELCH |
| KLEIP/KLHL20 | Q9Y2M5 | AB026190 | 27252 | NM_014458 | Hs.495035 | BTB; KELCH |
| KLHL1 | Q9NR64 | AF252283 | 57626 | NM_020866 | Hs.508201 | BTB; KELCH |
| KLHL10 | Q6JEL2 | AY495339 | 317719 | NM_152467 | Hs.127510 | BTB; KELCH |
| KLHL11 | Q9NVRO | AK001434 | 55175 | NM_018143 | Hs.13268 | BTB; KELCH |
| KLHL12 | Q9HBX5 | AF190900 | 59349 | NM_021633 | Hs.282878 | BTB; KELCH |
| KLHL13 | Q9P2N7 | AB037730 | 90293 | NM_033495 | Hs.348262 | BTB; KELCH |
| KLHL14 | Q9P2G3 | AB037805 | 57565 | NM_020805 | Hs.446164 | BTB; KELCH |
| KLHL15 | Q96M94 | AK057298 | none | XM_040383 | Hs.495854 | BTB; KELCH |
| KLHL17 | Q6TDP4 | AY423763 | 339451 | NM_198317 | Hs.109212 | BTB; KELCH |
| KLHL18 | O94889 | AB062478 | 23276 | NM_025010 | Hs.517946 | BTB; KELCH |
| KLHL2 | O95198 | BC036468 | 11275 | NM_007246 | Hs.388668 | BTB; KELCH |
| KLHL21 | Q9UJP4 | AB007938 | 9903 | NM_014851 | Hs.7764 | BTB; KELCH |
| KLHL3 | Q9UH77 | AB032955 | 26249 | NM_017415 | Hs.434434 | BTB; KELCH |
| KLHL4 | Q9C0H6 | AF284765 | 56062 | NM_019117; | Hs.49075 | BTB; KELCH |
| | | | | NM_057162 | | |
| KLHL5 | Q96PQ7 | BC053860 | 51088 | NM_199039; | Hs.272251 | BTB; KELCH |
| | | | | NM_001007075 | | |
| KLHL6 | Q8WZ60 | AK097125 | 89857 | NM_130446 | Hs.333181 | BTB; KELCH |
| KLHL7 | Q81XQ5 | BC039585 | 55975 | NM_018846 | Hs.385861 | BTB; KELCH |
| KLHL8 | Q9P2G9 | BC041384 | 57563 | NM_020803 | Hs.546415 | BTB; KELCH |
| KLHL9 | Q9P273 | AB037775 | 55958 | NM_018847 | Hs.522029 | BTB; KELCH |
| LGALS3BP | Q08380 | L13210 | 3959 | NM_005567 | Hs.514535 | SRCR; BTB |
| LOC149478 | GENSCAN00000 | AK056150 | none | XM_378860 | Hs.421430 | BTB |
| | 058813H | | | | | |
| LOC339745 | Q6IQ16 | BC071613 | 339745 | NM_001001664 | Hs.333297 | TRAF; BTB |
| LZTR1 | Q8N653 | BCO26214 | 8216 | NM_006767 | Hs.78788 | BTB; KELCH |
| MGC2610 | Q8NBE8 | AK090653 | 151230 | NM_144711 | Hs.470549 | BTB; KELCH |
| MYNN/ZBTB31 | Q86Z12 | AB079777 | 55892 | NM_018657 | Hs.507025 | BTB; ZFb |
| OTTHUMP00000016633 | Q9H511 | AK091177 | 401265 | NM_001003760 | Hs.376697 | BTB; KELCH |
| RCBTB1 | Q8NDN9 | AL833821 | 55213 | NM_018191 | Hs.508021 | BTB; RCC |
| RHOBTB1 | O94844 | AB018283 | 9886 | NM_198225; | Hs.148670 | GTPase; BTB |
| | | | | NM_014836 | | |
| RHOBTB2 | Q9BYZ6 | AB018260 | 23221 | NM_015178 | Hs.372688 | GTPase; BTB |
| RHOBTB3 | O94955 | AB020685 | 22836 | NM_014899 | Hs.445030 | BTB |
| SPOP | O43791 | A7000644 | 8405 | NM_003563; | Hs.463382 | TRAF; BTB |
| | | | | NM_001007226 | | |
| | | | | NM_001007227; | | |
| | | | | NM_001007230; | | |
| | | | | NM_001007229; | | |
| | | | | NM_001007228 | | |
| TA-KRP/KIAA1842 | Q967I5 | AB058745 | 84541 | NM_032505 | Hs.116665 | BTB; KELCH |
| TZFP/FAZF | Q9Y2Y4 | AF130255 | 27033 | NM_014383 | Hs.99430 | BTB; ZFb |
| ZBTB1 | Q9Y2K1 | BX248777 | 22890 | NM_014950 | Hs.400802 | BTB; ZFb |
| ZBTB10 | Q96DT7 | A7319673 | 65986 | NM_023929 | Hs.205742 | BTB; ZFb |
| ZBTB11 | O95625 | U69274 | 27107 | NM_014415 | Hs.301956 | BTB; ZFb |
| ZBTB12 | Q9Y330 | AF134726 | 221527 | NM_181842 | Hs.234027 | BTB; ZFb |
| ZBTB16 | Q05516 | Z19002 | 7704 | NM_006006 | Hs.171299 | BTB; ZFb |
| ZBTB17 | Q13105 | Y09723 | 7709 | NM_003443 | Hs.433764 | BTB; ZFb |
| ZBTB2 | Q8N680 | BC020172 | 57621 | NM_020861 | Hs.520073 | BTB; ZFb |
| ZBTB20 | Q9HC78 | AF139460 | 26137 | NM_015642 | Hs.477166 | BTB; ZFb |
| ZBTB24 | O43167 | AB007901 | 9841 | NM_014797 | Hs.409876 | BTB; ZFb |
| ZBTB26 | Q9HCK0 | AF323460 | 57684 | NM_020924 | Hs.5638 | BTB; ZFb |
| ZBTB3 | Q9H5J0 | AK027045 | 79842 | NM_024784 | Hs.147554 | BTB; ZFb |
| ZBTB33/kaiso | Q86T24 | AL833604 | 10009 | NM_006777 | Hs.143604 | BTB; ZFb |
| ZBTB34 | Q8NCN2 | AB082524 | none | none | Hs.177633 | BTB; ZFb |
| ZBTB37 | Q5TC79 | BC003116 | 84614 | NM_032522 | Hs.535229 | BTB; ZFb |
| ZBTB4 | Q9P1Z0 | AY302699 | 57659 | NM_020899 | Hs.35096 | BTB; ZFb |
| ZBTBS | O15062 | AB002352 | 9925 | NM_014872 | Hs.161276 | BTB; ZFb |
| ZBTB7A | O95365 | AF097916 | 51341 | NM_015898 | Hs.465623 | BTB; ZFb |
| ZBTB8a | Q8NAP8 | AK092326 | 127557 | NM_144621 | Hs.546479 | BTB; ZFb |
| ZBTB8b | Q96BR9 | BC015239 | 127557 | NM_144621 | Hs.546479 | BTB; ZFb |
| ZBTB9 | Q96C00 | BCO14978 | 221504 | NM_152735 | Hs.528028 | BTB; ZFb |
| ZFP161/ZBTB14 | O43829 | Y12726 | 7541 | NM_003409 | Hs.156000 | BTB; ZFb |
| ZFP67/ZBTB7B/ | O15156 | BC012070 | 51043 | NM_015872 | Hs.549155 | BTB; ZFb |
| ZBTB15 | | | | | | |
| ZNF131 | P52739 | AK057343 | none | none | Hs.97845 | BTB; ZFb |
| ZNF238/ZBTB18 | Q99592 | X95072 | 10472 | NM_205768; | Hs.69997 | BTB; ZFb |
| | | | | NM_006352 | | |
| ZNF278/ZBTB19 | Q9HBE1 | AF254085 | 23598 | NM_032051; | Hs.517557 | BTB; ZFb |
| | | | | NM_014323; | | |
| | | | | NM_032052; | | |
| | | | | NM_032050 | | |
| ZNF295/ZBTB21 | Q9ULJ3 | AB033053 | 49854 | NM_020727 | Hs.434947 | BTB; ZFb |
| ZNF297/ZBTB22A | O15209 | Z97184 | 9278 | NM_005453 | Hs.206770 | BTB; ZFb |
| ZNF297B/ZBTB22B | O43298 | AB007874 | 23099 | NM_014007 | Hs.355581 | BTB; ZFb |
| ZNF336/ZBTB23 | Q9H116 | AB100265 | 64412 | NM_022482 | Hs.28921 | BTB; ZFb |
| ZNF46/ZBTB25 | P24278 | X16576 | 7597 | NM_006977 | Hs.164347 | BTB; ZFb |
| ZNF482/ZBTB6 | Q15916 | X82018 | 10773 | NM_006626 | Hs.3053 | BTB; ZFb |
| ZNF499 | Q96K62 | AK027392 | 84878 | NM_032792 | Hs.515662 | BTB; ZFb |
| ZNF509/FLJ45653 | Q6ZSB9 | AK127560 | 166793 | NM_145291 | Hs.419997 | BTB; ZFb |
| ZNF651/FLJ45122 | Q6ZSY6 | AK127065 | 92999 | NM_145166 | Hs.409561 | BTB; ZFb |

TABLE 14
E3 Ligases, DDBI like type

| Name | UniProt | Genbank | LL | Refseq | Unigene | Domains |
|---|---|---|---|---|---|---|
| CPSF1 | Q10570 | BC017232 | 29894 | NM_013291 | Hs.493202 | DDBI |
| DDBI | Q16531 | UI8299 | 1642 | NM_001923 | Hs.290758 | DDBI |
| SF3B3 | Q15393 | AJ001443 | 23450 | NM_012426 | Hs.514435 | DDBI |

TABLE 15
E3 Ligases, APC/Cyclosome type

| Name | UniProt | Genbank | LL | Refseq | Unigene | Domains |
|---|---|---|---|---|---|---|
| ANAPCI | Q9H1A4 | AJ278357 | 64682 | NM_022662 | Hs.436527 | PC-rep |
| ANAPC2 | Q9UJX6 | BC032503 | 29882 | NM_013366 | Hs.533262 | CULLIN |
| CDC16 | Q13042 | AL540490 | 8881 | NM_003903 | Hs.374127 | TPR |
| CDC27 | P30260 | AU135593 | 996 | NM_001256 | Hs.463295 | TPR |
| ANAPCS | Q9UJX4 | BC006301 | 51433 | NM_016237 | Hs.7101 | |
| ANAPC4 | Q9UJX5 | AL353932 | 29945 | NM_013367 | Hs.152173 | |
| CDC23 | Q9UJX2 | AB011472 | 8697 | NM_004661 | Hs.153546 | TPR |
| ANAPC7 | Q9UJX3 | AF191340 | 51434 | NM_016238 | Hs.529280 | |
| ANAPC10 | Q9UM13 | AL080090 | 10393 | NM_014885 | Hs.480876 | |
| ANAPC11 | Q9NYG5 | BC000607 | 51529 | NM_001002249; | Hs.534456 | RF |
| | | | | NM_016476; | | |
| | | | | NM_001002246; | | |
| | | | | NM_001002248; | | |
| | | | | NM_001002244; | | |
| | | | | NM_001002247; | | |
| | | | | NM_001002245 | | |
| CDC26 | Q8NHZ8 | BC042534 | 246184 | NM_139286 | Hs.530284 | |
| CDC20 | Q12834 | U05340 | 991 | NM_001255 | Hs.524947 | WD |
| FZRI | Q9UM11 | AB033068 | 51343 | NM_016263 | Hs.413133 | WD |

Another exemplary E3 ubiquitin ligase is a von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1 alpha. (HIF-1 alpha), a transcription factor that upregulates genes such as the pro-angiogenic growth factor VEGF and the red blood cell inducing cytokine erythropoietin in response to low oxygen levels. Compounds that bind VHL may be hydroxyproline compounds such as those disclosed in WO 2013/106643, and other compounds described in US2016/0045607, WO 2014/187777, US20140356322A1, and U.S. Pat. No. 9,249,153. Another exemplary E3 ubiquitin ligase is MDM2. Examples of small molecular binding compounds for MDM2 include the “nutlin” compounds, e.g., nutlin 3a and nutlin 3, having the structure:

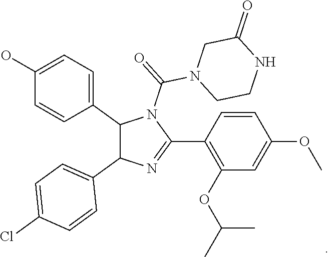 [SMILES: COc1ccc(C2=NC(c3ccc(Cl)cc3)C(c3ccc(O)cc3)N2C(=O)N2CCNC(=O)C2)c(OC(C)C)c1]

MDM2 binding compounds for use herein include, for example, those described in WO2012/121361; WO2014/038606; WO2010/082612; WO2014/044401; WO2009/151069; WO2008/072655; WO2014/100065; WO2014/100071; WO2014/123882; WO2014/120748; WO2013/096150; WO2015/161032; WO2012/155066; WO2012/065022; WO2011/060049; WO2008/036168; WO2006/091646; WO2012/155066; WO2012/065022; WO2011/153509; WO2013/049250; WO2014/151863; WO2014/130470; WO2014/134207; WO2014/200937; WO2015/070224; WO2015/158648; WO2014/082889; WO2013/178570; WO2013/135648; WO2012/116989; WO2012/076513; WO2012/038307; WO2012/034954; WO2012/022707; WO2012/007409; WO2011/134925; WO2011/098398; WO2011/101297; WO2011/067185; WO2011/061139; WO2011/045257; WO2010/121995; WO2010/091979; WO2010/094622; WO2010/084097; WO2009/115425; WO2009/080488; WO2009/077357; WO2009/047161A1; WO2008/141975A1; WO2008/141917A1; WO2008/125487A1; WO2008/034736A2; WO2008/055812A1; WO2007/104714A1; WO2007/104664A1; WO2007/082805A1; WO2007/063013A1; WO2006/136606A2; WO2006/097261A1; WO2005/123691A1; WO2005/110996A1; WO2005/003097A1; WO2005/002575A1; WO2004/080460A1; WO2003/051360A1; WO2003/051359A1; WO 1998/001467; WO2011/023677; WO2011/076786; WO2012/066095; WO2012/175487; WO2012/175520; WO2012/176123; WO2013/080141; WO2013/111105; WO2013/175417; WO2014/115080; WO2014/115077; WO2014/191896; WO2014/198266; WO2016/028391A9; WO2016/028391A2; WO2016/026937; WO2016/001376; WO2015/189799; WO2015/155332A1; WO2015/004610A8; WO2013/105037A1; WO2012/155066A3; WO2012/155066A2; WO2012/033525A3; WO2012/047587A2; WO2012/033525A2; WO2011/106650A3; WO2011/106650A2; WO2011/005219A1; WO2010/058819A1; WO2010/028862A1; WO2009/037343A1; WO2009/037308A1; WO2008/130614A3; WO2009/019274A1; WO2008/130614A2; WO2008/106507A3; WO2008/106507A2; WO2007/107545A1; WO2007/107543A1; WO2006032631A1; WO2000/015657A1; WO 1998/001467A2; WO1997/009343A3; WO1997/009343A2; WO1996/002642A1; US2007/0129416; Med Chem. Lett, 2013, 4, 466-469; J. Med Chem., 2015, 58, 1038-1052; Bioorg. Med Chem. Lett. 25 (2015) 3621-3625; Bioorg. Med Chem. Lett. 16 (2006) 3310-3314. Further specific examples of small molecular binding compounds for MDM2 contemplated for use with a DAC include, but are not limited to, RG7112, RG7388, MI 773/SAR 405838, AMG 232, DS-3032b, RO6839921, RO5045337, R05503781, Idasanutlin, CGM-097, and MK-8242.

Another exemplary E3 ubiquitin ligase is a X-linked inhibitor of apoptosis (XIAP). XIAP is a protein that stops apoptotic cell death. Examples of small molecular binding compounds for XIAP include compounds disclosed in U.S. Pat. No. 9,096,544; WO 2015187998; WO 2015071393; U.S. Pat. Nos. 9,278,978; 9,249,151; US 20160024055; US 20150307499; US 20140135270; US 20150284427; US 20150259359; US 20150266879; US 20150246882; US 20150252072; US 20150225449; U.S. Pat. No. 8,883,771, J. Med Chem., 2015, 58(16) 6574-6588 and Small-molecule Pan-IAP Antagonists: A Patent Review (2010) Expert Opin Ther Pat; 20: 251-67 (Flygare & Fairbrother). Exemplary compounds include all the tetrahydro-benzodiazinone compounds of the following formula:

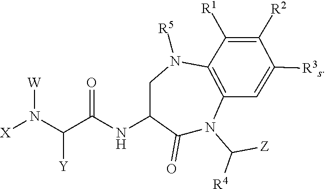

Other small molecular binding compounds for XIAP include, but are not limited to, AEG35156, Embelin, TWX006 and TWX024. When an XIAP binding moiety is used as part of a Degrader, the XIAP binding moiety can bind to the BIR2 or BIR3 domain of XIAP or both.

Another exemplary E3 ubiquitin ligase is cereblon.

Protein Binding (PB) Group

The PB component of a degrader refers to a molecule which binds to a target protein (e.g., an oligopeptide or a polypeptide) intended to be degraded. Targets for ubiquitination mediated by a compound described herein include any protein in a eukaryotic system or a microbial system, including a virus, bacteria, or fungus, as otherwise described herein.

PB groups include small molecule target protein moieties such as, for example, Heat Shock Protein 90 (HSP90) inhibitors; kinase inhibitors; Phosphatase inhibitors; MDM2 inhibitors; compounds targeting Human BET Bromodomain-containing proteins; HDAC inhibitors; human lysine methyltransferase inhibitors; angiogenesis inhibitors; immunosuppressive compounds; compounds targeting the aryl hydrocarbon receptor (AHR), REF receptor kinase, FKBP, Androgen Receptor (AR), Estrogen receptor (ER), Thyroid Hormone Receptor, HIV Protease, HIV Integrase, HCV Protease, Acyl-protein Thioesterase-1 and -2 (APT and APT2), pharmaceutically acceptable salts thereof, enantiomers thereof, solvates thereof, or polymorphs thereof, as well as other small molecules that may target a protein of interest.

Target proteins of interest include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity.

The PB component of a degrader molecule can be a peptide or small molecule that bind protein targets such as, for example, an intracellular protein, an extracellular protein, a cell surface protein, a disease-causing or a disease-related protein, a TNF-receptor-associated death-domain protein (TRADD), receptor interacting protein (RIP), TNF-receptor-associated factor 2 (TRAF2), IK-alpha, IK-beta, IK-epsilon, PLCγ, IQGAP1, Rac1, MEK1/2, ERK1/2, PI4K230, Akt1/2/3, Hsp90, GSK-3β, an HDAC protein, FoxO1, HDAC6, DP-1, E2F, ABL, AMPK, BRK, BRSK I, BRSK2, BTK, CAMKK1, CAMKK alpha, CAMKK beta, Rb, Suv39HI, SCF, p19INK4D, GSK-3, pi 8 INK4, myc, cyclin E, CDK2, CDK9, CDG4/6, Cycline D, p16 INK4A, cdc25A, BMI1, SCF, Akt, CHK1/2, C 1 delta, CK1 gamma, C 2, CLK2, CSK, DDR2, DYRK1A/2/3, EF2K, EPH-A2/A4/B/B2/B3/B4, EIF2A 3, Smad2, Smad3, Smad4, Smad7, p53, p21 Cip1, PAX, Fyn, CAS, C3G, SOS, Tal, Raptor, RACK-1, CRK, Rap1, Rac, KRas, NRas, HRas, GRB2, FAK, PI3K, spred, Spry, mTOR, MPK, LKB1, PAK 1/2/4/5/6, PDGFRA, PYK2, Src, SRPK1, PLC, PKC, PKA, PKB alpha/beta, PKC alpha/gamma/zeta, PKD, PLK1, PRAK, PRK2, WAVE-2, TSC2, DAPK1, BAD, IMP, C-TAK1, TAK1, TAO1, TBK1, TESK1, TGFBR1, TIE2, TLK1, TrkA, TSSK1, TTBK1/2, TK, Tpl2/cot1, MEK1, MEK2, PLDL Erk1, Erk2, Erk5, Erk8, p90RSK, PEA-15, SRF, p27 KIP1, TIF 1a, HMGN1, ER81, MKP-3, c-Fos, FGF-R1, GCK, GSK3 beta, HER4, HIPK1/2/3/, IGF-1R, cdc25, UBF, LAMTOR2, Stat1, StaO, CREB, JAK, Src, PTEN, NF-kappaB, HECTH9, Bax, HSP70, HSP90, Apaf-1, Cyto c, BCL-2, Bcl-xL, Smac, XIAP, Caspase-9, Caspase-3, Caspase-6, Caspase-7, CDC37, TAB, IKK, TRADD, TRAF2, R1P1, FLIP, TAK1, JNK1/2/3, Lck, A-Raf, B-Raf, C-Raf, MOS, MLK1/3, MN 1/2, MSK1, MST2/3/4, MPSK1, MEKK1, ME K4, MEL, ASK1, MINK1, MKK 1/2/3/4/6/7, NE 2a/6/7, NUAK1, OSR1, SAP, STK33, Syk, Lyn, PDK1, PHK, PIM 1/2/3, Ataxin-1, mTORC1, MDM2, p21 Waf1, Cyclin D1, Lamin A, Tpl2, Myc, catenin, Wnt, IKK-beta, IKK-gamma, IKK-alpha, IKK-epsilon, ELK, p65RelA, IRAKI, IRA 2, IRAK4, IRR, FADD, TRAF6, TRAF3, MKK3, MKK6, ROCK2, RSK1/2, SGK 1, SmMLCK, SIK2/3, ULK1/2, VEGFR1, WNK 1, YES1, ZAP70, MAP4K3, MAP4K5, MAPK1b, MAPKAP-K2 K3, p38 alpha/beta/delta/gamma MAPK, Aurora A, Aurora B, Aurora C, MCAK, Clip, MAPKAPK, FAK, MARK 1/2/3/4, Muc1, SHC, CXCR4, Gap-1, Myc, beta-catenin/TCF, Cbl, BRM, Mcl-1, BRD2, BRD3, BRD4, AR, RAS, ErbB3, EGFR, IRE1, HPK1, RIPK2, Sp20 protease, PDE4, ERRα, FKBP12, brd9, c-Met, Sirt2, ft3, BTK. ALK, TRIM24, GSPT1, IKZF1 (Ikaros), IKZF3 (Aiolos), CK1-alpha, TACC3, p85, MetAP-2, DHFR, BET, CRABP-I/II, HIF1-alpha, PCAF, GCN5, SMARCA2, SMARCA4, PBRM1, HER2, Akt, Hsp90, HDAC6, K-Ras, PI3K, BTK, B-Raf, ERK, MEK, P65 (RELA), p50 (NFKB1) of NFkB, Ras, Raf, eNOS, a Smad family protein, Smad2/3/4, and ERalpha, variants thereof, mutants thereof, splice variants thereof, indels thereof, and fusions thereof. In some embodiments, the PB group binds a protein selected from the group consisting of Akt, Hsp90, HDAC6, K-Ras, PI3K, BTK, B-Raf, ERK, MEK, P65 (RELA), p50 (NFKB1) of NFkB, Ras, Raf, eNOS, a Smad family protein, Smad2/3/4, and combinations thereof.

In some embodiments, the PB is a small molecule that binds Brd4, such as structure (or a pharmaceutically suitable salt or tautomer thereof) selected from the following:

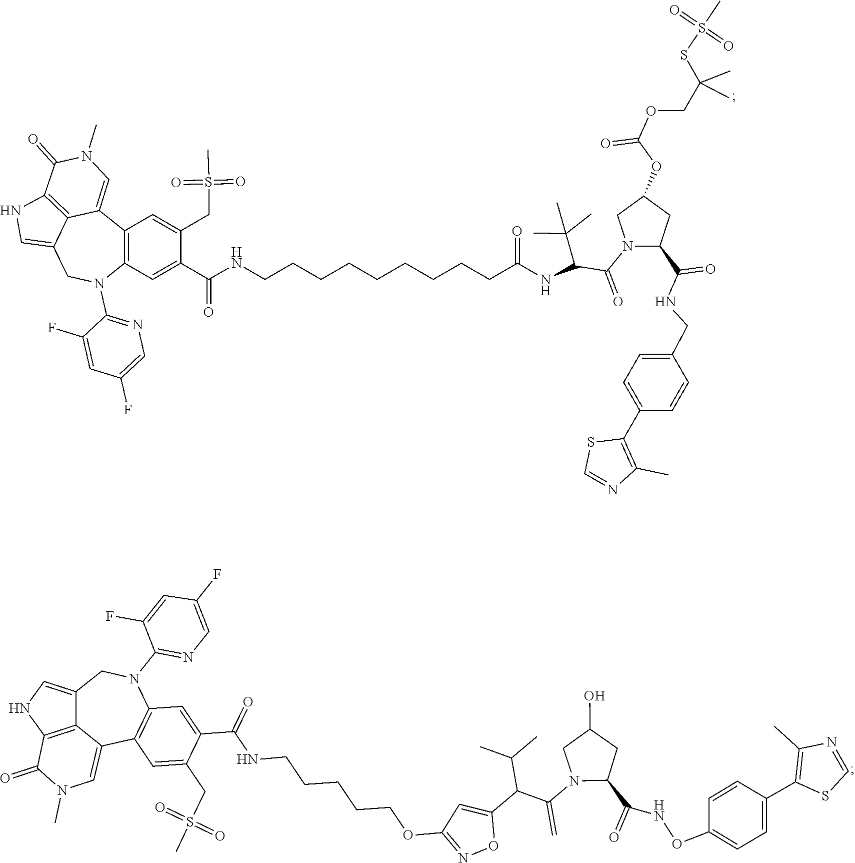

7-(3,5-Difluoropyridin-2-yl)-N-(5-((5-(1-((2S,4R)-4-hydroxy-2-((4-(4-methylthiazol-5-yl)phenoxy)carbamoyl)pyrrolidin-1-yl)-3-methyl-1-oxobutan-2-yl)isoxazol-3-yl)oxy)pentyl)-2-methyl-10-((methylsulfonyl)methyl)-3-oxo-3,4,6,7-tetrahydro-2H-2,4,7-triazadibenzo [cd,f] azulene-9-carboxamide (two single stereoisomers);

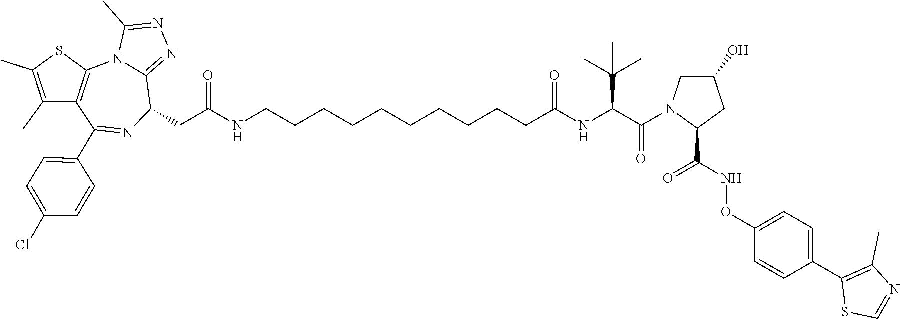 [SMILES: Cc1ncsc1-c1ccc(ONC(=O)[C@@H]2C[C@@H](O)CN2C(=O)[C@@H](NC(=O)CCCCCCCCCCNC(=O)C[C@@H]2N=C(c3ccc(Cl)cc3)c3c(sc(C)c3C)-n3c(C)nnc32)C(C)(C)C)cc1]

(2S,4R)-1-((S)-2-(H-(2-((S)-4-(4-chlorophenyl)-2,3,9-trimethyl-6H-thieno[3,2-f][1,2,4]triazolo[4,3-a][1,4]diazepin-6-yl)acetamido)undecanamido)-3,3-dimethylbutanoyl)-4-hydroxy-N-(4-(4-methylthiazol-5-yl)phenoxy)pyrrolidine-2-carboxamide;

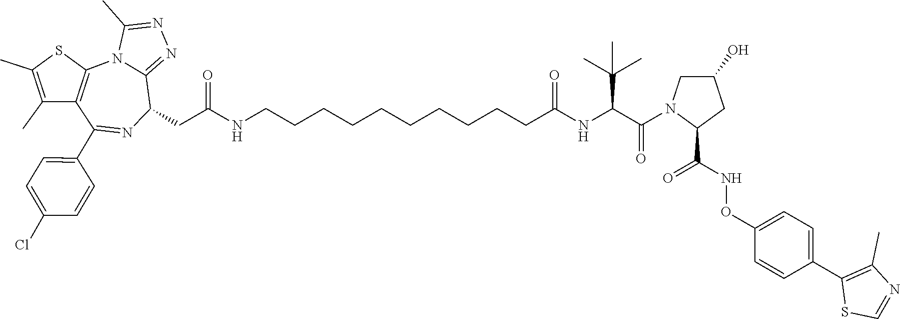 [SMILES: Cc1ncsc1-c1ccc(ONC(=O)[C@@H]2C[C@@H](O)CN2C(=O)[C@@H](NC(=O)CCCCCCCCCCNC(=O)C[C@@H]2N=C(c3ccc(Cl)cc3)c3c(sc(C)c3C)-n3c(C)nnc32)C(C)(C)C)cc1]

(2S,4R)-1-((S)-2-(H-(2-((S)-4-(4-chlorophenyl)-2,3,9-trimethyl-6H-thieno[3,2-f][1,2,4]triazolo[4,3-a][1,4]diazepin-6-yl)acetamido)undecanamido)-3,3-dimethylbutanoyl)-4-hydroxy-N-(4-(4-methylthiazol-5-yl)phenoxy)pyrrolidine-2-carboxamide;

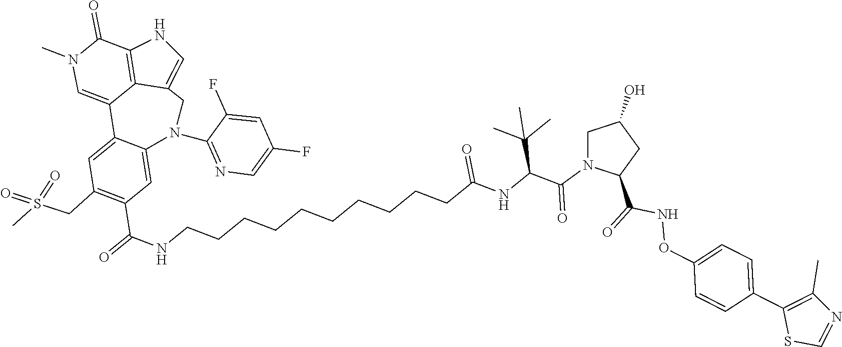 [SMILES: Cc1ncsc1-c1ccc(ONC(=O)[C@@H]2C[C@@H](O)CN2C(=O)[C@@H](NC(=O)CCCCCCCCCCNC(=O)c2cc3c(cc2CS(C)(=O)=O)-c2cn(C)c(=O)c4[nH]cc(c24)CN3c2ncc(F)cc2F)C(C)(C)C)cc1]

4-(3,5-difluoropyridin-2-yl)-N-(11-(((S)-1-((2S,4R)-4-hydroxy-2-((4-(4-methylthiazol-5-yl)phenoxy)carbamoyl)pyrrolidin-1-yl)-3,3-dimethyl-1-oxobutan-2-yl)amino)-11-oxoundecyl)-10-methyl-7-((methylsulfonyl)methyl)-11-oxo-3,4,10,11-tetrahydro-1H-1,4,10-triazadibenzo[cd,f]azulene-6-carboxamide;

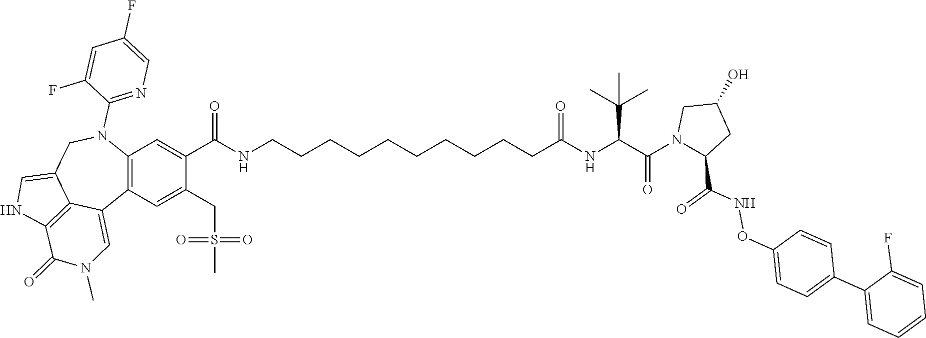 [SMILES: Cn1cc2c3c(c[nH]c3c1=O)CN(c1ncc(F)cc1F)c1cc(C(=O)NCCCCCCCCCCC(=O)N[C@H](C(=O)N3C[C@H](O)C[C@H]3C(=O)NOc3ccc(-c4ccccc4F)cc3)C(C)(C)C)c(CS(C)(=O)=O)cc1-2]

4-(3,5-difluoropyridin-2-yl)-N-(11-(((S)-1-((2S,4R)-2-(((2′-fluoro-[1,1′-biphenyl]-4-yl)oxy)carbamoyl)-4-hydroxypyrrolidin-1-yl)-3,3-dimethyl-1-oxobutan-2-yl)amino)-ll-oxoundecyl)-10-methyl-7-((methylsulfonyl)methyl)-11-oxo-3,4,10,11-tetrahydro-1H-1,4,10-triazadibenzo[cd,f]azulene-6-carboxamide; and

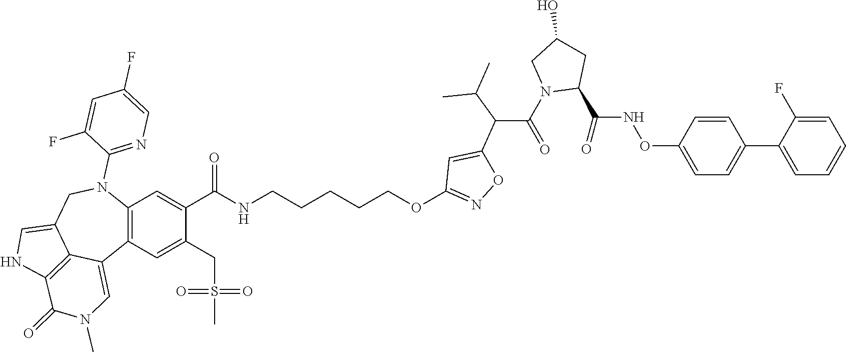 [SMILES: CC(C)C(C(=O)N1C[C@H](O)C[C@H]1C(=O)NOc1ccc(-c2ccccc2F)cc1)c1cc(OCCCCCNC(=O)c2cc3c(cc2CS(C)(=O)=O)-c2cn(C)c(=O)c4[nH]cc(c24)CN3c2ncc(F)cc2F)no1]

4-(3,5-difluoropyridin-2-yl)-N-(5-((5-(1-((2S,4R)-2-(((2′-fluoro-[1,1′-biphenyl]-4-yl)oxy)carbamoyl)-4-hydroxypyrrolidin-1-yl)-3-methyl-1-oxobutan-2-yl)isoxazol-3-yl)oxy)pentyl)-10-methyl-7-((methylsulfonyl)methyl)-11-oxo-3,4,10,11-tetrahydro-1H-1,4,10-triazadibenzo[cd,f]azulene-6-carboxamide (two single stereoisomers).

In some embodiments, the PB is a small molecule has the following structure, wherein R is an azide, DBCO, Tetrazine, BCN, Maleimide, BrAc, or any conjugation click handle and wherein the heteroatom can be located anywhere on the spacer/linker/chain:

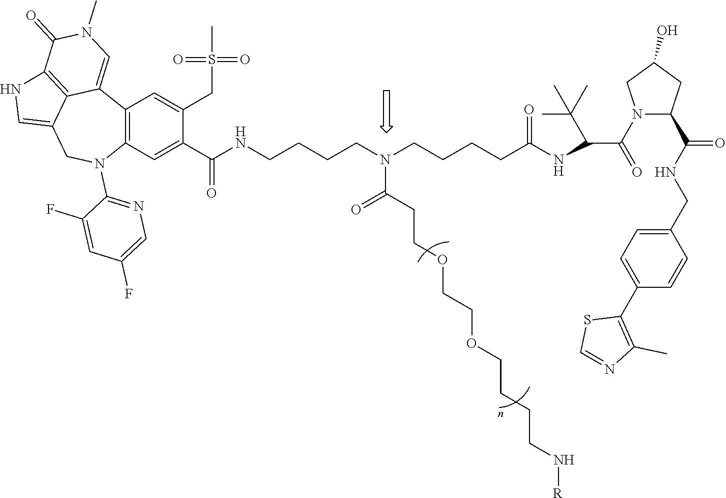

In some embodiments, the PB is a small molecule has the following structure, wherein X is S—R, benzyl, thioether, conjugation handle to antibody, disulfide solubilizing group, or disulfide auxiallary group and Y is a heteroatom, such as Nitrogen, Sulfur, or Oxygen:

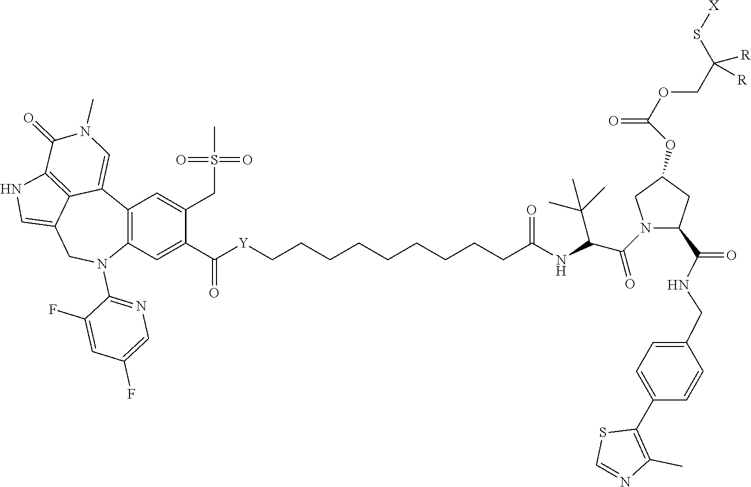

In some embodiments, the PB is a small molecule with one of the following structures, wherein R is a Peg spacer, polysarcosine, or terminating in any conjugation handle to antibody; X is S—R, benzyl, thioether, conjugation handle to antibody, disulfide solubilizing group, or disulfide auxiallary group; and Y is oxygen or nitrogen:

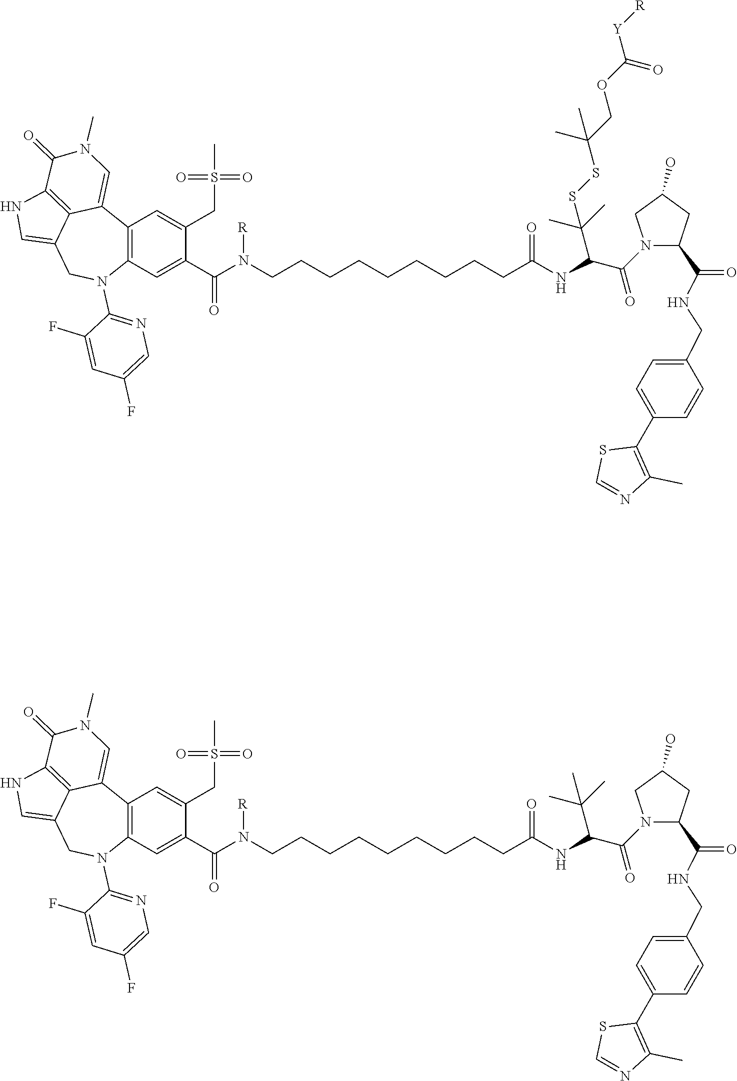

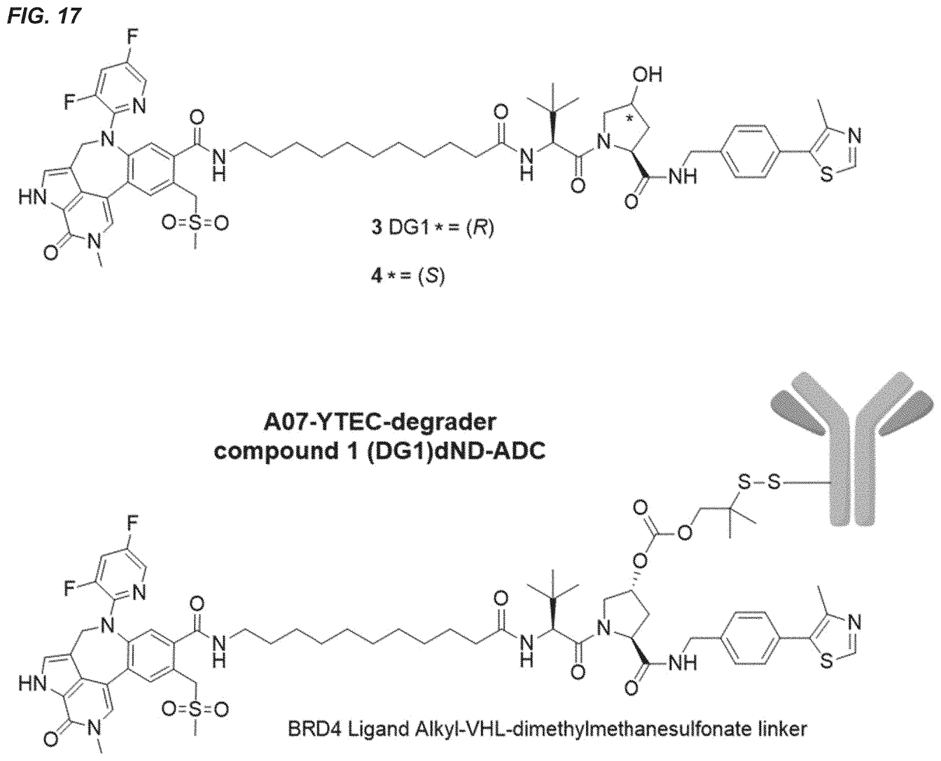

In some embodiments, the PB is a small molecule that binds Brd4, such as degrader compound 1, as shown in FIG. 17 and the degrader antibody conjugate comprises a structure also shown in FIG. 17.

In some embodiments, the PB is a small molecule that targets Human BET Bromodomain-containing proteins. Compounds targeting Human BET Bromodomain-containing proteins include, but are not limited to the compounds associated with the targets as described below, where “R” designates a site for linker group L or a -L-(VHL ligand moiety) group attachment. For example:

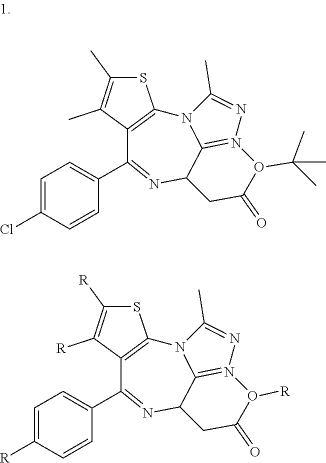

JQ1, Filippakopoulos et al. “Selective inhibition of BET bromodomains,” Nature (2010), 468, 1067-1073; Romero, et al, J. Med. Chem. 59, 1271-1298 (2016);

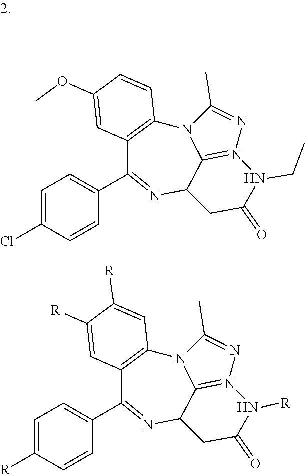

I-BET, Nicodeme et al, “Suppression of Inflammation by a Synthetic Histone Mimic,” Nature (2010), 468, 1119-1123; Chung et al, “Discovery and Characterization of Small Molecule Inhibitors of the BET Family Bromodomains,” J. Med Chem. (2011), 54, 3827-3838; Romero, et al, J. Med. Chem. 59, 1271-1298 (2016);

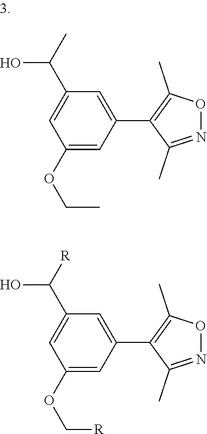

Hewings et al., “3,5-Dimethylisoxazoles Act as Acetyl-lysine Bromodomain Ligands,” J. Med. Chem., 2011), 54, 6761-6770;

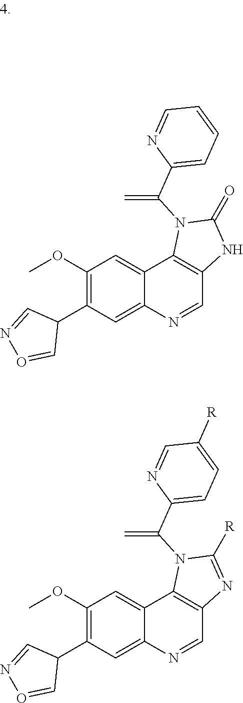

### I-BET151, Dawson et al., “Inhibition of BET Recruitment to Chromatin as an Effective Treatment for MLL-fusion Leukemia,” Nature (2011) 478, 529-523;

5. The BET bromodomain inhibitors identified in Romero, et al, J. Med. Chem. 59, 1271-1298 (2016), including, but not limited to:

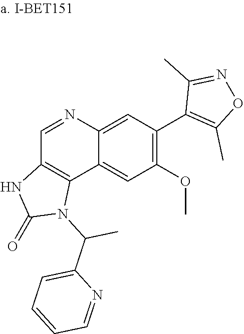 [SMILES: COc1cc2c(cc1-c1c(C)noc1C)ncc1[nH]c(=O)n(C(C)c3ccccn3)c12]

(derivatized such that a linker group L or a -L-(VHL ligand moiety) is attached);

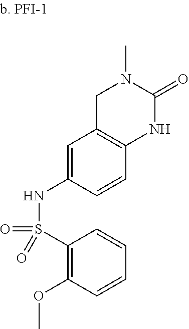 [SMILES: COc1ccccc1S(=O)(=O)Nc1ccc2c(c1)CN(C)C(=O)N2]

(derivatized such that a linker group L or a -L-(VHL ligand moiety) is attached);

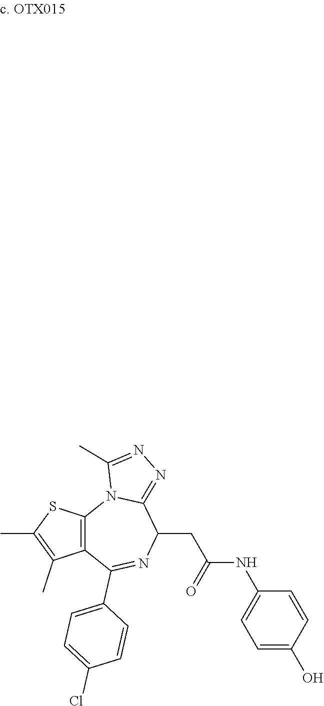 [SMILES: Cc1sc2c(c1C)C(c1ccc(Cl)cc1)=NC(CC(=O)Nc1ccc(O)cc1)c1nnc(C)n1-2]

(derivatized such that a linker group L or a -L-(VHL ligand moiety) is attached);

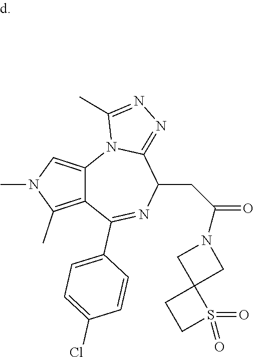 [SMILES: Cc1c2c(cn1C)-n1c(C)nnc1C(CC(=O)N1CC3(CCS3(=O)=O)C1)N=C2c1ccc(Cl)cc1]

(derivatized such that a linker group L or a -L-(VHL ligand moiety) is attached);

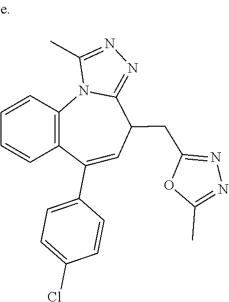 [SMILES: Cc1nnc(CC2C=C(c3ccc(Cl)cc3)c3ccccc3-n3c(C)nnc32)o1]

(derivatized such that a linker group L or a -L-(VHL ligand moiety) is attached);

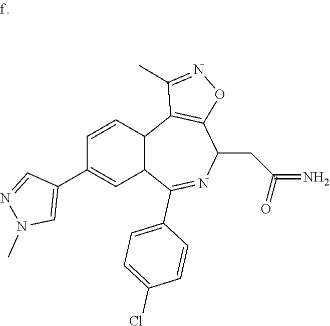

(derivatized such that a linker group L or a -L-(VHL ligand moiety) is attached);

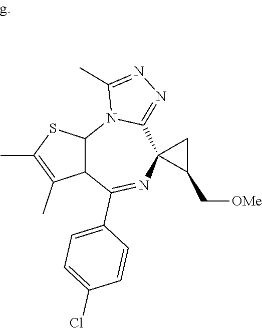

(derivatized such that a linker group L or a -L-(VHL ligand moiety) is attached);

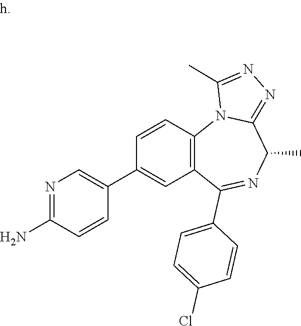 [SMILES: Cc1nnc2n1-c1ccc(-c3ccc(N)nc3)cc1C(c1ccc(Cl)cc1)=N[C@H]2C]

(derivatized such that a linker group L or a -L-(VHL ligand moiety) is attached);

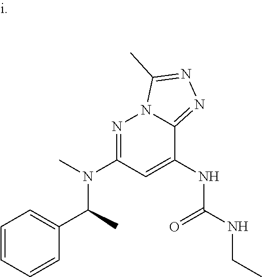 [SMILES: CCNC(=O)Nc1cc(N(C)[C@@H](C)c2ccccc2)nn2c(C)nnc12]

(derivatized such that a linker group L or a -L-(VHL ligand moiety) is attached);

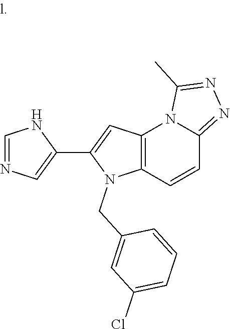 [SMILES: Cc1nnc2ccc3c(cc(-c4cnc[nH]4)n3Cc3cccc(Cl)c3)n12]

(derivatized such that a linker group L or a -L-(VHL ligand moiety) is attached);

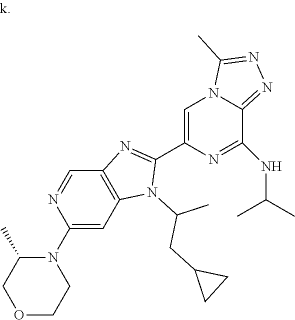 [SMILES: Cc1nnc2c(NC(C)C)nc(-c3nc4cnc(N5CCOC[C@@H]5C)cc4n3C(C)CC3CC3)cn12]

(derivatized such that a linker group L or a -L-(VHL ligand moiety) is attached);

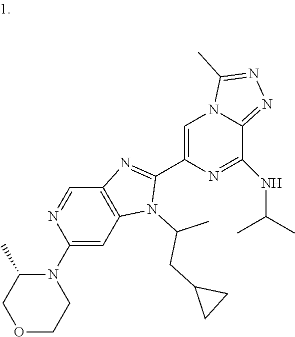 [SMILES: Cc1nnc2c(NC(C)C)nc(-c3nc4cnc(N5CCOC[C@@H]5C)cc4n3C(C)CC3CC3)cn12]

(derivatized such that a linker group L or a -L-(VHL ligand moiety) is attached);

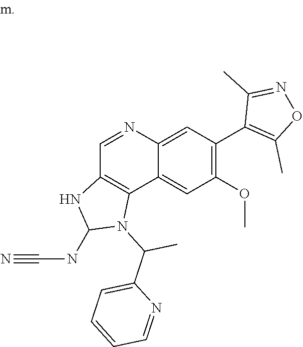 [SMILES: COc1cc2c3c(cnc2cc1-c1c(C)noc1C)NC(NC#N)N3C(C)c1ccccn1]

(derivatized such that a linker group L or a -L-(VHL ligand moiety) is attached);

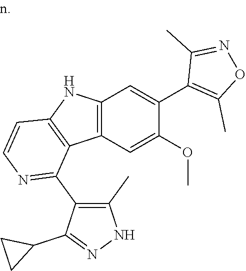 [SMILES: COc1cc2c(cc1-c1c(C)noc1C)[nH]c1ccnc(-c3c(C4CC4)n[nH]c3C)c12]

(derivatized such that a linker group L or a -L-(VHL ligand moiety) is attached);

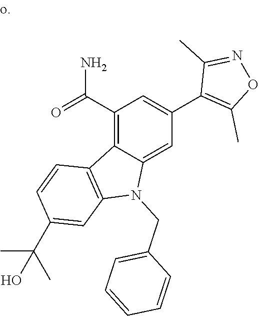 [SMILES: Cc1noc(C)c1-c1cc(C(N)=O)c2c3ccc(C(C)(C)O)cc3n(Cc3ccccc3)c2c1]

(derivatized such that a linker group L or a -L-(VHL ligand moiety) is attached);

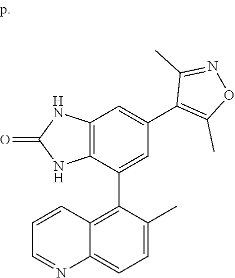 [SMILES: Cc1ccc2ncccc2c1-c1cc(-c2c(C)noc2C)cc2[nH]c(=O)[nH]c12]

(derivatized such that a linker group L or a -L-(VHL ligand moiety) is attached);

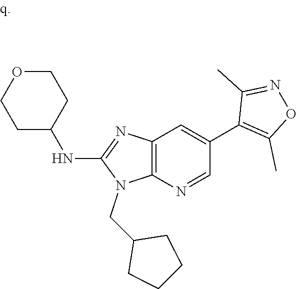 [SMILES: Cc1noc(C)c1-c1cnc2c(c1)nc(NC1CCOCC1)n2CC1CCCC1]

(derivatized such that a linker group L or a -L-(VHL ligand moiety) is attached);

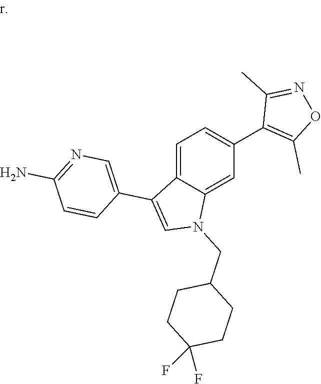 [SMILES: Cc1noc(C)c1-c1ccc2c(-c3ccc(N)nc3)cn(CC3CCC(F)(F)CC3)c2c1]

(derivatized such that a linker group L or a -L-(VHL ligand moiety) is attached);

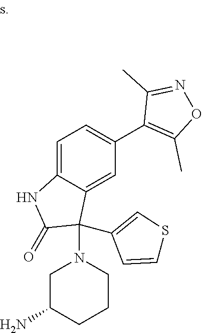 [SMILES: Cc1noc(C)c1-c1ccc2c(c1)C(c1ccsc1)(N1CCC[C@H](N)C1)C(=O)N2]

(derivatized such that a linker group L or a -L-(VHL ligand moiety) is attached);

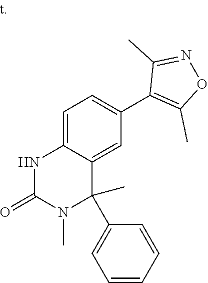 [SMILES: Cc1noc(C)c1-c1ccc2c(c1)C(C)(c1ccccc1)N(C)C(=O)N2]

(derivatized such that a linker group L or a -L-(VHL ligand moiety) is attached);

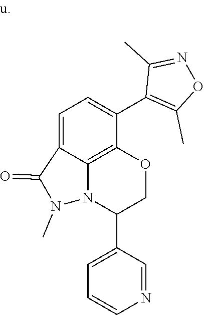 [SMILES: Cc1noc(C)c1-c1ccc2c(=O)n(C)n3c2c1OCC3c1cccnc1]

(derivatized such that a linker group L or a -L-(VHL ligand moiety) is attached);

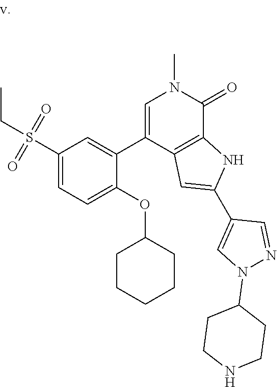 [SMILES: CCS(=O)(=O)c1ccc(OC2CCCCC2)c(-c2cn(C)c(=O)c3[nH]c(-c4cnn(C5CCNCC5)c4)cc23)c1]

(derivatized such that a linker group L or a -L-(VHL ligand moiety) is attached);

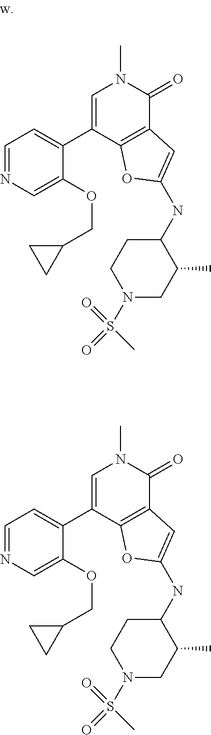 [SMILES: C[C@@H]1CN(S(C)(=O)=O)CCC1Nc1cc2c(=O)n(C)cc(-c3ccncc3OCC3CC3)c2o1 ; C[C@@H]1CN(S(C)(=O)=O)CCC1Nc1cc2c(=O)n(C)cc(-c3ccncc3OCC3CC3)c2o1]

(derivatized such that a linker group L or a -L-(VHL ligand moiety) is attached);

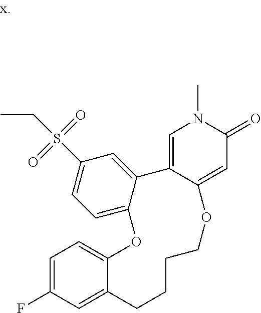 [SMILES: CCS(=O)(=O)c1ccc2c(c1)-c1cn(C)c(=O)cc1OCCCCc1cc(F)ccc1O2]

(derivatized such that a linker group L or a -L-(VHL ligand moiety) is attached);

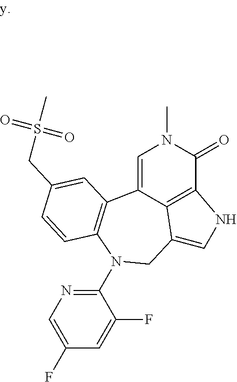 [SMILES: Cn1cc2c3c(c[nH]c3c1=O)CN(c1ncc(F)cc1F)c1ccc(CS(C)(=O)=O)cc1-2]

(derivatized such that a linker group L or a -L-(VHL ligand moiety) is attached);

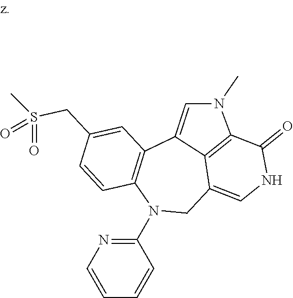 [SMILES: Cn1cc2c3c(c[nH]c(=O)c31)CN(c1ccccn1)c1ccc(CS(C)(=O)=O)cc1-2]

(derivatized such that a linker group L or a -L-(VHL ligand moiety) is attached);

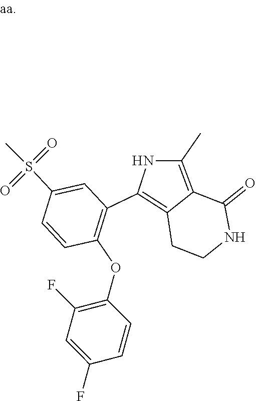 [SMILES: Cc1[nH]c(-c2cc(S(C)(=O)=O)ccc2Oc2ccc(F)cc2F)c2c1C(=O)NCC2]

(derivatized such that a linker group L or a -L-(VHL ligand moiety) is attached);

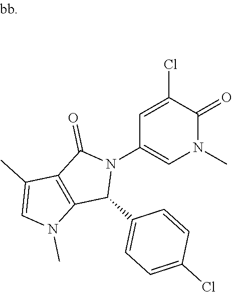 [SMILES: Cc1cn(C)c2c1C(=O)N(c1cc(Cl)c(=O)n(C)c1)[C@@H]2c1ccc(Cl)cc1]

(derivatized such that a linker group L or a -L-(VHL ligand moiety) is attached);

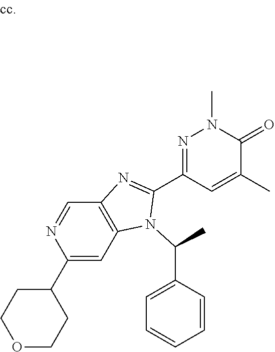 [SMILES: Cc1cc(-c2nc3cnc(C4CCOCC4)cc3n2[C@@H](C)c2ccccc2)nn(C)c1=O]

(derivatized such that a linker group L or a -L-(VHL ligand moiety) is attached);

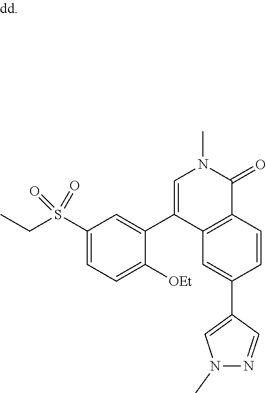 [SMILES: CCOc1ccc(S(=O)(=O)CC)cc1-c1cn(C)c(=O)c2ccc(-c3cnn(C)c3)cc12]

(derivatized such that a linker group L or a -L-(VHL ligand moiety) is attached);

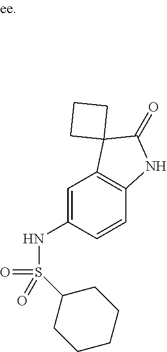 [SMILES: O=C1Nc2ccc(NS(=O)(=O)C3CCCCC3)cc2C12CCC2]

(derivatized such that a linker group L or a -L-(VHL ligand moiety) is attached);

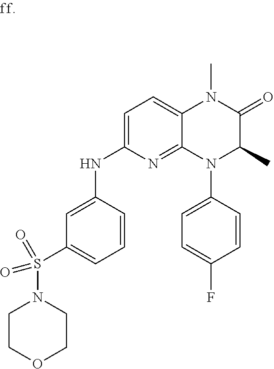 [SMILES: C[C@@H]1C(=O)N(C)c2ccc(Nc3cccc(S(=O)(=O)N4CCOCC4)c3)nc2N1c1ccc(F)cc1]

(derivatized such that a linker group L or a -L-(VHL ligand moiety) is attached);

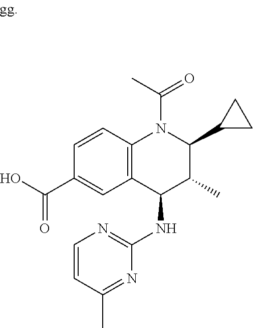 [SMILES: CC(=O)N1c2ccc(C(=O)O)cc2[C@H](Nc2nccc(C)n2)[C@@H](C)[C@@H]1C1CC1]

(derivatized such that a linker group L or a -L-(VHL ligand moiety) is attached);

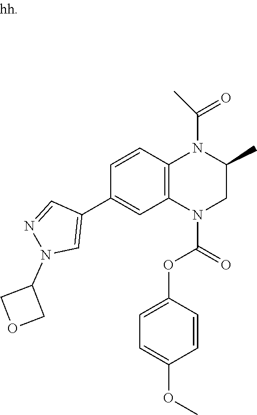 [SMILES: COc1ccc(OC(=O)N2C[C@H](C)N(C(C)=O)c3ccc(-c4cnn(C5COC5)c4)cc32)cc1]

(derivatized such that a linker group L or a -L-(VHL ligand moiety) is attached);

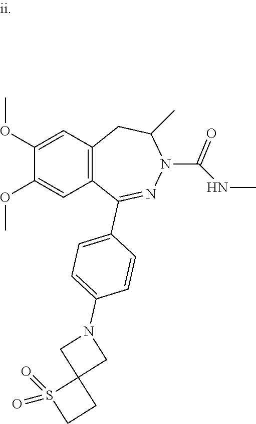 [SMILES: CNC(=O)N1N=C(c2ccc(N3CC4(CCS4(=O)=O)C3)cc2)c2cc(OC)c(OC)cc2CC1C]

(derivatized such that a linker group L or a -L-(VHL ligand moiety) is attached);

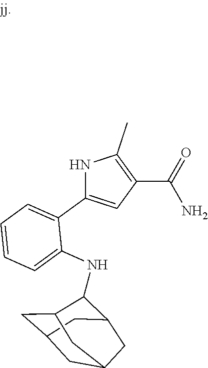 [SMILES: Cc1[nH]c(-c2ccccc2NC2C3CC4CC(C3)CC2C4)cc1C(N)=O]

(derivatized such that a linker group L or a -L-(VHL ligand moiety) is attached);

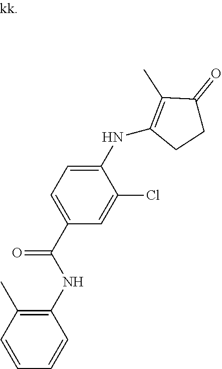 [SMILES: CC1=C(Nc2ccc(C(=O)Nc3ccccc3C)cc2Cl)CCC1=O]

(derivatized such that a linker group L or a -L-(L ligand moiety) is attached);

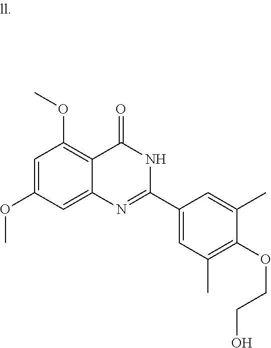 [SMILES: COc1cc(OC)c2c(=O)[nH]c(-c3cc(C)c(OCCO)c(C)c3)nc2c1]

(derivatized such that a linker group L or a -L-(VHL ligand moiety) is attached);

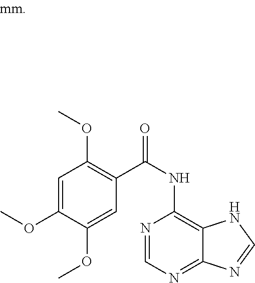 [SMILES: COc1cc(OC)c(C(=O)Nc2ncnc3nc[nH]c23)cc1OC]

(derivatized such that a linker group L or a -L-(VHL ligand moiety) is attached);

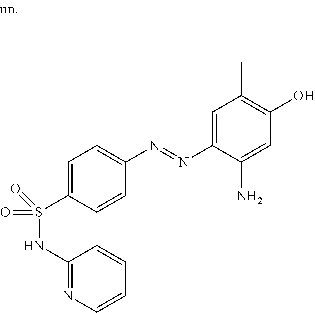 [SMILES: Cc1cc(/N=N/c2ccc(S(=O)(=O)Nc3ccccn3)cc2)c(N)cc1O]

(derivatized such that a linker group L or a -L-(VHL ligand moiety) is attached);

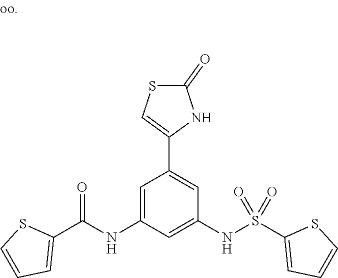 [SMILES: O=C(Nc1cc(NS(=O)(=O)c2cccs2)cc(-c2csc(=O)[nH]2)c1)c1cccs1]

(derivatized such that a linker group L or a -L-(VHL ligand moiety) is attached);

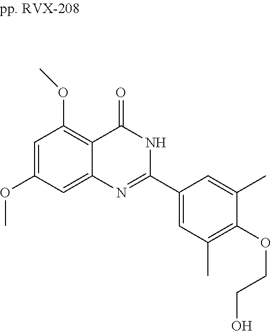 [SMILES: COc1cc(OC)c2c(=O)[nH]c(-c3cc(C)c(OCCO)c(C)c3)nc2c1]

(derivatized such that a linker group L or a -L-(VHL ligand moiety) is attached);

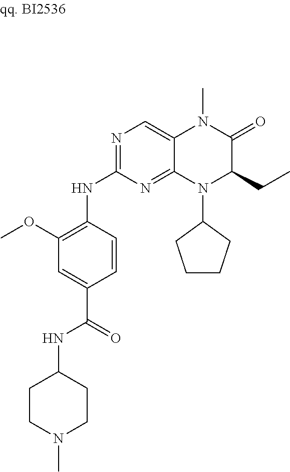 [SMILES: CC[C@@H]1C(=O)N(C)c2cnc(Nc3ccc(C(=O)NC4CCN(C)CC4)cc3OC)nc2N1C1CCCC1]

(derivatized such that a linker group L or a -L-(VHL ligand moiety) is attached);

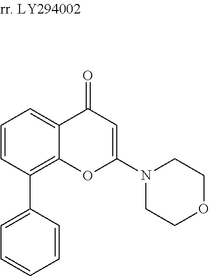 [SMILES: O=c1cc(N2CCOCC2)oc2c(-c3ccccc3)cccc12]

(derivatized such that a linker group L or a -L-(VHL ligand moiety) is attached); and

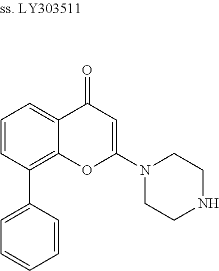 [SMILES: O=c1cc(N2CCNCC2)oc2c(-c3ccccc3)cccc12]

(derivatized such that a linker group L or a -L-(VHL ligand moiety) is attached); and

6. The BET inhibitors identified in Ghoshal, et al., “BET inhibitors in cancer therapeutics: a patent review,” Expert Opinion on Therapeutic Patents, 26:4, 505-522, (2016)) (hereinafter Ghoshal, et al. (2016)), including but not limited to:

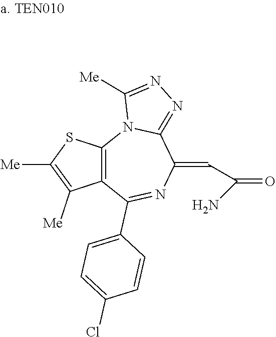 [SMILES: Cc1sc2c(c1C)C(c1ccc(Cl)cc1)=N/C(=C\C(N)=O)c1nnc(C)n1-2]

(derivatized such that a linker group L or a -L-(VHL ligand moiety) is attached);

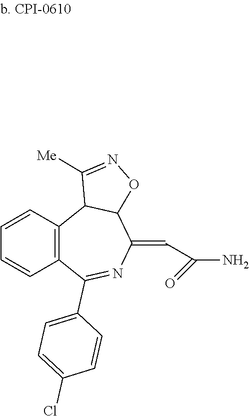 [SMILES: CC1=NOC2/C(=C/C(N)=O)N=C(c3ccc(Cl)cc3)c3ccccc3C12]

(derivatized such that a linker group L or a -L-(VHL ligand moiety) is attached);

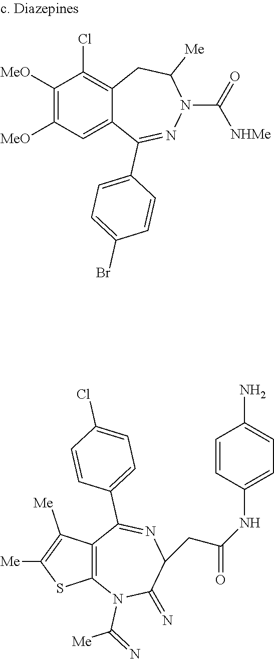 [SMILES: CNC(=O)N1N=C(c2ccc(Br)cc2)c2cc(OC)c(OC)c(Cl)c2CC1C ; CC(=N)N1C(=N)C(CC(=O)Nc2ccc(N)cc2)N=C(c2ccc(Cl)cc2)c2c1sc(C)c2C]

(derivatized such that a linker group L or a -L-(VHL ligand moiety) is attached);

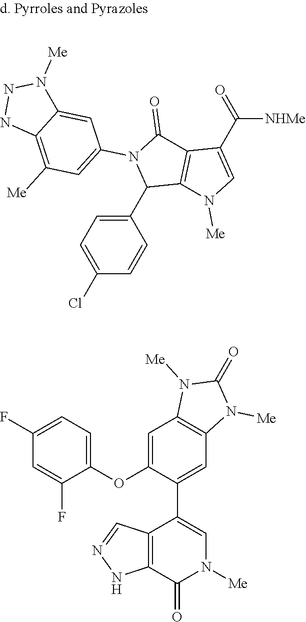 [SMILES: CNC(=O)c1cn(C)c2c1C(=O)N(c1cc(C)c3c(c1)N(C)NN3)C2c1ccc(Cl)cc1 ; Cn1cc(-c2cc3c(cc2Oc2ccc(F)cc2F)n(C)c(=O)n3C)c2cn[nH]c2c1=O]

(derivatized such that a linker group L or a -L-(VHL ligand moiety) is attached);

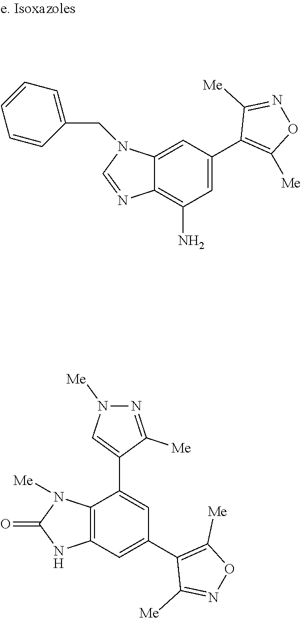 [SMILES: Cc1noc(C)c1-c1cc(N)c2ncn(Cc3ccccc3)c2c1 ; Cc1nn(C)cc1-c1cc(-c2c(C)noc2C)cc2[nH]c(=O)n(C)c12]

(derivatized such that a linker group L or a -L-(VHL ligand moiety) is attached);

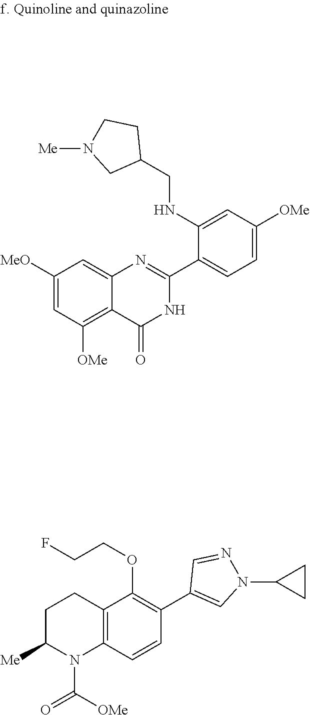 [SMILES: COc1ccc(-c2nc3cc(OC)cc(OC)c3c(=O)[nH]2)c(NCC2CCN(C)C2)c1 ; COC(=O)N1c2ccc(-c3cnn(C4CC4)c3)c(OCCF)c2CC[C@@H]1C]

(derivatized such that a linker group L or a -L-(VHL ligand moiety) is attached);

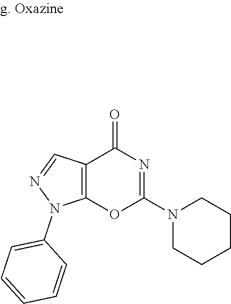 [SMILES: O=c1nc(N2CCCCC2)oc2c1cnn2-c1ccccc1]

(derivatized such that a linker group L or a -L-(VHL ligand moiety) is attached);

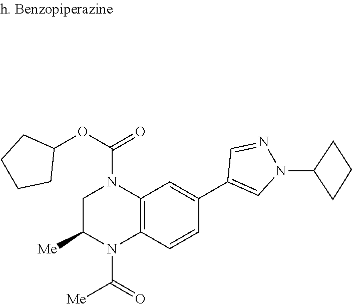 [SMILES: CC(=O)N1c2ccc(-c3cnn(C4CCC4)c3)cc2N(C(=O)OC2CCCC2)C[C@@H]1C]

(derivatized such that a linker group L or a -L-(VHL ligand moiety) is attached);

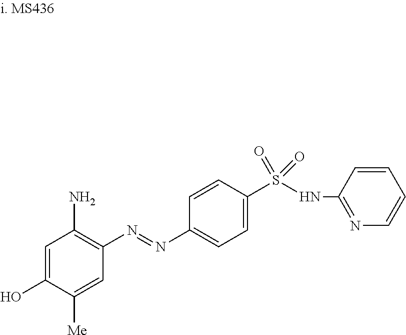 [SMILES: Cc1cc(/N=N/c2ccc(S(=O)(=O)Nc3ccccn3)cc2)c(N)cc1O]

(derivatized such that a linker group L or a -L-(VHL ligand moiety) is attached);

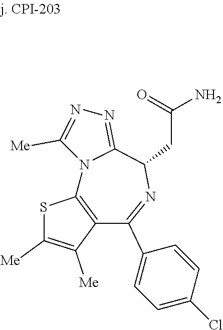 [SMILES: Cc1sc2c(c1C)C(c1ccc(Cl)cc1)=N[C@@H](CC(N)=O)c1nnc(C)n1-2]

(derivatized such that a linker group L or a -L-(VHL ligand moiety) is attached);

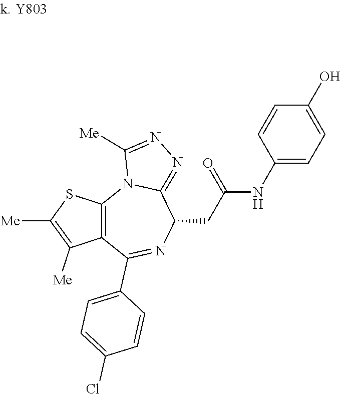 [SMILES: Cc1sc2c(c1C)C(c1ccc(Cl)cc1)=N[C@@H](CC(=O)Nc1ccc(O)cc1)c1nnc(C)n1-2]

(derivatized such that a linker group L or a -L-(VHL ligand moiety) is attached); and

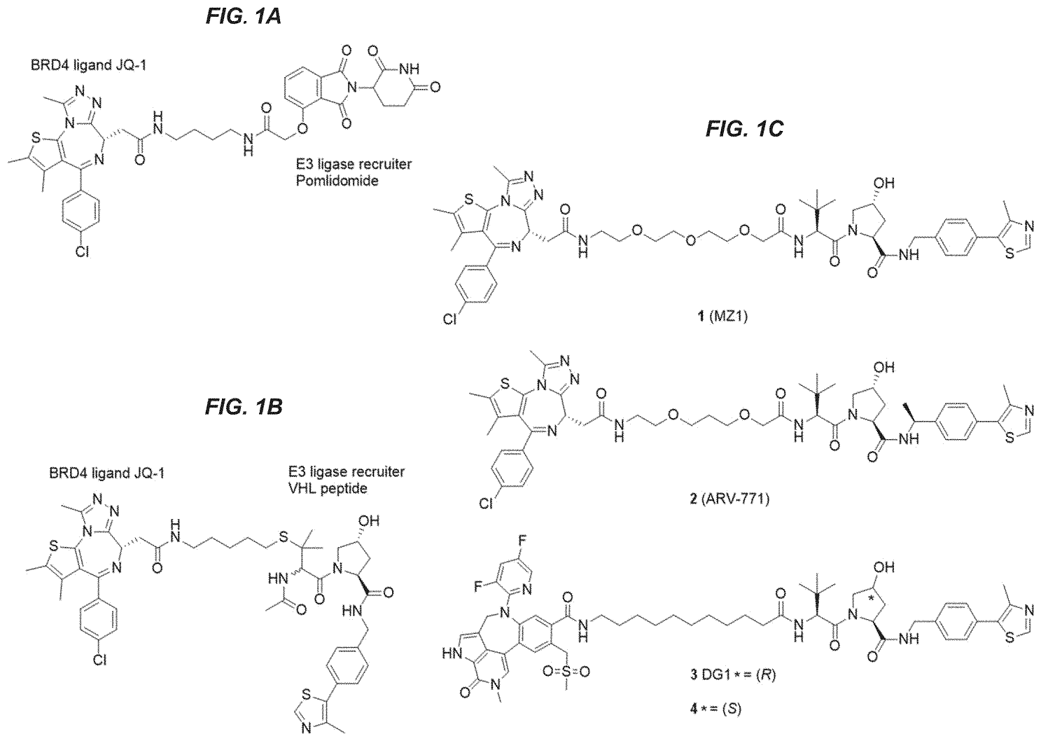

1. Benzodiazepine-based BET inhibitors reported in any one of FIG. 1 or 4-27 of Ghoshal, et al. (2016) (derivatized such that a linker group L or a -L-(VHL ligand moiety) is attached).

Linkers

The anti-TM4SF1 antibodies or antigen binding fragments described herein may be indirectly conjugated to a degrader molecule (e.g., by way of a linker (L1) with direct covalent or non-covalent interactions). Within a degrader molecule, the ubiquitin E3 ligase binding group (E3LB) may be indirectly conjugated a protein binding group (PB) molecule (e.g., by way of a linker (L2) with direct covalent or non-covalent interactions).

Linker L1

In some embodiments, a linker (“L1”) that conjugates one or more degrader molecule to an anti-TM4SF1 antibody, to form a DAC, is a bifunctional or multifunctional moiety. In some embodiments, the DACs can be prepared using a L1 having reactive functionalities for covalently attaching to the degrader and to the antibody. For example, in some embodiments, a cysteine thiol of an anti-TM4SF1 antibody (Ab) can form a bond with a reactive functional group of a linker or a linker L 1-degrader group to make a DAC.

The linker can be generally divided into two categories: cleavable (such as peptide, hydrzone, or disulfide) or non-cleavable (such as thioether). Peptide linkers, such as Valine-Citrulline (Val-Cit), that can be hydrolyzed by lysosomal enzymes (such as Cathepsin B) have been used to connect a drug with an antibody (U.S. Pat. No. 6,214,345). Such linker are, in some instances, particularly useful for their relative stability in systemic circulation and the ability to efficiently release the drug in tumor. In case of a DAC, the chemical space represented by natural peptides is limited; therefore, it is desirable to have a variety of non-peptide linkers which act like peptides and can be effectively cleaved by lysosomal proteases. As such, provided herein in some embodiments are different types of non-peptide linkers for use as the linker L1 that can be cleaved by lysosomal enzymes.

A) Peptidomimetic Linkers—Provided herein are different types of non-peptide, peptidomimetic linkers for PAC that are cleavable by lysosomal enzymes. For example, the amide bond in the middle of a dipeptide (e.g. Val-Cit) was replaced with an amide mimic; and/or entire amino acid (e.g., valine amino acid in Val-Cit dipeptide) was replaced with a non-amino acid moiety (e.g., cycloalkyl dicarbonyl structures (for example, ring size=4 or 5)).

when L1 is a peptidomimetic linker, it is represented by the following formula

-Str-(PM)-Sp-,

wherein:
Str is a stretcher unit covalently attached to Ab;
Sp is a bond or spacer unit covalently attached to a degrader moiety; and
PM is a non-peptide chemical moiety selected from the group consisting of:

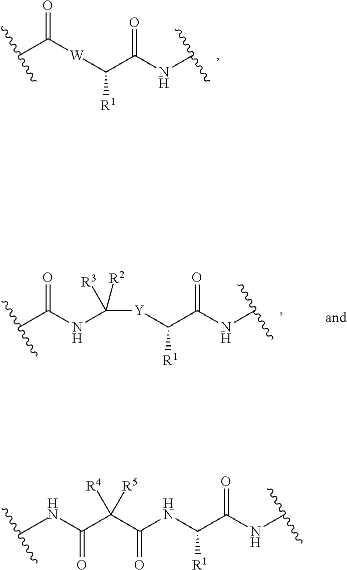

W is —NH-heterocycloalkyl- or heterocycloalkylene;
Y is heteroarylene, arylene, —C(═O)C₁-C₆ alkylene, C₁-C₆alkylene-NH—, C₁-C₆ alkylene-NH—CH₂—, C₁-C₆ alkylene-N(CH₃)—CH₂—, C₁-C₆ alkenylene, or C₁-C₆alkynylene;
each R¹ is independently C₁-C₁₀ alkyl, C₁-C₁₀ alkenyl, (C₁-C₁₀ alkyl)NHC(═NH)NH₂, or (C₁-C₁₀ alkyl)NHC(═O)NH₂;
R² and R³ are each independently —H, C₁-C₁₀ alkyl, C₁-C₁₀ alkenyl, arylalkyl, or heteroarylalkyl, or R² and
R³ together with atoms attached thereof form a C₃-C₇ cycloalkyl; and
R⁴ and R⁵ are each independently C₁-C₁₀ alkyl, C₁-C₁₀ alkenyl, arylalkyl, heteroarylalkyl, (C₁-C₁₀ alkyl)OCH₂—, or R⁴ and R⁵ together with atoms attached thereto form a C₃-C₇ cycloalkyl ring.

In some embodiments, the L1 is connected to the degrader molecule through any of the E3LB, L2, or PB groups. In some embodiments, Y is heteroaryl; R⁴ and R⁵ together form a cyclobutyl ring. In some embodiments, Y is a moiety selected from the group consisting of:

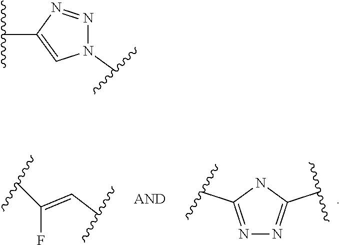 [SMILES: *c1cn(*)nn1 ; */C=C(/*)F ; *c1nnc(*)[nH]1]

In some embodiments, Str is a chemical moiety represented by the following formula:

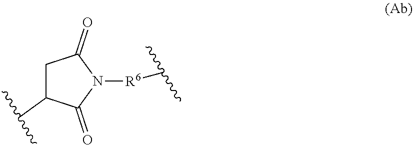

wherein R⁶ is selected from the group consisting of C₁-C₁₀ alkylene, C₁-C₁₀ alkenyl, C₃-C₈ cycloalkyl, (C₁-C₈ alkylene)O—, and C₁-C₁₀ alkylene-C(═O)N(R^{a})—C₂-C₆ alkylene, where each alkylene may be substituted by one to five substituents selected from the group consisting of halo, trifluoromethyl, difluoromethyl, amino, alkylamino, cyano, sulfonyl, sulfonamide, sulfoxide, hydroxy, alkoxy, ester, carboxylic acid, alkylthio, C₃-C₈ cycloalkyl, C₄-C₇ heterocycloalkyl, aryl, arylalkyl, heteroarylalkyl and heteroaryl each R^{a} is independently H or C₁-C₆ alkyl; Sp is —Ar—R^{b}—, wherein Ar is aryl or heteroaryl, R^{b} is (C₁-C₁₀ alkylene)O—.

In embodiments, Str has the formula:

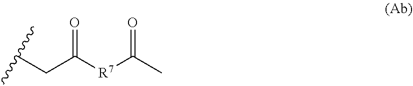

wherein R⁷ is selected from C₁-C₁₀ alkylene, C₁-C₁₀ alkenylene, —(C₁-C₁₀ alkylene)-O—, —N(R^{c})—(C₂-C₆ alkylene)-N(R^{c})— and —N(R^{c})—(C₂-C₆ alkylene)-; where each R^{c} is independently H or C₁-C₆ alkyl; Sp is —Ar—R^{b}—, wherein Ar is aryl or heteroaryl, R^{b} is (C₁-C₁₀ alkylene)O— or Sp —C₁-C₆ alkylene-C(═O)NH—.

In some embodiments, L1 is a non-peptide chemical moiety represented by the following formula

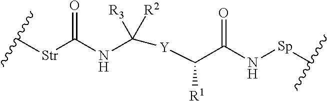

R¹ is C₁-C₆ alkyl, C₁-C₆ alkenyl, (C₁-C₆ alkyl)NHC(═NH)NH₂ or (C₁-C₆ alkyl)NHC(═O)NH₂; R³ and R² are each independently H or C₁-C₁₀ alkyl.

In some embodiments, L1 is a non-peptide chemical moiety represented by the following formula

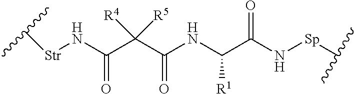

R¹ is C₁-C₆ alkyl, (C₁-C₆ alkyl)NHC(═NH)NH₂ or (C₁-C₆ alkyl)NHC(═O)NH₂;
R⁴ and R⁵ together form a C₃-C₇ cycloalkyl ring.
In some embodiments, L1 is a non-peptide chemical moiety represented by the following formula

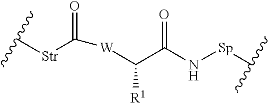

R¹ is C₁-C₆ alkyl, (C₁-C₆ alkyl)NHC(═NH)NH₂ or (C₁-C₆ alkyl)NHC(═O)NH₂ and W is as defined above.

In some embodiments, the linker may be a peptidomimetic linker such as those described in WO2015/095227, WO2015/095124 or WO2015/095223.

B) Non-peptidomimetic Linkers. In some embodiments, the linker L1 forms a disulfide bond with the antibody. In an aspect, the linker has the structure:

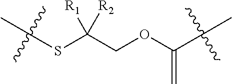

wherein, R¹ and R² are independently selected from H and C₁-C₆ alkyl, or R¹ and R² form a 3, 4, 5, or 6-membered cycloalkyl or heterocyclyl group. The linker is covalently bound to an antibody and a degrader as follows:

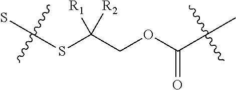

In one aspect the carbonyl group of the linker is connected to an amine group in the degrader molecule. It is also noted that the sulfur atom connected to Ab is a sulfur group from a cysteine in the antibody. In another aspect, a linker L1 has a functionality that is capable of reacting with a free cysteine present on an antibody to form a covalent bond. Nonlimiting examples of such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on an antibody. Examples of such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting examples of such reactive functionalities include, but are not limited to, hydrazide, oxime, amino, hydrazine, thiosemicarbazone, hydrazine carboxylate, and arylhydrazide.

A linker L1 may comprise one or more linker components. Exemplary linker components include 6-maleimidocaproyl (“MC”), maleimidopropanoyl (“MP”), valine-citrulline (“val-cit” or“vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below. In some embodiments, the linker L1 or a fragments thereof comprises MC (6-maleimidocaproyl), MCC (a maleimidomethyl cyclohexane-1-carboxylate), MP (maleimidopropanoyl), val-cit (valine-citrulline), val-ala (valine-alanine), ala-phe (alanine-phenylalanine), PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate. Further examples of linkers or fragments thereof include: BS3 ([Bis(sulfosuccinimidyl)suberate]; BS3 is a homobifunctional N-hydroxysuccinimideester that targets accessible primary amines), NHS/EDC (N-hydroxysuccinimide and N-ethyl-(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups). To form covalent bonds, a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA). Primary amines are the principal targets for NHS esters. Accessible a-amino groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the disclosure, the functional group on the protein may be a thiol group and the chemically reactive group may be a maleimido-containing group such as gamma-maleimide-butrylamide (GMBA or MPA). Such maleimide containing groups are referred to herein as maleido groups. The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In some embodiments, the linker L 1 comprises a lysine linker. In some embodiments, said linker comprises a MC (6-maleimidocaproyl), a MCC (a maleimidomethyl cyclohexane-1-carboxylate), a MP (maleimidopropanoyl), a val-cit (valine-citrulline), a val-ala (valine-alanine), an ala-phe (alanine-phenylalanine), a PAB (p-aminobenzyloxycarbonyl), a SPP (N-Succinimidyl 4-(2-pyridylthio) pentanoate), 2,5-dioxopyrrolidin-1-yl 4-(pyridin-2-ylthio)hexanoate, 2,5-dioxopyrrolidin-1-yl 5-methyl-4-(pyridin-2-ylthio)hexanoate, 2,5-dioxopyrrolidin-1-yl 5-methyl-4-(pyridin-2-ylthio)heptanoate, 2,5-dioxopyrrolidin-1-yl 5-ethyl-4-(pyridin-2-ylthio)heptanoate, 2,5-dioxopyrrolidin-1-yl 4-cyclopropyl-4-(pyridin-2-ylthio)butanoate, 2,5-dioxopyrrolidin-1-yl 4-cyclobutyl-4-(pyridin-2-ylthio)butanoate, 2,5-dioxopyrrolidin-1-yl 4-cyclopentyl-4-(pyridin-2-ylthio)butanoate, 2,5-dioxopyrrolidin-1-yl 4-cyclohexyl-4-(pyridin-2-ylthio)butanoate, a SMCC (N-Succinimidyl 4-(N-maleimidomethyl)cyclohexane-1 carboxylate), or a SIAB (N-Succinimidyl (4-iodo-acetyl)aminobenzoate). In some embodiments, said linker is derived from a cross-linking reagent, wherein the cross-linking reagent comprises N-succinimidyl-3-(2-pyridyldithio)propionate (SPDP), 2,5-dioxopyrrolidin-1-yl 3-cyclopropyl-3-(pyridin-2-yldisulfaneyl)propanoate, 2,5-dioxopyrrolidin-1-yl 3-cyclobutyl-3-(pyridin-2-yldisulfaneyl)propanoate, N-succinimidyl 4-(2-pyridyldithio)pentanoate (SPP), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl 4-(2-pyridyldithio)butanoate (SPDB), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC), or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

A linker may be a“cleavable linker,” facilitating release of a degrader. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker has the following Formula:

-Aₐ-W_{w}—Y_{y}—

wherein A is a “stretcher unit”, and a is an integer from 0 to 1; W is an “amino acid unit”, and w is an integer from 0 to 12; Y is a “spacer unit”, and y is 0, 1, or 2. Exemplary embodiments of such linkers are described in U.S. Pat. No. 7,498,298.

In some embodiments, a linker component comprises a “stretcher unit” that links an antibody to another linker component or to a degrader molecule. Nonlimiting exemplary stretcher units are shown below (wherein the wavy line indicates sites of covalent attachment to an antibody, degrader, or additional linker components):

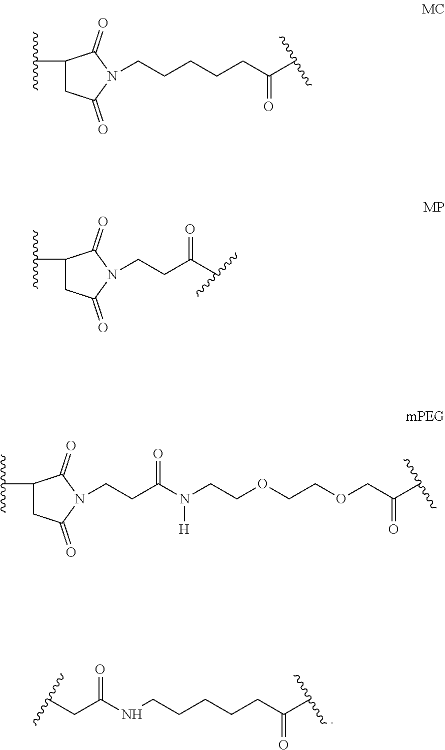 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

Linker L2

The E3LB and PB groups of degraders described herein may be connected with linker (L2) via any suitable means including, but not limited to, covalent linkage. In some instances, the linker group L2 is a group comprising one or more covalently connected structural units of A (e.g., -A₁ . . . A_{q}-), wherein A₁ is a group coupled to at least one of a E3LB, a PB, or a combination thereof. In certain embodiments, A₁ links a E3LB, a PB, or a combination thereof directly to another E3LB, PB, or combination thereof. In other instances, A₁ links a EL3B, a PB, or a combination thereof indirectly to another E3LB, PB, or combination thereof through A_{q}.

In certain instances, A₁ to A_{q} are, each independently, a bond, CR^{La}R^{Lb}, O, S, SO, SO₂, NR^{Lc}, SO₂NR^{Lc}, SONR^{Lc}, CONR^{Lc}, NR^{LC}CONR^{Ld}, NR^{Lc}SO₂NR^{Ld}, CO, CR^{La}—CR^{Lb}, C≡C, SiR^{La}R^{Lb}, P(O)R^{La}, P(O)OR^{La}, NR^{Lc}C═NCN)NR^{Ld}, NR^{Lc}C═NCN), NR^{Lc}C(═CNO₂)NR^{Ld}, C₃-11cycloalkyl optionally substituted with 0-6 R^{La} and/or R^{Lb} groups, C₃-11heterocyclyl optionally substituted with 0-6 R^{La} and/or R^{Lb} groups, aryl optionally substituted with 0-6 R^{La} and/or R^{Lb} groups, heteroaryl optionally substituted with 0-6 R^{La} and/or R^{Lb} groups, where R^{La} or R^{Lb}, each independently, can be linked to other A groups to form cycloalkyl and/or heterocyclyl moeity which can be further substituted with 0-4 R^{Le} groups; wherein R^{La}, R^{Lb}, R^{Lc}, R^{Ld} and R^{Le} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl(C₁₋₈alkyl), OH, NH₂, SH, SO₂C₁₋₈alkyl, P(O(OC₁₋₈alkyl)(C₁₋₈alkyl), P(O(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SFs, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In certain instances, q is an integer greater than or equal to 0. In certain instances, q is an integer greater than or equal to 1. In certain instances, e.g., where q is greater than 2, A_{q} is a group which is connected to an E3LB moiety, and A₁ and A_{q} are connected via structural units of A (number of such structural units of A: q-2). In certain instances, e.g., where q is 2, A_{q} is a group which is connected to A₁ and to an E3LB moiety. In certain instances, e.g., where q is 1, the structure of the linker group L2 is -A₁-, and A₁ is a group which is connected to an E3LB moiety and a PB moiety. In additional instances, q is an integer from 1 to 100, 1 to 90, 1 to 80, 1 to 70, 1 to 60, 1 to 50, 1 to 40, 1 to 30, 1 to 20, or 1 to 10.

In certain instances, the linker (L2) is selected from the group consisting of:

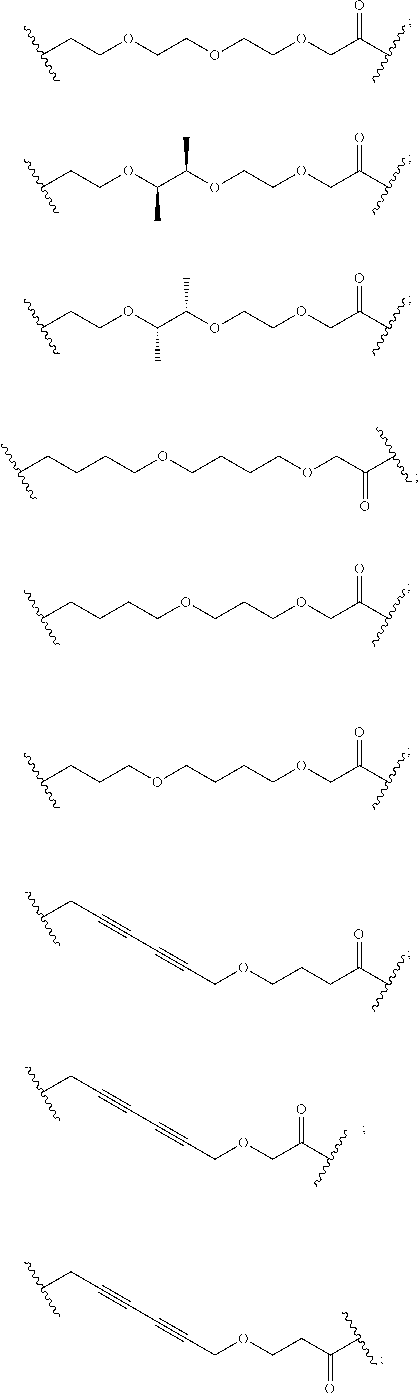

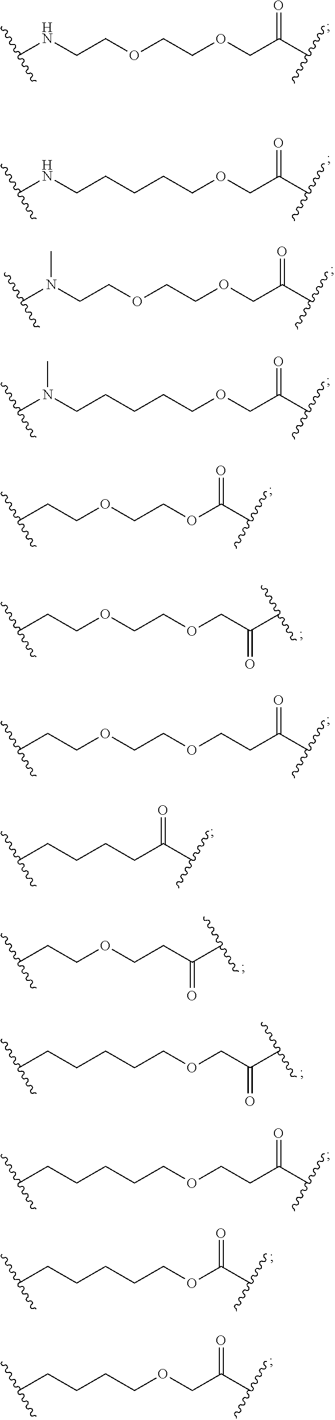

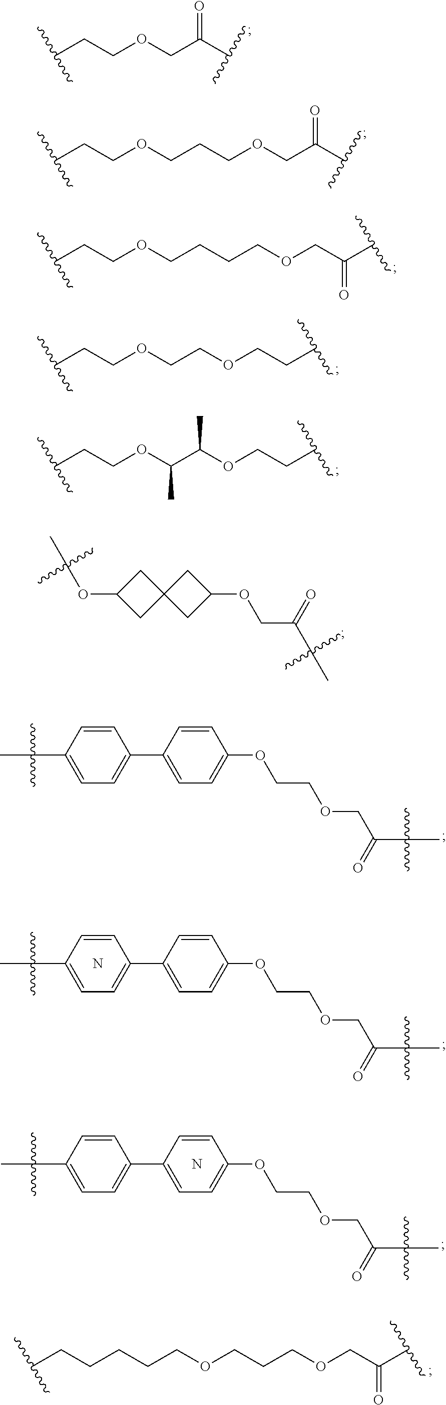

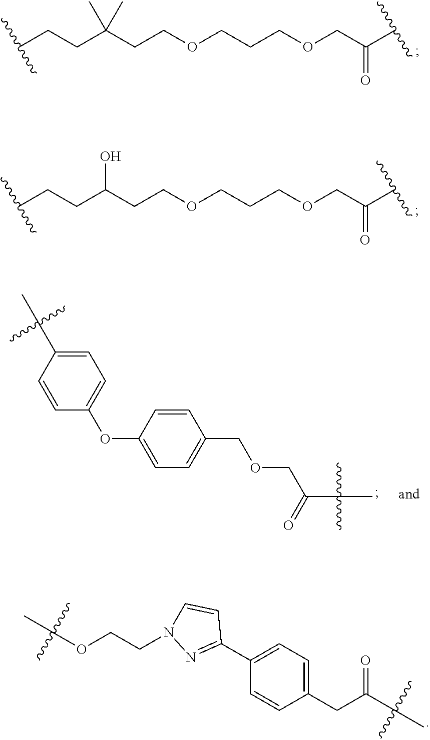 [SMILES: *CCC(C)(C)CCOCCCOCC(*)=O ; *CCC(O)CCOCCCOCC(*)=O ; *C(=O)COCc1ccc(Oc2ccc(*)cc2)cc1 ; *OCCn1ccc(-c2ccc(CC(*)=O)cc2)n1]

The linker group may, in some instances, be optionally a substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain instances, the linker is substituted with an aryl, phenyl, benzyl, alkyl, alkylene, or heterocycle group. The linker may be asymmetric or symmetrical. In some instances, the linker may be a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

The E3LB group and PB group may be covalently linked to the linker group through any group which is appropriate and stable to the chemistry of the linker. The linker may be independently covalently bonded to the E3LB group and the PB group through an amide, ester, thioester, keto group, carbamate (urethane), carbon or ether, each of which groups may be inserted anywhere on the E3LB group and PB group to provide maximum binding of the E3LB group on the ubiquitin ligase and the PB group on the target protein to be degraded. In certain aspects where the PB group is an E3LB group, the target protein for degradation may be the ubiquitin ligase itself. In certain aspects, the linker may be linked to an optionally substituted alkyl, alkylene, alkene or alkyne group, an aryl group or a heterocyclic group on the E3LB and/or PB groups. An E3LB group or a PB group may, in some instances, be derivatized to make a chemical functional group that is reactive with a chemical functional group on the linker. Alternatively, the linker may need to be derivatized to include a chemical functional group that can react with a functional group found on E3LB and/or PB.

L2 can also be represented by the formula:

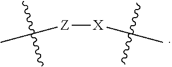

Where Z is a group which links E3LB to X; and X is a group linking Z to group PB.
In embodiments, Z is absent (a bond), —(CH₂)i-O, —(CH₂)i-S, —(CH₂)i-N—R, a (CH₂)—X₁Y₁ group wherein X₁Y₁ forms an amide group, or a urethane group, ester or thioester group, or a

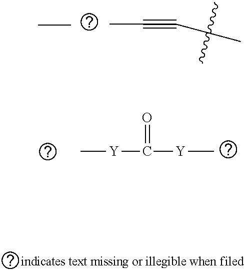

where, each R is H, or a C₁-C₃ alkyl, an alkanol group or a heterocycle (including a water soluble heterocycle, preferably, a morpholino, piperidine or piperazine group to promote water solubility of the linker group); each Y is independently a bond, O, S or N—R; and each i is independently 0 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
In embodiments, X is a

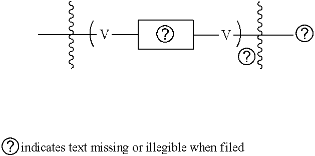

where each V is independently a bond (absent),

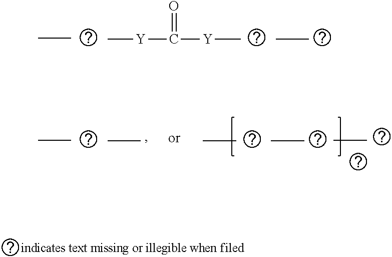

j is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3,4 or 5;
k is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; preferably k is 1, 2, 3, 4, or 5;
m′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1,2,3,4 or 5;
n is 1 to 100, 1 to 75, 1 to 60, 1 to 55, 1 to 50, 1 to 45, 1 to 40, 2 to 35, 3 to 30, 1 to 15, 1 to 10, to 8, 1 to 6, 1, 2, 3, 4 or 5;
X₁ is O, S or N—R, preferably O;
Y is the same as above;
and CON is a connector group (which may be a bond) which connects Z to X, when present in the linker group.

In embodiments, CON is a bond (absent), a heterocycle including a water-soluble heterocycle such as a piperazinyl or other group or a group,

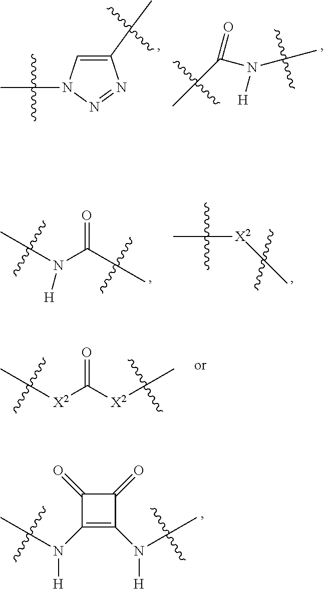

where X² is O, S, NR⁴, S(O), S(O)₂, —S(O)₂), —OS(O)₂, or OS(O)₂);
X³ is O, S, CHR⁴, NR⁴; and
R is H or a C₁-C₃ alkyl group optionally substituted with one or two hydroxyl groups, or a pharmaceutically acceptable salt, enantiomer or stereoisomer thereof.

In some aspects, the linker group is a (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units.

In embodiments, CON is

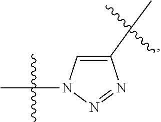 [SMILES: *c1cn(*)nn1]

or an amide group.

Although the E3LB group and PB group may be covalently linked to the linker group through any group which is appropriate and stable to the chemistry of the linker, in some aspects, the linker is independently covalently bonded to the E3LB group and the PB group through an amide, ester, thioester, keto group, carbamate (urethane) or ether, each of which groups may be inserted anywhere on the E3LB group and PB group to allow binding of the E3LB group to the ubiquitin ligase and the PB group to the target protein to be degraded. For example, as shown herein, the linker can be designed and connected to E3LB and PB to minimize, eliminate, or neutralize any impact its presence might have on the binding of E3LB and PB to their respective binding partners. In certain aspects, the targeted protein for degradation may be a ubiquitin ligase.

Methods of Use

The disclosure further provides a method for inhibiting cell-cell interactions that are endothelial cell (EC) specific, for example, but not limited to EC-EC, EC-mesenchymal stem cell, EC-fibroblast, EC-smooth muscle cell, EC-tumor cell, EC-leukocyte, EC-adipose cell and EC-neuronal cell interactions. In certain embodiments, the DACs containing the anti-TM4SF1 antibodies and fragments of the present disclosure, can be used to treat any human disease or disorder with a pathology that is characterized by abnormal EC-cell interactions. In certain embodiments, the EC-cell interaction is an EC-leukocyte interaction, where inhibition of the EC-leukocyte interaction is used to prevent inflammation.

In other embodiments, the disclosure features a method of treating or preventing a disease or disorder in a subject, wherein the disease or disorder is characterized by abnormal endothelial cell (EC)-cell interactions, said method comprising administering the antibody, or antigen-binding fragment thereof, as described herein. In certain embodiments, the EC-cell interactions include one or more of EC-mesenchymal stem cell, EC-fibroblast, EC-smooth muscle cell, EC-tumor cell, EC-leukocyte, EC-adipose cell and EC-neuronal cell interactions. In exemplary embodiments, the disease is an inflammatory disease or disorder, and the antibodies and fragments of the disclosure are used to inhibit EC-leukocyte interactions. In another exemplary embodiment, the disease or disorder is selected from an inflammatory disease or cancer. The adhesion of leukocytes to vascular endothelium is a hallmark of the inflammatory process. Accordingly, in one embodiment, a DAC containing an anti-TM4SF1 antibody, or an antigen binding fragment thereof, of the present disclosure is used to treat an inflammatory disease in which inhibiting leukocyte attachment to endothelial cells, or leukocyte transmigration across the endothelium is helpful for treatment (see, e.g. Rychly et al., Curr Pharm Des. 2006; 12(29):3799-806, incorporated by reference in its entirety herein). Examples include, but are not limited to, sepsis, inflammatory bowel disease, psoriasis or multiple sclerosis.

Each year approximately half a million patients die from cancer in the United States alone. Tumor metastasis is responsible for ˜90% of these deaths. No therapy that blocks metastasis is known. The present disclosure provides antibodies, and antigen-binding fragments thereof, that can treat cancer and inhibit metastatic cells based on immunoblockade of tumor cell (TC)—endothelial cell (EC) interactions mediated by a novel target, TM4SF1.

As described above, TM4SF1 is a small, tetraspanin-like, cell surface glycoprotein originally discovered as a TC antigen with roles in TC invasion and metastasis. TM4SF1 is selectively expressed by TCs and ECs. TM4SF1 is expressed at low levels on the vascular ECs supplying normal tissues in both mice and humans. It has been shown that TM4SF1 is expressed at ˜10-20 fold higher levels on the vascular ECs lining the blood vessels supplying many human cancers, and at equivalent high levels on cultured ECs. TM4SF1-enriched microdomains (TMED) recruit cell surface proteins like integrins to assist the formation of nanopodia, thin membrane channels that extend from the cell surface and mediate cell-cell interactions. Thus, in certain instances, DACs containing anti-TM4SF1 antibodies and fragments described herein interfere with nanopodia-mediated interactions and inhibit TC interactions with EC that are necessary for TC extravasation.

DACs of this disclosure may be formulated for treating a subject (e.g., a human) having a disorder associated with pathological angiogenesis (e.g., cancer, such as breast cancer, ovarian cancer, renal cancer, colorectal cancer, liver cancer, gastric cancer, and lung cancer; obesity; macular degeneration; diabetic retinopathy; psoriasis; rheumatoid arthritis; cellular immunity; and rosacea.

TM4SF1 is highly expressed on the surface of most epithelial TCs, and, is also highly expressed on the EC lining tumor blood vessels and on cultured EC. It is expressed at ˜10-20 fold lower levels on the surface of normal vascular ECs. In mouse models, tumor metastasis to lungs is related to TM4SF1 expression on both ECs and TCs. Metastasis requires initial attachment of TC to vascular EC and their subsequent migration across ECs to enter the lung or other metastatic sites. The examples below show that, in some instances, the anti-TM4SF1 antibodies of the present disclosure interfere with TC-EC interactions in culture and can also inhibit tumor metastasis in vivo.

Thus, the DACs of the present disclosure can be used to block one or both of the earliest steps in metastasis, namely, TC attachment to vascular ECs and/or transmigration of TCs across ECs, and thereby prevent or substantially reduce the number of metastases in at risk cancer patients.

The present disclosure further provides a method for preventing metastasis. Human tumors typically shed TCs into the blood and lymphatics at early stages of growth; hence, early treatment of primary tumors provides no guarantee that metastasis has not already taken place. Thus, immunoblockade of TM4SF1 can be used to treat or prevent hematogenous metastases or to treat or prevent lymphatic metastases.

The methods of this disclosure are, in some embodiments, directed to inhibiting metastatic cells in a subject. In one embodiment, the subject has a cancer, e.g., a cancer that is associated with metastasis or a cancer that has already metastasized. In other embodiments, the subject was already treated for cancer and is in remission or partial remission, wherein the benefits of administering DACs containing the anti-TM4SF1 antibodies or fragments described herein are that they work to prevent metastasis and maintain remission or partial remission.

In certain embodiments, the disclosure provides a method of treating a person having a greater risk of developing metastasis, wherein administration of the DACs containing the anti-TM4SF1 antibodies and fragments described herein can be used to inhibit or delay onset of metastasis.

Included in the disclosure is a method of blocking tumor metastasis, particularly metastasis to the lung, by administering an anti-TM4SF1 antibody to a subject in need thereof. In some examples, the anti-TM4SF1 antibody is a human anti-TM4SF1 antibody, also referred to herein as anti-hTM4SF1. In certain embodiments, the methods can include administration of an effective amount of an DAC containing an anti-hTM4SF1 antibody to a subject in need thereof, wherein the effective amount of the antibody prevents tumor cell (TC) attachment to and migration across vascular endothelial cells (ECs).

In certain embodiments, an DAC containing an anti-TM4SF1 antibody is administered to a subject having cancer or at risk of having metastasis such that the dose amount and frequency maintains long term TM4SF1 immunoblockade. The dosing regimen may maximally inhibit TM4SF1-mediated metastasis by administering an DAC containing an anti-TM4SF1 antibody to a subject in an amount sufficient to saturate TM4SF1 expressed on normal vascular ECs of the subject.

In certain embodiments, the effective amount of an DAC containing an anti-TM4SF1 antibody, or an antigen binding fragment thereof, that is administered is an amount sufficient to, at one week, achieve circulating antibody concentrations >1 pg/ml.

In certain embodiments, the effective amount of an DAC containing an anti-TM4SF1 antibody, or an antigen binding fragment thereof that is administered is an amount sufficient to maintain serum concentrations of the antibody at or above 1 pg/ml continuously for about 1 month.

In one embodiment, the disclosure provides a method of treating or preventing metastasis in a human subject comprising administering to the subject an effective amount of an DAC containing an anti-TM4SF1 antibody, or an antigen binding fragment thereof, wherein the effective amount of the antibody, or antigen binding fragment thereof, comprises 1 to 80 mg/kg of the amount of the antibody, or antigen binding fragment thereof.

The mode of administration for therapeutic use of the DACs of the disclosure may be any suitable route that delivers the antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the DACs of the disclosure may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours. The dose given to a subject in some embodiments is about 0.005 mg to about 100 mg/kg, e.g., about 0.05 mg to about 30 mg/kg or about 5 mg to about 25 mg/kg, or about 4 mg/kg, about 8 mg/kg, about 16 mg/kg or about 24 mg/kg, or for example about 0.1, 0.2, 0.3, 0.4, 0.5, 0.6, 0.7, 0.8, 0.9, 1, 2, 3, 4, 5, 6, 7, 8, 9 or 10 mg/kg. In certain embodiments, the dose given to a subject is, for example about 15, 16, 17, 18, 19, 20, 21, 22, 23, 24, 25, 30, 40, 50, 60, 70, 80, 90 or 100 mg/kg. In some instances, the dose of the antibodies of the disclosure given to a subject may be about 0.1 mg/kg to 10 mg/kg via intravenous administration. In some instances, the dose of the antibodies of the disclosure given to a subject is about 0.1 mg/kg to 10 mg/kg via subcutaneous administration. In some instances, the dose of the antibodies of the disclosure given to a subject is about 0.1 mg/kg via intravenous administration. In some instances, the dose of the antibodies of the disclosure given to a subject is about 0.1 mg/kg via subcutaneous administration. In some embodiments, the dose of the antibodies of the disclosure given to a subject is about 0.3 mg/kg via intravenous administration. In some examples, the dose of the antibodies of the disclosure given to a subject is about 0.3 mg/kg via subcutaneous administration. In some examples, the dose of the antibodies of the disclosure given to a subject is about 1.0 mg/kg via intravenous administration. In some examples, the dose of the antibodies of the disclosure given to a subject is about 1.0 mg/kg via subcutaneous administration. In some examples, the dose of the antibodies of the disclosure given to a subject is about 3.0 mg/kg via intravenous administration. In some examples, the dose of the antibodies of the disclosure given to a subject is about 3.0 mg/kg via subcutaneous administration. In some examples, the dose of the antibodies of the disclosure given to a subject may be about 10.0 mg/kg via intravenous administration. In some examples, the dose of the antibodies of the disclosure given to a subject is about 10.0 mg/kg via subcutaneous administration.

In certain embodiments, a fixed unit dose of the antibodies of the disclosure is given, for example, 50, 100, 200, 500 or 1000 mg, or the dose may be based on the patient's surface area, e.g., 500, 400, 300, 250, 200, or 100 mg/m². In some instances, between 1 and 8 doses, (e.g., 1, 2, 3, 4, 5, 6, 7 or 8) is administered to treat the patient, but 9, 10, 11, 12, 13, 14, 15, 16, 17, 18, 19, 20 or more doses are given.

The administration of the DACs of the disclosure described herein, in some embodiments, is repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration is at the same dose or at a different dose. In some examples, the DACs of the disclosure described herein is administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. Alternatively, in some embodiments, the DACs of the disclosure described herein are administered at between 0.1 mg/kg to about 10 mg/kg at weekly interval for 17 weeks. For example, in some cases the antibodies of the disclosure are provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof. In some embodiments, the antibodies of the disclosure described herein is administered prophylactically in order to reduce the risk of developing an inflammatory disease such as RA, psoriatic arthritis or psoriasis, delay the onset of the occurrence of an event in progression of the inflammatory disease such as RA, psoriatic arthritis or psoriasis. In some examples, the DACs of the disclosure are lyophilized for storage and reconstituted in a suitable carrier prior to use. In some cases, the antibodies of the disclosure are supplied as a sterile, frozen liquid in a glass vial with stopper and aluminum seal with flip-off cap. In some examples, each vial might contain DAC containing 3.3 mL of a 50 mg/mL solution of the antibody (including a 10% overfill) in a formulation of 10 mM histidine, 8.5% (w/v) sucrose, and 0.04% (w/v) Polysorbate 80 at pH 5.8. In some examples, the vials contain no preservatives and are for single use. Vials may be stored frozen and protected from light. To prepare for IV administration, the DAC formulations, in some examples, are filtered with a 0.22 micron filter before being diluted in sterile diluent. In some examples, diluted DACs at volumes up to approximately 100 mL are administered by IV infusion over a period of at least 30 minutes using an in-line 0.22 micron filter. Alternatively, in some embodiments, the DACs are administered as 1 or 2 subcutaneous injections containing about 50 mg/mL antibody in about 3.3 mL. The subcutaneous injection site may be, for example, within the abdominal area.

Pharmaceutical Compositions

The DACs of this disclosure, can, in some embodiments, be included in compositions (e.g., pharmaceutical compositions). The pharmaceutical compositions of the disclosure may further include a pharmaceutically acceptable carrier, excipient, or diluent.

The term “pharmaceutical composition” as used herein refers to a composition containing a TM4SF1 binding protein described herein formulated with a pharmaceutically acceptable carrier, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable carrier” as used herein refers to a carrier which is physiologically acceptable to a treated mammal (e.g., a human) while retaining the therapeutic properties of the protein with which it is administered. One exemplary pharmaceutically acceptable carrier is physiological saline. Other physiologically acceptable carriers and their formulations are known to one skilled in the art and described, for example, in Remington's Pharmaceutical Sciences (18th edition, A. Gennaro, 1990, Mack Publishing Company, Easton, Pa.), incorporated herein by reference.

Pharmaceutical compositions containing an DAC containing an TM4SF1 antibody or antigen-binding fragment thereof, are, in some embodiments, prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

A pharmaceutically acceptable excipient is, in some examples, an excipient described in the Handbook of Pharmaceutical Excipients, American Pharmaceutical Association (1986). Non-limiting examples of suitable excipients include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient is a buffering agent. Non-limiting examples of suitable buffering agents include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof is used, in some embodiments, in a pharmaceutical composition of the present disclosure.

In some embodiments an excipient comprises a preservative. Non-limiting examples of suitable preservatives include antioxidants, such as alpha-tocopherol and ascorbate, and antimicrobials, such as parabens, chlorobutanol, and phenol. In some examples, antioxidants further include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof. The binders used in a pharmaceutical formulation are, in some examples, selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or any combinations thereof.

In some embodiments a pharmaceutical composition as described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation, in some embodiments, are be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation comprises a dispersion enhancer as an excipient. Non-limiting examples of suitable dispersants include, in some examples, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein comprises a disintegrant as an excipient. In some embodiments a disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant is an effervescent disintegrant. Non-limiting examples of suitable effervescent disintegrants include sodium bicarbonate in combination with citric acid, and sodium bicarbonate in combination with tartaric acid.

In some embodiments an excipient comprises a flavoring agent. Flavoring agents incorporated into an outer layer are, in some examples, chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition as described herein comprises a coloring agent. Non-limiting examples of suitable color agents include food, drug and cosmetic colors (FD&C), drug and cosmetic colors (D&C), and external drug and cosmetic colors (Ext. D&C). A coloring agents can be used as dyes or their corresponding lakes.

In some instances, a pharmaceutical composition as described herein comprises a chelator. In some cases, a chelator is a fungicidal chelator. Examples include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

Also contemplated are combination products that include an anti-TM4SF1 antibody as disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some instances, a pharmaceutical composition comprises an additional agent. In some cases, an additional agent is present in a therapeutically effective amount in a pharmaceutical composition.

Under ordinary conditions of storage and use, the pharmaceutical compositions as described herein comprise a preservative to prevent the growth of microorganisms. In certain examples, the pharmaceutical compositions as described herein do not comprise a preservative. The pharmaceutical forms suitable for injectable use include sterile aqueous solutions or dispersions and sterile powders for the extemporaneous preparation of sterile injectable solutions or dispersions. The pharmaceutical compositions comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form is suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed may be available in light of the present disclosure. For example, one dosage is dissolved, in certain cases, in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions is prepared by incorporating a immunotherapy agent, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein are, in some instances, formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups are, in some cases, derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions are administered, in some embodiments, in a manner compatible with the dosage formulation and in such amount as is therapeutically effective.

In certain embodiments, a pharmaceutical composition of this disclosure comprises an effective amount of an anti-TM4SF1 antibody, as disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the immunotherapeutic agents or any other suitable vehicle, delivery or dispensing means or material. Such carriers are formulated, for example, by conventional methods and administered to the subject at an effective amount.

Combination Therapies

In certain embodiments, the methods of this disclosure comprise administering an DAC as disclosed herein, followed by, preceded by or in combination with one or more further therapy. Examples of the further therapy can include, but are not limited to, chemotherapy, radiation, an anti-cancer agent, or any combinations thereof. The further therapy can be administered concurrently or sequentially with respect to administration of the immunotherapy. In certain embodiments, the methods of this disclosure comprise administering an immunotherapy as disclosed herein, followed by, preceded by, or in combination with one or more anti-cancer agents or cancer therapies. Anti-cancer agents include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies include chemotherapy, biological therapy, radiotherapy, immunotherapy, hormone therapy, anti-vascular therapy, cryotherapy, toxin therapy and/or surgery or combinations thereof. In certain embodiments, the methods of this disclosure include administering an immunotherapy, as disclosed herein, followed by, preceded by or in combination with one or more further immunomodulatory agents. An immunomodulatory agent includes, in some examples, any compound, molecule or substance capable of suppressing antiviral immunity associated with a tumor or cancer. Non-limiting examples of the further immunomodulatory agents include an agent that binds to a protein selected from the group consisting of: A2AR, B7-H3, B7-H4, BTLA, CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, and VISTA; an anti-CD33 antibody or variable region thereof; an anti-CD11b antibody or variable region thereof; a COX2 inhibitor, e.g., celecoxib, cytokines, such as IL-12, GM-CSF, IL-2, IFN3 and 1FNy, and chemokines, such as MIP-1, MCP-1 and IL-8.

In certain examples, where the further therapy is radiation exemplary doses are 5,000 Rads (50 Gy) to 100,000 Rads (1000 Gy), or 50,000 Rads (500 Gy), or other appropriate doses within the recited ranges. Alternatively, the radiation dose are about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines can include, without limitation, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors can, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708); delanzomib (CEP-18770); and 0-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

“In combination with,” as used herein, means that the anti-TM4SF1 antibody and the further therapy are administered to a subject as part of a treatment regimen or plan. In certain embodiments, being used in combination does not require that the anti-TM4SF1 antibody and the further therapy are physically combined prior to administration or that they be administered over the same time frame. For example, and not by way of limitation, the anti-TM4SF1 antibody and the one or more agents are administered concurrently to the subject being treated, or are administered at the same time or sequentially in any order or at different points in time.

Kits

In some embodiments, the disclosure provides kits that include a composition (e.g., a pharmaceutical composition) of the disclosure (e.g., a composition including an DAC containing an anti-TM4SF1 antibody or antigen binding fragment thereof). The kits include instructions to allow a clinician (e.g., a physician or nurse) to administer the composition contained therein to a subject to treat a disorder associated with pathological angiogenesis (e.g., cancer).

In certain embodiments, the kits include a package of a single-dose pharmaceutical composition(s) containing an effective amount of an antibody of the disclosure. Optionally, instruments or devices necessary for administering the pharmaceutical composition(s) may be included in the kits. For instance, a kit of this disclosure may provide one or more pre-filled syringes containing an effective amount of a vaccine, vector, stabilized timer, or optimized viral polypeptide of the disclosure. Furthermore, the kits may also include additional components such as instructions regarding administration schedules for a subject having a disorder associated with pathological angiogenesis (e.g., cancer) to use the pharmaceutical composition(s) containing a TM4SF1 binding protein or polynucleotide of the disclosure.

TABLE 16
SEQUENCE DESCRIPTION

| | | |
|---|---|---|
| SEQ ID | | |
| NO | Description | Sequence |

Antibody AGX-A01

| | | |
|---|---|---|
| 1 | AGX-A01 | EVILVESGGGLVKPGGSLKLSCAASGFTFSSFAMS |
| | Variable heavy (VH) | WVRQTPEKRLEWVATISSGSIYIYYTDGVKGRFTI |
| | chain-amino acid | SRDNAKNTVHLQMSSLRSEDTAMYYCARRGIYY |
| | | GYDGYAMDYWGQGTSVTVS |
| 2 | AGX-A01 | AVVMTQTPLSLPVSLGDQASISCRSSQSLVHSNG |
| | Variable light (VL) | NTYLHWYMQKPGQSPKVLIYKVSNRFSGVPDRF |
| | chain-amino acid | SGSGSGTDFTLKISRVEADDLGIYFCSQSTHIPLAF |
| | | GAGTKLELK |

Antibody AGX-A03

| | | |
|---|---|---|
| 3 | AGX-A03 | QIQLVQSGPELKKPGETVKISCKASGYSFRDYGM |
| | Variable heavy (VH) | NWVKQAPGRTFKWMGWINTYTGAPVYAADFK |
| | chain-amino acid | GRFAFSLDTSASAAFLQINNLKNEDTATYFCARW |
| | | VSYGNNRNWFFDFWGAGTTVTVSS |
| 4 | AGX-A03 | CAGATCCAGTTGGTGCAGTCTGGACCTGAGCT |
| | Variable heavy (VH) | GAAGAAGCCTGGAGAGACAGTCAAGATCTCCT |
| | chain-nucleic acid | GCAAGGCTTCTGGGTATTCCTTCAGAGACTATG |
| | | GAATGAACTGGGTGAAGCAGGCTCCAGGAAGG |
| | | ACTTTTAAGTGGATGGGCTGGATAAACACCTA |
| | | CACTGGAGCGCCAGTATATGCTGCTGACTTCA |
| | | AGGGACGGTTTGCCTTCTCTTTGGACACCTCTG |
| | | CCAGCGCTGCCTTTTTGCAGATCAACAACCTCA |
| | | AAAATGAAGACACGGCTACATATTTCTGTGCA |
| | | AGATGGGTCTCCTACGGTAATAACCGCAACTG |
| | | GTTCTTCGATTTTTGGGGCGCAGGGACCACGGT |
| | | CACCGTCTCCTCA |
| 5 | AGX-A03 | CAAATTCAGTTGGTTCAATCCGGCCCTGAGCTC |
| | Variable heavy (VH) | AAGAAGCCTGGAGAGACAGTGAAGATAAGTTG |
| | chain-codon optimized | TAAGGCTAGTGGCTATTCATTTCGAGATTATGG |
| | nucleic acid | GATGAATTGGGTCAAGCAGGCCCCAGGGCGGA |
| | | CCTTCAAATGGATGGGGTGGATCAATACTTAC |
| | | ACTGGCGCACCAGTATATGCAGCTGATTTTAA |
| | | GGGTCGCTTTGCATTTTCACTTGATACTTCAGC |
| | | CAGTGCCGCTTTTTTGCAAATCAACAATCTCAA |
| | | AAATGAAGACACTGCTACATATTTCTGCGCCA |
| | | GGTGGGTGAGCTATGGCAATAACAGAAATTGG |
| | | TTCTTTGACTTTTGGGGCGCAGGCACCACCGTC |
| | | ACTGTCTCATCA |
| 6 | VH-CDR1 | GYSFRDYGMN |
| 7 | VH-CDR2 | WINTYTGAPVYAADFKG |
| 8 | VH-CDR3 | WVSYGNNRNWFFDF |
| 9 | AGX-A03 | DVLMTQTPLSLPVRLGDQASISCRSSQTLVHSNG |
| | Variable light (VL) | NTYLEWYLQKPGQSPKLLIYKVSNRLSGVPDRFS |
| | chain-amino acid | GSGSGTDFTLKISRVETEDLGVYYCFQGSHGPWT |
| | | FGGGTKLEIK |
| 10 | AGX-A03 | GATGTTTTGATGACCCAAACTCCACTCTCCCTG |
| | Variable light (VL) | CCTGTCCGTCTTGGAGATCAGGCCTCCATCTCT |
| | chain-nucleic acid | TGTAGATCTAGTCAGACCCTTGTACATAGTAAT |
| | | GGAAACACCTATTTAGAATGGTACCTGCAGAA |
| | | ACCAGGCCAGTCTCCAAAACTCTTGATCTACA |
| | | AAGTTTCCAATCGACTTTCTGGGGTCCCAGACA |
| | | GGTTCAGTGGCAGTGGATCAGGGACAGATTTC |
| | | ACACTCAAGATCAGCAGAGTGGAGACTGAGGA |
| | | TCTGGGAGTTTATTACTGCTTTCAAGGTTCACA |
| | | TGGTCCGTGGACGTTCGGTGGAGGCACCAAGC |
| | | TGGAAATCAAA |
| 11 | AGX-A03 | GACGTACTTATGACACAAACTCCCTTGAGCTTG |
| | Variable light (VL) | CCAGTACGGCTTGGCGATCAAGCTTCAATTTCA |
| | chain-codon optimized | TGTCGTTCTTCTCAAACACTTGTCCACTCAAAT |
| | nucleic acid | GGGAATACATATTTGGAATGGTATCTCCAAAA |
| | | GCCCGGCCAATCCCCAAAATTGTTGATTTACAA |
| | | GGTGTCTAATCGACTCTCAGGCGTCCCCGACCG |
| | | ATTCTCCGGGAGCGGGTCCGGTACAGACTTCA |
| | | CCTTGAAAATCTCCAGGGTAGAAACTGAAGAC |
| | | CTCGGAGTCTACTATTGTTTCCAGGGGTCACAC |
| | | GGCCCCTGGACATTTGGAGGAGGAACTAAGCT |
| | | CGAGATCAAA |
| 12 | VL-CDR1 | RSSQTLVHSNGNTYLE |
| 13 | VL-CDR2 | KVSNRLS |
| 14 | VL-CDR3 | FQGSHGPWT |

Antibody AGX-A04

| | | |
|---|---|---|
| 15 | AGX-A04 | EVQLQQSGPELVKPGASVKISCKTSGYTFTDYTM |
| | Variable heavy (VH) | HWVRQSHGKSLEWIGSFNPNNGGLTNYNQKFKG |
| | chain-amino acid | KATLTVDKSSSTVYMDLRSLTSEDSAVYYCTRIR |
| | | ATGFDSWGQGTTLTVSS |
| 16 | AGX-A04 | GAGGTCCAGCTGCAACAGTCTGGACCTGAGCT |
| | Variable heavy (VH) | GGTGAAGCCTGGGGCTTCAGTGAAGATATCCT |
| | chain-nucleic acid | GCAAGACTTCTGGATACACATTCACTGATTACA |
| | | CCATGCACTGGGTGAGGCAGAGCCATGGAAAG |
| | | AGCCTTGAGTGGATTGGAAGTTTTAATCCTAAC |
| | | AATGGTGGTCTTACTAACTACAACCAGAAGTT |
| | | CAAGGGCAAGGCCACATTGACTGTGGACAAGT |
| | | CTTCCAGCACAGTGTACATGGACCTCCGCAGC |
| | | CTGACATCTGAGGATTCTGCAGTCTATTACTGT |
| | | ACAAGAATCCGGGCTACGGGCTTTGACTCCTG |
| | | GGGCCAGGGCACCACTCTCACAGTCTCCTCA |
| 17 | AGX-A04 | GAGGTACAACTGCAACAGAGTGGACCTGAACT |
| | Variable heavy (VH) | TGTCAAACCTGGAGCAAGTGTGAAGATTAGCT |
| | chain-codon optimized | GTAAAACCAGTGGCTACACATTTACCGATTAT |
| | nucleic acid | ACTATGCACTGGGTAAGACAGAGCCACGGAAA |
| | | ATCACTGGAGTGGATTGGTAGTTTCAATCCTAA |
| | | CAACGGAGGATTGACAAATTACAACCAGAAGT |
| | | TCAAAGGGAAAGCCACCTTGACAGTTGATAAG |
| | | TCCTCAAGTACCGTGTATATGGATCTGCGTTCT |
| | | CTCACAAGTGAAGATAGCGCAGTTTACTACTG |
| | | TACCCGCATCCGAGCCACCGGGTTCGATTCATG |
| | | GGGTCAGGGGACAACACTGACTGTTTCTTCT |
| 18 | VH-CDR1 | GYTFTDYTMH |
| 19 | VH-CDR2 | SFNPNNGGLTNYNQKFKG |
| 20 | VH-CDR3 | IRATGFDS |
| 21 | AGX-A04 | DIVMSQSPSSLAVSAGEKVTMSCKSSQSLLNSRT |
| | Variable light (VL) | RKNYLAWYQQKPGQSPKLLIYWASTRESGVPDR |
| | chain-amino acid | FTGSGSGTDFTLTISNVQAEDLTVYYCKQSYNPP |
| | | WTFGGGTKLEIK |
| 22 | AGX-A04 | GACATTGTGATGTCACAGTCTCCATCCTCCCTG |
| | Variable light (VL) | GCTGTGTCAGCAGGAGAGAAGGTCACTATGAG |
| | chain-nucleic acid | CTGCAAATCCAGTCAGAGTCTGCTCAACAGTA |
| | | GAACCCGAAAGAACTACTTGGCTTGGTACCAG |
| | | CAGAAACCAGGGCAGTCTCCTAAACTGCTGAT |
| | | CTACTGGGCATCCACTAGGGAATCTGGGGTCC |
| | | CTGATCGCTTCACAGGCAGTGGATCTGGGACA |
| | | GATTTCACTCTCACCATCAGCAATGTGCAGGCT |
| | | GAAGACCTGACAGTTTATTACTGCAAGCAATC |
| | | TTATAATCCTCCGTGGACGTTCGGTGGAGGCAC |
| | | CAAGCTGGAAATCAAA |
| 23 | AGX-A04 | GACATAGTTATGTCCCAGTCTCCATCCAGCTTG |
| | Variable light (VL) | GCTGTCAGCGCCGGAGAGAAAGTGACTATGAG |
| | chain-codon optimized | TTGTAAATCTTCCCAGTCCCTGCTTAACTCACG |
| | nucleic acid | TACTCGGAAGAATTATCTTGCCTGGTATCAACA |
| | | AAAGCCAGGTCAAAGTCCTAAGCTCCTTATTTA |
| | | CTGGGCCTCAACACGGGAGTCAGGTGTCCCCG |
| | | ATCGCTTCACAGGTAGTGGGAGTGGTACTGAC |
| | | TTCACTCTCACCATTTCAAATGTCCAAGCAGAA |
| | | GACTTGACTGTGTATTACTGTAAGCAGAGTTAC |
| | | AACCCTCCTTGGACCTTTGGTGGGGGGACCAA |
| | | ACTGGAGATCAAG |
| 24 | VL-CDR1 | KSSQSLLNSRTRKNYLA |
| 25 | VL-CDR2 | WASTRES |
| 26 | VL-CDR3 | KQSYNPPWT |

Antibody AGX-A05

| | | |
|---|---|---|
| 27 | AGX-A05 | EVQVQQSGPELVKPGASVKMSCKASGYTFTSYV |
| | Variable heavy (VH) | MHWVKQKPGQGLEWIGYINPNNDNINYNEKFK |
| | chain-amino acid | GKASLTSDKSSNTVYMELSSLTSEDSAVYYCAG |
| | | YGNSGANWGQGTLVTVSA |
| 28 | AGX-A05 | GAGGTCCAGGTACAGCAGTCTGGACCTGAACT |
| | Variable heavy (VH) | GGTAAAGCCTGGGGCTTCAGTGAAGATGTCCT |
| | chain-nucleic acid | GTAAGGCTTCTGGATACACATTCACTAGCTATG |
| | | TCATGCACTGGGTGAAGCAGAAGCCTGGGCAG |
| | | GGCCTTGAGTGGATTGGATATATTAATCCTAAC |
| | | AATGATAATATTAACTACAATGAGAAGTTCAA |
| | | AGGCAAGGCCTCACTGACTTCAGACAAATCCT |
| | | CCAACACAGTCTACATGGAGCTCAGCAGCCTG |
| | | ACCTCTGAGGACTCTGCGGTCTATTACTGTGCA |
| | | GGCTATGGTAACTCCGGAGCTAACTGGGGCCA |
| | | AGGGACTCTGGTCACTGTCTCTGCA |
| 29 | AGX-A05 | GAAGTTCAAGTTCAGCAAAGCGGGCCTGAGCT |
| | Variable heavy (VH) | TGTCAAGCCAGGCGCATCAGTCAAAATGAGCT |
| | chain-codon optimized | GTAAGGCTTCCGGGTACACCTTCACCAGTTATG |
| | nucleic acid | TCATGCATTGGGTAAAACAAAAGCCAGGACAG |
| | | GGACTCGAGTGGATAGGATACATTAACCCAAA |
| | | TAACGACAACATTAACTACAACGAGAAATTCA |
| | | AGGGCAAAGCATCATTGACTTCCGATAAATCC |
| | | TCTAACACCGTGTACATGGAGCTGAGTTCATTG |
| | | ACCAGCGAGGATTCTGCCGTGTACTACTGTGC |
| | | AGGTTATGGCAACTCTGGTGCTAACTGGGGGC |
| | | AGGGGACTCTGGTCACAGTCAGCGCA |
| 30 | VH-CDR1 | GYTFTSYVMH |
| 31 | VH-CDR2 | YINPNNDNINYNEKFKG |
| 32 | VH-CDR3 | YGNSGAN |
| 33 | AGX-A05 | DIQMTQSPASLSASVGETVTITCRTSKNIFNFLAW |
| | Variable light (VL) | YHQKQGRSPRLLVSHTKTLAAGVPSRFSGSGSGT |
| | chain-amino acid | QFSLKINSLQPEDFGIYYCQHHYGTPWTFGGGTK |
| | | LEIK |
| 34 | AGX-A05 | GACATCCAGATGACTCAGTCTCCAGCCTCCCTA |
| | Variable light (VL) | TCTGCATCTGTGGGAGAAACTGTCACCATCAC |
| | chain-nucleic acid | ATGTCGAACAAGTAAAAATATTTTCAATTTTTT |
| | | AGCATGGTATCACCAGAAACAGGGAAGATCTC |
| | | CTCGACTCCTGGTCTCTCATACAAAAACCTTAG |
| | | CAGCAGGTGTGCCATCAAGGTTCAGTGGCAGT |
| | | GGCTCAGGCACACAGTTTTCTCTGAAGATCAA |
| | | CAGCCTGCAGCCTGAAGATTTTGGGATTTATTA |
| | | CTGTCAACATCATTATGGTACTCCGTGGACGTT |
| | | CGGTGGAGGCACCAAACTGGAAATCAAA |
| 35 | AGX-A05 | GACATTCAGATGACCCAGTCACCAGCATCTTTG |
| | Variable light (VL) | AGCGCATCCGTTGGGGAGACTGTGACAATCAC |
| | chain-codon optimized | ATGCCGAACCAGTAAGAACATCTTCAACTTCCT |
| | nucleic acid | CGCATGGTACCATCAAAAGCAGGGCAGGTCTC |
| | | CCAGACTGCTTGTCTCTCACACCAAGACACTGG |
| | | CAGCAGGCGTCCCCAGCCGGTTTAGTGGTAGT |
| | | GGATCTGGCACACAGTTTAGTTTGAAAATCAA |
| | | TTCCCTGCAACCCGAAGACTTCGGCATATACTA |
| | | TTGCCAGCACCACTATGGGACACCTTGGACTTT |
| | | CGGAGGTGGTACTAAACTTGAGATTAAA |
| 36 | VL-CDR1 | RTSKNIFNFLA |
| 37 | VL-CDR2 | HTKTLAA |
| 38 | VL-CDR3 | QHHYGTPWT |

Antibody AGX-A07

| | | |
|---|---|---|
| 39 | AGX-A07 | QIQLVQSGPELKKPGETVKISCKASGYTFTNYGV |
| | Variable heavy (VH) | KWVKQAPGKDLKWMGWINTYTGNPIYAADFKG |
| | chain-amino acid | RFAFSLETSASTAFLQINNLKNEDTATYFCVRFQY |
| | | GDYRYFDVWGAGTTVTVSS |
| 40 | AGX-A07 | CAGATCCAGTTGGTGCAGTCTGGACCTGAGCT |
| | Variable heavy (VH) | GAAGAAGCCTGGAGAGACAGTCAAGATCTCCT |
| | chain-nucleic acid | GCAAGGCTTCTGGGTATACCTTCACAAACTATG |
| | | GAGTGAAGTGGGTGAAGCAGGCTCCAGGAAA |
| | | GGATTTAAAGTGGATGGGCTGGATAAACACCT |
| | | ACACTGGAAATCCAATTTATGCTGCTGACTTCA |
| | | AGGGACGGTTTGCCTTCTCTTTGGAGACCTCTG |
| | | CCAGCACTGCCTTTTTGCAGATCAACAACCTCA |
| | | AAAATGAGGACACGGCTACATATTTCTGTGTA |
| | | AGATTCCAATATGGCGATTACCGGTACTTCGAT |
| | | GTCTGGGGCGCAGGGACCACGGTCACCGTCTC |
| | | CTCA |
| 41 | AGX-A07 | CAAATCCAACTTGTCCAGAGCGGTCCCGAGTT |
| | Variable heavy (VH) | GAAGAAGCCTGGCGAAACCGTGAAAATCTCAT |
| | chain-codon optimized | GCAAGGCCAGTGGATATACATTTACAAACTAT |
| | nucleic acid | GGCGTCAAGTGGGTGAAACAAGCCCCAGGTAA |
| | | AGACTTGAAATGGATGGGATGGATCAACACAT |
| | | ACACAGGGAATCCTATCTATGCAGCCGACTTT |
| | | AAAGGCAGATTTGCCTTCAGTTTGGAGACATCT |
| | | GCCTCCACCGCTTTCCTGCAAATAAATAACCTG |
| | | AAAAATGAAGATACCGCTACATACTTCTGTGT |
| | | ACGGTTCCAGTACGGAGATTACCGCTATTTCGA |
| | | TGTGTGGGGCGCAGGTACCACAGTAACCGTCT |
| | | CCTCA |
| 42 | VH-CDR1 | GYTFTNYGVK |
| 43 | VH-CDR2 | WINTYTGNPIYAADFKG |
| 44 | VH-CDR3 | FQYGDYRYFDV |
| 45 | AGX-A07 | QIILSQSPAILSASPGEKVTMTCRANSGISFINWYQ |
| | Variable light (VL) | QKPGSSPKPWIYGTANLASGVPARFGGSGSGTSY |
| | chain-amino acid | SLTISRVEAEDAATYYCQQWSSNPLTFGAGTKLE |
| | | LR |
| 46 | AGX-A07 | CAAATTATTCTCTCCCAGTCTCCAGCAATCCTG |
| | Variable light (VL) | TCTGCATCTCCAGGGGAGAAGGTCACGATGAC |
| | chain-nucleic acid | TTGCAGGGCCAACTCAGGTATTAGTTTCATCAA |
| | | CTGGTACCAGCAGAAGCCAGGATCCTCCCCCA |
| | | AACCCTGGATTTATGGCACAGCCAACCTGGCTT |
| | | CTGGAGTCCCTGCTCGCTTCGGTGGCAGTGGGT |
| | | CTGGGACTTCTTACTCTCTCACAATCAGCAGAG |
| | | TGGAGGCTGAAGACGCTGCCACTTATTACTGC |
| | | CAGCAGTGGAGTAGTAACCCGCTCACGTTCGG |
| | | TGCTGGGACCAAGCTGGAGTTGAGA |
| 47 | AGX-A07 | CAAATAATTCTGTCACAGTCCCCCGCTATACTT |
| | Variable light (VL) | AGTGCTTCACCAGGAGAAAAAGTGACCATGAC |
| | chain-codon optimized | TTGTAGAGCTAATTCTGGCATATCATTCATCAA |
| | nucleic acid | CTGGTATCAACAAAAGCCAGGTTCCTCCCCCA |
| | | AGCCATGGATTTACGGGACCGCCAACCTTGCTT |
| | | CTGGGGTACCCGCTCGTTTCGGCGGATCAGGTT |
| | | CAGGAACTTCCTATAGCCTCACTATCAGTCGGG |
| | | TTGAAGCTGAGGATGCCGCTACATATTACTGCC |
| | | AGCAATGGTCTAGTAATCCACTTACCTTTGGAG |
| | | CTGGCACCAAATTGGAACTTCGT |
| 48 | VL-CDR1 | RANSGISFIN |
| 49 | VL-CDR2 | GTANLAS |
| 50 | VL-CDR3 | QQWSSNPLT |

Antibody AGX-A08

| | | |
|---|---|---|
| 51 | AGX-A08 | EVQLQQSGPELVKPGASVKLSCKASGYTVTSYV |
| | Variable heavy (VH) | MHWVKQKPGQGLEWIGYINPYSDVTNCNEKFK |
| | chain-amino acid | GKATLTSDKTSSTAYMELSSLTSEDSAVYYCSSY |
| | | GGGFAYWGQGTLVTVSA |
| 52 | AGX-A08 | GAGGTCCAGCTGCAGCAGTCTGGACCTGAGCT |
| | Variable heavy (VH) | GGTAAAGCCTGGGGCTTCAGTGAAGCTGTCCT |
| | chain-nucleic acid | GCAAGGCTTCTGGATACACAGTCACTAGCTAT |
| | | GTTATGCACTGGGTGAAGCAGAAGCCTGGGCA |
| | | GGGCCTTGAGTGGATTGGATATATTAATCCTTA |
| | | CAGTGATGTTACTAACTGCAATGAGAAGTTCA |
| | | AAGGCAAGGCCACACTGACTTCAGACAAAACC |
| | | TCCAGCACAGCCTACATGGAGCTCAGCAGCCT |
| | | GACCTCTGAGGACTCTGCGGTCTATTACTGTTC |
| | | CTCCTACGGTGGGGGGTTTGCTTACTGGGGCCA |
| | | AGGGACTCTGGTCACTGTCTCTGCA |
| 53 | AGX-A08 | GAAGTCCAGCTTCAGCAATCCGGCCCAGAACT |
| | Variable heavy (VH) | GGTAAAACCAGGCGCAAGTGTTAAGTTGAGTT |
| | chain-codon optimized | GCAAAGCCAGTGGTTATACCGTTACTTCATACG |
| | nucleic acid | TCATGCATTGGGTAAAACAAAAGCCCGGCCAA |
| | | GGGCTTGAATGGATCGGCTACATCAACCCTTA |
| | | CTCTGACGTCACCAACTGCAACGAGAAATTCA |
| | | AAGGGAAAGCCACATTGACCTCTGACAAGACA |
| | | AGCAGTACCGCCTACATGGAGCTTTCTAGTTTG |
| | | ACTTCTGAAGACTCTGCTGTCTACTACTGTAGC |
| | | AGCTACGGCGGCGGCTTTGCTTACTGGGGCCA |
| | | GGGTACATTGGTGACTGTGAGTGCA |
| 54 | VH-CDR1 | GYTVTSYVMH |
| 55 | VH-CDR2 | YINPYSDVTNCNEKFKG |
| 56 | VH-CDR3 | YGGGFAY |
| 57 | AGX-A08 | DIQMTQSPASLSASVGEPVTITCRASKNIYTYLA |
| | Variable light chain | WYHQKQGKSPQFLVYNARTLAGGVPSRLSGSGS |
| | (VL)-amino acid | VTQFSLNINTLHREDLGTYFCQHHYDTPYTFGGG |
| | | TNLEIK |
| 58 | AGX-A08 | GACATCCAGATGACTCAGTCTCCAGCCTCCCTA |
| | Variable light (VL) | TCTGCATCTGTGGGAGAACCTGTCACCATCACA |
| | chain-nucleic acid | TGTCGAGCAAGTAAGAATATTTACACATATTTA |
| | | GCATGGTATCACCAGAAACAGGGAAAATCTCC |
| | | TCAGTTCCTGGTCTATAATGCAAGAACCTTAGC |
| | | AGGAGGTGTGCCATCAAGGCTCAGTGGCAGTG |
| | | GATCAGTCACGCAGTTTTCTCTAAACATCAACA |
| | | CCTTGCATCGAGAAGATTTAGGGACTTACTTCT |
| | | GTCAACATCATTATGATACTCCGTACACGTTCG |
| | | GAGGGGGGACCAACCTGGAAATAAAA |
| 59 | AGX-A08 | GACATCCAGATGACACAGTCACCAGCATCCCT |
| | Variable light (VL) | GTCCGCCTCAGTTGGGGAGCCTGTTACCATAAC |
| | chain-codon optimized | TTGTCGGGCAAGCAAAAACATATACACCTATT |
| | nucleic acid | TGGCTTGGTATCACCAAAAGCAAGGTAAGTCA |
| | | CCTCAGTTTCTTGTATATAATGCCCGCACACTT |
| | | GCTGGCGGAGTACCCTCTCGATTGTCTGGATCT |
| | | GGCAGCGTTACCCAATTCAGCCTGAACATCAA |
| | | CACCCTCCATCGGGAAGATTTGGGTACCTATTT |
| | | CTGTCAACATCACTACGACACCCCATACACCTT |
| | | CGGAGGCGGCACAAATTTGGAAATTAAA |
| 60 | VL-CDR1 | RASKNIYTYLA |
| 61 | VL-CDR2 | NARTLAG |
| 62 | VL-CDR3 | QHHYDTPYT |

Antibody AGX-A09

| | | |
|---|---|---|
| 63 | AGX-A09 | EVQLQQSGPELVKPGASVKMSCKASGYTFSSYV |
| | Variable heavy (VH) | MHWVKQKPGQGLEWIGYINPYSDVTNYNEKFK |
| | chain-amino acid | GKATLTSDRSSNTAYMELSSLTSEDSAVYYCARN |
| | | YFDWGRGTLVTVSA |
| 64 | AGX-A09 | GAGGTCCAGCTGCAGCAGTCTGGACCTGAGCT |
| | Variable heavy (VH) | GGTAAAGCCTGGGGCTTCAGTGAAGATGTCCT |
| | chain-nucleic acid | GCAAGGCTTCTGGATACACATTCTCTAGCTATG |
| | | TTATGCACTGGGTGAAGCAGAAGCCTGGGCAG |
| | | GGCCTTGAGTGGATTGGATATATTAATCCTTAC |
| | | AGTGATGTCACTAACTACAATGAGAAGTTCAA |
| | | AGGCAAGGCCACACTGACTTCAGACAGATCCT |
| | | CCAACACAGCCTACATGGAACTCAGCAGCCTG |
| | | ACCTCTGAGGACTCTGCGGTCTATTACTGTGCA |
| | | AGAAATTACTTCGACTGGGGCCGAGGGACTCT |
| | | GGTCACAGTCTCTGCA |
| 65 | AGX-A09 | GAGGTACAGCTTCAGCAGAGTGGTCCAGAACT |
| | Variable heavy (VH) | CGTCAAGCCTGGGGCAAGCGTTAAGATGAGTT |
| | chain-codon optimized | GTAAAGCATCCGGTTACACATTCAGTAGCTAT |
| | nucleic acid | GTTATGCACTGGGTCAAACAGAAGCCTGGGCA |
| | | GGGGTTGGAGTGGATCGGATATATAAATCCCT |
| | | ATTCAGACGTAACTAATTATAATGAAAAGTTC |
| | | AAGGGGAAAGCAACCTTGACAAGTGACCGGTC |
| | | ATCTAATACCGCATACATGGAGCTGAGCTCATT |
| | | GACAAGTGAGGACTCTGCTGTGTATTACTGTGC |
| | | CCGGAACTACTTCGACTGGGGTAGGGGCACAC |
| | | TGGTAACTGTTAGTGCA |
| 66 | VH-CDR1 | GYTFSSYVMH |
| 67 | VH-CDR2 | YINPYSDVTNYNEKFKG |
| 68 | VH-CDR3 | NYFD |
| 69 | AGX-A09 | DIQMTQSPASLSASVGETVTITCRASKNVYSYLA |
| | Variable light (VL) | WFQQKQGKSPQLLVYNAKTLAEGVPSRFSGGGS |
| | chain-amino acid | GTQFSLKINSLQPADFGSYYCQHHYNIPFTFGSGT |
| | | KLEIK |
| 70 | AGX-A09 | GACATCCAGATGACTCAGTCTCCAGCCTCCCTA |
| | Variable light (VL) | TCTGCATCTGTGGGAGAAACTGTCACCATCAC |
| | chain-nucleic acid | ATGTCGAGCAAGTAAAAATGTTTACAGTTATTT |
| | | AGCATGGTTTCAACAGAAACAGGGGAAATCTC |
| | | CTCAGCTCCTGGTCTATAATGCTAAAACCTTAG |
| | | CAGAAGGTGTGCCATCAAGGTTCAGTGGCGGG |
| | | GGATCAGGCACACAGTTTTCTCTGAAGATCAA |
| | | CAGCCTGCAGCCTGCAGATTTTGGGAGTTATTA |
| | | CTGTCAACATCATTATAATATTCCATTCACGTT |
| | | CGGCTCGGGGACAAAGTTGGAAATAAAA |
| 71 | AGX-A09 | GACATACAAATGACACAAAGTCCCGCTAGTCT |
| | Variable light (VL) | TTCAGCCAGTGTTGGTGAGACTGTGACAATAA |
| | chain-codon optimized | CCTGTAGAGCTAGCAAAAATGTCTACTCCTATC |
| | nucleic acid | TGGCTTGGTTCCAGCAGAAACAAGGAAAGAGT |
| | | CCTCAGTTGCTCGTATATAATGCTAAAACTTTG |
| | | GCAGAAGGCGTCCCTTCTCGTTTCAGTGGCGG |
| | | AGGAAGTGGGACTCAATTCTCACTGAAGATCA |
| | | ATAGCCTCCAGCCCGCCGACTTTGGGAGCTACT |
| | | ATTGCCAACATCATTACAACATACCATTCACCT |
| | | TTGGCTCAGGTACTAAACTCGAAATTAAA |
| 72 | VL-CDR1 | RASKNVYSYLA |
| 73 | VL-CDR2 | NAKTLAE |
| 74 | VL-CDR3 | QHHYNIPFT |

Antibody AGX-A11

| | | |
|---|---|---|
| 75 | AGX-A11 | QIQLVQSGPELKKPGETVKISCKASGFTFTNYPM |
| | Variable heavy (VH) | HWVKQAPGKGLKWMGWINTYSGVPTYADDFK |
| | chain-amino acid | GRFAFSLETSASTAYLQINNLKNEDMATYFCARG |
| | | GYDGSREFAYWGQGTLVTVS |
| 76 | AGX-A11 | CAGATCCAGTTGGTGCAGTCTGGACCTGAGCT |
| | Variable heavy (VH) | GAAGAAGCCTGGAGAGACAGTCAAGATCTCCT |
| | chain-nucleic acid | GCAAGGCTTCTGGGTTTACCTTCACAAACTATC |
| | | CAATGCACTGGGTGAAGCAGGCTCCAGGAAAG |
| | | GGTTTAAAGTGGATGGGCTGGATAAACACCTA |
| | | CTCTGGAGTGCCAACATATGCAGATGACTTCA |
| | | AGGGACGGTTTGCCTTCTCTTTGGAAACCTCTG |
| | | CCAGCACTGCATATTTGCAGATCAACAACCTC |
| | | AAAAATGAGGACATGGCTACATATTTCTGTGC |
| | | AAGAGGGGGCTACGATGGTAGCAGGGAGTTTG |
| | | CTTACTGGGGCCAAGGGACTCTGGTCACTGTCT |
| | | CT |
| 77 | AGX-A11 | CAGATACAACTCGTCCAGTCAGGTCCAGAGTT |
| | Variable heavy (VH) | GAAGAAACCCGGAGAAACTGTGAAGATATCCT |
| | chain-codon optimized | GTAAAGCCAGCGGCTTTACTTTCACAAACTACC |
| | nucleic acid | CCATGCATTGGGTGAAGCAGGCCCCCGGAAAA |
| | | GGACTCAAATGGATGGGATGGATCAACACATA |
| | | CAGTGGGGTGCCTACTTACGCAGACGATTTCA |
| | | AAGGAAGGTTCGCATTTAGCTTGGAAACTAGC |
| | | GCATCTACAGCATATCTCCAGATTAACAATCTT |
| | | AAAAATGAGGATATGGCAACATACTTCTGCGC |
| | | TAGGGGAGGTTACGATGGGAGCAGGGAGTTCG |
| | | CTTATTGGGGGCAAGGGACTCTTGTGACTGTA |
| | | AGT |
| 78 | VH-CDR1 | GFTFTNYPMH |
| 79 | VH-CDR2 | WINTYSGVPTYADDFKG |
| 80 | VH-CDR3 | GGYDGSREFAY |
| 81 | AGX-A11 | DIVLTQSPASLAASLGQRATTSYRASKSVSTSGYS |
| | Variable light (VL) | YMHWNQQKPGQPPRLLIYLVSNLESGVPARFSGS |
| | chain-amino acid | GSGTDFTLNIHPVEEEDAATYYCQHIRELTTFGG |
| | | GTKLEIK |
| 82 | AGX-A11 | GACATTGTGCTGACACAGTCTCCTGCTTCCTTA |
| | Variable light (VL) | GCTGCATCTCTGGGGCAGAGGGCCACCACCTC |
| | chain-nucleic acid | ATACAGGGCCAGCAAAAGTGTCAGTACATCTG |
| | | GCTATAGTTATATGCACTGGAACCAACAGAAA |
| | | CCAGGACAGCCACCCAGACTCCTCATCTATCTT |
| | | GTATCCAACCTAGAATCTGGGGTCCCTGCCAG |
| | | GTTCAGTGGCAGTGGGTCTGGGACAGACTTCA |
| | | CCCTCAACATCCATCCTGTGGAGGAGGAGGAT |
| | | GCTGCAACCTATTACTGTCAGCACATTAGGGA |
| | | GCTTACCACGTTCGGAGGGGGGACCAAGCTGG |
| | | AAATAAAA |
| 83 | AGX-A11 | GACATAGTGCTCACTCAGAGCCCTGCATCCCTT |
| | Variable light (VL) | GCCGCCTCCCTCGGACAACGAGCTACTACAAG |
| | chain-codon optimized | CTACCGGGCATCAAAGTCCGTTAGCACATCAG |
| | nucleic acid | GATACAGCTATATGCACTGGAATCAGCAAAAG |
| | | CCAGGCCAACCACCCCGTCTTCTCATCTACCTC |
| | | GTAAGTAATCTGGAATCAGGCGTGCCAGCCCG |
| | | ATTCAGTGGGTCAGGGTCTGGGACAGATTTCA |
| | | CCCTCAACATCCATCCAGTAGAGGAAGAGGAC |
| | | GCAGCAACATATTACTGCCAACACATTAGAGA |
| | | ACTTACCACTTTCGGAGGAGGAACTAAATTGG |
| | | AGATCAAA |
| 84 | VL-CDR1 | RASKSVSTSGYSYMH |
| 85 | VL-CDR2 | LVSNLES |
| 86 | VL-CDR3 | QHIRELT |

Constant Region Sequences

| | | |
|---|---|---|
| 87 | IgG1 G1m17* (heavy chain | ASTKGPSVFPLAPSSKSTSGGTAALGCLVKDYFP |
| | constant region) | EPVTVSWNSGALTSGVHTFPAVLQSSGLYSLSSV |
| | *with L234A/L235A/G237A | VTVPSSSLGTQTYICNVNHKPSNTKVDKKVEPKS |
| | mutations | CDKTHTCPPCPAPEAAGAPSVFLFPPKPKDTLMIS |
| | SEQ ID NO: 88 is sequence | RTPEVTCVVVDVSHEDPEVKFNWYVDGVEVHN |
| | without the terminal | AKTKPREEQYNSTYRVVSVLTVLHQDWLNGKE |
| | lysine | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 88 | IgG1 G1m17* (heavy chain | ASTKGPSVFPLAPSSKSTSGGTAALGCLVKDYFP |
| | constant region) | EPVTVSWNSGALTSGVHTFPAVLQSSGLYSLSSV |
| | *with L234A/L235A/G237A | VTVPSSSLGTQTYICNVNHKPSNTKVDKKVEPKS |
| | mutations | CDKTHTCPPCPAPEAAGAPSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPG |
| 89 | IgG1 Km3 (light chain | RTVAAPSVFIFPPSDEQLKSGTASVVCLLNNFYPR |
| | constant region) | EAKVQWKVDNALQSGNSQESVTEQDSKDSTYSL |
| | | SSTLTLSKADYEKHKVYACEVTHQGLSSPVTKSF |
| | | NRGEC |

Humanized AGX-A07 sequences

| | | |
|---|---|---|
| 90 | AGX-A07 (humanized) H2 | QVQLVQSGAEVKKPGASVKVSCKASGYTFTNYG |
| | Heavy chain amino acid | VKWVRQAPGQDLEWMGWINTYTGNPIYAADFK |
| | | GRVTMTTDTSTSTAFMELRSLRSDDTAVYYCVR |
| | | FQYGDYRYFDVWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGAPSVFLFPPKPKDTLMISRTPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVYTLPPSREEMTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| 91 | AGX-A07 (humanized) H2 | TCTACCGGACAGGTGCAGTTGGTTCAGTCTGGC |
| | Heavy chain nucleic | GCCGAAGTGAAGAAACCTGGCGCTTCTGTGAA |
| | acid | GGTGTCCTGCAAGGCCTCTGGCTACACCTTTAC |
| | | CAACTACGGCGTGAAATGGGTCCGACAGGCTC |
| | | CTGGACAGGATCTGGAATGGATGGGCTGGATC |
| | | AACACCTACACCGGCAATCCTATCTACGCCGC |
| | | CGACTTCAAGGGCAGAGTGACCATGACCACCG |
| | | ACACCTCTACCTCCACCGCCTTCATGGAACTGC |
| | | GGTCCCTGAGATCTGACGACACCGCCGTGTAC |
| | | TACTGCGTGCGGTTTCAGTACGGCGACTACCG |
| | | GTACTTTGATGTGTGGGGCCAGGGCACACTGG |
| | | TCACCGTTTCTTCCGCTTCTACCAAGGGACCCA |
| | | GCGTGTTCCCTCTGGCTCCTTCCTCTAAATCCA |
| | | CCTCTGGCGGAACCGCTGCTCTGGGCTGTCTGG |
| | | TCAAGGATTACTTCCCTGAGCCTGTGACCGTGT |
| | | CCTGGAACTCTGGTGCTCTGACATCCGGCGTGC |
| | | ACACCTTTCCAGCTGTGCTGCAGTCCTCTGGCC |
| | | TGTACTCTCTGTCCTCTGTCGTGACCGTGCCTT |
| | | CTAGCTCTCTGGGCACCCAGACCTACATCTGCA |
| | | ACGTGAACCACAAGCCTTCCAACACCAAGGTG |
| | | GACAAGAAGGTGGAACCCAAGTCCTGCGACAA |
| | | GACCCACACCTGTCCTCCATGTCCTGCTCCAGA |
| | | AGCTGCTGGCGCTCCCTCTGTGTTCCTGTTTCC |
| | | TCCAAAGCCTAAGGACACCCTGATGATCTCTC |
| | | GGACCCCTGAAGTGACCTGCGTGGTGGTGGAT |
| | | GTGTCTCACGAGGACCCAGAAGTGAAGTTCAA |
| | | TTGGTACGTGGACGGCGTGGAAGTGCACAACG |
| | | CCAAGACCAAGCCTAGAGAGGAACAGTACAAC |
| | | TCCACCTACAGAGTGGTGTCCGTGCTGACCGTG |
| | | CTGCACCAGGATTGGCTGAACGGCAAAGAGTA |
| | | CAAGTGCAAGGTGTCCAACAAGGCACTGCCCG |
| | | CTCCTATCGAAAAGACCATCTCCAAGGCTAAG |
| | | GGCCAGCCTCGGGAACCTCAGGTTTACACCCT |
| | | GCCTCCATCTCGGGAAGAGATGACCAAGAACC |
| | | AGGTGTCCCTGACCTGCCTCGTGAAGGGCTTCT |
| | | ACCCTTCCGATATCGCCGTGGAATGGGAGTCC |
| | | AATGGCCAGCCTGAGAACAACTACAAGACAAC |
| | | CCCTCCTGTGCTGGACTCCGACGGCTCATTCTT |
| | | CCTGTACTCCAAGCTGACAGTGGACAAGTCTC |
| | | GGTGGCAGCAGGGCAACGTGTTCTCCTGTTCTG |
| | | TGATGCACGAGGCCCTGCACAACCACTACACA |
| | | CAGAAGTCCCTGTCTCTGTCCCCTGGCAAGTGA |
| 92 | AGX-A07 H2v1 | EVQLVQSGAEVKKPGASVKVSCKASGYTFTNYG |
| | Heavy chain amino | VKWVRQAPGQGLEWMGWINTYTGNPIYAADFK |
| | acid | GRVTMTTDTSTSTAYMELRSLRSDDTAVYYCVR |
| | | FQYGDYRYFDVWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGAPSVFLFPPKPKDTLMISRTPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVYTLPPSREEMTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| 93 | AGX-A07 H2v1 | GAAGTGCAGTTGGTGCAGTCTGGCGCCGAAGT |
| | Heavy chain nucleic | GAAGAAACCTGGCGCTTCTGTGAAGGTGTCCT |
| | acid | GCAAGGCCTCTGGCTACACCTTTACCAACTACG |
| | | GCGTGAAATGGGTCCGACAGGCTCCTGGACAA |
| | | GGCCTGGAATGGATGGGCTGGATCAACACCTA |
| | | CACCGGCAATCCTATCTACGCCGCCGACTTCAA |
| | | GGGCAGAGTGACCATGACCACCGACACCTCTA |
| | | CCTCCACCGCCTACATGGAACTGCGGTCCCTGA |
| | | GATCTGACGACACCGCCGTGTACTACTGCGTG |
| | | CGGTTTCAGTACGGCGACTACCGGTACTTTGAT |
| | | GTGTGGGGCCAGGGCACACTGGTCACCGTTTC |
| | | TTCCGCTTCTACCAAGGGACCCAGCGTGTTCCC |
| | | TCTGGCTCCTTCCTCTAAATCCACCTCTGGCGG |
| | | AACCGCTGCTCTGGGCTGTCTGGTCAAGGATTA |
| | | CTTCCCTGAGCCTGTGACCGTGTCCTGGAATTC |
| | | TGGTGCTCTGACATCCGGCGTGCACACCTTTCC |
| | | AGCTGTGCTGCAGTCCTCTGGCCTGTACTCTCT |
| | | GTCCTCTGTCGTGACCGTGCCTTCTAGCTCTCT |
| | | GGGCACCCAGACCTACATCTGCAACGTGAACC |
| | | ACAAGCCTTCCAACACCAAGGTGGACAAGAAG |
| | | GTGGAACCCAAGTCCTGCGACAAGACCCACAC |
| | | CTGTCCTCCATGTCCTGCTCCAGAAGCTGCTGG |
| | | CGCTCCCTCTGTGTTCCTGTTTCCTCCAAAGCC |
| | | TAAGGACACCCTGATGATCTCTCGGACCCCTG |
| | | AAGTGACCTGCGTGGTGGTGGATGTGTCTCAC |
| | | GAGGACCCAGAAGTGAAGTTCAATTGGTACGT |
| | | GGACGGCGTGGAAGTGCACAACGCCAAGACCA |
| | | AGCCTAGAGAGGAACAGTACAACTCCACCTAC |
| | | AGAGTGGTGTCCGTGCTGACCGTGCTGCACCA |
| | | GGATTGGCTGAACGGCAAAGAGTACAAGTGCA |
| | | AGGTGTCCAACAAGGCACTGCCCGCTCCTATC |
| | | GAAAAGACCATCTCCAAGGCTAAGGGCCAGCC |
| | | TCGGGAACCTCAGGTTTACACCCTGCCTCCATC |
| | | TCGGGAAGAGATGACCAAGAACCAGGTGTCCC |
| | | TGACCTGCCTCGTGAAGGGCTTCTACCCTTCCG |
| | | ATATCGCCGTGGAATGGGAGTCCAATGGCCAG |
| | | CCTGAGAACAACTACAAGACAACCCCTCCTGT |
| | | GCTGGACTCCGACGGCTCATTCTTCCTGTACTC |
| | | CAAGCTGACAGTGGACAAGTCTCGGTGGCAGC |
| | | AGGGCAACGTGTTCTCCTGTTCTGTGATGCACG |
| | | AGGCCCTGCACAACCACTACACACAGAAGTCC |
| | | CTGTCTCTGTCCCCTGGCAAGTGA |
| 94 | VH-CDR1 | GYTFTNYGVK |
| 95 | VH-CDR2 | WINTYTGNPIYAADFK |
| 96 | VH-CDR3 | FQYGDYRYFDV |
| 97 | AGX-A07 L5 | EIILTQSPATLSLSPGERATLSCRANSGISFINWYQ |
| | Light chain amino | QKPGQAPRLLIYGTANLASGIPARFGGSGSGRDF |
| | acid | TLTISSLEPEDFAVYYCQQWSSNPLTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 98 | AGX-A07 L5 | AAGCTTGCCACCATGGAAACCGACACACTGCT |
| | Light chain nucleic | GCTGTGGGTGCTGTTGTTGTGGGTGCCAGGATC |
| | acid | TACCGGAGAGATCATCCTGACACAGAGCCCCG |
| | | CCACATTGTCTCTGAGTCCTGGCGAGAGAGCT |
| | | ACCCTGTCCTGTAGAGCCAACTCCGGCATCTCC |
| | | TTCATCAACTGGTATCAGCAGAAGCCCGGCCA |
| | | GGCTCCTAGACTGCTGATCTATGGCACCGCTAA |
| | | CCTGGCCTCTGGCATCCCTGCTAGATTTGGCGG |
| | | CTCTGGCTCTGGCAGAGACTTCACCCTGACCAT |
| | | CTCTAGCCTGGAACCTGAGGACTTCGCCGTGTA |
| | | CTACTGCCAGCAGTGGTCTAGCAACCCTCTGAC |
| | | CTTTGGCGGAGGCACCAAGGTGGAAATCAAGA |
| | | GAACCGTGGCCGCTCCTTCCGTGTTCATCTTCC |
| | | CACCATCTGACGAGCAGCTGAAGTCTGGCACA |
| | | GCCTCTGTCGTGTGCCTGCTGAACAACTTCTAC |
| | | CCTCGGGAAGCCAAGGTGCAGTGGAAGGTGGA |
| | | CAATGCCCTGCAGTCCGGCAACTCCCAAGAGT |
| | | CTGTGACCGAGCAGGACTCCAAGGACTCTACC |
| | | TACAGCCTGTCCTCCACACTGACCCTGTCTAAG |
| | | GCCGACTACGAGAAGCACAAGGTGTACGCCTG |
| | | TGAAGTGACCCACCAGGGACTGTCTAGCCCCG |
| | | TGACCAAGTCTTTCAACCGGGGCGAGTGCTGA |
| 99 | AGX-A07 L5v1 | EIVLTQSPATLSLSPGERATLSCRANSGISFINWYQ |
| | Light chain amino | QKPGQAPRLLIYGTANLASGIPARFSGSGSGRDFT |
| | acid | LTISSLEPEDFAVYYCQQWSSNPLTFGGGTKVEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| 100 | AGX-A07 L5v1 | TCTACAGGCGAGATCGTGCTGACCCAGTCTCCT |
| | Light chain nucleic | GCCACATTGTCTCTGAGTCCTGGCGAGAGAGC |
| | acid | TACCCTGTCCTGTAGAGCCAACTCCGGCATCTC |
| | | CTTCATCAACTGGTATCAGCAGAAGCCCGGCC |
| | | AGGCTCCTAGACTGCTGATCTATGGCACCGCTA |
| | | ACCTGGCCTCTGGCATCCCTGCTAGATTTTCCG |
| | | GCTCTGGCTCTGGCAGAGACTTCACCCTGACCA |
| | | TCTCTAGCCTGGAACCTGAGGACTTCGCCGTGT |
| | | ACTACTGCCAGCAGTGGTCTAGCAACCCTCTG |
| | | ACCTTTGGCGGAGGCACCAAGGTGGAAATCAA |
| | | GAGAACCGTGGCCGCTCCTTCCGTGTTCATCTT |
| | | CCCACCATCTGACGAGCAGCTGAAGTCTGGCA |
| | | CAGCCTCTGTCGTGTGCCTGCTGAACAACTTCT |
| | | ACCCTCGGGAAGCCAAGGTGCAGTGGAAGGTG |
| | | GACAATGCCCTGCAGTCCGGCAACTCCCAAGA |
| | | GTCTGTGACCGAGCAGGACTCCAAGGACTCTA |
| | | CCTACAGCCTGTCCTCCACACTGACCCTGTCTA |
| | | AGGCCGACTACGAGAAGCACAAGGTGTACGCC |
| | | TGTGAAGTGACCCACCAGGGACTGTCTAGCCC |
| | | CGTGACCAAGTCTTTCAACCGGGGCGAGTGCT |
| | | GA |
| 101 | AGX-A07 L5v2 | EIVLTQSPATLSLSPGERATLSCRAQSGISFINWYQ |
| | Light chain amino | QKPGQAPRLLIYGTANLASGIPARFSGSGSGRDFT |
| | acid | LTISSLEPEDFAVYYCQQWSSNPLTFGGGTKVEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| 102 | AGX-A07 L5v2 | TCTACAGGCGAGATCGTGCTGACCCAGTCTCCT |
| | Light chain nucleic | GCCACATTGTCTCTGAGTCCTGGCGAGAGAGC |
| | acid | TACCCTGTCTTGTAGAGCCCAGTCCGGCATCTC |
| | | CTTCATCAACTGGTATCAGCAGAAGCCCGGCC |
| | | AGGCTCCTAGACTGCTGATCTATGGCACCGCTA |
| | | ACCTGGCCTCTGGCATCCCTGCTAGATTTTCCG |
| | | GCTCTGGCTCTGGCAGAGACTTCACCCTGACCA |
| | | TCTCTAGCCTGGAACCTGAGGACTTCGCCGTGT |
| | | ACTACTGCCAGCAGTGGTCTAGCAACCCTCTG |
| | | ACCTTTGGCGGAGGCACCAAGGTGGAAATCAA |
| | | GAGAACCGTGGCCGCTCCTTCCGTGTTCATCTT |
| | | CCCACCATCTGACGAGCAGCTGAAGTCTGGCA |
| | | CAGCCTCTGTCGTGTGCCTGCTGAACAACTTCT |
| | | ACCCTCGGGAAGCCAAGGTGCAGTGGAAGGTG |
| | | GACAATGCCCTGCAGTCTGGCAACTCCCAAGA |
| | | GTCTGTGACCGAGCAGGACTCCAAGGACTCTA |
| | | CCTACAGCCTGTCCTCCACACTGACCCTGTCTA |
| | | AGGCCGACTACGAGAAGCACAAGGTGTACGCC |
| | | TGTGAAGTGACCCACCAGGGACTGTCTAGCCC |
| | | CGTGACCAAGTCTTTCAACCGGGGCGAGTGCT |
| | | GA |
| 103 | AGX-A07 L5v3 | EIVLTQSPATLSLSPGERATLSCRANSGISFINWYQ |
| | Light chain amino | QKPGQAPRLLIYGTANLASGIPARFSGSGSGRDFT |
| | acid | LTISSLEPEDFAVYYCQQYSSNPLTFGGGTKVEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| 104 | AGX-A07 L5v3 | TCTACAGGCGAGATCGTGCTGACCCAGTCTCCT |
| | Light chain nucleic | GCCACATTGTCTCTGAGTCCTGGCGAGAGAGC |
| | acid | TACCCTGTCCTGTAGAGCCAACTCCGGCATCTC |
| | | CTTCATCAACTGGTATCAGCAGAAGCCCGGCC |
| | | AGGCTCCTAGACTGCTGATCTATGGCACCGCTA |
| | | ACCTGGCCTCTGGCATCCCTGCTAGATTTTCCG |
| | | GCTCTGGCTCTGGCAGAGACTTCACCCTGACCA |
| | | TCTCTAGCCTGGAACCTGAGGACTTCGCCGTGT |
| | | ACTACTGCCAGCAGTACAGCAGCAACCCTCTG |
| | | ACCTTTGGCGGAGGCACCAAGGTGGAAATCAA |
| | | GAGAACCGTGGCCGCTCCTTCCGTGTTCATCTT |
| | | CCCACCATCTGACGAGCAGCTGAAGTCTGGCA |
| | | CAGCCTCTGTCGTGTGCCTGCTGAACAACTTCT |
| | | ACCCTCGGGAAGCCAAGGTGCAGTGGAAGGTG |
| | | GACAATGCCCTGCAGTCCGGCAACTCCCAAGA |
| | | GTCTGTGACCGAGCAGGACTCCAAGGACTCTA |
| | | CCTACAGCCTGTCCTCCACACTGACCCTGTCTA |
| | | AGGCCGACTACGAGAAGCACAAGGTGTACGCC |
| | | TGTGAAGTGACCCACCAGGGACTGTCTAGCCC |
| | | CGTGACCAAGTCTTTCAACCGGGGCGAGTGCT |
| | | GA |
| 105 | AGX-A07 L5v4 | EIVLTQSPATLSLSPGERATLSCRAQSGISFINWYQ |
| | Light chain amino | QKPGQAPRLLIYGTANLASGIPARFSGSGSGRDFT |
| | acid | LTISSLEPEDFAVYYCQQYSSNPLTFGGGTKVEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| 106 | AGX-A07 L5v4 | TCTACAGGCGAGATCGTGCTGACCCAGTCTCCT |
| | Light chain nucleic | GCCACATTGTCTCTGAGTCCTGGCGAGAGAGC |
| | acid | TACCCTGTCTTGTAGAGCCCAGTCCGGCATCTC |
| | | CTTCATCAACTGGTATCAGCAGAAGCCCGGCC |
| | | AGGCTCCTAGACTGCTGATCTATGGCACCGCTA |
| | | ACCTGGCCTCTGGCATCCCTGCTAGATTTTCCG |
| | | GCTCTGGCTCTGGCAGAGACTTCACCCTGACCA |
| | | TCTCTAGCCTGGAACCTGAGGACTTCGCCGTGT |
| | | ACTACTGCCAGCAGTACAGCAGCAACCCTCTG |
| | | ACCTTTGGCGGAGGCACCAAGGTGGAAATCAA |
| | | GAGAACCGTGGCCGCTCCTTCCGTGTTCATCTT |
| | | CCCACCATCTGACGAGCAGCTGAAGTCTGGCA |
| | | CAGCCTCTGTCGTGTGCCTGCTGAACAACTTCT |
| | | ACCCTCGGGAAGCCAAGGTGCAGTGGAAGGTG |
| | | GACAATGCCCTGCAGTCTGGCAACTCCCAAGA |
| | | GTCTGTGACCGAGCAGGACTCCAAGGACTCTA |
| | | CCTACAGCCTGTCCTCCACACTGACCCTGTCTA |
| | | AGGCCGACTACGAGAAGCACAAGGTGTACGCC |
| | | TGTGAAGTGACCCACCAGGGACTGTCTAGCCC |
| | | CGTGACCAAGTCTTTCAACCGGGGCGAGTGCT |
| | | GA |
| 107 | VL-CDR1 (variant 1) | RANSGISFIN |
| 108 | VL-CDR1 (variant 2) | RAQSGISFIN |
| 109 | VL-CDR2 | GTANLAS |
| 110 | VL-CDR3 (variant 1) | QQWSSNPLT |
| 111 | VL-CDR3 (variant 2) | QQYSSNPLT |

Humanized AGX-A01 sequences

| | | |
|---|---|---|
| 112 | AGX-A01 H1 | EVQLVESGGGLVKPGGSLRLSCAASGFTFSSFAM |
| | Heavy chain amino | SWVRQAPGKGLEWVSTISSGSIYIYYTDGVKGRF |
| | acid | TISRDNAKNSLYLQMNSLRAEDTAVYYCARRGI |
| | | YYGYDGYAMDYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGAPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | PAPIEKTISKAKGQPREPQVYTLPPSREEMTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| 113 | AGX-A01 H1 | GAGGTGCAGCTGGTTGAATCTGGCGGAGGACT |
| | Heavy chain nucleic | TGTGAAGCCTGGCGGCTCTCTGAGACTGTCTTG |
| | acid | TGCCGCCTCTGGCTTCACCTTCTCCAGCTTTGC |
| | | CATGTCCTGGGTCCGACAGGCTCCTGGCAAAG |
| | | GACTGGAATGGGTGTCCACCATCTCCTCCGGCT |
| | | CCATCTACATCTACTACACCGACGGCGTGAAG |
| | | GGCAGATTCACCATCAGCAGAGACAACGCCAA |
| | | GAACTCCCTGTACCTGCAGATGAACAGCCTGA |
| | | GAGCCGAGGACACCGCCGTGTACTATTGTGCC |
| | | AGACGGGGCATCTACTATGGCTACGACGGCTA |
| | | CGCTATGGACTATTGGGGACAGGGCACACTGG |
| | | TCACCGTGTCCTCTGCTTCTACCAAGGGACCCA |
| | | GCGTGTTCCCTCTGGCTCCTTCCTCTAAATCCA |
| | | CCTCTGGCGGAACCGCTGCTCTGGGCTGTCTGG |
| | | TCAAGGATTACTTCCCTGAGCCTGTGACCGTGT |
| | | CCTGGAACTCTGGTGCTCTGACATCCGGCGTGC |
| | | ACACCTTTCCAGCTGTGCTGCAGTCCTCTGGCC |
| | | TGTACTCTCTGTCCTCTGTCGTGACCGTGCCTT |
| | | CTAGCTCTCTGGGCACCCAGACCTACATCTGCA |
| | | ACGTGAACCACAAGCCTTCCAACACCAAGGTG |
| | | GACAAGAAGGTGGAACCCAAGTCCTGCGACAA |
| | | GACCCACACCTGTCCTCCATGTCCTGCTCCAGA |
| | | AGCTGCTGGCGCTCCCTCTGTGTTCCTGTTTCC |
| | | TCCAAAGCCTAAGGACACCCTGATGATCTCTC |
| | | GGACCCCTGAAGTGACCTGCGTGGTGGTGGAT |
| | | GTGTCTCACGAGGACCCAGAAGTGAAGTTCAA |
| | | TTGGTACGTGGACGGCGTGGAAGTGCACAACG |
| | | CCAAGACCAAGCCTAGAGAGGAACAGTACAAC |
| | | TCCACCTACAGAGTGGTGTCCGTGCTGACCGTG |
| | | CTGCACCAGGATTGGCTGAACGGCAAAGAGTA |
| | | CAAGTGCAAGGTGTCCAACAAGGCACTGCCCG |
| | | CTCCTATCGAAAAGACCATCTCCAAGGCTAAG |
| | | GGCCAGCCTCGGGAACCTCAGGTTTACACCCT |
| | | GCCTCCATCTCGGGAAGAGATGACCAAGAACC |
| | | AGGTGTCCCTGACCTGCCTCGTGAAGGGCTTCT |
| | | ACCCTTCCGATATCGCCGTGGAATGGGAGTCC |
| | | AATGGCCAGCCTGAGAACAACTACAAGACAAC |
| | | CCCTCCTGTGCTGGACTCCGACGGCTCATTCTT |
| | | CCTGTACTCCAAGCTGACAGTGGACAAGTCTC |
| | | GGTGGCAGCAGGGCAACGTGTTCTCCTGTTCTG |
| | | TGATGCACGAGGCCCTGCACAACCACTACACA |
| | | CAGAAGTCCCTGTCTCTGTCCCCTGGCAAGTGA |
| 114 | AGX-A01 H1v1 | EVQLVESGGGLVKPGGSLRLSCAASGFTFSSFAM |
| | Heavy chain amino | SWVRQAPGKGLEWVSTISSGSIYIYYTDSVKGRF |
| | acid | TISRDNAKNSLYLQMNSLRAEDTAVYYCARRGI |
| | | YYGYEGYAMDYWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGAPSVFLFPPKPKDTLMISRTPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVYTLPPSREEMTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| 115 | VH-CDR1 | GFTFSSFAMS |
| 116 | VH-CDR2 (variant 1) | TISSGSIYIYYTDGVKG |
| 117 | VH-CDR2 (variant 2) | TISSGSIYIYYTDSVKG |
| 118 | VH-CDR3 (variant 1) | RGIYYGYDGYAMDY |
| 119 | VH-CDR3 (variant 2) | RGIYYGYEGYAMDY |
| 120 | VH-CDR3 (variant 3) | RGIYYGYSGYAMDY |
| 121 | VH-CDR3 (variant 4) | RGIYYGYAGYAMDY |
| 122 | AGX-A01 L10 | AIVLTQSPGTLSLSPGERATLSCRSSQSLVHSNGN |
| | Light chain amino | TYLHWYMQKPGQAPRVLIYKVSNRFSGIPDRFSG |
| | acid | SGSGTDFTLTISRLEPDDFAIYYCSQSTHIPLAFGQ |
| | | GTKLEIKRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| 123 | AGX-A01 L10 | GCCATCGTGTTGACCCAGTCTCCAGGCACATTG |
| | Light chain nucleic | TCTCTGAGCCCTGGCGAGAGAGCTACCCTGTCC |
| | acid | TGCAGATCTTCTCAGTCCCTGGTGCACTCCAAC |
| | | GGCAACACCTACCTGCACTGGTACATGCAGAA |
| | | GCCCGGACAGGCTCCCAGAGTGCTGATCTACA |
| | | AGGTGTCCAACCGGTTCTCTGGCATCCCCGACA |
| | | GATTTTCCGGCTCTGGCTCTGGCACCGACTTCA |
| | | CCCTGACCATCTCTAGACTGGAACCCGACGAC |
| | | TTCGCCATCTACTACTGCTCCCAGTCCACACAC |
| | | ATCCCTCTGGCTTTTGGCCAGGGCACCAAGCTG |
| | | GAAATCAAGAGAACCGTGGCCGCTCCTTCCGT |
| | | GTTCATCTTCCCACCATCTGACGAGCAGCTGAA |
| | | GTCCGGCACAGCTTCTGTCGTGTGCCTGCTGAA |
| | | CAACTTCTACCCTCGGGAAGCCAAGGTGCAGT |
| | | GGAAGGTGGACAATGCCCTGCAGTCCGGCAAC |
| | | TCCCAAGAGTCTGTGACCGAGCAGGACTCCAA |
| | | GGACTCTACCTACAGCCTGTCCTCCACACTGAC |
| | | CCTGTCTAAGGCCGACTACGAGAAGCACAAGG |
| | | TGTACGCCTGTGAAGTGACCCACCAGGGCCTG |
| | | TCTAGCCCTGTGACCAAGTCTTTCAACCGGGGC |
| | | GAGTGTTGA |
| 124 | VL-CDR1 (variant 1) | RSSQSLVHSNGNTYLH |
| 125 | VL-CDR1 (variant 2) | RSSQSLVHSSGNTYLH |
| 126 | VL-CDR1 (variant 3) | RSSQSLVHSTGNTYLH |
| 127 | VL-CDR1 (variant 4) | RSSQSLVHSQGNTYLH |
| 128 | VL-CDR2 | KVSNRFS |
| 129 | VL-CDR3 | SQSTHIPLA |

Humanized AGX-A07 H2v1L5v2

| | | |
|---|---|---|
| 130 | AGX-A07 H2v1 | EVQLVQSGAEVKKPGASVKVSCKASGYTFTNYG |
| | Heavy chain variable | VKWVROAPGQGLEWMGWINTYTGNPIYAADFK |
| | region amino acid | GRVTMTTDTSTSTAYMELRSLRSDDTAVYYCVR |
| | | FQYGDYRYFDVWGQGTLVTVSS |
| 131 | AGX-A07 H2v1L5v2 | EIVLTQSPATLSLSPGERATLSCRAQSGISFINWYQ |
| | Light chain variable | QKPGQAPRLLIYGTANLASGIPARFSGSGSGRDFT |
| | region amino acid | LTISSLEPEDFAVYYCQQWSSNPLTFGGGTKVEIK |

Humanized AGX-A07 H2L5

| | | |
|---|---|---|
| 132 | AGX-A07 H2 | QVQLVQSGAEVKKPGASVKVSCKASGYTFTNYG |
| | Heavy chain variable | VKWVRQAPGQDLEWMGWINTYTGNPIYAADFK |
| | region amino acid | GRVTMTTDTSTSTAFMELRSLRSDDTAVYYCVR |
| | | FQYGDYRYFDVWGQGTLVTVSS |
| 133 | AGX-A07 L5 | EIILTQSPATLSLSPGERATLSCRANSGISFINWYQ |
| | Light chain variable | QKPGQAPRLLIYGTANLASGIPARFGGSGSGRDF |
| | region amino acid | TLTISSLEPEDFAVYYCQQWSSNPLTFGGGTKVEI |
| | | K |

Fc Region Sequences

| | | |
|---|---|---|
| 135 | IgG1 L234A/L235A/G237A | ASTKGPSVFPLAPSSKSTSGGTAALGCLVKDYFP |
| | | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPE [symbol] G [symbol] PSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 136 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | N297C | VTVPSSSLGTQTYICNVNHKPSNTKVDKKVEPKS |
| | | CDKTHTCPPCPAPE [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KCKVSNKALPAPIEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 137 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | P331G | VTVPSSSLGTQTYICNVNHKPSNTKVDKKVEPKS |
| | | CDKTHTCPPCPAPE [symbol] G [symbol] PSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPA [symbol] IEKTISKAKGQPREPQVYTLP |
| | | PSREEMTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| 138 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | N297C/P331G | VTVPSSSLGTQTYICNVNHKPSNTKVDKKVEPKS |
| | | CDKTHTCPPCPAPE [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KCKVSNKALPA [symbol] IEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 139 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | K322A/P331G | VTVPSSSLGTQTYICNVNHKPSNTKVDKKVEPKS |
| | | CDKTHTCPPCPAPE [symbol] G [symbol] PSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKC [symbol] VSNKALPA [symbol] IEKTISKAKGQPREPQVYTLP |
| | | PSREEMTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| 140 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | E233P/P331G | VTVPSSSLGTQTYICNVNHKPSNTKVDKKVEPKS |
| | | CDKTHTCPPCPAP [symbol] G [symbol] PSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPA [symbol] IEKTISKAKGQPREPQVYTLP |
| | | PSREEMTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| 141 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | E233P/N297C | VTVPSSSLGTQTYICNVNHKPSNTKVDKKVEPKS |
| | | CDKTHTCPPCPAP [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KCKVSNKALPAPIEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 142 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | N297C/K322A/P331G | VTVPSSSLGTQTYICNVNHKPSNTKVDKKVEPKS |
| | | CDKTHTCPPCPAP [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KC [symbol] VSNKALPA [symbol] IEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 143 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | E233P/N297C/P331G | VTVPSSSLGTQTYICNVNHKPSNTKVDKKVEPKS |
| | | CDKTHTCPPCPAP [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KCKVSNKALPA [symbol] IEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 144 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | E233P/D265A/N297C/ | VTVPSSSLGTQTYICNVNHKPSNTKVDKKVEPKS |
| | K322A/P331G | CDKTHTCPPCPAP [symbol] G [symbol] PSVFLFPPKPKDTLMIS |
| | | RTPEVTCVVVAVSHEDPEVKFNWYVDGVEVHN |
| | | AKTKPREEQY [symbol] STYRVVSVLTVLHQDWLNGKEY |
| | | KC [symbol] VSNKALPA [symbol] IEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 145 | IgG1 | ASTKGPSVFPLAPSSKSTSGGTAALGCLVKDYFP |
| | L234A/L235A/G237A + | EPVTVSWNSGALTSGVHTFPAVLQSSGLYSLSSV |
| | E233P/D265A/N297C/ | VTVPSSSLGTQTYICNVNHKPSNTKVDKKVEPKS |
| | K322A/P331G-PGKKP | CDKTHTCPPCPAP [symbol] G [symbol] PSVFLFPPKPKDTLMIS |
| | | RTPEVTCVVVDVSHEDPEVKFNWYVDGVEVHN |
| | | AKTKPREEQY [symbol] STYRVVSVLTVLHQDWLNGKEY |
| | | KC [symbol] VSNKALPA [symbol] IEKTISKAKGQPREPQVYTLPPS |
| | | REEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGKKP |
| 146 | IgG4 | EVQLVQSGAEVKKPGASVKVSCKASGYTFTNYG |
| | S228P (sequence includes | VKWVRQAPGQGLEWMGWINTYTGNPIYAADFK |
| | AGX-A07 H2v1 heavy chain | GRVTMTTDTSTSTAYMELRSLRSDDTAVYYCVR |
| | variable region amino | FQYGDYRYFDVWGQGTLVTVSSASTKGPSVFPL |
| | acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCP [symbol] CPAPEF |
| | | LGGPSVFLFPPKPKDTLMISRTPEVTCVVVDVSQE |
| | | DPEVQFNWYVDGVEVHNAKTKPREEQFNSTYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSVMHEALHNH |
| | | YTQKSLSLSLGK |
| 147 | IgG4 | EVQLVQSGAEVKKPGASVKVSCKASGYTFTNYG |
| | S228P/L235E | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCP [symbol] CPAPEF |
| | | [symbol] GGPSVFLFPPKPKDTLMISRTPEVTCVVVDVSQ |
| | | EDPEVQFNWYVDGVEVHNAKTKPREEQFNSTYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSVMHEALHNH |
| | | YTQKSLSLSLGK |
| 148 | IgG4 | EVQLVQSGAEVKKPGASVKVSCKASGYTFTNYG |
| | S228P/L235E/N297C | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCP [symbol] CPAPEF |
| | | [symbol] GGPSVFLFPPKPKDTLMISRTPEVTCVVVDVSQ |
| | | EDPEVQFNWYVDGVEVHNAKTKPREEQF [symbol] STYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSVMHEALHNH |
| | | YTQKSLSLSLGK |
| 149 | IgG4 | EVQLVQSGAEVKKPGASVKVSCKASGYTFTNYG |
| | S228P/F234A/L235E/N297C | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCP [symbol] CPAPE [symbol] |
| | | [symbol] GGPSVFLFPPKPKDTLMISRTPEVTCVVVDVSQ |
| | | EDPEVQFNWYVDGVEVHNAKTKPREEQF [symbol] STYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSVMHEALHNH |
| | | YTQKSLSLSLGK |
| 150 | IgG4 | EVQLVQSGAEVKKPGASVKVSCKASGYTFTNYG |
| | S228P/L235E/N297C-LGKKP | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCP [symbol] CPAPEF |
| | | [symbol] GGPSVFLFPPKPKDTLMISRTPEVTCVVVDVSQ |
| | | EDPEVQFNWYVDGVEVHNAKTKPREEQF [symbol] STYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSVMHEALHNH |
| | | YTQKSLSLSLGKKP |
| 151 | IgG1 | ASTKGPSVFPLAPSSKSTSGGTAALGCLVKDYFP |
| | M252Y/S254T/T256E | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPELLGGPSVFLFPPKPKDTL [symbol] I [symbol] |
| | | R [symbol] PEVTCVVVDVSHEDPEVKFNWYVDGVEVHN |
| | | AKTKPREEQYNSTYRVVSVLTVLHQDWLNGKE |
| | | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSVMHEALHNHYTQKSLSLSPGK |
| 152 | IgG1 | ASTKGPSVFPLAPSSKSTSGGTAALGCLVKDYFP |
| | T252Q/M428L | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPELLGGPSVFLFPPKPKD [symbol] LMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSV [symbol] HEALHNHYTQKSLSLSPGK |
| 153 | IgG1 | ASTKGPSVFPLAPSSKSTSGGTAALGCLVKDYFP |
| | M428L/N434S | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPELLGGPSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPAPIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQG |
| | | NVFSCSV [symbol] HEALH [symbol] HYTQKSLSLSPGK |
| 154 | IgG4 | EVQLVQSGAEVKKPGASVKVSCKASGYTFTNYG |
| | T250Q/M428L | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCPSCPAPEF |
| | | LGGPSVFLFPPKPKD [symbol] LMISRTPEVTCVVVDVSQ |
| | | EDPEVQFNWYVDGVEVHNAKTKPREEQFNSTYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSV [symbol] HEALHNH |
| | | YTQKSLSLSLGK |
| 155 | IgG4 | EVQLVQSGAEVKKPGASVKVSCKASGYTFTNYG |
| | M428L/N434S | VKWVRQAPGQGLEWMGWINTYTGNPIYAADFK |
| | (sequence includes AGX-A07 | GRVTMTTDTSTSTAYMELRSLRSDDTAVYYCVR |
| | H2v1 heavy chain region | FQYGDYRYFDVWGQGTLVTVSSASTKGPSVFPL |
| | amino acid) | APCSRSTSESTAALGCLVKDYFPEPVTVSWNSGA |
| | | LTSGVHTFPAVLQSSGLYSLSSVVTVPSSSLGTKT |
| | | YTCNVDHKPSNTKVDKRVESKYGPPCPSCPAPEF |
| | | LGGPSVFLFPPKPKDTLMISRTPEVTCVVVDVSQE |
| | | DPEVQFNWYVDGVEVHNAKTKPREEQFNSTYR |
| | | VVSVLTVLHQDWLNGKEYKCKVSNKGLPSSIEK |
| | | TISKAKGQPREPQVYTLPPSQEEMTKNQVSLTCL |
| | | VKGFYPSDIAVEWESNGQPENNYKTTPPVLDSDG |
| | | SFFLYSRLTVDKSRWQEGNVFSCSV [symbol] HEALH [symbol] HY |
| | | TQKSLSLSLGK |
| 156 | IgG1 | EVQLVQSGAEVKKPGASVKVSCKASGYTFTNYG |
| | M252Y/S254T/T256E | VKWVRQAPGQGLEWMGWINTYTGNPIYAADFK |
| | (sequence includes AGXA07 | GRVTMTTDTSTSTAYMELRSLRSDDTAVYYCVR |
| | H2v1 heavy chain variable | FQYGDYRYFDVWGQGTLVTVSSASTKGPSVFPL |
| | region amino acid) | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PELLGGPSVFLFPPKPKDTL [symbol] I [symbol] R [symbol] PEVTCVVVDV |
| | | SHEDPEVKFNWYVDGVEVHNAKTKPREEQYNST |
| | | YRVVSVLTVLHQDWLNGKEYKCKVSNKALPAPI |
| | | EKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| 157 | Brd-4 protein sequence | MSAESGPGTRLRNLPVMGDGLETSQMSTTQAQA |
| | >sp\|O60885\|BRD4_HUMAN | QPQPANAASTNPPPPETSNPNKPKRQTNQLQYLL |
| | Bromodomain-containing | RVVLKTLWKHQFAWPFQQPVDAVKLNLPDYYK |
| | protein 4 | IIKTPMDMGTIKKRLENNYYWNAQECIQDFNTM |
| | OS = Homo sapiens | FTNCYIYNKPGDDIVLMAEALEKLFLQKINELPTE |
| | OX = 9606 GN = BRD4 | ETEIMIVQAKGRGRGRKETGTAKPGVSTVPNTTQ |
| | PE = 1 SV = 2 | ASTPPQTQTPQPNPPPVQATPHPFPAVTPDLIVQT |
| | | PVMTVVPPQPLQTPPPVPPQPQPPPAPAPQPVQSH |
| | | PPIIAATPQPVKTKKGVKRKADTTTPTTIDPIHEPP |
| | | SLPPEPKTTKLGQRRESSRPVKPPKKDVPDSQQH |
| | | PAPEKSSKVSEQLKCCSGILKEMFAKKHAAYAW |
| | | PFYKPVDVEALGLHDYCDIIKHPMDMSTIKSKLE |
| | | AREYRDAQEFGADVRLMFSNCYKYNPPDHEVV |
| | | AMARKLQDVFEMRFAKMPDEPEEPVVAVSSPAV |
| | | PPPTKVVAPPSSSDSSSDSSSDSDSSTDDSEEERAQ |
| | | RLAELQEQLKAVHEQLAALSQPQQNKPKKKE |
| | | KDKKEKKKEKHKRKEEVEENKKSKAKEPPPKKT |
| | | KKNNSSNSNVSKKEPAPMKSKPPPTYESEEEDKC |
| | | KPMSYEEKRQLSLDINKLPGEKLGRVVHIIQSREP |
| | | SLKNSNPDEIEIDFETLKPSTLRELERYVTSCLRKK |
| | | RKPQAEKVDVIAGSSKMKGFSSSESESSSESSSSD |
| | | SEDSETEMAPKSKKKGHPGREQKKHHHHHHQQ |
| | | MQQAPAPVPQQPPPPPQQPPPPPPPQQQQQPPPPP |
| | | PPPSMPQQAAPAMKSSPPPFIATQVPVLEPQLPGS |
| | | VFDPIGHFTQPILHLPQPELPPHLPQPPEHSTPPHL |
| | | NQHAVVSPPALHNALPQQPSRPSNRAAALPPKPA |
| | | RPPAVSPALTQTPLLPQPPMAQPPQVLLEDEEPPA |
| | | PPLTSMQMQLYLQQLQKVQPPTPLLPSVKVQSQP |
| | | PPPLPPPPHPSVQQQLQQQPPPPPPPQPQPPPQQQ |
| | | HQPPPRPVHLQPMQFSTHIQQPPPPQGQQPP |
| | | HPPPGQQPPPPQPAKPQQVIQHHHSPRHHKSDPY |
| | | STGHLREAPSPLMIHSPQMSQFQSLTHQSPPQQN |
| | | VQPKKQELRAASVVQPQPLVVVKEEKIHSPIIRSE |
| | | PFSPSLRPEPPKHPESIKAPVHLPQRPEMKPVDVG |
| | | RPVIRPPEQNAPPPGAPDKDKQKQEPKTPVAPKK |
| | | DLKIKNMGSWASLVQKHPTTPSSTAKSSSDSFEQ |
| | | FRRAAREKEEREKALKAQAEHAEKEKERLRQER |
| | | MRSREDEDALEQARRAHEEARRRQEQQQQQRQ |
| | | EQQQQQQQQAAAVAAAATPQAQSSQPQSM |
| | | LDQQRELARKREQERRRREAMAATIDMNFQSDL |
| | | LSIFEENLF |
| 158 | Akt-1 amino acid sequence | QLMKTERPRPNTFIIRCLQWTTVIERTFHVETPEE |
| | >sp\|P31749\|AKT1_HUMAN RAC- | REEWTTAIQTVADGLKKQEEEEMDFRSGSPSDNS |
| | alpha serine/threonine-protein | GAEEMEVSLAKPKHRVTMNEFEYLKLLGKGTFG |
| | kinase OS = Homo sapiens | KVILVKEKATGRYYAMKILKKEVIVAKDEVAHT |
| | OX = 9606 GN = AKT1 | LTENRVLQNSRHPFLTALKYSFQTHDRLCFVMEY |
| | PE = 1 SV = 2 | ANGGELFFHLSRERVFSEDRARFYGAEIVSALDY |
| | | LHSEKNVVYRDLKLENLMLDKDGHIKITDFGLC |
| | | KEGIKDGATMKTFCGTPEYLAPEVLEDNDYGRA |
| | | VDWWGLGVVMYEMMCGRLPFYNQDHEKLFEL |
| | | ILMEEIRFPRTLGPEAKSLLSGLLKKDPKQRLGGG |
| | | SEDAKEIMQHRFFAGIVWQHVYEKKLSPPFKPQV |
| | | TSETDTRYFDEEFTAQMITITPPDQDDSMECVDSE |
| | | RRPHFPQFSYSASGTA |
| 159 | Akt-2 amino acid sequence | MNEVSVIKEGWLHKRGEYIKTWRPRYFLLKSDG |
| | >sp\|P31751\|AKT2_HUMAN RAC- | SFIGYKERPEAPDQTLPPLNNFSVAECQLMKTERP |
| | beta serine/ | RPNTFVIRCLQWTTVIERTFHVDSPDEREEWMRA |
| | threonine-protein kinase | IQMVANSLKQRAPGEDPMDYKCGSPSDSSTTEE |
| | OS = Homo sapiens | MEVAVSKARAKVTMNDFDYLKLLGKGTFGKVI |
| | OX = 9606 GN = AKT2 | LVREKATGRYYAMKILRKEVIIAKDEVAHTVTES |
| | PE = 1 SV = 2 | RVLQNTRHPFLTALKYAFQTHDRLCFVMEYANG |
| | | GELFFHLSRERVFTEERARFYGAEIVSALEYLHSR |
| | | DVVYRDIKLENLMLDKDGHIKITDFGLCKEGISD |
| | | GATMKTFCGTPEYLAPEVLEDNDYGRAVDWWG |
| | | LGVVMYEMMCGRLPFYNQDHERLFELILMEEIR |
| | | FPRTLSPEAKSLLAGLLKKDPKQRLGGGPSDAKE |
| | | VMEHRFFLSINWQDVVQKKLLPPFKPQVTSEVDT |
| | | RYFDDEFTAQSITITPPDRYDSLGLLELDQRTHFP |
| | | QFSYSASIRE |
| 160 | Akt-3 amino acid sequence | MSDVTIVKEGWVQKRGEYIKNWRPRYFLLKTDG |
| | >sp\|Q9Y243\|AKT3_HUMAN | SFIGYKEKPQDVDLPYPLNNFSVAKCQLMKTERP |
| | RAC-gamma serine/threonine- | KPNTFIIRCLQWTTVIERTFHVDTPEEREEWTEAI |
| | protein kinase | QAVADRLQRQEEERMNCSPTSQIDNIGEEEMDAS |
| | OS = Homo sapiens | TTHHKRKTMNDFDYLKLLGKGTFGKVILVREKA |
| | OX = 9606 GN = AKT3 | SGKYYAMKILKKEVIIAKDEVAHTLTESRVLKNT |
| | PE = 1 SV = 1 | RHPFLTSLKYSFQTKDRLCFVMEYVNGGELFFHL |
| | | SRERVFSEDRTRFYGAEIVSALDYLHSGKIVYRD |
| | | LKLENLMLDKDGHIKITDFGLCKEGITDAATMKT |
| | | FCGTPEYLAPEVLEDNDYGRAVDWWGLGVVMY |
| | | EMMCGRLPFYNQDHEKLFELILMEDIKFPRTLSS |
| | | DAKSLLSGLLIKDPNKRLGGGPDDAKEIMRHSFF |
| | | SGVNWQDVYDKKLVPPFKPQVTSETDTRYFDEE |
| | | FTAQTITITPPEKYDEDGMDCMDNERRPHFPQFS |
| | | YSASGRE |
| 161 | >sp\|Q07817\|B2CL1_HUMAN Bcl- | MSQSNRELVVDFLSYKLSQKGYSWSQFSDVEEN |
| | 2-like protein 1 | RTEAPEGTESEMETPSAINGNPSWHLA |
| | OS = Homo sapiens | DSPAVNGATGHSSSLDAREVIPMAAVKQALREA |
| | OX = 9606 GN = BCL2L1 | GDEFELRYRRAFSDLTSQLHITPGTAY |
| | PE = 1 SV = 1 | QSFEQVVNELFRDGVNWGRIVAFFSFGGALCVES |
| | BCL-X(L) amino acid sequence | VDKEMQVLVSRIAAWMATYLNDHLEP |
| | | WIQENGGWDTFVELYGNNAAAESRKGQERFNR |
| | | WFLTGMTVAGVVLLGSLFSRK |

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Conjugation of Degraders to an Anti-TM4SF1 Antibody

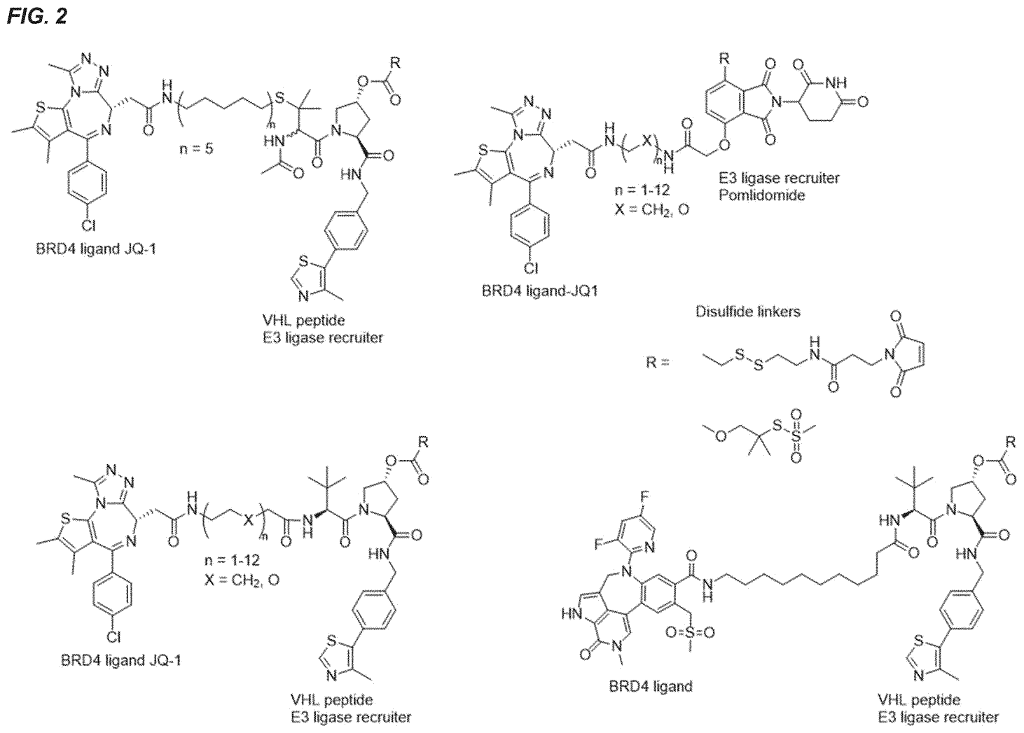

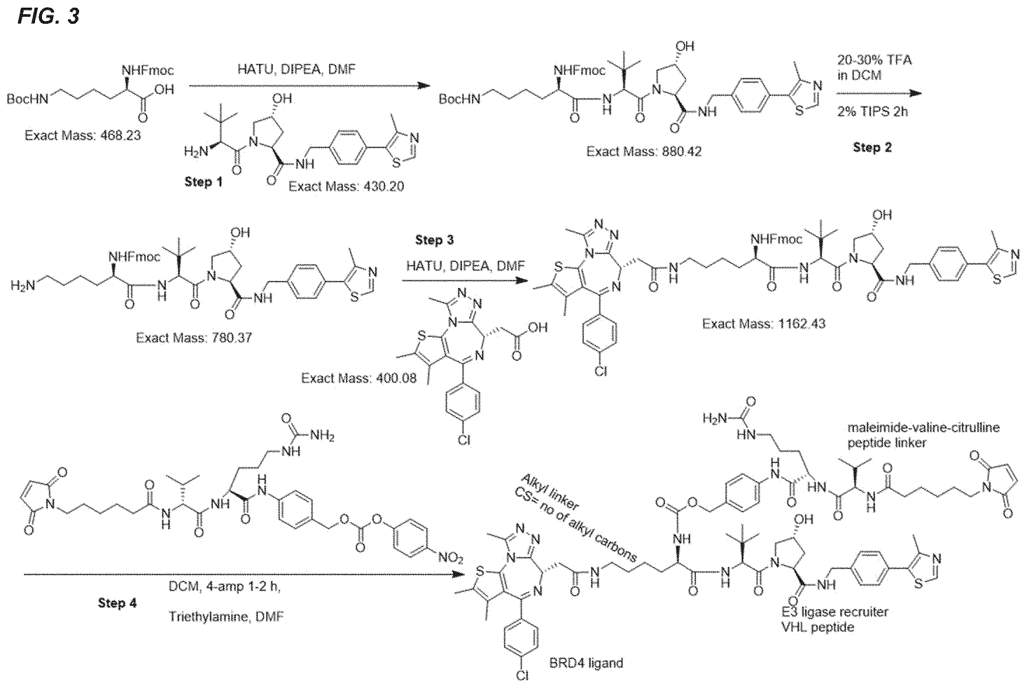

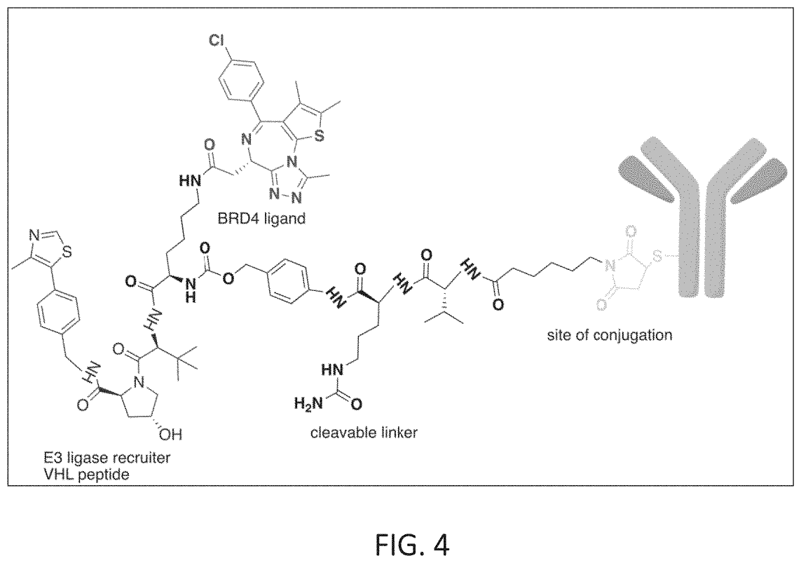

Various exemplary degraders (Brd4, BCL-XL, Akt) are conjugated to anti-TM4SF1 antibodies. Example structures for Brd4 degraders are provided in FIGS. 1A, 1B, 1C, and FIG. 2. One of the exemplary Brd4 degraders were conjugated to AGX-A07 anti-TM4SF1 antibody via a maleimide-valine-citrulline cleavable linker, using a chemical synthesis route as shown in FIG. 3. The resulting antibody-degrader conjugate is shown in FIG. 4.

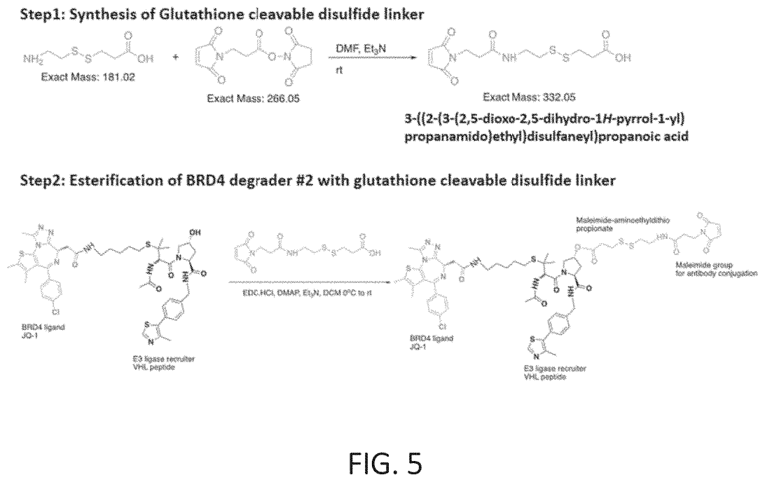

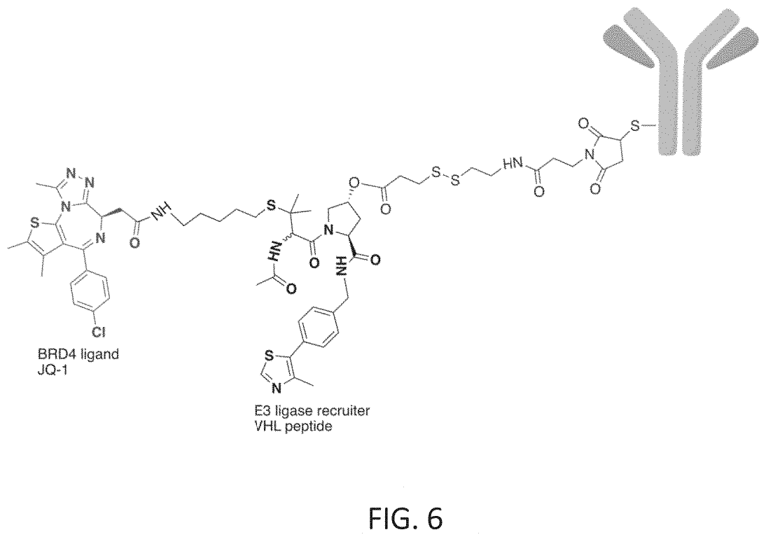

A further exemplary degrader conjugate is synthesized using the steps illustrated in FIG. 5. In the first step, a glutathione cleavable disulfide linker is synthesized. The Brd4 degrader (FIG. 1B) is esterified with the glutathione cleavable disulfide linker, as shown in step 2 of FIG. 5. The resultant degrader-antibody conjugate is shown in FIG. 6.

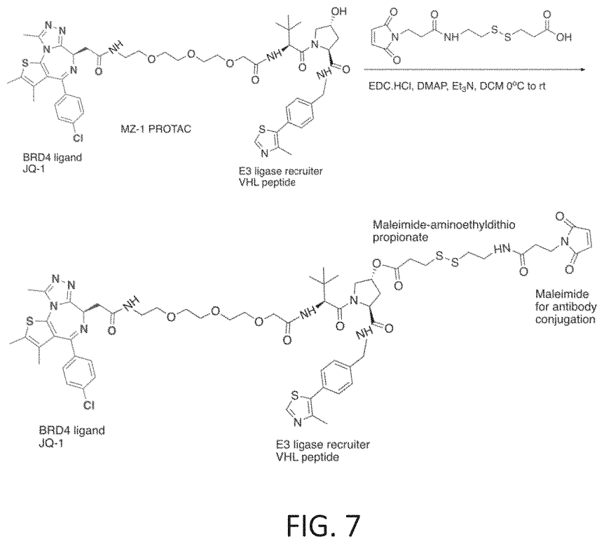

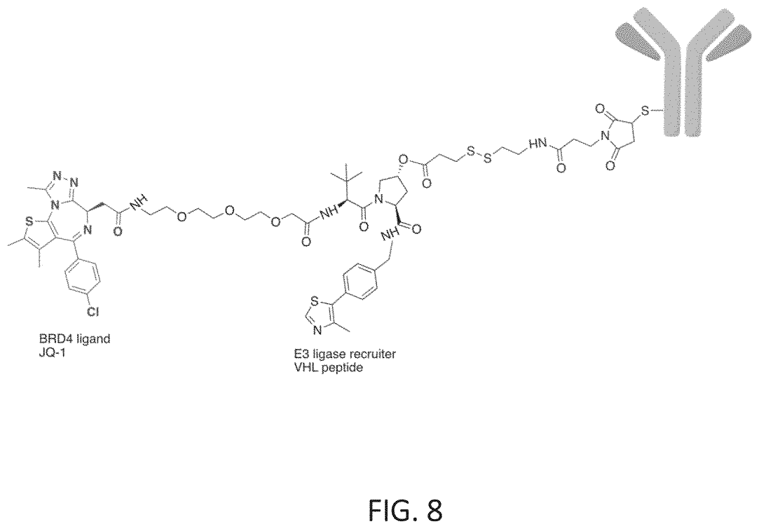

Another exemplary degrader conjugate is synthesized as shown in FIG. 7, by conjugating the Brd4 degrader of FIG. 1C (top panel) to AGX-A07 antibody, via a glutathione cleavable linker. The resultant structure is shown in FIG. 8.

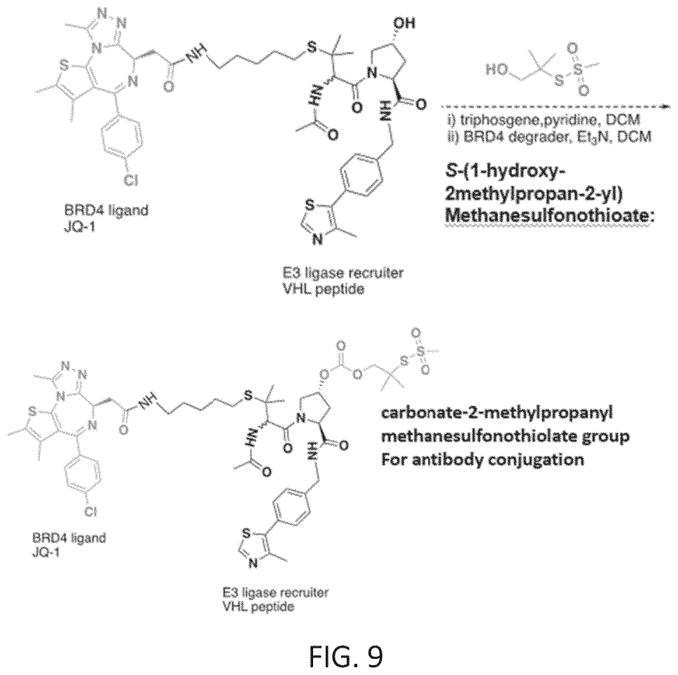

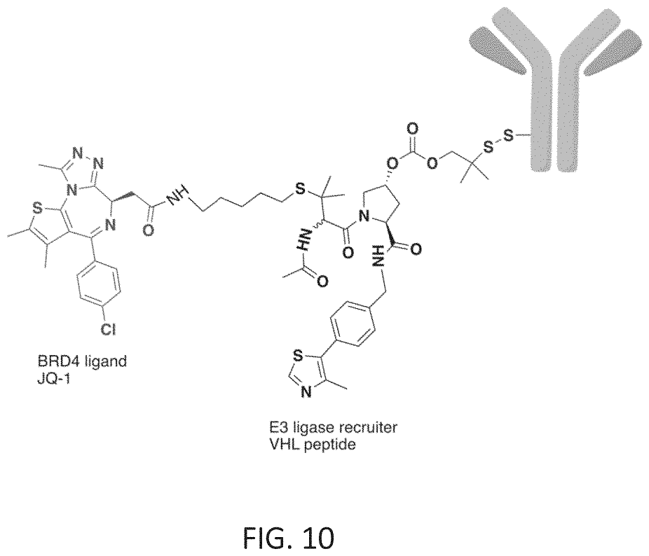

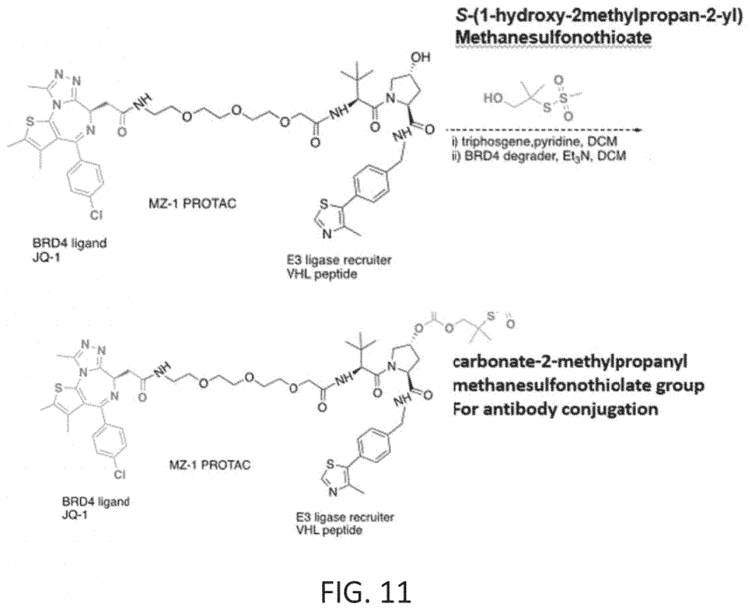

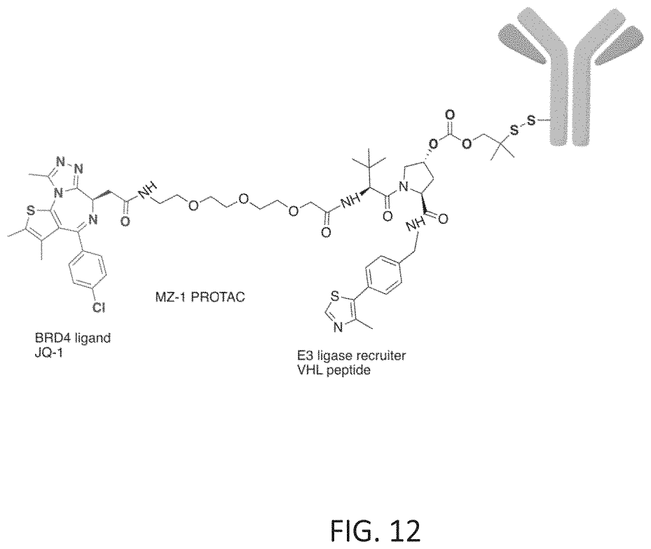

An additional approach used for conjugation of a Brd4 degrader to AGX-A07 antibody is shown in FIG. 9, where the Brd4 degrader shown in FIG. 1B is conjugated using a cleavable disulfide linker. The resultant structure is provided in FIG. 10. Similarly, the Brd4 degrader of FIG. 1C (top panel) is conjugated to AGX-A07 via a cleavable disulfide linker, the synthetic scheme being shown in FIG. 11, followed by the resultant conjugate structure in FIG. 12.

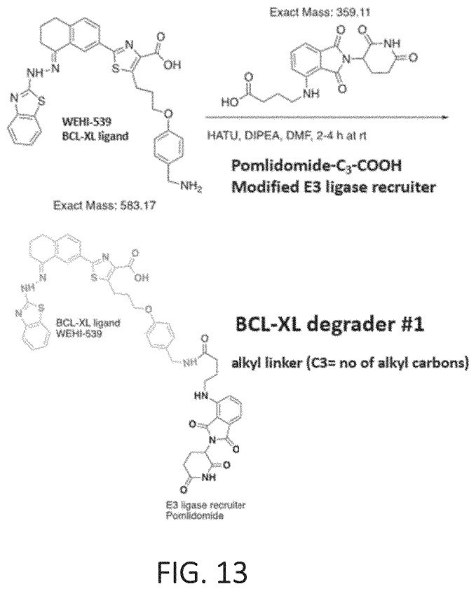

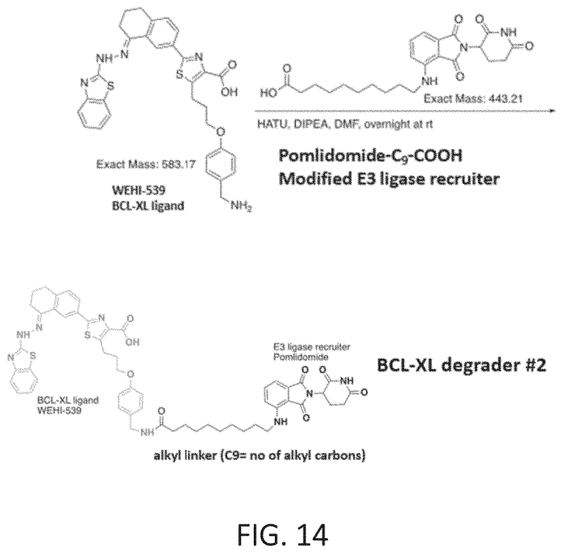

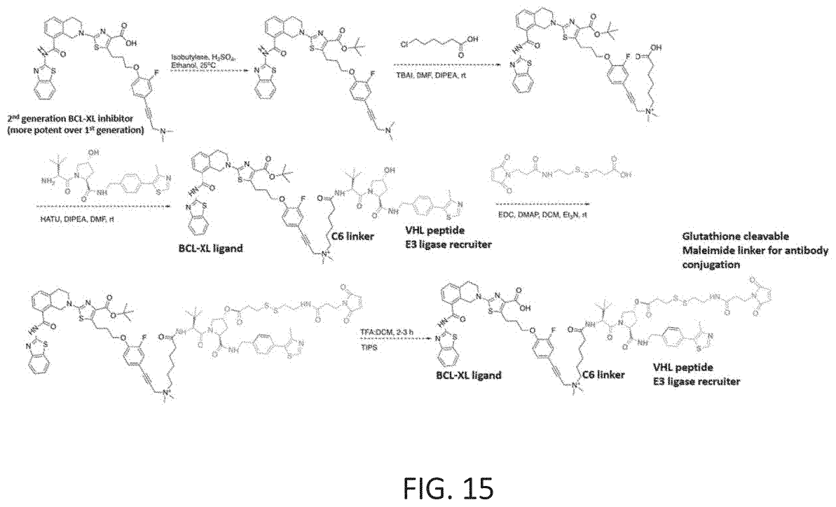

In a further study, BCL-XL degraders were synthesized using the synthetic schemes shown in FIG. 13 and FIG. 14. To enhance the potency of BCL-XL degraders, second generation BCL-XL degraders are synthesized, as illustrated in FIG. 15. BCL-XL is a mitochondrial protein. Inhibition BCL-XL proteins with small molecules has been extensively investigated as a therapeutic strategy for cancers. In the clinic, small molecule inhibitors of BCL-XL including WEHI-539 (FIG. 14) (See Lessene, G. et al. Structure-guided design of a selective Bcl-XL inhibitor. Nat. Chem. Biol. 9, 390-397 (2013)), A115463 (FIG. 15) (See Z F Tao, et al, Discovery of a Potent and Selective BCL-XL Inhibitor with in Vivo Activity, ACS Med Chem Lett. 2014 Aug. 26; 5(10):1088-93), and ABT 263 (See Khan, S.; Zhang, X.; Lv, D.; Zhang, Q.; He, Y.; Zhang, P.; Liu, X.; Thummuri, D.; Yuan, Y.; Wiegand, J. S.; et al. A selective BCL-XL PROTAC degrader achieves safe and potent anti-tumor activity. Nat. Med. 2019, 25, 1938-1947) induces on-target and dose-limiting thrombocytopenia.

In recent studies, Khan et al. found that BCL-XL degrader was more potent than the BCL-XL inhibitor and caused less toxicity to platelets. However, BCL-XL degrader designed in this work lack selectivity and was not significantly potent (DC₅₀ of 333 nM). The aim of the present example study is to design degrader antibody conjugates (DACs) of BCL-XL that may selectively deliver potent degrader molecule of BCL-XL to the target cell. To achieve this goal, BCL-XL degraders may be designed based on the highly potent second generation BCL-XL inhibitor (A115463, shown in FIG. 15). The new BCL-XL degrader may be conjugated to exemplary anti-TM4SF1 antibodies, as described herein, through cleavable and/or uncleavable linkers.

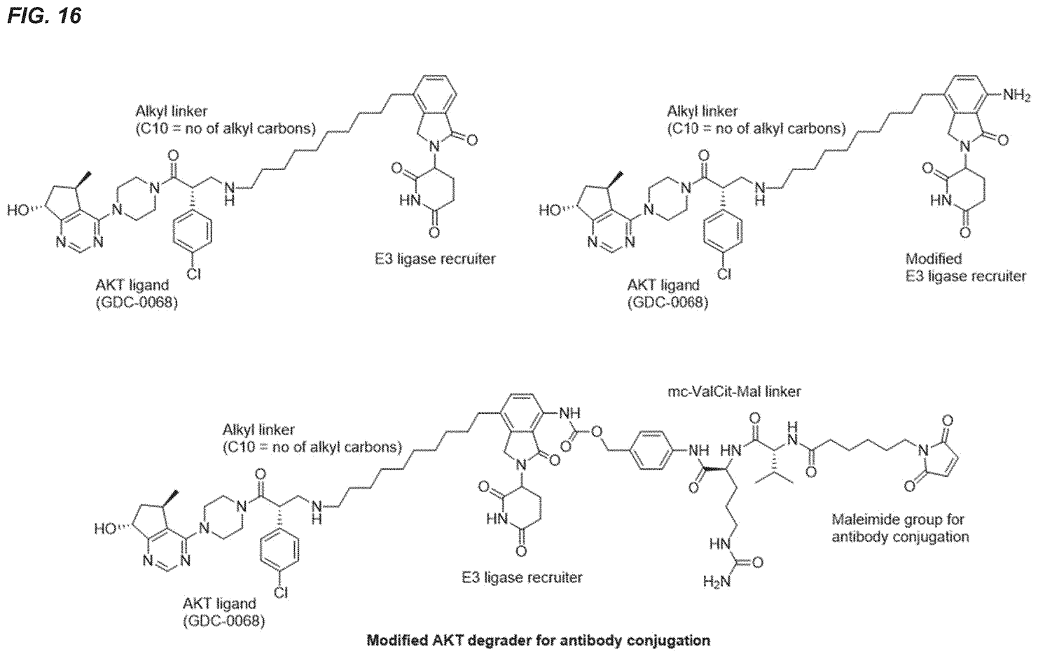

The serine/threonine kinase AKT is the main component of phosphoinositide 3-kinase (PI3K) signaling cascade. PI3K/AKT is one of the most dysregulated signaling pathway in cancer, with a large proportion of tumors exhibiting aberrant AKT activation. Although potent small molecules of AKT have entered clinical trials, robust and durable therapeutic responses have not been observed. You et al. designed pan-AKT degrader as shown in FIG. 16 that make use of GDC0068 as an AKT ligand. The above molecule showed degradation of all three AKTs from 10 to 1000 nM. Interestingly, AKT degradation had stronger anti-proliferative effects than inhibitor alone and loss/inhibition of downstream signaling was sustained even after washout.

AKT is the protein transported by TMED. Hence, it is expected that a DAC comprising an AKT degrader and anti-TM4SF1 antibody or antigen binding fragment thereof may have an efficacy advantage in targeting AKT due to this physical proximity. The proximity effects is likely to improve the potency of the current generation degraders to up to 10,000 fold and might provide longer duration of action. A modified version of the AKT degrader molecule shown in FIG. 16 is to be designed and expected to have a chemical handle to install both cleavable and uncleavable linkers for conjugation of an antibody.

Akt degraders are also synthesized, designed to have enhanced potency, relative to Akt inhibitor GDC-0068. FIG. 16 illustrates structures for some modified Akt degraders.

### Example 2: Efficacy of Exemplary Degrader Anti-TM4SF1 Conjugates

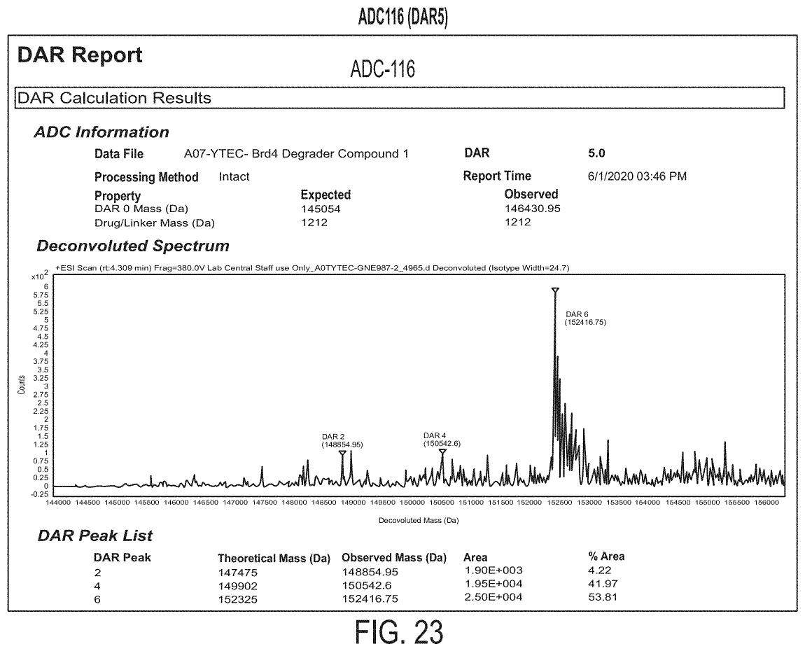

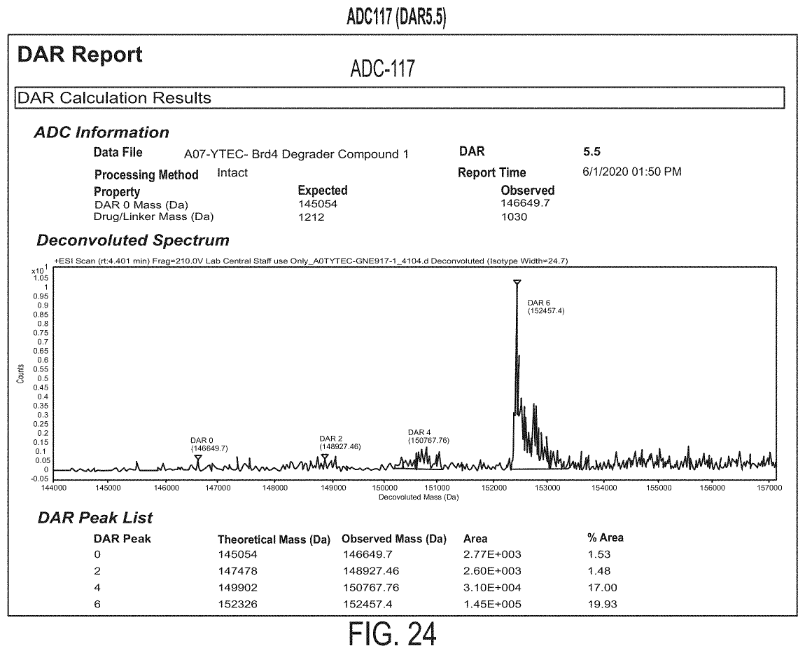

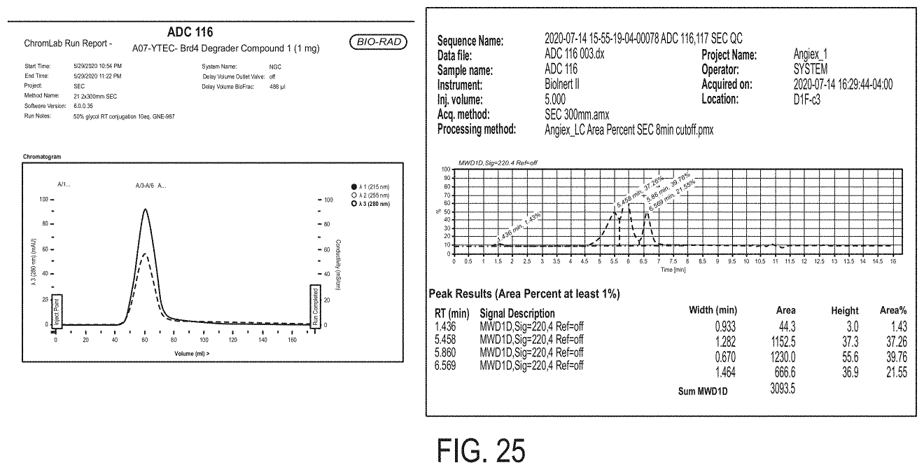

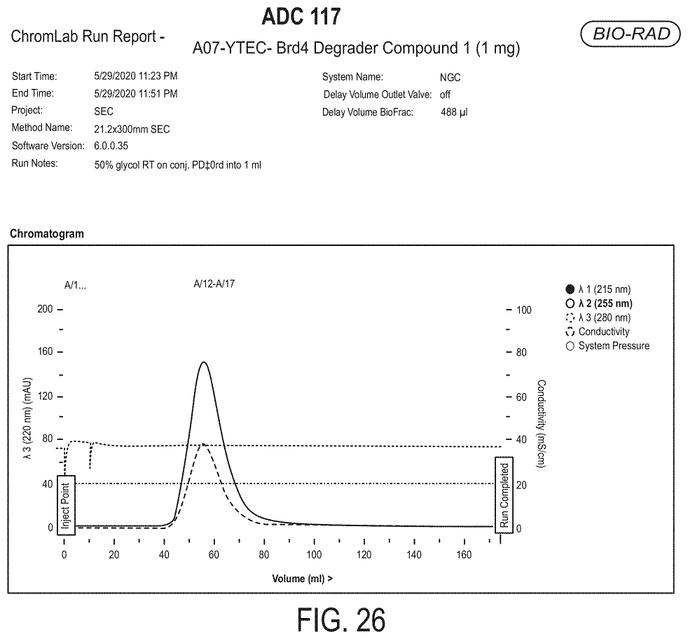

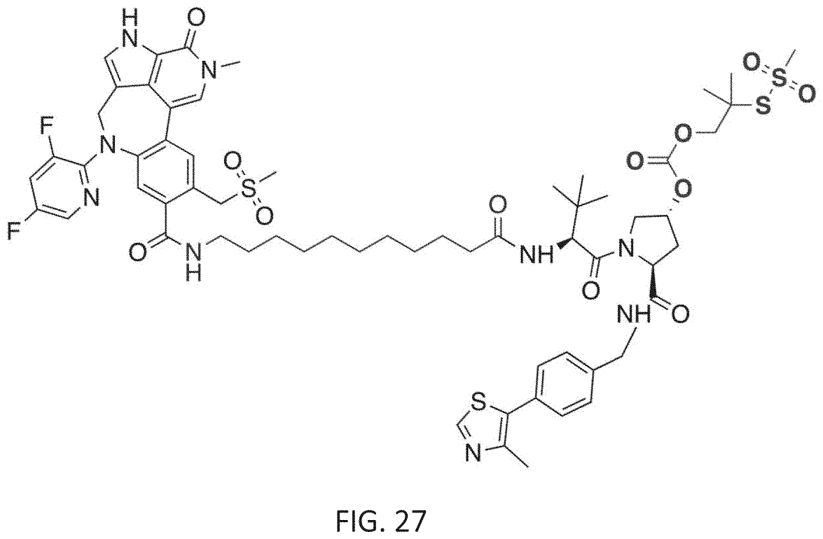

The goal of these studies was to characterize and evaluate the efficacy of an exemplary degrader antibody conjugate according to this disclosure, comprising an anti-TM4SF1 antibody with modifications in the Fc region (A07-YTEC) conjugated to a Brd4 degrader compound 1 (structure shown in FIG. 27). Brd4 is a nuclear protein that regulates gene expression via histone acetylation for chromatin de-compaction and topo-I activation. The exemplary degrader antibody conjugate (denoted as A07-YTEC-degrader compound 1) was characterized to assess the degrader to antibody ration (DAR) and further characterized by size exclusion chromatography, Q-ToF intact liquid chromatography mass spectroscopy. To measure DAR values for each respective exemplary degrader antibody conjugates, deconvoluted spectrums of DAC15 and DAC14 were generated (FIG. 23 and FIG. 24, respectively). The deconvoluted spectrum may be used to calculate or confirm DAR values based on the total ion chromatograph (TIC) peaks. Additionally, a chromatogram was generated for both DAC15 (FIG. 25) and DAC14 (FIG. 26) using a size exclusion column (21.2 mm by 300 mm) and phosphate buffered saline pH 7.4 as the mobile phase. Appropriate buffers were used to maximize stability and minimize aggregates of the target protein conjugates. For the chromatograms, the samples were synthesized in various buffers at different pH values then placed in a PD-10 desalting column into the chosen final formulation buffer. For purposes of comparison, additional in vitro assays were conducted using a conjugate comprising an anti-human TM4SF1 antibody containing a modified Fc region (A07-YTEC) conjugated to maytansinoid payload as its cytotoxic agent (referred to as ABZ or A07-YTEC-PEG4Ahx-DM1).

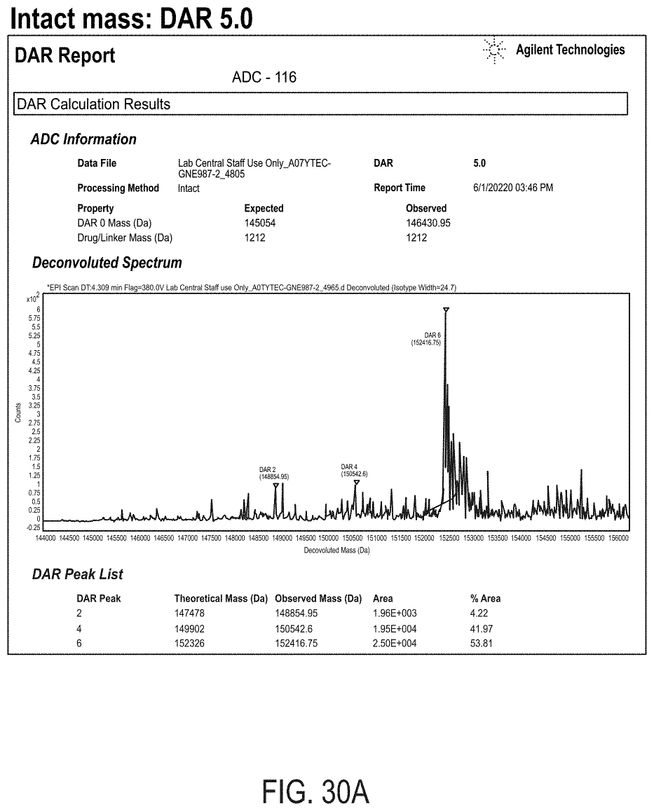

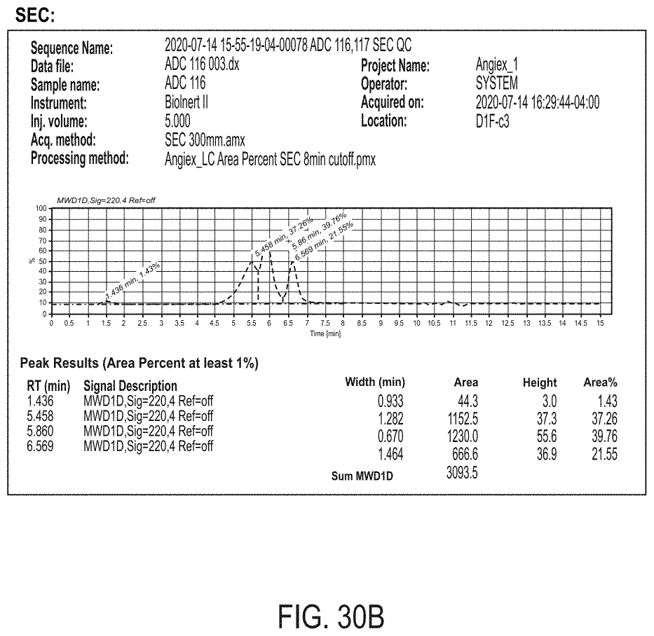

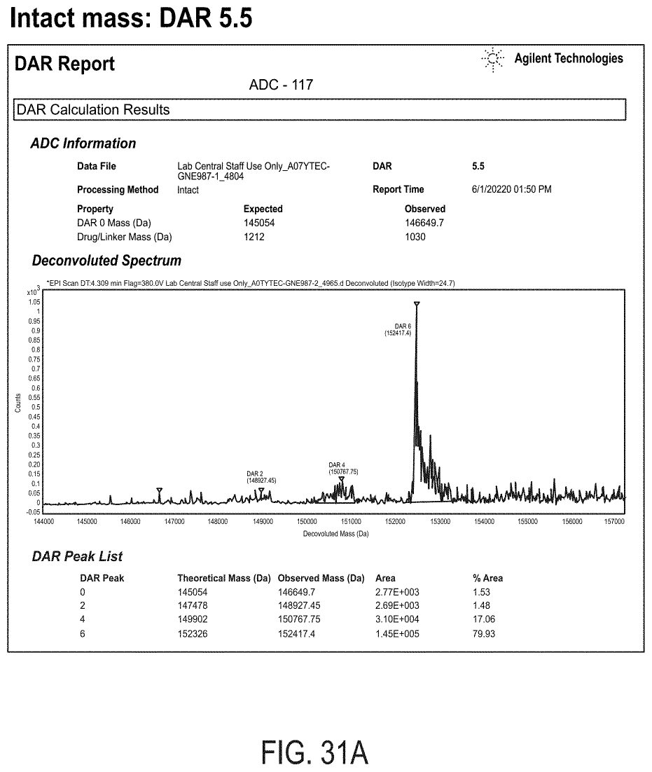

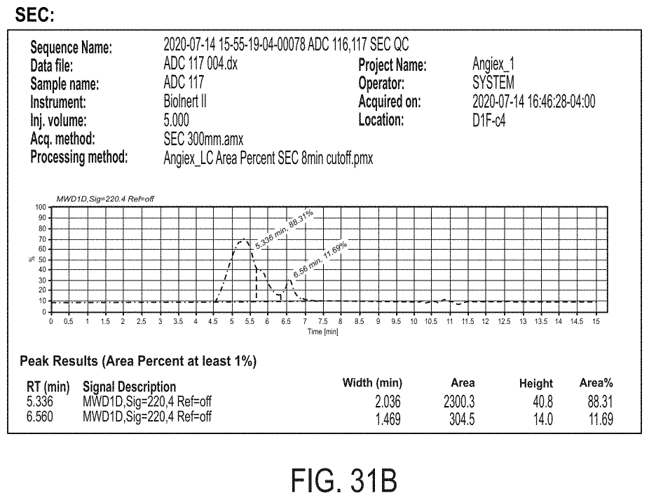

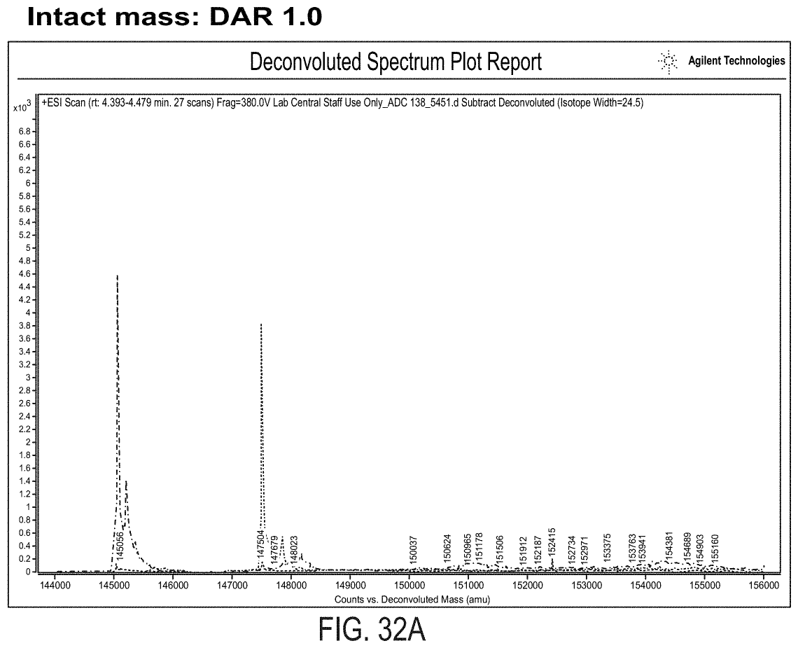

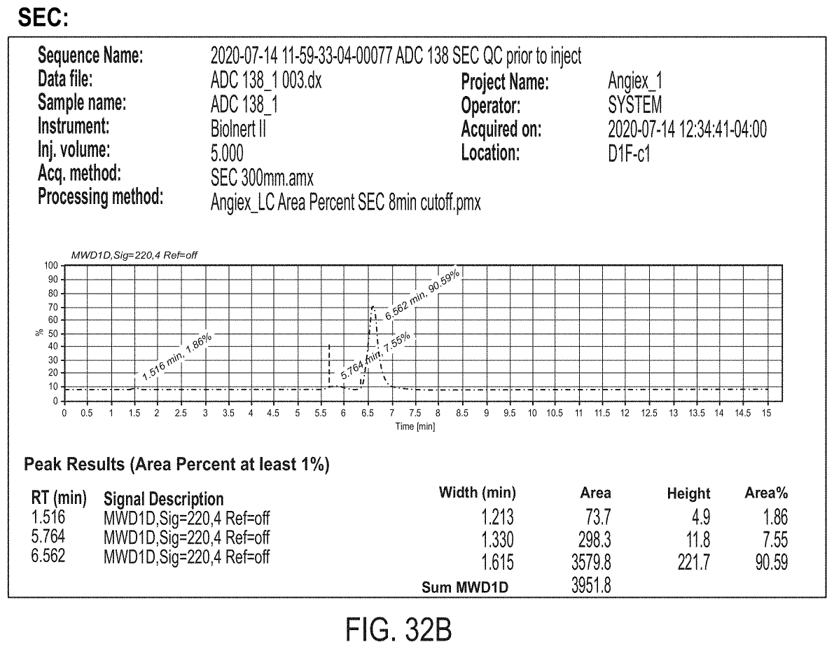

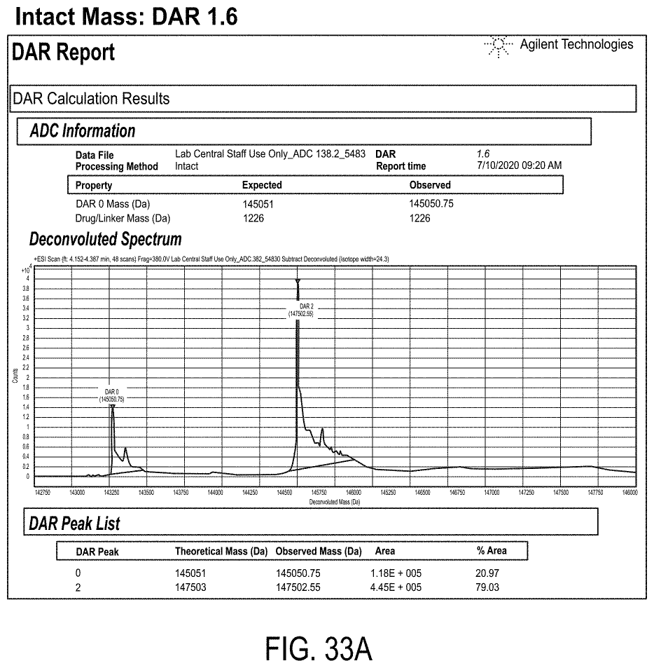

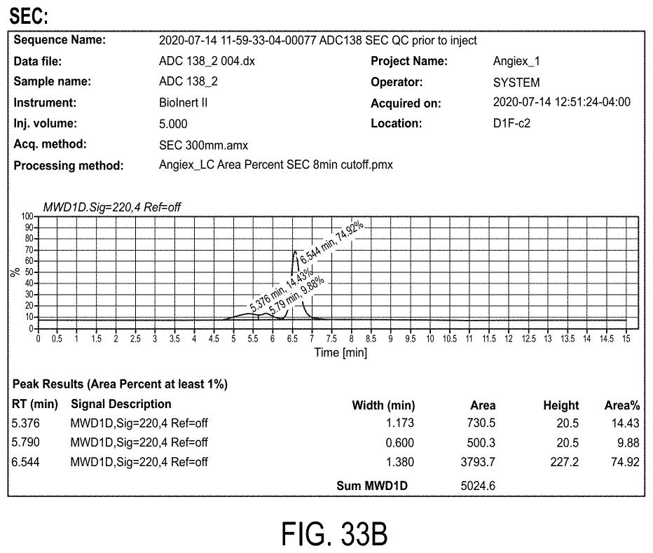

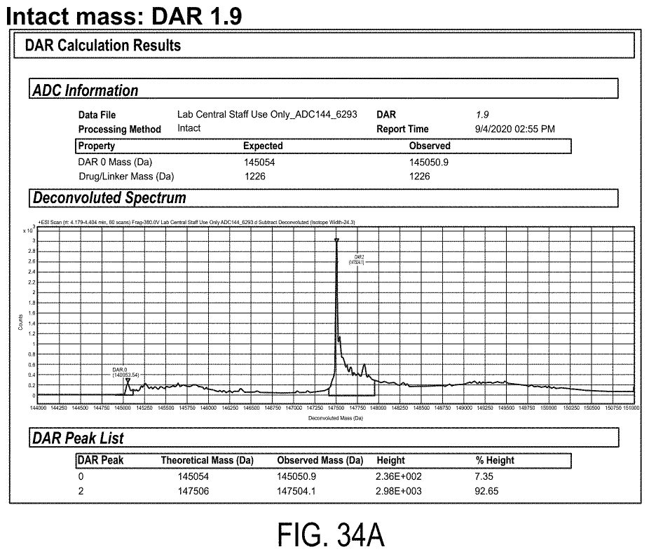

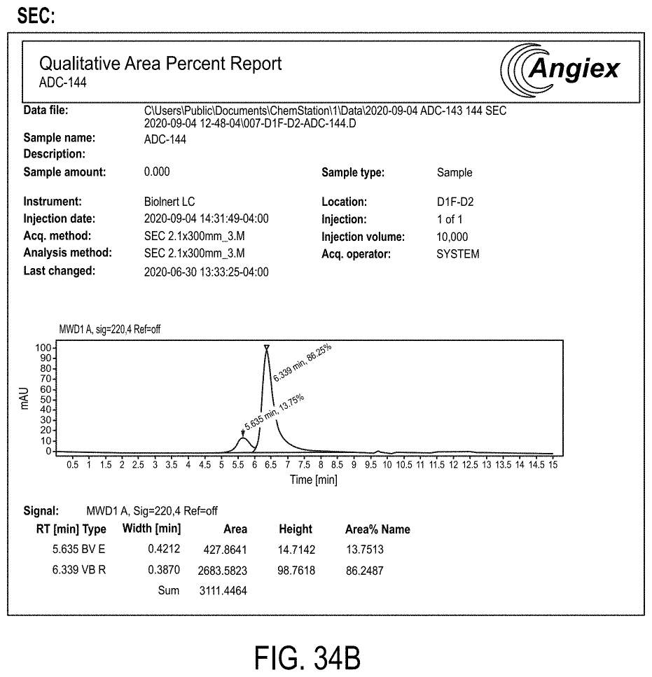

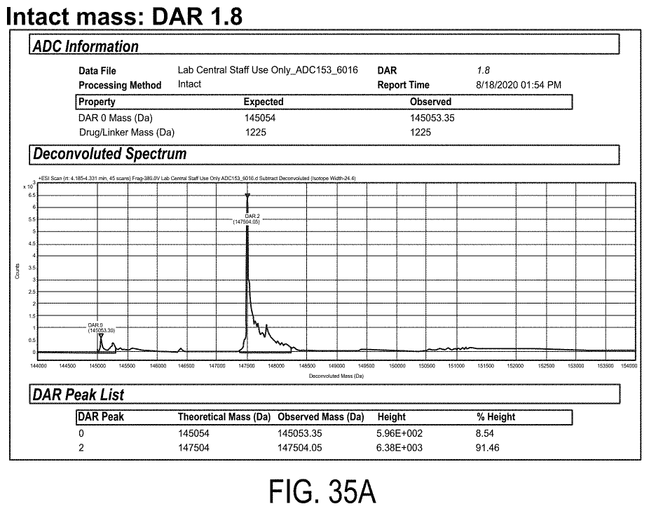

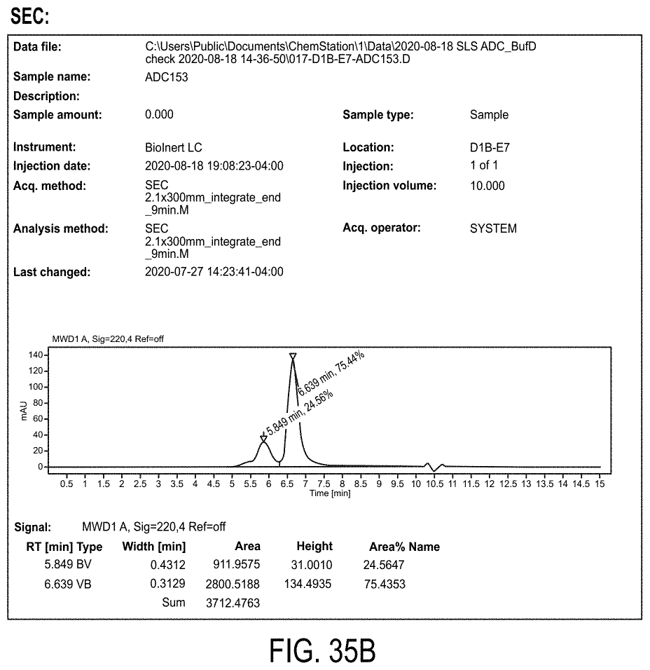

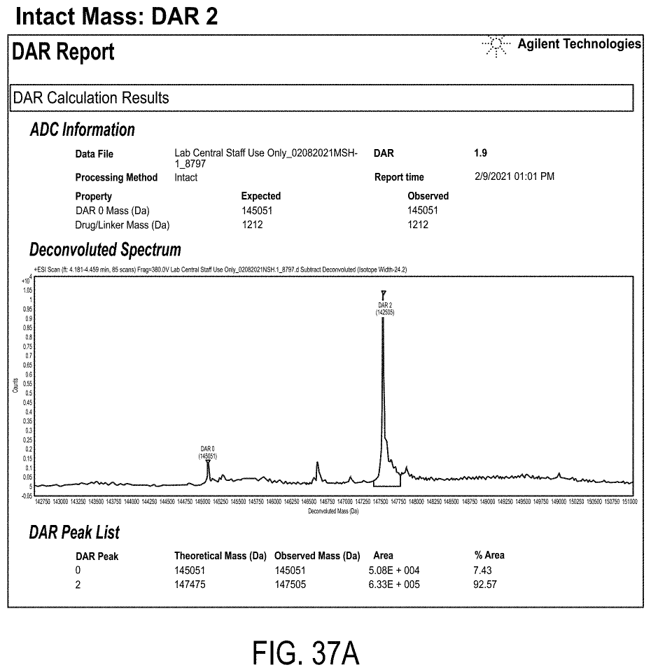

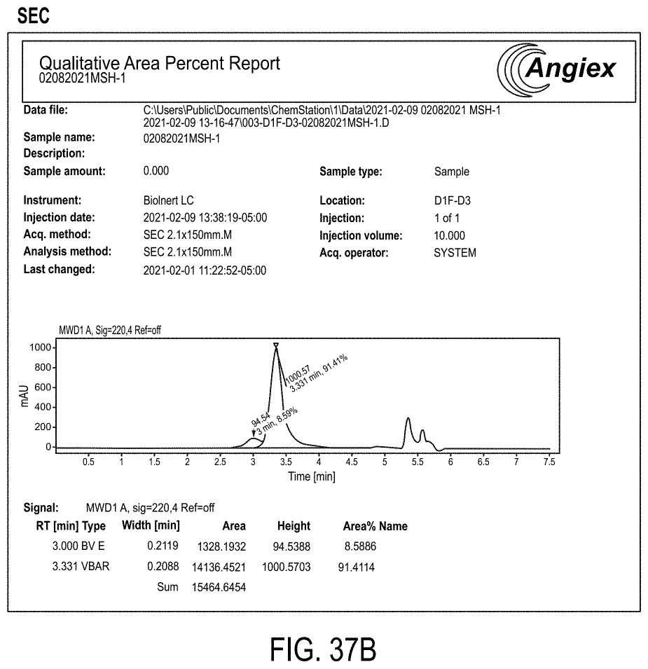

Other anti-TM4SF1 antibodies conjugated with the BRD4 degrader compound were assessed through QTOF and SEC in order to determine the DAR and any formation of aggregates. Exemplary spectra are shown in FIGS. 30A-B (DAC15), FIGS. 31A-B (DAC14), FIGS. 32A-B (DAC12), FIGS. 33A-B (DAC11), FIGS. 34A-B (DAC9), FIGS. 35A-B (DAC8), and FIGS. 37A-B (DAR of about 2.0 (Site-specific)).

An in vitro assay was carried out using an exemplary conjugate comprising A07-YTEC-degrader compound 1. The purpose of this assay was to check for efficacy of the degrader antibody conjugate in degrading Brd4, using HUVEC cells (human umbilical vein endothelial cells). At a concentration of 20 ng/mL, robust killing of the cells tested was observed.

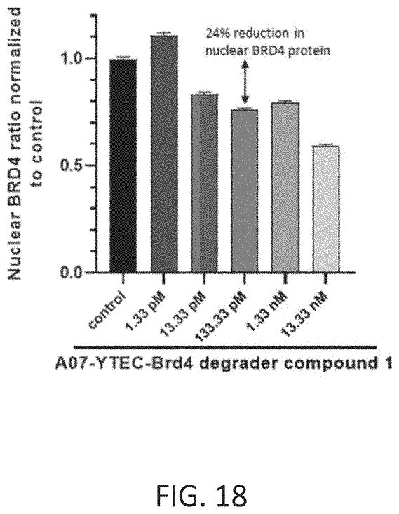

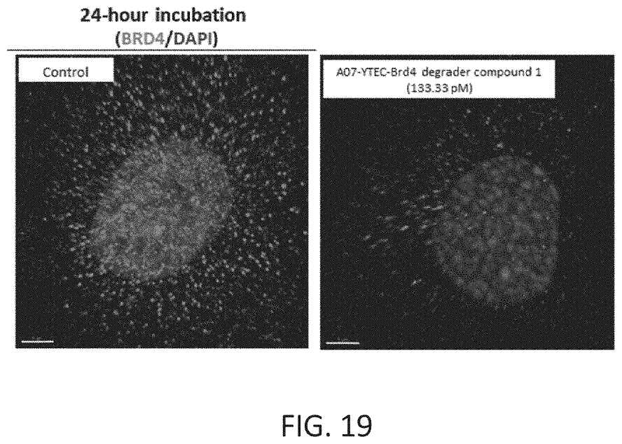

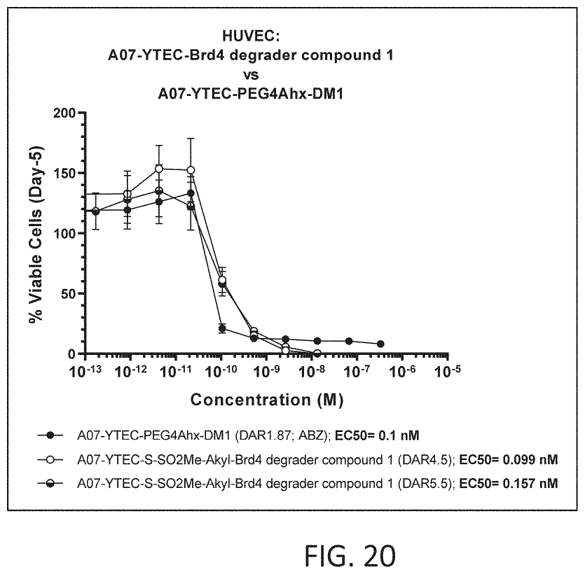

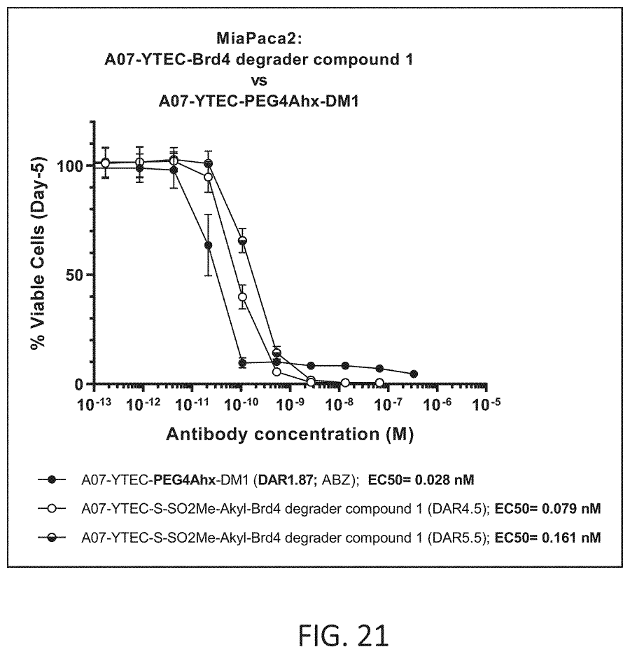

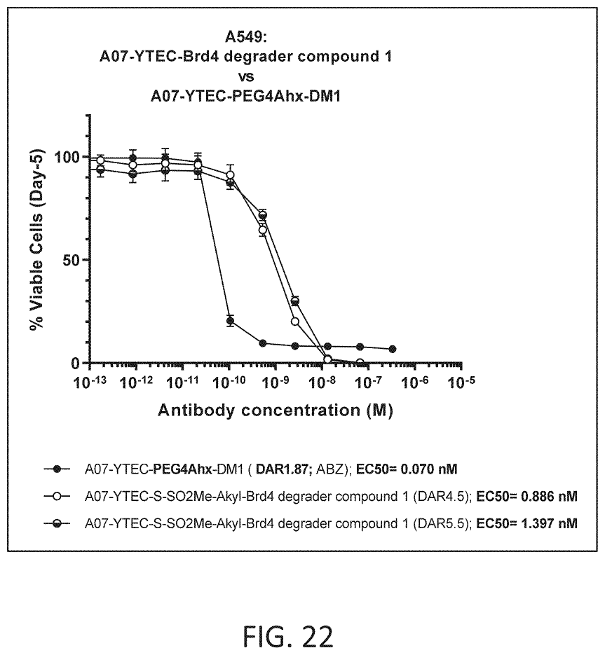

The HUVEC cells were incubated with the exemplary degrader antibody conjugate (denoted as A07-YTEC-degrader compound 1), at various concentrations. The concentrations evaluated included a control, 1.33 pM, 13.33 pM, 133.33 pM (0.13333 nM), 1.33 nM, and 13.33 nM, as shown in FIG. 18. After four hours of incubation, the ratio of nuclear Brd4 normalized to the control sample was evaluated at various molar concentrations of the exemplary antibody-degrader conjugate (A07-YTEC-degrader compound 1). The nuclear concentration of Brd4 was again evaluated after 24 hours of incubation, by confocal microscopy, and compared with a control sample (FIG. 19). After five days of incubation, EC₅₀ values were determined to evaluate the potency of A07-YTEC-degrader compound 1 (at DAR of about 5.0 and about 5.5) in killing the HUVEC cells, and further tested using MiaPaca2 (pancreatic cancer cells) and A549 cells (alveolar carcinoma cells). Additionally, the five-day killing activity of A07-YTEC anti-human antibodies conjugated to a solubilizing group was evaluated for comparison. The five-day cell killing activities are shown in FIG. 20 (HUVEC cells), FIG. 21 (MiaPaca2 cells), and FIG. 22 (A549 cells).

The results of the in vitro assays indicate robust killing of the cells tested where the exemplary antibody-degrader conjugate was introduced at a concentration of about 20 ng/ml (133.33 pM). After four hours of incubation, the ratio of nuclear Brd4 normalized to the control sample was evaluated at various molar concentrations of the exemplary antibody-degrader conjugate (A07-YTEC-degrader compound 1). The results at 1.33 pM, 13.33 pM, 133.33 pM, 1.33 nM, and 13.33 nM indicated partial degradation (20%-40%), with a 24% reduction in nuclear Brd4 protein at 133.33 pM. The reduction was greatest in the 13.33 nM concentration (40%), but none of the other tested concentrations showed a reduction exceeding 24%.

After 24 hours, Brd4 and DAPI levels in the 133.33 pM sample were again evaluated and compared to a control. The results indicated substantial (>50%) degradation in the nucleus (FIG. 19). After five days, the EC₅₀ values (for killing of HUVEC cells) were evaluated for A07-YTEC-degrader compound 1 at a DAR of 5.5 (DAC15), A07-YTEC-degrader compound 1 at a DAR of 4.5 (DAC14), and A07-YTEC-PEG4Ahx-DM1 (ABZ), which had a DAR of about 2. The results indicated ECs values of 0.157 nM for DAC15, 0.099 nM for DAC14, and 0.1 nM for ABZ, as shown in FIG. 20. ABZ, which included a maytansinoid-linker payload, was used as a positive control and as a means of comparison to show efficacy of a non-cytotoxin that produces a similar level of activity as a cytotoxic molecule.

The efficacy was also evaluated again using exemplary tumor cells. The EC₅₀ values after 5 days of incubation in both pancreatic carcinoma cells (MiaPaca2) and lung carcinoma cells (A549) were determined for exemplary DAC15 and exemplary DAC16, along with EC₅₀ values for A07-YTEC-PEG4Ahx-DM1. The results are in Table 17 and also shown in FIGS. 21 and 22. For both tumor cell lines tested, the results indicated that the A07-YTEC-degrader compound 1 at a DAR of 4.5 (DAC14) had improved EC₅₀ values, compared to that of the conjugate having a DAR of 5.5 (DAC 1). This was similar to the results of the in vitro assays conducted using the HUVEC cells.

TABLE 17
KILLING ACTIVITY (EC₅₀) FOR ENDOTHELIAL
AND EXEMPLARY TUMOR CELLS

| | | Killing Activity (EC50 nM) | | |
|---|---|---|---|---|
| | | | Tumor Cells | |
| | | Endothelial Cells | MiaPaca2 | A549 |
| | | HUVEC | (pancreatic | (lung |
| | | (umbilical vein) | carcinoma) | carcinoma) |
| A07-YTEC- | DAR 2.0 | 0.010 | 0.028 | 0.070 |
| PEG4Ahx-DM1 | | | | |
| A07-YTEC- | DAR 5.5 | 0.099 | 0.079 | 0.886 |
| degrader | DAR 4.5 | 0.157 | 0.161 | 1.397 |
| compound 1 | | | | |

The results of cell viability studies of additional exemplary DACs are shown are in Table 18. Out of the exemplary DACs tested in in vitro cell killing assays, some conjugates included murine surrogate antibodies directed to TM4SF1 (DAC16, DAC17), and rest included humanized anti-TM4SF1 antibodies as described herein (e.g., AGX-A07 with mutations in Fc regions, e.g., YTEC mutations).

TABLE 18
SUMMARY OF CELL KILLING ACTIVITY FOR EXEMPLARY DACS

| | | | | | conjugation | killing in vitro (EC₅₀ nM) | | | |
|---|---|---|---|---|---|---|---|---|---|
| No. | DAR | linker | spacer | payload | site | HUVEC | MiaPaca2 | A549 | SKOV3 |
| DAC 1 | 0.887 | S-SO₂,-Me | NA | BRD4 | N297C | | | 12.35 | |
| | | | | degrader | | | | | |
| DAC 2 | 1 | S-SO₂,-Me | NA | BRD4 | N297C | | | 4.898 | |
| | | | | degrader | | | | | |
| DAC 3 | 1.9 | S-SO₂,-Me | NA | Brd4 | N297C | 0.495 | | 6.559 | |
| | | | | Degrader | | | | | |
| DAC 4 | 1.9 | S-SO₂,-Me | NA | Brd4 | N297C | 0.423/ | 1.752 | 3.295/ | 1.191 |
| | | | | degrader | | 0.681 | | 5.223 | |
| DAC 5 | 1.9 | S-SO₂,-Me | NA | Brd4 | N297C | 1.336 | | 5.28 | 1.949 |
| | | | | degrader | | | | | |
| DAC 6 | 0.5 | S-SO₂,-Me | Alkyl | Brd4 | N297C | No | | | |
| | | | | degrader | | killing | | | |
| DAC 7 | 0.9 | S-SO₂,-Me | Alkyl | Brd4 | N297C | Not | | | |
| | | | | degrader | | tested | | | |
| DAC 8 | 2.27 | S-SO₂,-Me | Alkyl | Brd4 | N297C & | 1.55 | | | |
| | | | | degrader | Hinge | | | | |
| DAC 9 | 1.9 | S-SO₂,-Me | Alkyl | Brd4 | N297C | 3.48 | 14.16 | | 6.670 |
| | | | | degrader | | | | | |
| DAC 10 | 1.9 | S-SO₂,-Me | Alkyl | Brd4 | N297C & | | | | |
| | | | | degrader | Hinge | | | | |
| DAC 11 | 1.6 | S-SO₂,-Me | alkyl | Brd4 | N297C | | 0.82 | | |
| | | | | degrader | | | | | |
| DAC 12 | 1.0 | S-SO₂,-Me | Alkyl | Brd4 | N297C | | 5.49 | | |
| | | | | degrader | | | | | |
| DAC 13 | 0.4 | S-SO₂,-Me | Alkyl | Brd4 | N297C | 36.1 | | | |
| | | | | degrader | and Hinge | | | | |
| DAC 14 | 4.5 | S-SO₂,-Me | alkyl | Brd4 | N297C & | 0.099 | 0.079 | | |
| | | | | degrader | Hinge | | | | |
| DAC 15 | 5.5 | S-SO₂,-Me | Alkyl | Brd4 | N297C & | 0.157/ | 0.161 | | |
| | | | | degrader | Hinge | 0.115 | | | |
| DAC 16 | 1.8 | S-SO₂,-Me | Alkyl | Brd4 | N297C | 4.03 | 18.84 | | 6.24 |
| | | | | degrader | | | | | |
| DAC 17 | 2.62 | S-SO₂,-Me | Alkyl | Brd4 | N297C | 2.18 | | | |
| | | | | degrader | & Hinge | | | | |
| DAC 18 | 0.5 | S-SO₂,-Me | Alkyl | Brd4 | | | | | |
| | | | | degrader | | | | | |

| | | |
|---|---|---|
| Brd4 degrader | S-SO₂-Me-Alkyl- | 3.78 |
| free linker | Brd4 degrader (the | |
| payload | degrader having | |
| | the structure as | |
| | shown in FIG. 27) | |

### Example 3. Brd4 Degradation Assays

Cell killing assay. Cells (HUVEC, MiaPaca2, and A549) are seeded in a density of 10,000 cells/mL in 100 μl/well in a 96 well Flat-Bottom black microplate (Corning, part #3904) in Assay Medium (EGM2 complete medium for HUVEC; RPMI/10% FBS for MiaPaca2 and A549). Cells are cultured for overnight at 37° C. degrees. On day 2, exemplary anti-TM4SF1 degrader conjugates containing Brd4 degrader compound 1 are serially diluted 5-fold in the culture media. and transferred 100 μl of the diluted compounds to the cell plates. The final top concentration of the test exemplary anti-TM4SF1 degrader conjugates in the cell plates is 333.335 nM and the lowest concentration is 0.0043 nM. Cell plates are incubated at 37° C. for 5 days. 10 μl PrestoBlue HS cell viability reagent (ThermoFisher cat #P50201) is added to each well and incubated for 1 hour in CO₂ incubator before reading the absorbance at 570 nm/600 nm excitation and emission through a plate reader (Varioskan™ LUX multimode microplate reader).

BRD4 degradation assay via confocal fluorescence imaging. Immunofluorescence staining of Brd4 is carried out using a Cellvis glass bottom plate (P24-1.5P) as follows. The well supernatant is aspirated from the wells and 300 μL/well of rabbit mAb Anti-Brd4 [BL-149-2H5] (Bethyl Laboratories, A700-004) diluted 1:500 in PBS/0.1% Triton is dispensed. Samples are incubated for 2 hours at room temperature. Samples are washed 4 times with 100 μL/well of PBS. 300 μL/well of secondary antibody solution [Donkey anti-Rabbit IgG (H+L) Highly Cross-Absorbed Secondary Antibody, Alexa Fluor 647 (ThermoFisher Catalog #A-31573) and DAPI in PBS/0.1% Triton] are dispensed into each well. Fluorescence images of Brd4 is captured using an Olympus confocal microscope (FV3000). Nuclear Brd4 fluorescence signal intensities are calculated via ImageJ and data are presented as nuclear Brd4 ratio normalized to a control.

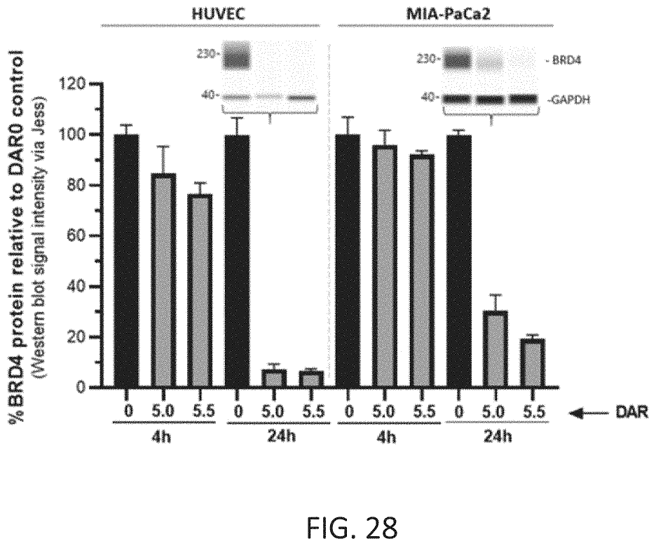

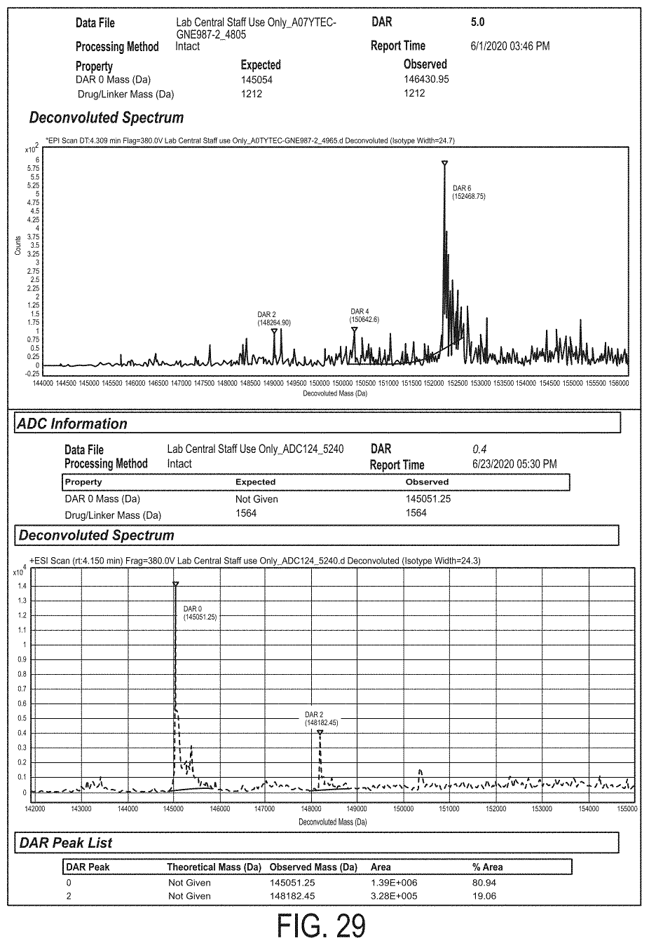

As shown in FIG. 28, BRD4 degradation was also evaluated by Western Blot by treating HUVEC or MiaPaca2 cells with an exemplary anti-TM4S1 DACs. BRD4 degradation was assessed 4 hours or 24 hours after incubation of the degrader antibody conjugate. The exemplary degrader antibody conjugates (which included the Brd4 degrader structure as shown in FIG. 27) were observed to be highly effective in BRD4 degradation in both HUVEC endothelial cells and MiaPaca2 tumor cells in vitro. Measurable BRD4 protein degradation was seen early as 4 hours after the treatment with the exemplary conjugates, with up to 90% (in HUVEC) and 70-80% (in MiaPaca2) degradation at 24 hours.

### Example 4: Site Specific Labeling of Degrader Compounds

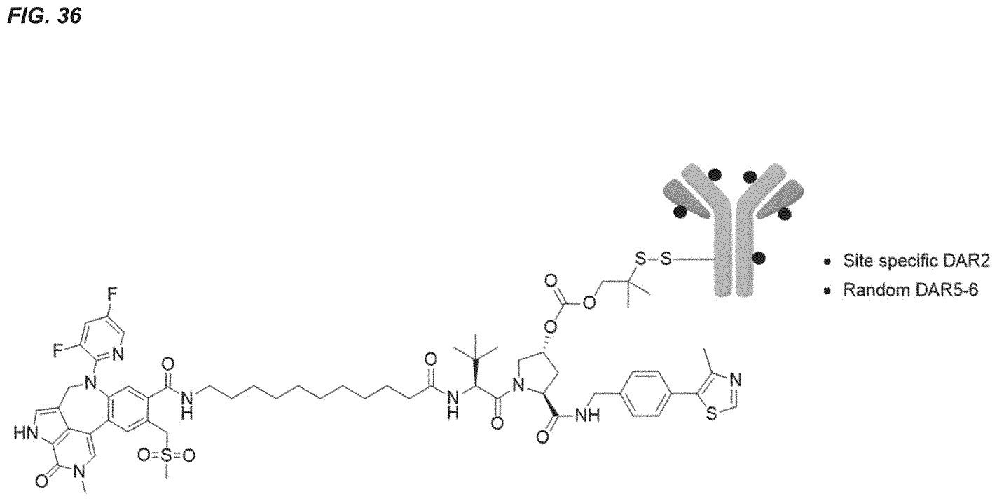

In some cases, site-specific conjugation of the degrader compound to the anti-TM4SF1 antibody can be performed as an alternative to semi-random conjugation described in the previous examples. Here were describe site-specific conjugation of the anti-TM4SF1 antibody with the BRD4 degrader compound. FIG. 36 shows a schematic of an exemplary DAC (DAC4) generated through site-specific conjugation in the method disclosed herein.

### Site Specific Conjugation and Synthesis

For this process, a 10 mM stock solution of BRD4 degrader compound 1 was solubilized in DMSO. A 5 mM DTT solution in PBS 7.4 was prepared. The exemplary anti-TM4SF1 antibody (14.66 mg/mL) was prepared. To the 10 mg the antibody at 14.66 mg/mL, 672.38 μL for 10 mg in 5 mL eppendorf was added 1.267 mL of 5 mM EDTA, 50 mM Tris 8.5 to make final concentration of 5 mg/ml; ii) The anti-TM4SF1 antibody was reduced for 2 hours at 37° C. on a thermomixer by adding 4.5 eq. of DTT (from 5 mM DTT stock, 60.03 μL); iii) After reducing the anti-TM4SF1 antibody for 2 hours, the excess DTT was removed using 4 ml, 50k MWCO amicon filtration columns and washing the column 3 times; iv) The resulting anti-TM4SF1 antibody was diluted using appropriate volume of 5 mM EDTA, 50 mM Tris 8.5; v) 10 eq. of BRD4 Degrader Compound 1 (66.7 μL from 10 mM stock) degrader in DMSO was added slowly followed by addition of 25-30% ethylene glycol to achieve a final concentration of 5 mg/mL; vi) The eppendorf was incubated on thermomixer at room temperature overnight; vii) The following days, ethylene glycol from the reaction mixture was removed using a PD-10 column (conditions: 2.5 ml sample load and 3.5 ml elution using 5 mM EDTA, 50 mM Tris 8.5); viii) anti-TM4SF1 antibody-BRD4 Degrader Compound 1 was then buffered exchanged into 10 mM arginine, PBS 7.4 buffer using 4 ml, 50k MWCO amicon filtration columns; ix) anti-TM4SF1 antibody-BRD4 Degrader Compound 1, drug to antibody ratio (DAR) was analyzed by Q-TOF Intact mass spec analysis showing DAR 2 species; x) 10 mg of anti-TM4SF1 antibody yielded 7.5 mg of anti-TM4SF1 antibody-BRD4 Degrader Compound 1 (75% yield). The exemplary conjugate was characterized to assess the degrader to antibody ration (DAR) and further characterized by size exclusion chromatography, Q-ToF intact liquid chromatography mass spectroscopy. To measure DAR values for each respective exemplary degrader antibody conjugates, deconvoluted spectrums were generated (FIG. 37A). The deconvoluted spectrum may be used to calculate or confirm DAR values based on the total ion chromatograph (TIC) peaks. Additionally, a chromatogram was generated for both (FIG. 37B) using a size exclusion column (21.2 mm by 300 mm) and phosphate buffered saline pH 7.4 as the mobile phase. As seen in FIG. 37A, the DAR value for the exemplary conjugate generated through site specific conjugation was about 2.

### BRD4 Degradation

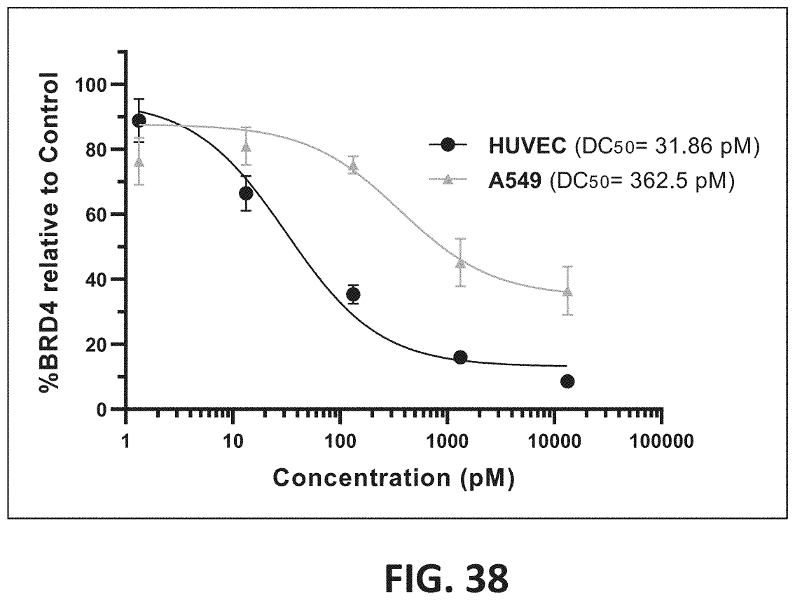

BRD4 degradation was assessed using the exemplary conjugate DAC4 generated through site-specific conjugation strategies using the protocol described above. HUVEC and A549 cells were assessed using this these DACs in order to determine the DC₅₀ (DC₅₀: 50% BRD4 protein degradation in 24 hours). The exemplary conjugate was shown to have a DC₅₀ of 31.86 pM in HUVEC cells and a DC₅₀ of 362.5 pM in A549 cells (FIG. 38).

### In Vitro Cell Proliferation Assay

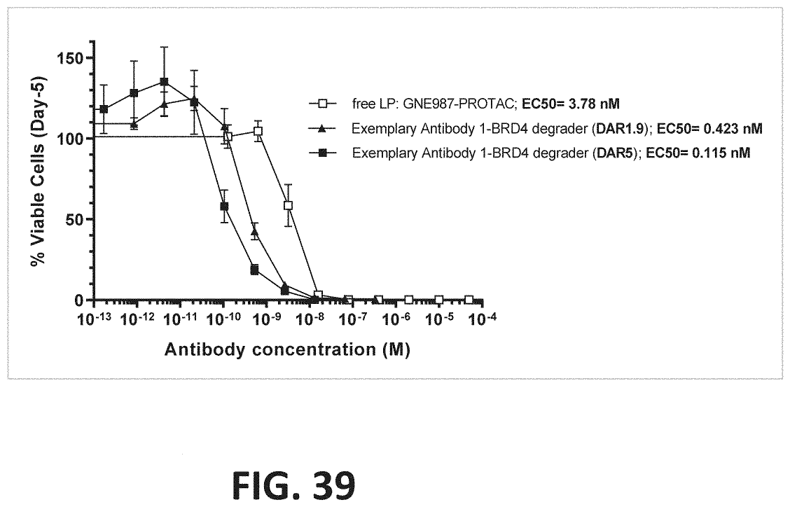

An in vitro assay to assess the efficacy of the exemplary conjugate DAC4 was carried out using the cell viability assay previously described. In FIG. 39, exemplary conjugate DAC4 was found to have an EC₅₀ of 0.423 nM in endothelial cell HUVEC. Same antibody showed an EC₅₀ of 3-5 nM in A549, 1.191 nM in SKOV3 tumor cells (TABLE 18). In addition, the exemplary conjugate DAC4, with a DAR of about 2, had improved properties pertaining to developability, including, better solubility compared to a conjugate that had a DAR of about 5 but otherwise identical. In vivo tumor regression

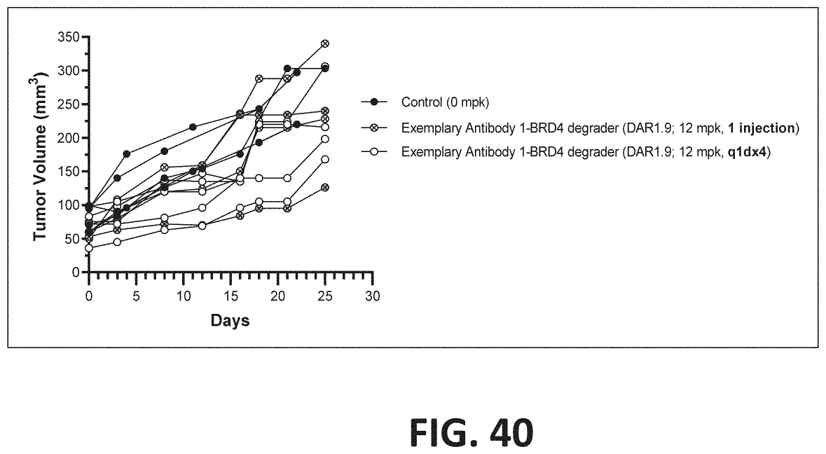

In this study, the ability for the exemplary conjugate DAC4 were assessed in their ability to affect A549 xenograft tumor growth in a mouse model. In FIG. 40, two treatment regimens consisting of 1 injection or 1 injection every day for four days (q1dx4) was assessed. The treatments were administered as 12 mg/kg to the animal subject.

Tumor volume was measured at different periods after dosing with exemplary conjugates as shown in FIG. 40. In comparison to no drug injected control, the exemplary conjugate DAC4 showed promising tumor regression, in particular with q1dx4 injection scheme.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-125. (canceled)

126. A heterobifunctional compound that comprises:
(a) an anti-TM4SF1 antibody or an antigen binding fragment thereof;
(b) a degrader molecule; and
(c) a first linker L1 between the anti-TM4SF1 antibody and the degrader molecule wherein the degrader molecule comprises:
(i) a single-ligand molecule that directly interacts with a target protein to induce degradation of the target protein; or
(ii) a chimeric degrader molecule comprising a specific and nongenetic inhibitor of apoptosis protein (IAP)-dependent protein eraser (SNIPER); or
(iii) a single-ligand molecule that interacts with an E3 ubiquitin ligases to modulate substrate selectivity of the E3 ubiquitin ligase; or
(iv) a ubiquitin E3 ligase binding group (E3LB) and a target protein binding group (PB).

127. The heterobifunctional compound of claim 126, wherein the degrader molecule comprises the E3LB, the PB, and a second linker L2 between the E3LB and the PB.

128. The heterobifunctional compound of claim 127, wherein the PB comprises a peptide or a small molecule that binds to a protein selected from the group consisting of an intracellular protein, an extracellular protein, a cell surface protein, a disease-causing or a disease-related protein, a TNF-receptor-associated death-domain protein (TRADD), receptor interacting protein (RIP), TNF-receptor-associated factor 2 (TRAF2), IK-alpha, IK-beta, IK-epsilon, PLCγ, IQGAP1, Rac1, MEK1/2, ERK1/2, PI4K230, Akt1/2/3, Hsp90, GSK-3β, an HDAC protein, FoxO1, HDAC6, DP-1, E2F, ABL, AMPK, BRK, BRSK I, BRSK2, BTK, CAMKK1, CAMKK alpha, CAMKK beta, Rb, Suv39HI, SCF, p19INK4D, GSK-3, pi 8 INK4, myc, cyclin E, CDK2, CDK9, CDG4/6, Cycline D, p16 INK4A, cdc25A, BMI1, SCF, Akt, CHK1/2, C 1 delta, CK1 gamma, C 2, CLK2, CSK, DDR2, DYRK1A/2/3, EF2K, EPH-A2/A4/B/B2/B3/B4, EIF2A 3, Smad2, Smad3, Smad4, Smad7, p53, p21 Cip1, PAX, Fyn, CAS, C3G, SOS, Tal, Raptor, RACK-1, CRK, Rap1, Rac, KRas, NRas, HRas, GRB2, FAK, PI3K, spred, Spry, mTOR, MPK, LKB1, PAK 1/2/4/5/6, PDGFRA, PYK2, Src, SRPK1, PLC, PKC, PKA, PKB alpha/beta, PKC alpha/gamma/zeta, PKD, PLK1, PRAK, PRK2, WAVE-2, TSC2, DAPK1, BAD, IMP, C-TAK1, TAK1, TAO1, TBK1, TESK1, TGFBR1, TIE2, TLK1, TrkA, TSSK1, TTBK1/2, TTK, Tpl2/cot1, MEK1, MEK2, PLDL Erk1, Erk2, Erk5, Erk8, p90RSK, PEA-15, SRF, p27 KIP1, TIF 1a, HMGN1, ER81, MKP-3, c-Fos, FGF-R1, GCK, GSK3 beta, HER4, HIPK1/2/3/, IGF-IR, cdc25, UBF, LAMTOR2, Stat1, StaO, CREB, JAK, Src, PTEN, NF-kappaB, HECTH9, Bax, HSP70, HSP90, Apaf-1, Cyto c, BCL-2, Bcl-xL, Smac, XIAP, Caspase-9, Caspase-3, Caspase-6, Caspase-7, CDC37, TAB, IKK, TRADD, TRAF2, R1P1, FLIP, TAK1, JNK1/2/3, Lck, A-Raf, B-Raf, C-Raf, MOS, MLK1/3, MN 1/2, MSK1, MST2/3/4, MPSK1, MEKK1, ME K4, MEL, ASK1, MINK1, MKK 1/2/3/4/6/7, NE 2a/6/7, NUAK1, OSR1, SAP, STK33, Syk, Lyn, PDK1, PHK, PIM 1/2/3, Ataxin-1, mTORC1, MDM2, p21 Waf1, Cyclin D1, Lamin A, Tpl2, Myc, catenin, Wnt, IKK-beta, IKK-gamma, IKK-alpha, IKK-epsilon, ELK, p65RelA, IRAKI, IRA 2, IRAK4, IRR, FADD, TRAF6, TRAF3, MKK3, MKK6, ROCK2, RSK1/2, SGK 1, SmMLCK, SIK2/3, ULK1/2, VEGFR1, WNK 1, YES1, ZAP70, MAP4K3, MAP4K5, MAPK1b, MAPKAP-K2 K3, p38 alpha/beta/delta/gamma MAPK, Aurora A, Aurora B, Aurora C, MCAK, Clip, MAPKAPK, FAK, MARK 1/2/3/4, Muc1, SHC, CXCR4, Gap-1, Myc, beta-catenin/TCF, Cbl, BRM, Mcl-1, BRD2, BRD3, BRD4, BRDt, BRD7, BRD9, AR, RAS, ErbB3, EGFR, IRE1, HPK1, RIPK2, PDE4, ERRα, FKBP12, brd9, c-Met, Sirt1, Sirt2, Sirt3, Sirt4, Sirt5, Sirt6, Sirt7, flt3, BTK. ALK, TRIM24, GSPT1, IKZF1 (Ikaros), IKZF3 (Aiolos), CK1-alpha, TACC3, p85, MetAP-2, DHFR, BET, CRABP-I/II, HIF1-alpha, PCAF, GCN5L2 (GCN5), SMARCA2, SMARCA4, PBRM1, HER2, Akt, Hsp90, HDAC1, HDAC2, HDAC3, HDAC8, HDAC4, HDAC5, HDAC6, HDAC7, HDAC9, HDAC10, HDAC11, DNMT1, DNMT3a, DNMT3b, MeCP2, MBD1, MBD2, MBD4, KAISO (ZBTB33), ZBTB4, ZBTB38, UHRF1, UHRF2, TET1, TET2, TET3, HATI, HTATIP (TIP60), MYST1 (MOF), MYST2 (HBO1), MYST3 (MOZ), MYST4 (MORF), P300 (EP300, KAT3B), CBP (CREBBP, KAT3A), NCOA1 (SRC1), NCOA2 (TIF2), NCOA3 (AIB1, ACTR), ATF-2 (CREB2, CREBP1), TFIIIC, TAF1 (TAFII250), CLOCK (KIAA0334), CIITA (MHC2TA), MGEA5 (NCOAT), CDY, KMT1A, KMT1B, KMT1C, KMT1E, KMT2A, KMT2B, KMT2C, KMT2D, KMT2E, KMT2F, EZH1, EZH2, KMT3A, WHSC1, WHSC1L1, PRDM1, PRDM2, PRDM3, PRDM4, PRDM5, PRDM9, PRDM14, PRDM16, KMT3C, KMT3E, SMYD4, DOT1L, SET8, SUV4-20H2, SetD6, SET7/9, PRMT1, PRMT2, PRMT4, PRMT5, PRMT6, PRMT7, PRMT8, PRMT9, HP1, Chd1, WDR5, BPTF, L3MBTL1, ING2, BHC80, JMJD2A, KDM1A, KDM1B, KDM2A, KDM2B, KDM3A, KDM3C, KDM4A, KDM4B, KDM4C, KDM4D, KDM5A, KDM5B, KDM5C, KDM5D, JARID2, KDM6A, KDM6B, KDM6C, KDM7A, KDM7C, KDM7B, JMJD5, RSBN1, JMJD6, PADI4, K-Ras, PI3K, BTK, B-Raf, ERK, MEK, P65 (RELA), p50 (NFKB1) of NFkB, Ras, Raf, eNOS, a Smad family protein, Smad2/3/4, and ERalpha, variants thereof, mutants thereof, splice variants thereof, indels thereof, and fusions thereof.

128. The heterobifunctional compound of claim 127, wherein the PB comprises a PLCγ inhibitor; an IQGAP1 inhibitor; a Rac1 inhibitor; an MEK1/2 inhibitor; an ERK1/2 inhibitor; a PI4K230 inhibitor; an Akt1 inhibitor; an Akt2 inhibitor; an Akt3 inhibitor; a GSK-3β inhibitor; an HDAC6a inhibitor; a Heat Shock Protein 90 (HSP90) inhibitor; a kinase inhibitor; a Phosphatase inhibitor; a MDM2 inhibitor; a compounds targeting Human BET Bromodomain-containing protein; a HDAC inhibitor; a human lysine methyltransferase inhibitor; an angiogenesis inhibitor; an immunosuppressive compound; a compound targeting the aryl hydrocarbon receptor (AHR), a REF receptor kinase, a FKBP, an Androgen Receptor (AR), an Estrogen receptor (ER), a Thyroid Hormone Receptor, a HIV Protease, a HIV Integrase, a HCV Protease, an Acyl-protein Thioesterase-1 (APT), an Acyl-protein Thioesterase-2 (APT2), a pharmaceutically acceptable salt of any thereof, an enantiomer of any thereof, a solvate of any thereof, or a polymorph of any thereof.

129. The heterobifunctional compound of claim 127, wherein the first linker L1 comprises: a MC (6-maleimidocaproyl), a MCC (a maleimidomethyl cyclohexane-1-carboxylate), a MP (maleimidopropanoyl), a val-cit (valine-citrulline), a val-ala (valine-alanine), an ala-phe (alanine-phenylalanine), a PAB (p-aminobenzyloxycarbonyl), a SPP (N-Succinimidyl 4-(2-pyridylthio) pentanoate), 2,5-dioxopyrrolidin-1-yl 4-(pyridin-2-ylthio)hexanoate, 2,5-dioxopyrrolidin-1-yl 5-methyl-4-(pyridin-2-ylthio)hexanoate, 2,5-dioxopyrrolidin-1-yl 5-methyl-4-(pyridin-2-ylthio)heptanoate, 2,5-dioxopyrrolidin-1-yl 5-ethyl-4-(pyridin-2-ylthio)heptanoate, 2,5-dioxopyrrolidin-1-yl 4-cyclopropyl-4-(pyridin-2-ylthio)butanoate, 2,5-dioxopyrrolidin-1-yl 4-cyclobutyl-4-(pyridin-2-ylthio)butanoate, 2,5-dioxopyrrolidin-1-yl 4-cyclopentyl-4-(pyridin-2-ylthio)butanoate, 2,5-dioxopyrrolidin-1-yl 4-cyclohexyl-4-(pyridin-2-ylthio)butanoate, a SMCC (N-Succinimidyl 4-(N-maleimidomethyl)cyclohexane-1 carboxylate), a SIAB (N-Succinimidyl (4-iodo-acetyl)aminobenzoate), a disulfide linker, a glutathione cleavable linker,
a formula of -Str-(PM)-Sp, wherein Str is a stretcher unit covalently attached to Ab; Sp is a bond or spacer unit covalently attached to a degrader moiety; and PM is a non-peptide chemical moiety selected from the group consisting of: 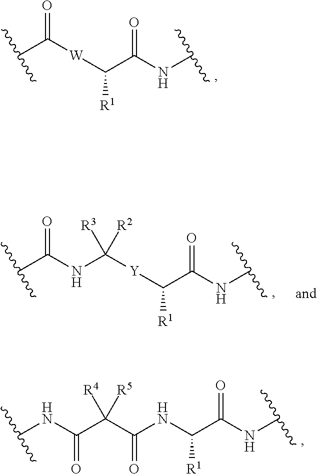
wherein W is —NH-heterocycloalkyl- or -heterocycloalkyl-; Y is heteroarylene, arylene, —C(O)(C₁-C₆alkylene)-, —(C₁-C₆ alkylene)-NH—, —(C₁-C₆ alkylene)-NH—CH₂—, —(C₁-C₆ alkylene)-N—(CH₃)—, —(C₁-C₆ alkenyl)- or —(C₁-C₆ alkylenyl)-; each R¹ is independently C₁-C₁₀ alkyl, C₁-C₁₀ alkenyl, —(C₁-C₁₀ alkyl)NHC(NH)NH₂ or —(C₁-C₁₀alkyl)NHC(O)NH₂; R³ and R² are each independently H, C₁-C₁₀alkyl, C₁-C₁₀alkenyl, arylalkyl or heteroarylalkyl, or R³ and R² together form a C₃-C₇ cycloalkyl; and R⁴ and R⁵ are each independently C₁-C₁₀ alkyl, C₁-C10 alkenyl, arylalkyl, heteroarylalkyl, (C1-C₁₀alkyl)OCH₂—, or R⁴ and R⁵ form a C₃-C₇ cycloalkyl ring,
or a formula of 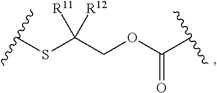
wherein, R¹¹ and R¹² are independently selected from H and C₁-C₆ alkyl, or R¹¹ and R¹² form a 3, 4, 5, or 6-membered cycloalkyl or heterocyclyl group.

130. The heterobifunctional compound of claim 127, wherein the first linker L1 is derived from a cross-linking reagent, wherein the cross-linking reagent comprises N-succinimidyl-3-(2-pyridyldithio)propionate (SPDP), 2,5-dioxopyrrolidin-1-yl 3-cyclopropyl-3-(pyridin-2-yldisulfaneyl)propanoate, 2,5-dioxopyrrolidin-1-yl 3-cyclobutyl-3-(pyridin-2-yldisulfaneyl)propanoate, N-succinimidyl 4-(2-pyridyldithio)pentanoate (SPP), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl 4-(2-pyridyldithio)butanoate (SPDB), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC), or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

131. The heterobifunctional compound of claim 127, wherein the second linker L2 comprises an alkyl linker comprising (CH₂)ₙ, wherein n is an integer 1-12; a polyethylene glycol (PEG) linker comprising —(O—CH₂—CH₂)ₘ—O—, wherein m is 1-4.

132. The heterobifunctional compound of claim 126, wherein the anti-TM4SF1 antibody or the antigen binding fragment thereof comprising a modified IgG Fc region, wherein the modified IgG Fc region comprises one or more substitutions relative to a wild-type IgG Fc region, wherein the wild-type IgG Fc region is a wild-type IgG1, IgG2, IgG3, or IgG4 Fc region, wherein the modified IgG Fc region comprises an IgG1 Fc region comprising mutation at one or more positions selected from the group consisting of E233, L234, L235, G237, M252, S254, T250, T256, D265, N297, K322, P331, M428, and N434 of the wild-type IgG1 Fc region; as numbered by the EU index as set forth in Kabat.

133. The heterobifunctional compound of claim 132, wherein the IgG1 Fc region comprises one or more mutations of N297C, E233P, L234A, L235A, G237A, M252Y, S254T, T256E, M428L, N434S OR N434A, T250Q, D265A, K322A, P331G, or M428L.

134. The heterobifunctional compound of claim 132, wherein the IgG1 Fc region comprises:
(a) T250Q and M428L; or
(b) T250Q and M428L; or
(c) L234A, L235A, and G237A; or
(d) L234A, L235A, G237A, and P331G; or
(e) L234A, L235A, G237A, N297C, and P331G; or
(d) E233P, L234A, L235A, G237A, and P331G; or
(f) E233P, L234A, L235A, G237A, and N297C; or
(g) L234A, L235A, G237A, N297C, K322A, and P331G; or
(h) E233P, L234A, L235A, G237A, D265A, N297C, K322A, and P331G; or
(i) E233P and D265A; or
(j) M252Y, S254T, and T256E; or
(k) M252Y, S254T, T256E, and N297C; or
(l) a combination thereof.

135. The heterobifunctional compound of claim 132, wherein the IgG1 Fc region comprises an amino acid sequence selected from the group consisting of SEQ ID Nos. 87-88, 135-145, and 151-153.

136. The heterobifunctional compound of claim 126, wherein the anti-TM4SF1 antibody or the antigen-binding fragment thereof comprises:
(a) a heavy chain comprising a CDR3 domain comprising an amino acid sequence that has at least 75% identity to a sequence selected from the group consisting of SEQ ID NO: 8, 20, 32, 44, 56, 68, 80, 96, 118, 119, 120, or 121; a CDR2 domain comprising an amino acid sequence that has at least 75% identity to a sequence selected from the group consisting of SEQ ID NO: 7, 19, 31, 43, 55, 67, 79, 95, 116, or 117; and a CDR1 domain comprising an amino acid sequence that has at least 75% identity to a sequence selected from the group consisting of SEQ ID NO: 6, 18, 30, 42, 54, 66, 78, 94, or 115; and
(b) a light chain comprising a CDR3 domain comprising an amino acid sequence that has at least 75% identity to a sequence selected from the group consisting of SEQ ID NO: 14, 26, 38, 50, 62, 74, 86, 110, 111, or 129; a CDR2 domain comprising an amino acid sequence that has at least 75% identity to a sequence selected from the group consisting of SEQ ID NO: 13, 25, 37, 49, 61, 73, 85, 109, or 128; and a CDR1 domain comprising an amino acid sequence that has at least 75% identity to a sequence selected from the group consisting of SEQ ID NO: 12, 24, 36, 48, 60, 72, 84, 107, 108, 124, 125, 126, or 127.

137. The heterobifunctional compound of claim 136, wherein the heavy chain comprises an amino acid sequence that has at least 75% identity to SEQ ID NO: 3, 15, 27, 39, 51, 63, 75, 90, 92, 112, 114, 130, or 132, and a light chain comprises an amino acid sequence that has at least 75% identity to SEQ ID NO: 9, 21, 33, 45, 57, 69, 81, 97, 99, 101, 122, 131, or 133.

138. The heterobifunctional compound of claim 137, wherein the heavy chain comprises an amino acid sequence as set forth in any one of: SEQ ID NO: 3, 15, 27, 39, 51, 63, 75, 90, 92, 112, 114, 130, or 132, and a light chain comprises an amino acid sequence as set forth in any one of: SEQ ID NO: 9, 21, 33, 45, 57, 69, 81, 97, 99, 101, 122, 131, or 133.

139. The heterobifunctional compound of claim 126, wherein the degrader molecule comprises a compound having a structure selected from the group consisting of:

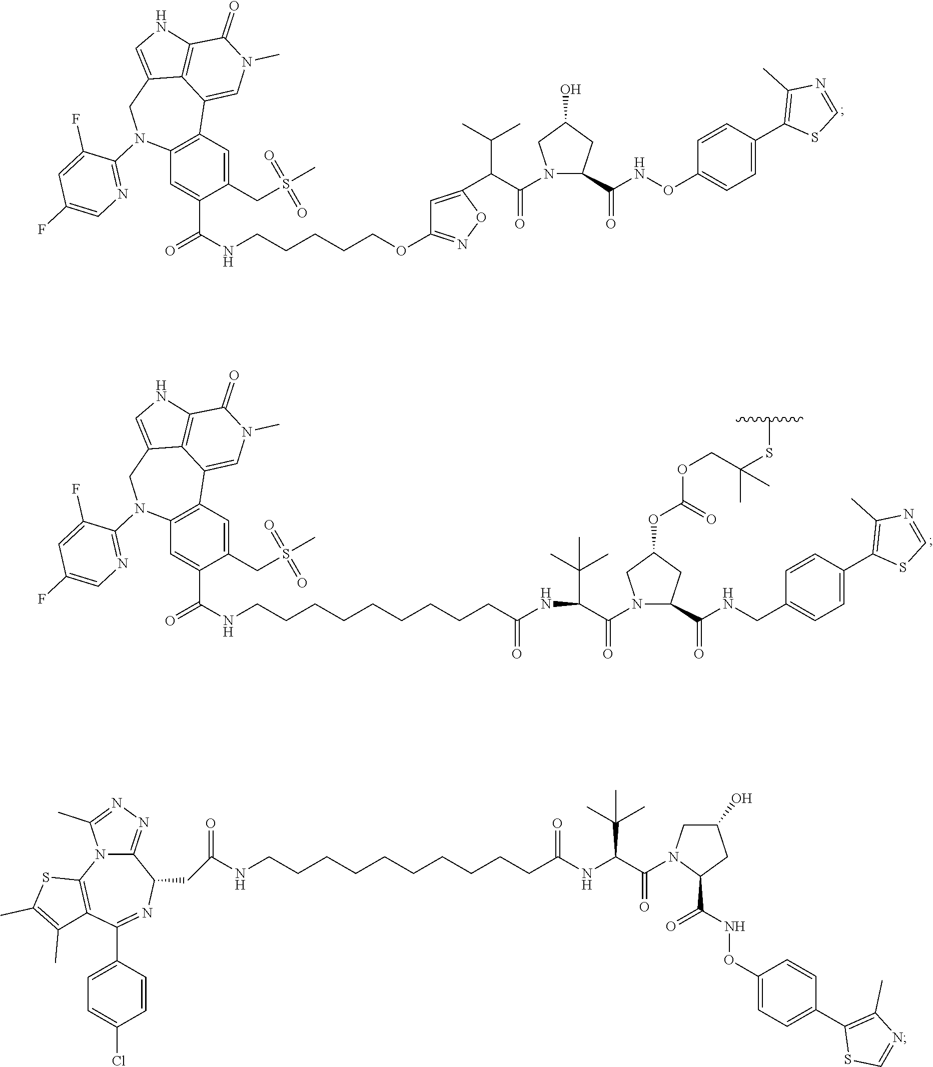 [SMILES: Cc1ncsc1-c1ccc(ONC(=O)[C@@H]2C[C@@H](O)CN2C(=O)C(c2cc(OCCCCCNC(=O)c3cc4c(cc3CS(C)(=O)=O)-c3cn(C)c(=O)c5[nH]cc(c35)CN4c3ncc(F)cc3F)no2)C(C)C)cc1 ; *SC(C)(C)COC(=O)O[C@@H]1C[C@@H](C(=O)NCc2ccc(-c3scnc3C)cc2)N(C(=O)[C@@H](NC(=O)CCCCCCCCCNC(=O)c2cc3c(cc2CS(C)(=O)=O)-c2cn(C)c(=O)c4[nH]cc(c24)CN3c2ncc(F)cc2F)C(C)(C)C)C1 ; Cc1ncsc1-c1ccc(ONC(=O)[C@@H]2C[C@@H](O)CN2C(=O)[C@@H](NC(=O)CCCCCCCCCCNC(=O)C[C@@H]2N=C(c3ccc(Cl)cc3)c3c(sc(C)c3C)-n3c(C)nnc32)C(C)(C)C)cc1]

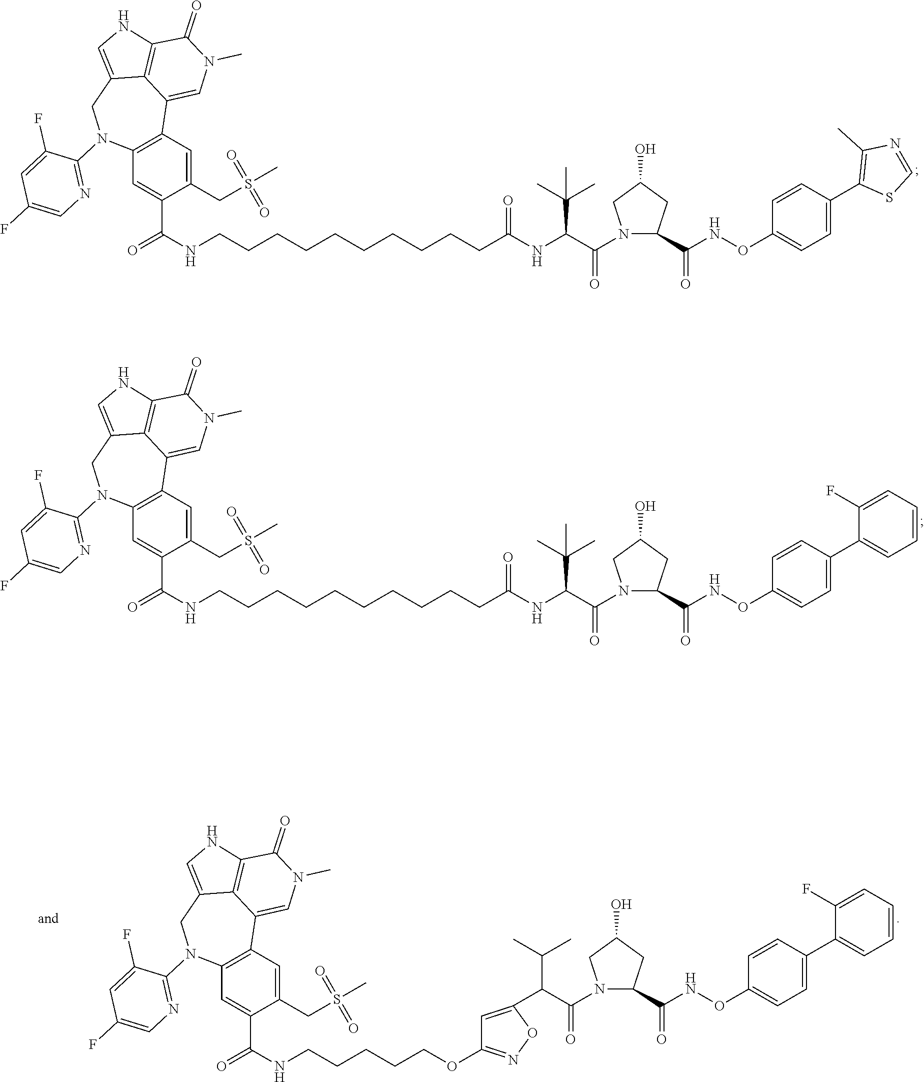 [SMILES: Cc1ncsc1-c1ccc(ONC(=O)[C@@H]2C[C@@H](O)CN2C(=O)[C@@H](NC(=O)CCCCCCCCCCNC(=O)c2cc3c(cc2CS(C)(=O)=O)-c2cn(C)c(=O)c4[nH]cc(c24)CN3c2ncc(F)cc2F)C(C)(C)C)cc1 ; Cn1cc2c3c(c[nH]c3c1=O)CN(c1ncc(F)cc1F)c1cc(C(=O)NCCCCCCCCCCC(=O)N[C@H](C(=O)N3C[C@H](O)C[C@H]3C(=O)NOc3ccc(-c4ccccc4F)cc3)C(C)(C)C)c(CS(C)(=O)=O)cc1-2 ; CC(C)C(C(=O)N1C[C@H](O)C[C@H]1C(=O)NOc1ccc(-c2ccccc2F)cc1)c1cc(OCCCCCNC(=O)c2cc3c(cc2CS(C)(=O)=O)-c2cn(C)c(=O)c4[nH]cc(c24)CN3c2ncc(F)cc2F)no1]

140. A heterobifunctional compound that comprises:
(a) an anti-TM4SF 1 antibody or an antigen binding fragment thereof;
(b) a degrader molecule, wherein the degrader molecule comprises the following structure:

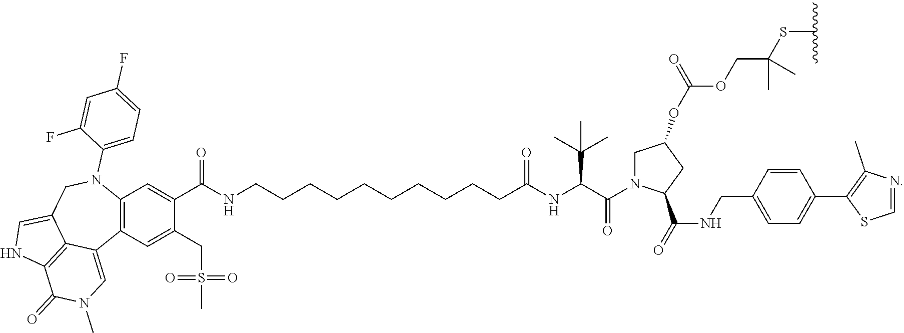 [SMILES: *SC(C)(C)COC(=O)O[C@@H]1C[C@@H](C(=O)NCc2ccc(-c3scnc3C)cc2)N(C(=O)[C@@H](NC(=O)CCCCCCCCCCNC(=O)c2cc3c(cc2CS(C)(=O)=O)-c2cn(C)c(=O)c4[nH]cc(c24)CN3c2ccc(F)cc2F)C(C)(C)C)C1]

141. The heterobifunctional compound of claim 140, comprising a degrader to antibody ratio (DAR) of about 2.0.

142. The heterobifunctional compound of claim 140, wherein the anti-TM4SF1 antibody or the antigen binding fragment thereof comprises an IgG1 Fc region comprising the following mutations: M252Y, S254T, T256E, and N297C, as numbered by the EU index as set forth in Kabat.

143. The heterobifunctional compound of claim 140, wherein the anti-TM4SF1 antibody comprises:
(a) a heavy chain comprising a CDR3 domain comprising an amino acid sequence that has at least 75% identity to a sequence selected from the group consisting of SEQ ID NO: 8, 20, 32, 44, 56, 68, 80, 96, 118, 119, 120, or 121; a CDR2 domain comprising an amino acid sequence that has at least 75% identity to a sequence selected from the group consisting of SEQ ID NO: 7, 19, 31, 43, 55, 67, 79, 95, 116, or 117; and a CDR1 domain comprising an amino acid sequence that has at least 75% identity to a sequence selected from the group consisting of SEQ ID NO: 6, 18, 30, 42, 54, 66, 78, 94, or 115; and
(b) a light chain comprising a CDR3 domain comprising an amino acid sequence that has at least 75% identity to a sequence selected from the group consisting of SEQ ID NO: 14, 26, 38, 50, 62, 74, 86, 110, 111, or 129; a CDR2 domain comprising an amino acid sequence that has at least 75% identity to a sequence selected from the group consisting of SEQ ID NO: 13, 25, 37, 49, 61, 73, 85, 109, or 128; and a CDR1 domain comprising an amino acid sequence that has at least 75% identity to a sequence selected from the group consisting of SEQ ID NO: 12, 24, 36, 48, 60, 72, 84, 107, 108, 124, 125, 126, or 127.

144. A method of treating cancer in a subject, the method comprising administering a heterobifunctional compound according to claim 140.

145. The method of claim 140, further comprising administering the heterobifunctional compound in combination with an immunomodulatory agent.


SEQUENCE LISTING (164 sequences)

SEQ ID NO: 1 - protein, 122 aa, synthetic construct
  REGION 1..122: note = Description of Artificial Sequence: Syntheticpolypeptide
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVS

SEQ ID NO: 2 - protein, 112 aa, synthetic construct
  REGION 1..112: note = Description of Artificial Sequence: Syntheticpolypeptide
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 3 - protein, 123 aa, synthetic construct
  REGION 1..123: note = Description of Artificial Sequence: Syntheticpolypeptide
QIQLVQSGPELKKPGETVKISCKASGYSFRDYGMNWVKQAPGRTFKWMGWINTYTGAPVYAADFKGRFAFSLDTSASAAFLQINNLKNEDTATYFCARWVSYGNNRNWFFDFWGAGTTVTVSS

SEQ ID NO: 4 - DNA, 369 bp, synthetic construct
  misc_feature 1..369: note = Description of Artificial Sequence: Syntheticpolynucleotide
  source 1..369: mol_type = other DNA
cagatccagttggtgcagtctggacctgagctgaagaagcctggagagacagtcaagatctcctgcaaggcttctgggtattccttcagagactatggaatgaactgggtgaagcaggctccaggaaggacttttaagtggatgggctggataaacacctacactggagcgccagtatatgctgctgacttcaagggacggtttgccttctctttggacacctctgccagcgctgcctttttgcagatcaacaacctcaaaaatgaagacacggctacatatttctgtgcaagatgggtctcctacggtaataaccgcaactggttcttcgatttttggggcgcagggaccacggtcaccgtctcctca

SEQ ID NO: 5 - DNA, 369 bp, synthetic construct
  misc_feature 1..369: note = Description of Artificial Sequence: Syntheticpolynucleotide
  source 1..369: mol_type = other DNA
caaattcagttggttcaatccggccctgagctcaagaagcctggagagacagtgaagataagttgtaaggctagtggctattcatttcgagattatgggatgaattgggtcaagcaggccccagggcggaccttcaaatggatggggtggatcaatacttacactggcgcaccagtatatgcagctgattttaagggtcgctttgcattttcacttgatacttcagccagtgccgcttttttgcaaatcaacaatctcaaaaatgaagacactgctacatatttctgcgccaggtgggtgagctatggcaataacagaaattggttctttgacttttggggcgcaggcaccaccgtcactgtctcatca

SEQ ID NO: 6 - protein, 10 aa, synthetic construct
  REGION 1..10: note = Description of Artificial Sequence: Syntheticpeptide
GYSFRDYGMN

SEQ ID NO: 7 - protein, 17 aa, synthetic construct
  REGION 1..17: note = Description of Artificial Sequence: Syntheticpeptide
WINTYTGAPVYAADFKG

SEQ ID NO: 8 - protein, 14 aa, synthetic construct
  REGION 1..14: note = Description of Artificial Sequence: Syntheticpeptide
WVSYGNNRNWFFDF

SEQ ID NO: 9 - protein, 112 aa, synthetic construct
  REGION 1..112: note = Description of Artificial Sequence: Syntheticpolypeptide
DVLMTQTPLSLPVRLGDQASISCRSSQTLVHSNGNTYLEWYLQKPGQSPKLLIYKVSNRLSGVPDRFSGSGSGTDFTLKISRVETEDLGVYYCFQGSHGPWTFGGGTKLEIK

SEQ ID NO: 10 - DNA, 336 bp, synthetic construct
  misc_feature 1..336: note = Description of Artificial Sequence: Syntheticpolynucleotide
  source 1..336: mol_type = other DNA
gatgttttgatgacccaaactccactctccctgcctgtccgtcttggagatcaggcctccatctcttgtagatctagtcagacccttgtacatagtaatggaaacacctatttagaatggtacctgcagaaaccaggccagtctccaaaactcttgatctacaaagtttccaatcgactttctggggtcccagacaggttcagtggcagtggatcagggacagatttcacactcaagatcagcagagtggagactgaggatctgggagtttattactgctttcaaggttcacatggtccgtggacgttcggtggaggcaccaagctggaaatcaaa

SEQ ID NO: 11 - DNA, 336 bp, synthetic construct
  misc_feature 1..336: note = Description of Artificial Sequence: Syntheticpolynucleotide
  source 1..336: mol_type = other DNA
gacgtacttatgacacaaactcccttgagcttgccagtacggcttggcgatcaagcttcaatttcatgtcgttcttctcaaacacttgtccactcaaatgggaatacatatttggaatggtatctccaaaagcccggccaatccccaaaattgttgatttacaaggtgtctaatcgactctcaggcgtccccgaccgattctccgggagcgggtccggtacagacttcaccttgaaaatctccagggtagaaactgaagacctcggagtctactattgtttccaggggtcacacggcccctggacatttggaggaggaactaagctcgagatcaaa

SEQ ID NO: 12 - protein, 16 aa, synthetic construct
  REGION 1..16: note = Description of Artificial Sequence: Syntheticpeptide
RSSQTLVHSNGNTYLE

SEQ ID NO: 13 - protein, 7 aa, synthetic construct
  REGION 1..7: note = Description of Artificial Sequence: Syntheticpeptide
KVSNRLS

SEQ ID NO: 14 - protein, 9 aa, synthetic construct
  REGION 1..9: note = Description of Artificial Sequence: Syntheticpeptide
FQGSHGPWT

SEQ ID NO: 15 - protein, 118 aa, synthetic construct
  REGION 1..118: note = Description of Artificial Sequence: Syntheticpolypeptide
EVQLQQSGPELVKPGASVKISCKTSGYTFTDYTMHWVRQSHGKSLEWIGSFNPNNGGLTNYNQKFKGKATLTVDKSSSTVYMDLRSLTSEDSAVYYCTRIRATGFDSWGQGTTLTVSS

SEQ ID NO: 16 - DNA, 354 bp, synthetic construct
  misc_feature 1..354: note = Description of Artificial Sequence: Syntheticpolynucleotide
  source 1..354: mol_type = other DNA
gaggtccagctgcaacagtctggacctgagctggtgaagcctggggcttcagtgaagatatcctgcaagacttctggatacacattcactgattacaccatgcactgggtgaggcagagccatggaaagagccttgagtggattggaagttttaatcctaacaatggtggtcttactaactacaaccagaagttcaagggcaaggccacattgactgtggacaagtcttccagcacagtgtacatggacctccgcagcctgacatctgaggattctgcagtctattactgtacaagaatccgggctacgggctttgactcctggggccagggcaccactctcacagtctcctca

SEQ ID NO: 17 - DNA, 354 bp, synthetic construct
  misc_feature 1..354: note = Description of Artificial Sequence: Syntheticpolynucleotide
  source 1..354: mol_type = other DNA
gaggtacaactgcaacagagtggacctgaacttgtcaaacctggagcaagtgtgaagattagctgtaaaaccagtggctacacatttaccgattatactatgcactgggtaagacagagccacggaaaatcactggagtggattggtagtttcaatcctaacaacggaggattgacaaattacaaccagaagttcaaagggaaagccaccttgacagttgataagtcctcaagtaccgtgtatatggatctgcgttctctcacaagtgaagatagcgcagtttactactgtacccgcatccgagccaccgggttcgattcatggggtcaggggacaacactgactgtttcttct

SEQ ID NO: 18 - protein, 10 aa, synthetic construct
  REGION 1..10: note = Description of Artificial Sequence: Syntheticpeptide
GYTFTDYTMH

SEQ ID NO: 19 - protein, 18 aa, synthetic construct
  REGION 1..18: note = Description of Artificial Sequence: Syntheticpeptide
SFNPNNGGLTNYNQKFKG

SEQ ID NO: 20 - protein, 8 aa, synthetic construct
  REGION 1..8: note = Description of Artificial Sequence: Syntheticpeptide
IRATGFDS

SEQ ID NO: 21 - protein, 113 aa, synthetic construct
  REGION 1..113: note = Description of Artificial Sequence: Syntheticpolypeptide
DIVMSQSPSSLAVSAGEKVTMSCKSSQSLLNSRTRKNYLAWYQQKPGQSPKLLIYWASTRESGVPDRFTGSGSGTDFTLTISNVQAEDLTVYYCKQSYNPPWTFGGGTKLEIK

SEQ ID NO: 22 - DNA, 339 bp, synthetic construct
  misc_feature 1..339: note = Description of Artificial Sequence: Syntheticpolynucleotide
  source 1..339: mol_type = other DNA
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcaatgtgcaggctgaagacctgacagtttattactgcaagcaatcttataatcctccgtggacgttcggtggaggcaccaagctggaaatcaaa

SEQ ID NO: 23 - DNA, 339 bp, synthetic construct
  misc_feature 1..339: note = Description of Artificial Sequence: Syntheticpolynucleotide
  source 1..339: mol_type = other DNA
gacatagttatgtcccagtctccatccagcttggctgtcagcgccggagagaaagtgactatgagttgtaaatcttcccagtccctgcttaactcacgtactcggaagaattatcttgcctggtatcaacaaaagccaggtcaaagtcctaagctccttatttactgggcctcaacacgggagtcaggtgtccccgatcgcttcacaggtagtgggagtggtactgacttcactctcaccatttcaaatgtccaagcagaagacttgactgtgtattactgtaagcagagttacaaccctccttggacctttggtggggggaccaaactggagatcaag

SEQ ID NO: 24 - protein, 17 aa, synthetic construct
  REGION 1..17: note = Description of Artificial Sequence: Syntheticpeptide
KSSQSLLNSRTRKNYLA

SEQ ID NO: 25 - protein, 7 aa, synthetic construct
  REGION 1..7: note = Description of Artificial Sequence: Syntheticpeptide
WASTRES

SEQ ID NO: 26 - protein, 9 aa, synthetic construct
  REGION 1..9: note = Description of Artificial Sequence: Syntheticpeptide
KQSYNPPWT

SEQ ID NO: 27 - protein, 116 aa, synthetic construct
  REGION 1..116: note = Description of Artificial Sequence: Syntheticpolypeptide
EVQVQQSGPELVKPGASVKMSCKASGYTFTSYVMHWVKQKPGQGLEWIGYINPNNDNINYNEKFKGKASLTSDKSSNTVYMELSSLTSEDSAVYYCAGYGNSGANWGQGTLVTVSA

SEQ ID NO: 28 - DNA, 348 bp, synthetic construct
  misc_feature 1..348: note = Description of Artificial Sequence: Syntheticpolynucleotide
  source 1..348: mol_type = other DNA
gaggtccaggtacagcagtctggacctgaactggtaaagcctggggcttcagtgaagatgtcctgtaaggcttctggatacacattcactagctatgtcatgcactgggtgaagcagaagcctgggcagggccttgagtggattggatatattaatcctaacaatgataatattaactacaatgagaagttcaaaggcaaggcctcactgacttcagacaaatcctccaacacagtctacatggagctcagcagcctgacctctgaggactctgcggtctattactgtgcaggctatggtaactccggagctaactggggccaagggactctggtcactgtctctgca

SEQ ID NO: 29 - DNA, 348 bp, synthetic construct
  misc_feature 1..348: note = Description of Artificial Sequence: Syntheticpolynucleotide
  source 1..348: mol_type = other DNA
gaagttcaagttcagcaaagcgggcctgagcttgtcaagccaggcgcatcagtcaaaatgagctgtaaggcttccgggtacaccttcaccagttatgtcatgcattgggtaaaacaaaagccaggacagggactcgagtggataggatacattaacccaaataacgacaacattaactacaacgagaaattcaagggcaaagcatcattgacttccgataaatcctctaacaccgtgtacatggagctgagttcattgaccagcgaggattctgccgtgtactactgtgcaggttatggcaactctggtgctaactgggggcaggggactctggtcacagtcagcgca

SEQ ID NO: 30 - protein, 10 aa, synthetic construct
  REGION 1..10: note = Description of Artificial Sequence: Syntheticpeptide
GYTFTSYVMH

SEQ ID NO: 31 - protein, 17 aa, synthetic construct
  REGION 1..17: note = Description of Artificial Sequence: Syntheticpeptide
YINPNNDNINYNEKFKG

SEQ ID NO: 32 - protein, 7 aa, synthetic construct
  REGION 1..7: note = Description of Artificial Sequence: Syntheticpeptide
YGNSGAN

SEQ ID NO: 33 - protein, 107 aa, synthetic construct
  REGION 1..107: note = Description of Artificial Sequence: Syntheticpolypeptide
DIQMTQSPASLSASVGETVTITCRTSKNIFNFLAWYHQKQGRSPRLLVSHTKTLAAGVPSRFSGSGSGTQFSLKINSLQPEDFGIYYCQHHYGTPWTFGGGTKLEIK

SEQ ID NO: 34 - DNA, 321 bp, synthetic construct
  misc_feature 1..321: note = Description of Artificial Sequence: Syntheticpolynucleotide
  source 1..321: mol_type = other DNA
gacatccagatgactcagtctccagcctccctatctgcatctgtgggagaaactgtcaccatcacatgtcgaacaagtaaaaatattttcaattttttagcatggtatcaccagaaacagggaagatctcctcgactcctggtctctcatacaaaaaccttagcagcaggtgtgccatcaaggttcagtggcagtggctcaggcacacagttttctctgaagatcaacagcctgcagcctgaagattttgggatttattactgtcaacatcattatggtactccgtggacgttcggtggaggcaccaaactggaaatcaaa

SEQ ID NO: 35 - DNA, 321 bp, synthetic construct
  misc_feature 1..321: note = Description of Artificial Sequence: Syntheticpolynucleotide
  source 1..321: mol_type = other DNA
gacattcagatgacccagtcaccagcatctttgagcgcatccgttggggagactgtgacaatcacatgccgaaccagtaagaacatcttcaacttcctcgcatggtaccatcaaaagcagggcaggtctcccagactgcttgtctctcacaccaagacactggcagcaggcgtccccagccggtttagtggtagtggatctggcacacagtttagtttgaaaatcaattccctgcaacccgaagacttcggcatatactattgccagcaccactatgggacaccttggactttcggaggtggtactaaacttgagattaaa

SEQ ID NO: 36 - protein, 11 aa, synthetic construct
  REGION 1..11: note = Description of Artificial Sequence: Syntheticpeptide
RTSKNIFNFLA

SEQ ID NO: 37 - protein, 7 aa, synthetic construct
  REGION 1..7: note = Description of Artificial Sequence: Syntheticpeptide
HTKTLAA

SEQ ID NO: 38 - protein, 9 aa, synthetic construct
  REGION 1..9: note = Description of Artificial Sequence: Syntheticpeptide
QHHYGTPWT

SEQ ID NO: 39 - protein, 120 aa, synthetic construct
  REGION 1..120: note = Description of Artificial Sequence: Syntheticpolypeptide
QIQLVQSGPELKKPGETVKISCKASGYTFTNYGVKWVKQAPGKDLKWMGWINTYTGNPIYAADFKGRFAFSLETSASTAFLQINNLKNEDTATYFCVRFQYGDYRYFDVWGAGTTVTVSS

SEQ ID NO: 40 - DNA, 360 bp, synthetic construct
  misc_feature 1..360: note = Description of Artificial Sequence: Syntheticpolynucleotide
  source 1..360: mol_type = other DNA
cagatccagttggtgcagtctggacctgagctgaagaagcctggagagacagtcaagatctcctgcaaggcttctgggtataccttcacaaactatggagtgaagtgggtgaagcaggctccaggaaaggatttaaagtggatgggctggataaacacctacactggaaatccaatttatgctgctgacttcaagggacggtttgccttctctttggagacctctgccagcactgcctttttgcagatcaacaacctcaaaaatgaggacacggctacatatttctgtgtaagattccaatatggcgattaccggtacttcgatgtctggggcgcagggaccacggtcaccgtctcctca

SEQ ID NO: 41 - DNA, 360 bp, synthetic construct
  misc_feature 1..360: note = Description of Artificial Sequence: Syntheticpolynucleotide
  source 1..360: mol_type = other DNA
caaatccaacttgtccagagcggtcccgagttgaagaagcctggcgaaaccgtgaaaatctcatgcaaggccagtggatatacatttacaaactatggcgtcaagtgggtgaaacaagccccaggtaaagacttgaaatggatgggatggatcaacacatacacagggaatcctatctatgcagccgactttaaaggcagatttgccttcagtttggagacatctgcctccaccgctttcctgcaaataaataacctgaaaaatgaagataccgctacatacttctgtgtacggttccagtacggagattaccgctatttcgatgtgtggggcgcaggtaccacagtaaccgtctcctca

SEQ ID NO: 42 - protein, 10 aa, synthetic construct
  REGION 1..10: note = Description of Artificial Sequence: Syntheticpeptide
GYTFTNYGVK

SEQ ID NO: 43 - protein, 17 aa, synthetic construct
  REGION 1..17: note = Description of Artificial Sequence: Syntheticpeptide
WINTYTGNPIYAADFKG

SEQ ID NO: 44 - protein, 11 aa, synthetic construct
  REGION 1..11: note = Description of Artificial Sequence: Syntheticpeptide
FQYGDYRYFDV

SEQ ID NO: 45 - protein, 106 aa, synthetic construct
  REGION 1..106: note = Description of Artificial Sequence: Syntheticpolypeptide
QIILSQSPAILSASPGEKVTMTCRANSGISFINWYQQKPGSSPKPWIYGTANLASGVPARFGGSGSGTSYSLTISRVEAEDAATYYCQQWSSNPLTFGAGTKLELR

SEQ ID NO: 46 - DNA, 318 bp, synthetic construct
  misc_feature 1..318: note = Description of Artificial Sequence: Syntheticpolynucleotide
  source 1..318: mol_type = other DNA
caaattattctctcccagtctccagcaatcctgtctgcatctccaggggagaaggtcacgatgacttgcagggccaactcaggtattagtttcatcaactggtaccagcagaagccaggatcctcccccaaaccctggatttatggcacagccaacctggcttctggagtccctgctcgcttcggtggcagtgggtctgggacttcttactctctcacaatcagcagagtggaggctgaagacgctgccacttattactgccagcagtggagtagtaacccgctcacgttcggtgctgggaccaagctggagttgaga

SEQ ID NO: 47 - DNA, 318 bp, synthetic construct
  misc_feature 1..318: note = Description of Artificial Sequence: Syntheticpolynucleotide
  source 1..318: mol_type = other DNA
caaataattctgtcacagtcccccgctatacttagtgcttcaccaggagaaaaagtgaccatgacttgtagagctaattctggcatatcattcatcaactggtatcaacaaaagccaggttcctcccccaagccatggatttacgggaccgccaaccttgcttctggggtacccgctcgtttcggcggatcaggttcaggaacttcctatagcctcactatcagtcgggttgaagctgaggatgccgctacatattactgccagcaatggtctagtaatccacttacctttggagctggcaccaaattggaacttcgt

SEQ ID NO: 48 - protein, 10 aa, synthetic construct
  REGION 1..10: note = Description of Artificial Sequence: Syntheticpeptide
RANSGISFIN

SEQ ID NO: 49 - protein, 7 aa, synthetic construct
  REGION 1..7: note = Description of Artificial Sequence: Syntheticpeptide
GTANLAS

SEQ ID NO: 50 - protein, 9 aa, synthetic construct
  REGION 1..9: note = Description of Artificial Sequence: Syntheticpeptide
QQWSSNPLT

SEQ ID NO: 51 - protein, 116 aa, synthetic construct
  REGION 1..116: note = Description of Artificial Sequence: Syntheticpolypeptide
EVQLQQSGPELVKPGASVKLSCKASGYTVTSYVMHWVKQKPGQGLEWIGYINPYSDVTNCNEKFKGKATLTSDKTSSTAYMELSSLTSEDSAVYYCSSYGGGFAYWGQGTLVTVSA

SEQ ID NO: 52 - DNA, 348 bp, synthetic construct
  misc_feature 1..348: note = Description of Artificial Sequence: Syntheticpolynucleotide
  source 1..348: mol_type = other DNA
gaggtccagctgcagcagtctggacctgagctggtaaagcctggggcttcagtgaagctgtcctgcaaggcttctggatacacagtcactagctatgttatgcactgggtgaagcagaagcctgggcagggccttgagtggattggatatattaatccttacagtgatgttactaactgcaatgagaagttcaaaggcaaggccacactgacttcagacaaaacctccagcacagcctacatggagctcagcagcctgacctctgaggactctgcggtctattactgttcctcctacggtggggggtttgcttactggggccaagggactctggtcactgtctctgca

SEQ ID NO: 53 - DNA, 348 bp, synthetic construct
  misc_feature 1..348: note = Description of Artificial Sequence: Syntheticpolynucleotide
  source 1..348: mol_type = other DNA
gaagtccagcttcagcaatccggcccagaactggtaaaaccaggcgcaagtgttaagttgagttgcaaagccagtggttataccgttacttcatacgtcatgcattgggtaaaacaaaagcccggccaagggcttgaatggatcggctacatcaacccttactctgacgtcaccaactgcaacgagaaattcaaagggaaagccacattgacctctgacaagacaagcagtaccgcctacatggagctttctagtttgacttctgaagactctgctgtctactactgtagcagctacggcggcggctttgcttactggggccagggtacattggtgactgtgagtgca

SEQ ID NO: 54 - protein, 10 aa, synthetic construct
  REGION 1..10: note = Description of Artificial Sequence: Syntheticpeptide
GYTVTSYVMH

SEQ ID NO: 55 - protein, 17 aa, synthetic construct
  REGION 1..17: note = Description of Artificial Sequence: Syntheticpeptide
YINPYSDVTNCNEKFKG

SEQ ID NO: 56 - protein, 7 aa, synthetic construct
  REGION 1..7: note = Description of Artificial Sequence: Syntheticpeptide
YGGGFAY

SEQ ID NO: 57 - protein, 107 aa, synthetic construct
  REGION 1..107: note = Description of Artificial Sequence: Syntheticpolypeptide
DIQMTQSPASLSASVGEPVTITCRASKNIYTYLAWYHQKQGKSPQFLVYNARTLAGGVPSRLSGSGSVTQFSLNINTLHREDLGTYFCQHHYDTPYTFGGGTNLEIK

SEQ ID NO: 58 - DNA, 321 bp, synthetic construct
  misc_feature 1..321: note = Description of Artificial Sequence: Syntheticpolynucleotide
  source 1..321: mol_type = other DNA
gacatccagatgactcagtctccagcctccctatctgcatctgtgggagaacctgtcaccatcacatgtcgagcaagtaagaatatttacacatatttagcatggtatcaccagaaacagggaaaatctcctcagttcctggtctataatgcaagaaccttagcaggaggtgtgccatcaaggctcagtggcagtggatcagtcacgcagttttctctaaacatcaacaccttgcatcgagaagatttagggacttacttctgtcaacatcattatgatactccgtacacgttcggaggggggaccaacctggaaataaaa

SEQ ID NO: 59 - DNA, 321 bp, synthetic construct
  misc_feature 1..321: note = Description of Artificial Sequence: Syntheticpolynucleotide
  source 1..321: mol_type = other DNA
gacatccagatgacacagtcaccagcatccctgtccgcctcagttggggagcctgttaccataacttgtcgggcaagcaaaaacatatacacctatttggcttggtatcaccaaaagcaaggtaagtcacctcagtttcttgtatataatgcccgcacacttgctggcggagtaccctctcgattgtctggatctggcagcgttacccaattcagcctgaacatcaacaccctccatcgggaagatttgggtacctatttctgtcaacatcactacgacaccccatacaccttcggaggcggcacaaatttggaaattaaa

SEQ ID NO: 60 - protein, 11 aa, synthetic construct
  REGION 1..11: note = Description of Artificial Sequence: Syntheticpeptide
RASKNIYTYLA

SEQ ID NO: 61 - protein, 7 aa, synthetic construct
  REGION 1..7: note = Description of Artificial Sequence: Syntheticpeptide
NARTLAG

SEQ ID NO: 62 - protein, 9 aa, synthetic construct
  REGION 1..9: note = Description of Artificial Sequence: Syntheticpeptide
QHHYDTPYT

SEQ ID NO: 63 - protein, 113 aa, synthetic construct
  REGION 1..113: note = Description of Artificial Sequence: Syntheticpolypeptide
EVQLQQSGPELVKPGASVKMSCKASGYTFSSYVMHWVKQKPGQGLEWIGYINPYSDVTNYNEKFKGKATLTSDRSSNTAYMELSSLTSEDSAVYYCARNYFDWGRGTLVTVSA

SEQ ID NO: 64 - DNA, 339 bp, synthetic construct
  misc_feature 1..339: note = Description of Artificial Sequence: Syntheticpolynucleotide
  source 1..339: mol_type = other DNA
gaggtccagctgcagcagtctggacctgagctggtaaagcctggggcttcagtgaagatgtcctgcaaggcttctggatacacattctctagctatgttatgcactgggtgaagcagaagcctgggcagggccttgagtggattggatatattaatccttacagtgatgtcactaactacaatgagaagttcaaaggcaaggccacactgacttcagacagatcctccaacacagcctacatggaactcagcagcctgacctctgaggactctgcggtctattactgtgcaagaaattacttcgactggggccgagggactctggtcacagtctctgca

SEQ ID NO: 65 - DNA, 339 bp, synthetic construct
  misc_feature 1..339: note = Description of Artificial Sequence: Syntheticpolynucleotide
  source 1..339: mol_type = other DNA
gaggtacagcttcagcagagtggtccagaactcgtcaagcctggggcaagcgttaagatgagttgtaaagcatccggttacacattcagtagctatgttatgcactgggtcaaacagaagcctgggcaggggttggagtggatcggatatataaatccctattcagacgtaactaattataatgaaaagttcaaggggaaagcaaccttgacaagtgaccggtcatctaataccgcatacatggagctgagctcattgacaagtgaggactctgctgtgtattactgtgcccggaactacttcgactggggtaggggcacactggtaactgttagtgca

SEQ ID NO: 66 - protein, 10 aa, synthetic construct
  REGION 1..10: note = Description of Artificial Sequence: Syntheticpeptide
GYTFSSYVMH

SEQ ID NO: 67 - protein, 17 aa, synthetic construct
  REGION 1..17: note = Description of Artificial Sequence: Syntheticpeptide
YINPYSDVTNYNEKFKG

SEQ ID NO: 68 - protein, 4 aa, synthetic construct
  REGION 1..4: note = Description of Artificial Sequence: Syntheticpeptide
NYFD

SEQ ID NO: 69 - protein, 107 aa, synthetic construct
  REGION 1..107: note = Description of Artificial Sequence: Syntheticpolypeptide
DIQMTQSPASLSASVGETVTITCRASKNVYSYLAWFQQKQGKSPQLLVYNAKTLAEGVPSRFSGGGSGTQFSLKINSLQPADFGSYYCQHHYNIPFTFGSGTKLEIK

SEQ ID NO: 70 - DNA, 321 bp, synthetic construct
  misc_feature 1..321: note = Description of Artificial Sequence: Syntheticpolynucleotide
  source 1..321: mol_type = other DNA
gacatccagatgactcagtctccagcctccctatctgcatctgtgggagaaactgtcaccatcacatgtcgagcaagtaaaaatgtttacagttatttagcatggtttcaacagaaacaggggaaatctcctcagctcctggtctataatgctaaaaccttagcagaaggtgtgccatcaaggttcagtggcgggggatcaggcacacagttttctctgaagatcaacagcctgcagcctgcagattttgggagttattactgtcaacatcattataatattccattcacgttcggctcggggacaaagttggaaataaaa

SEQ ID NO: 71 - DNA, 321 bp, synthetic construct
  misc_feature 1..321: note = Description of Artificial Sequence: Syntheticpolynucleotide
  source 1..321: mol_type = other DNA
gacatacaaatgacacaaagtcccgctagtctttcagccagtgttggtgagactgtgacaataacctgtagagctagcaaaaatgtctactcctatctggcttggttccagcagaaacaaggaaagagtcctcagttgctcgtatataatgctaaaactttggcagaaggcgtcccttctcgtttcagtggcggaggaagtgggactcaattctcactgaagatcaatagcctccagcccgccgactttgggagctactattgccaacatcattacaacataccattcacctttggctcaggtactaaactcgaaattaaa

SEQ ID NO: 72 - protein, 11 aa, synthetic construct
  REGION 1..11: note = Description of Artificial Sequence: Syntheticpeptide
RASKNVYSYLA

SEQ ID NO: 73 - protein, 7 aa, synthetic construct
  REGION 1..7: note = Description of Artificial Sequence: Syntheticpeptide
NAKTLAE

SEQ ID NO: 74 - protein, 9 aa, synthetic construct
  REGION 1..9: note = Description of Artificial Sequence: Syntheticpeptide
QHHYNIPFT

SEQ ID NO: 75 - protein, 119 aa, synthetic construct
  REGION 1..119: note = Description of Artificial Sequence: Syntheticpolypeptide
QIQLVQSGPELKKPGETVKISCKASGFTFTNYPMHWVKQAPGKGLKWMGWINTYSGVPTYADDFKGRFAFSLETSASTAYLQINNLKNEDMATYFCARGGYDGSREFAYWGQGTLVTVS

SEQ ID NO: 76 - DNA, 357 bp, synthetic construct
  misc_feature 1..357: note = Description of Artificial Sequence: Syntheticpolynucleotide
  source 1..357: mol_type = other DNA
cagatccagttggtgcagtctggacctgagctgaagaagcctggagagacagtcaagatctcctgcaaggcttctgggtttaccttcacaaactatccaatgcactgggtgaagcaggctccaggaaagggtttaaagtggatgggctggataaacacctactctggagtgccaacatatgcagatgacttcaagggacggtttgccttctctttggaaacctctgccagcactgcatatttgcagatcaacaacctcaaaaatgaggacatggctacatatttctgtgcaagagggggctacgatggtagcagggagtttgcttactggggccaagggactctggtcactgtctct

SEQ ID NO: 77 - DNA, 357 bp, synthetic construct
  misc_feature 1..357: note = Description of Artificial Sequence: Syntheticpolynucleotide
  source 1..357: mol_type = other DNA
cagatacaactcgtccagtcaggtccagagttgaagaaacccggagaaactgtgaagatatcctgtaaagccagcggctttactttcacaaactaccccatgcattgggtgaagcaggcccccggaaaaggactcaaatggatgggatggatcaacacatacagtggggtgcctacttacgcagacgatttcaaaggaaggttcgcatttagcttggaaactagcgcatctacagcatatctccagattaacaatcttaaaaatgaggatatggcaacatacttctgcgctaggggaggttacgatgggagcagggagttcgcttattgggggcaagggactcttgtgactgtaagt

SEQ ID NO: 78 - protein, 10 aa, synthetic construct
  REGION 1..10: note = Description of Artificial Sequence: Syntheticpeptide
GFTFTNYPMH

SEQ ID NO: 79 - protein, 17 aa, synthetic construct
  REGION 1..17: note = Description of Artificial Sequence: Syntheticpeptide
WINTYSGVPTYADDFKG

SEQ ID NO: 80 - protein, 11 aa, synthetic construct
  REGION 1..11: note = Description of Artificial Sequence: Syntheticpeptide
GGYDGSREFAY

SEQ ID NO: 81 - protein, 110 aa, synthetic construct
  REGION 1..110: note = Description of Artificial Sequence: Syntheticpolypeptide
DIVLTQSPASLAASLGQRATTSYRASKSVSTSGYSYMHWNQQKPGQPPRLLIYLVSNLESGVPARFSGSGSGTDFTLNIHPVEEEDAATYYCQHIRELTTFGGGTKLEIK

SEQ ID NO: 82 - DNA, 330 bp, synthetic construct
  misc_feature 1..330: note = Description of Artificial Sequence: Syntheticpolynucleotide
  source 1..330: mol_type = other DNA
gacattgtgctgacacagtctcctgcttccttagctgcatctctggggcagagggccaccacctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttaccacgttcggaggggggaccaagctggaaataaaa

SEQ ID NO: 83 - DNA, 330 bp, synthetic construct
  misc_feature 1..330: note = Description of Artificial Sequence: Syntheticpolynucleotide
  source 1..330: mol_type = other DNA
gacatagtgctcactcagagccctgcatcccttgccgcctccctcggacaacgagctactacaagctaccgggcatcaaagtccgttagcacatcaggatacagctatatgcactggaatcagcaaaagccaggccaaccaccccgtcttctcatctacctcgtaagtaatctggaatcaggcgtgccagcccgattcagtgggtcagggtctgggacagatttcaccctcaacatccatccagtagaggaagaggacgcagcaacatattactgccaacacattagagaacttaccactttcggaggaggaactaaattggagatcaaa

SEQ ID NO: 84 - protein, 15 aa, synthetic construct
  REGION 1..15: note = Description of Artificial Sequence: Syntheticpeptide
RASKSVSTSGYSYMH

SEQ ID NO: 85 - protein, 7 aa, synthetic construct
  REGION 1..7: note = Description of Artificial Sequence: Syntheticpeptide
LVSNLES

SEQ ID NO: 86 - protein, 7 aa, synthetic construct
  REGION 1..7: note = Description of Artificial Sequence: Syntheticpeptide
QHIRELT

SEQ ID NO: 87 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 107 aa, synthetic construct [sequence omitted]

SEQ ID NO: 90 - protein, 450 aa, synthetic construct
  REGION 1..450: note = Description of Artificial Sequence: Syntheticpolypeptide
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1362 bp, synthetic construct
  misc_feature 1..1362: note = Description of Artificial Sequence: Syntheticpolynucleotide
  source 1..1362: mol_type = other DNA
tctaccggacaggtgcagttggttcagtctggcgccgaagtgaagaaacctggcgcttctgtgaaggtgtcctgcaaggcctctggctacacctttaccaactacggcgtgaaatgggtccgacaggctcctggacaggatctggaatggatgggctggatcaacacctacaccggcaatcctatctacgccgccgacttcaagggcagagtgaccatgaccaccgacacctctacctccaccgccttcatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 92 - protein, 450 aa, synthetic construct
  REGION 1..450: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1353 bp, synthetic construct
  misc_feature 1..1353: note = Description of Artificial Sequence: Syntheticpolynucleotide
  source 1..1353: mol_type = other DNA
gaagtgcagttggtgcagtctggcgccgaagtgaagaaacctggcgcttctgtgaaggtgtcctgcaaggcctctggctacacctttaccaactacggcgtgaaatgggtccgacaggctcctggacaaggcctggaatggatgggctggatcaacacctacaccggcaatcctatctacgccgccgacttcaagggcagagtgaccatgaccaccgacacctctacctccaccgcctacatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaattctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 94 - protein, 10 aa, synthetic construct
  REGION 1..10: note = Description of Artificial Sequence: Syntheticpeptide
GYTFTNYGVK

SEQ ID NO: 95 - protein, 16 aa, synthetic construct
  REGION 1..16: note = Description of Artificial Sequence: Syntheticpeptide
WINTYTGNPIYAADFK

SEQ ID NO: 96 - protein, 11 aa, synthetic construct
  REGION 1..11: note = Description of Artificial Sequence: Syntheticpeptide
FQYGDYRYFDV

SEQ ID NO: 97 - protein, 213 aa, synthetic construct
  REGION 1..213: note = Description of Artificial Sequence: Syntheticpolypeptide
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - DNA, 714 bp, synthetic construct
  misc_feature 1..714: note = Description of Artificial Sequence: Syntheticpolynucleotide
  source 1..714: mol_type = other DNA
aagcttgccaccatggaaaccgacacactgctgctgtgggtgctgttgttgtgggtgccaggatctaccggagagatcatcctgacacagagccccgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagatttggcggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 99 - protein, 213 aa, synthetic construct
  REGION 1..213: note = Description of Artificial Sequence: Syntheticpolypeptide
EIVLTQSPATLSLSPGERATLSCRANSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 651 bp, synthetic construct
  misc_feature 1..651: note = Description of Artificial Sequence: Syntheticpolynucleotide
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 101 - protein, 213 aa, synthetic construct
  REGION 1..213: note = Description of Artificial Sequence: Syntheticpolypeptide
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 651 bp, synthetic construct
  misc_feature 1..651: note = Description of Artificial Sequence: Syntheticpolynucleotide
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 103 - protein, 213 aa, synthetic construct
  REGION 1..213: note = Description of Artificial Sequence: Syntheticpolypeptide
EIVLTQSPATLSLSPGERATLSCRANSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 651 bp, synthetic construct
  misc_feature 1..651: note = Description of Artificial Sequence: Syntheticpolynucleotide
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 105 - protein, 213 aa, synthetic construct
  REGION 1..213: note = Description of Artificial Sequence: Syntheticpolypeptide
EIVLTQSPATLSLSPGERATLSCRAQSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - DNA, 651 bp, synthetic construct
  misc_feature 1..651: note = Description of Artificial Sequence: Syntheticpolynucleotide
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 107 - protein, 10 aa, synthetic construct
  REGION 1..10: note = Description of Artificial Sequence: Syntheticpeptide
RANSGISFIN

SEQ ID NO: 108 - protein, 10 aa, synthetic construct
  REGION 1..10: note = Description of Artificial Sequence: Syntheticpeptide
RAQSGISFIN

SEQ ID NO: 109 - protein, 7 aa, synthetic construct
  REGION 1..7: note = Description of Artificial Sequence: Syntheticpeptide
GTANLAS

SEQ ID NO: 110 - protein, 9 aa, synthetic construct
  REGION 1..9: note = Description of Artificial Sequence: Syntheticpeptide
QQWSSNPLT

SEQ ID NO: 111 - protein, 9 aa, synthetic construct
  REGION 1..9: note = Description of Artificial Sequence: Syntheticpeptide
QQYSSNPLT

SEQ ID NO: 112 - protein, 453 aa, synthetic construct
  REGION 1..453: note = Description of Artificial Sequence: Syntheticpolypeptide
EVQLVESGGGLVKPGGSLRLSCAASGFTFSSFAMSWVRQAPGKGLEWVSTISSGSIYIYYTDGVKGRFTISRDNAKNSLYLQMNSLRAEDTAVYYCARRGIYYGYDGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 1362 bp, synthetic construct
  misc_feature 1..1362: note = Description of Artificial Sequence: Syntheticpolynucleotide
  source 1..1362: mol_type = other DNA
gaggtgcagctggttgaatctggcggaggacttgtgaagcctggcggctctctgagactgtcttgtgccgcctctggcttcaccttctccagctttgccatgtcctgggtccgacaggctcctggcaaaggactggaatgggtgtccaccatctcctccggctccatctacatctactacaccgacggcgtgaagggcagattcaccatcagcagagacaacgccaagaactccctgtacctgcagatgaacagcctgagagccgaggacaccgccgtgtactattgtgccagacggggcatctactatggctacgacggctacgctatggactattggggacagggcacactggtcaccgtgtcctctgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 114 - protein, 453 aa, synthetic construct
  REGION 1..453: note = Description of Artificial Sequence: Syntheticpolypeptide
EVQLVESGGGLVKPGGSLRLSCAASGFTFSSFAMSWVRQAPGKGLEWVSTISSGSIYIYYTDSVKGRFTISRDNAKNSLYLQMNSLRAEDTAVYYCARRGIYYGYEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 10 aa, synthetic construct
  REGION 1..10: note = Description of Artificial Sequence: Syntheticpeptide
GFTFSSFAMS

SEQ ID NO: 116 - protein, 17 aa, synthetic construct
  REGION 1..17: note = Description of Artificial Sequence: Syntheticpeptide
TISSGSIYIYYTDGVKG

SEQ ID NO: 117 - protein, 17 aa, synthetic construct
  REGION 1..17: note = Description of Artificial Sequence: Syntheticpeptide
TISSGSIYIYYTDSVKG

SEQ ID NO: 118 - protein, 14 aa, synthetic construct
  REGION 1..14: note = Description of Artificial Sequence: Syntheticpeptide
RGIYYGYDGYAMDY

SEQ ID NO: 119 - protein, 14 aa, synthetic construct
  REGION 1..14: note = Description of Artificial Sequence: Syntheticpeptide
RGIYYGYEGYAMDY

SEQ ID NO: 120 - protein, 14 aa, synthetic construct
  REGION 1..14: note = Description of Artificial Sequence: Syntheticpeptide
RGIYYGYSGYAMDY

SEQ ID NO: 121 - protein, 14 aa, synthetic construct
  REGION 1..14: note = Description of Artificial Sequence: Syntheticpeptide
RGIYYGYAGYAMDY

SEQ ID NO: 122 - protein, 219 aa, synthetic construct
  REGION 1..219: note = Description of Artificial Sequence: Syntheticpolypeptide
AIVLTQSPGTLSLSPGERATLSCRSSQSLVHSNGNTYLHWYMQKPGQAPRVLIYKVSNRFSGIPDRFSGSGSGTDFTLTISRLEPDDFAIYYCSQSTHIPLAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 660 bp, synthetic construct
  misc_feature 1..660: note = Description of Artificial Sequence: Syntheticpolynucleotide
  source 1..660: mol_type = other DNA
gccatcgtgttgacccagtctccaggcacattgtctctgagccctggcgagagagctaccctgtcctgcagatcttctcagtccctggtgcactccaacggcaacacctacctgcactggtacatgcagaagcccggacaggctcccagagtgctgatctacaaggtgtccaaccggttctctggcatccccgacagattttccggctctggctctggcaccgacttcaccctgaccatctctagactggaacccgacgacttcgccatctactactgctcccagtccacacacatccctctggcttttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggcctgtctagccctgtgaccaagtctttcaaccggggcgagtgttga

SEQ ID NO: 124 - protein, 16 aa, synthetic construct
  REGION 1..16: note = Description of Artificial Sequence: Syntheticpeptide
RSSQSLVHSNGNTYLH

SEQ ID NO: 125 - protein, 16 aa, synthetic construct
  REGION 1..16: note = Description of Artificial Sequence: Syntheticpeptide
RSSQSLVHSSGNTYLH

SEQ ID NO: 126 - protein, 16 aa, synthetic construct
  REGION 1..16: note = Description of Artificial Sequence: Syntheticpeptide
RSSQSLVHSTGNTYLH

SEQ ID NO: 127 - protein, 16 aa, synthetic construct
  REGION 1..16: note = Description of Artificial Sequence: Syntheticpeptide
RSSQSLVHSQGNTYLH

SEQ ID NO: 128 - protein, 7 aa, synthetic construct
  REGION 1..7: note = Description of Artificial Sequence: Syntheticpeptide
KVSNRFS

SEQ ID NO: 129 - protein, 9 aa, synthetic construct
  REGION 1..9: note = Description of Artificial Sequence: Syntheticpeptide
SQSTHIPLA

SEQ ID NO: 130 - protein, 120 aa, synthetic construct
  REGION 1..120: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSS

SEQ ID NO: 131 - protein, 106 aa, synthetic construct
  REGION 1..106: note = Description of Artificial Sequence: Syntheticpolypeptide
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIK

SEQ ID NO: 132 - protein, 120 aa, synthetic construct
  REGION 1..120: note = Description of Artificial Sequence: Syntheticpolypeptide
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSS

SEQ ID NO: 133 - protein, 106 aa, synthetic construct
  REGION 1..106: note = Description of Artificial Sequence: Syntheticpolypeptide
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIK

SEQ ID NO: 134 - protein, 202 aa, Homo sapiens
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 135 - protein, 330 aa, synthetic construct; identical to SEQ ID NO: 87
  REGION 1..330: note = Description of Artificial Sequence: Syntheticpolypeptide

SEQ ID NO: 136 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 330 aa, synthetic construct; identical to SEQ ID NO: 136
  REGION 1..330: note = Description of Artificial Sequence: Syntheticpolypeptide

SEQ ID NO: 142 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 330 aa, synthetic construct; identical to SEQ ID NO: 138
  REGION 1..330: note = Description of Artificial Sequence: Syntheticpolypeptide

SEQ ID NO: 144 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVAVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 332 aa, synthetic construct
  REGION 1..332: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKP

SEQ ID NO: 146 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 449 aa, synthetic construct
  REGION 1..449: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKKP

SEQ ID NO: 151 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 153 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 155 - protein, 447 aa, synthetic construct
  REGION 1..447: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 156 - protein, 450 aa, synthetic construct
  REGION 1..450: note = Description of Artificial Sequence: Syntheticpolypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 158 - protein, 420 aa, Homo sapiens
QLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA

SEQ ID NO: 159 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 160 - protein, 479 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

SEQ ID NO: 161 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 162 - protein, 48 aa, Homo sapiens
EGPLCLDSLGQWNYTFASTEGQYLLDTSTWSECTEPKHIVEWNVSLFS

SEQ ID NO: 163 - protein, 12 aa, synthetic construct
  REGION 1..12: note = Description of Artificial Sequence: Syntheticpeptide
REEQYCSTYRVV

SEQ ID NO: 164 - protein, 12 aa, synthetic construct
  REGION 1..12: note = Description of Artificial Sequence: Syntheticpeptide
REEQFCSTYRVV